(19) United States
(12) Patent Application Publication
(10) Pub. No.: US 2026/0355000 A1
(43) Pub. Date: Nov. 12, 2026

(54) REALLY ADVANCED EXTERNAL ANKLE BRACE

(71) Applicant: TayCo Brace, Inc., South Bend, IN (US)

(72) Inventors: Nate LAZENGA, Round Rock, TX (US); Mike BEAN, South Bend, IN (US); Frederick John FERLIC, South Bend, IN (US)

(21) Appl. No.: 19/674,500

(22) Filed: May 12, 2026

Related U.S. Application Data
(60) Provisional application No. 63/804,088, filed on May 12, 2025.
(60) Provisional application No. 63/824,623, filed on Jun. 16, 2025.
(60) Provisional application No. 63/827,528, filed on Jun. 20, 2025.
(60) Provisional application No. 63/829,989, filed on Jun. 25, 2025.

(51) Int. Cl. A61F 5/01

(52) U.S. Cl. CPC A61F 5/0127; A61F 2005/0137

(57) ABSTRACT

An external ankle brace is disposed on the exterior of a shoe having a heel portion, a toe portion, a sole, and oppositely disposed sides. The external ankle brace comprises a rigid heel enclosure having a rear portion, configured to receive the heel of the shoe, and a forward portion having a medial sidewall and a lateral sidewall for collectively and concurrently at least partially encircling the sides of the shoe. A lateral upright extension is present. A medial upright extension is present.

### CROSS-REFERENCE TO RELATED APPLICATIONS

This application claims priority to U.S. Provisional Patent Application No. 63/804,088, filed on May 12, 2025, in the United States Patent and Trademark Office, and U.S. Provisional Patent Application No. 63/824,623, filed on Jun. 16, 2025, in the United States Patent and Trademark Office U.S. Provisional Patent Application No. 63/827,528, filed on Jun. 20, 2025, in the United States Patent and Trademark Office, and U.S. Provisional Patent Application No. 63/829,989 filed on Jun. 25, 2025, in the United States Patent and Trademark Office, the entire contents of these applications being incorporated herein by reference in their entirety.

### TECHNICAL FIELD

The disclosure pertains generally to preventative and rehabilitative equipment and/or support equipment, and more particularly to an ankle brace.

### BACKGROUND

In the world of sports, ankle injuries are among the most common cause of lost playing time in a sporting career, with a typical ankle injury leaving the athlete out of competition for up to a month. Ankle sprains occur when there is a rapid shifting of weight from one direction to another. The force generated from the movement causes the foot to roll either inwards, which is known as inversion rotation; or outwards, which is known as eversion rotation. Both the inversion and eversion motion of the ankle cause the ligaments on the outside of the ankle to stretch or tear depending on the force that was generated during the movement.

Current braces vary from woven fabric that acts as a glove and wraps around the ankle, to rigid plastic uprights that are strapped around the ankle. The woven fabric braces typically are made of a thin fabric that envelope the ankle and are laced together to support the ankle from both inversion and eversion rotation. The main drawback with these types of braces is that the material lacks the resistance to prevent the ankle from rolling under intense forces. Further, fabric braces also have to be worn within the shoe, which causes the shoe to fit tighter or, in some cases, forces the user to move up a shoe size in order to wear the brace. In terms of the rigid uprights braces, these braces are typically much heavier than the fabric braces and also much larger. Fitting a rigid brace into a tight shoe almost never works, which forces the user to move up to the next shoe size to accommodate for the bulkiness of the brace. When the user moves up a shoe size, the shoe is no longer sized correctly for the foot and thus loses a portion of its intended use and purpose. These braces leave the user at risk for further injury because either the brace isn't strong enough to support the ankle or the shoe isn't fitted properly to the foot.

### SUMMARY

In an aspect, an external ankle brace for selectively restricting movement of an ankle in at least one of a first direction and a rotation direction, and selectively permitting movement of the ankle in a second direction is provided. The external ankle brace is disposed on the exterior of a shoe. The shoe has a heel portion, a toe portion longitudinally spaced from the heel portion, a sole, and oppositely disposed sides. The external ankle brace comprises a rigid heel enclosure having a rear portion and a forward portion. The rear portion is configured to receive and at least partially encircle the heel portion of the shoe. The forward portion has a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe. The lateral and medial sidewalls each extend from the rear portion toward a toe of a wearer's foot and each extend beyond a talus of the wearer's foot. A lateral upright extension is selectively perpendicular to the rigid heel enclosure and is pivotally attached to the lateral sidewall. The lateral upright extension includes a lateral reinforcing strut. A medial upright extension is selectively perpendicular to the rigid heel enclosure and is pivotally attached to the medial sidewall. The medial upright extension includes a medial reinforcing strut.

### BRIEF DESCRIPTION OF THE DRAWINGS

For a better understanding, reference may be made to the accompanying drawings, in which:

FIG. 1 is a lateral side view showing a first aspect of the external ankle brace with an athletic shoe.

FIG. 2 is a perspective view of the external ankle brace of FIG. 1 from the medial side.

FIG. 3 is a perspective view showing the underside of the external ankle brace of FIG. 1.

FIG. 4 is a perspective view of the external ankle brace of FIG. 1 from the lateral side.

FIG. 5 is a top view of the external ankle brace of FIG. 1.

FIG. 6 is a rear view of the external ankle brace of FIG. 1.

FIG. 7 is a lateral side view showing a second aspect of the external ankle brace, in a first configuration, with an athletic shoe.

FIG. 8 is a lateral side view showing a second aspect of the external ankle brace, in a second configuration, with an athletic shoe.

FIG. 9 is a perspective rear view of a component of the external braces of FIGS. 7-8.

FIG. 10 is a partial perspective front view of the component of FIG. 9.

FIG. 11 is a partial front view of the component of FIG. 9.

FIG. 12 is a side view of the component of FIG. 9.

FIG. 13 is a bottom view of the component of FIG. 9.

FIGS. 14-20 depict via CAD images details of a different embodiment, a third embodiment, different than the first and second embodiments of FIGS. 1-13 above.

FIGS. 21-37A depict views of some embodiments;

FIGS. 39-56 and 58-103 depict views of additional embodiments;

FIGS. 38 and 57 and 103 depict flowcharts for exemplary methods.

FIGS. 104 and 105 depict flowcharts for exemplary methods.

FIGS. 106-116 depict views for exemplary embodiments.

FIGS. 117-120 depict flowcharts for exemplary methods.

### DETAILED DESCRIPTION

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, the term “and/or” can include any and all combinations of one or more of the associated listed items.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

A “wearer” or “user”, as described herein, is a person who has the external ankle brace on his or her own foot/ankle.

Ankle injuries are among the most common cause of lost playing time in a sporting career and although there are current preventative solutions, those current braces leave the user at risk for further injury because either the brace isn't strong enough to support the ankle or the shoe isn't fitted properly to the foot since “inside the shoe” braces tend to force the user to use a bigger shoe size. In addition, outside the athletic context, ankle braces can be used by the general population while recovering from ankle injury or attempting to prevent reinjury, to wear while going about daily activities.

The present disclosure provides a rigid support and a much faster application time, all without compromising the fit of the shoe. Essentially, the external ankle brace described herein assists anyone recovering from an ankle injury and/or seeking to avoid injury to limit inversion/eversion and rotation, and can also selectively limit plantar flexion/dorsiflexion. This at least partial motion limiting can help an ankle to heal or avoid (re) injury; thus, the patient can return to function and normal daily activities (including athletic activities, as desired) without the need for specialized or temporary-use footwear (e.g., a larger shoe on the braced side). The external ankle brace of the present disclosure may provide superior stability to an internal ankle brace and improved functionality over known solutions, such as a walking boot or internal ankle foot orthoses.

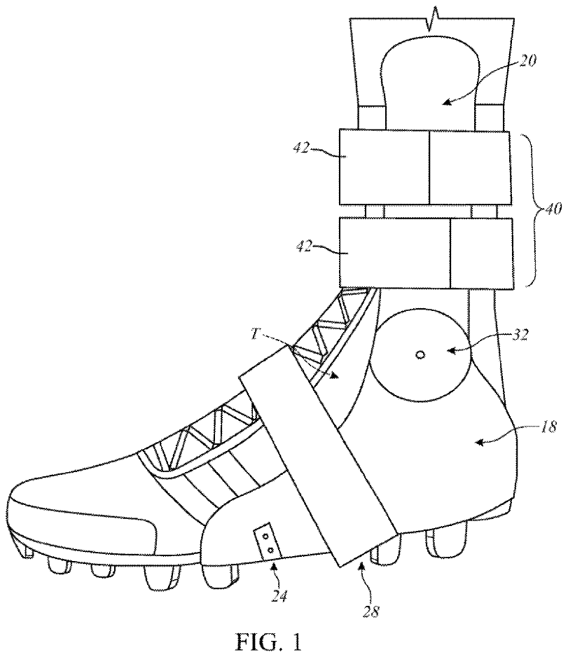

The present disclosure relates to an external ankle brace that is adapted to fit around a shoe to prevent and minimize injury to an ankle. The shoe has a heel portion, a toe portion longitudinally spaced from the heel portion, a sole, and oppositely disposed sides. The term “longitudinal” is used herein to reference a direction oriented along the foot between the heel and toe. The interaction between the external ankle brace and the shoe can be seen in FIG. 1.

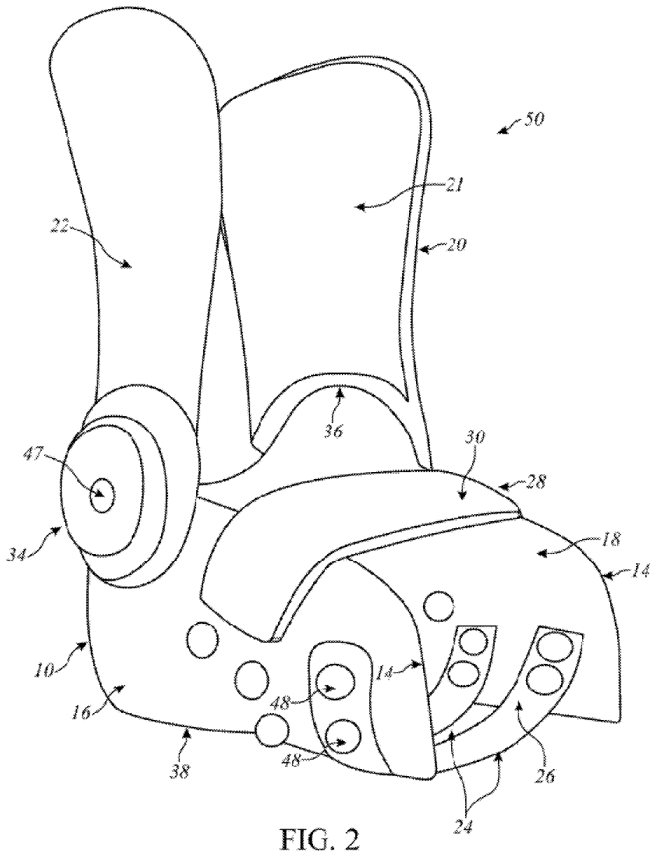

The external ankle brace of the present disclosure is generally indicated at 50 in FIG. 2. The external ankle brace 50 includes a rigid heel enclosure 10, a lateral upright extension 20, a medial upright extension 22, a lower fastening system 24, and an upper fastening system 28.

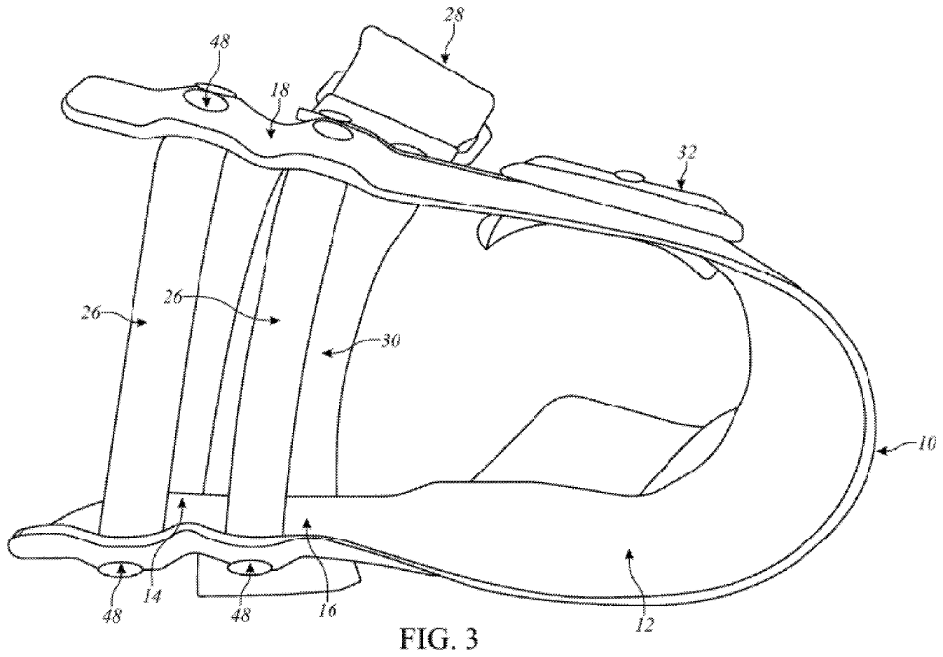

The rigid heel enclosure 10 has a rear portion 12 (FIG. 3), for receiving and at least partially encircling the heel of the shoe, and a forward portion 14, for surrounding, e.g., collectively and concurrently at least partially surrounding, the sides of the shoe. The forward portion 14 at least partially encircles the sides of the shoe concurrent with the rear portion 12 connecting the medial and lateral sidewalls 18 and 16 to collectively at least partially encircle the side, and fully encircle the heel, of the shoe. The heel enclosure 10 may be at least partially made from rigid plastic pieces and/or any other suitable material. The forward portion 14 further includes a medial sidewall 16 and a lateral sidewall 18, which may also be at least partially made from rigid plastic pieces and/or any other suitable material.

As shown in the Figures, it is contemplated that the lateral and/or medial sidewalls 16 and 18 may each extend from the rear portion 12 in a longitudinal direction toward a toe of the wearer's foot and may each extend beyond a talus (shown as “T” in FIG. 1) of the wearer's foot—i.e., may be located lateral/medial to the lace portion of the shoe. (It is recognized that a particular wearer may have nonstandard bone structure to which this “landmark” would not apply—this description presumes a foot structure which is anatomically normal, in which the talus marks the “inflection point” at which the lower leg turns into the top of the foot.) Stated differently, the rigid heel enclosure 10 has a rear portion 12, and a forward portion 14 partially located on both sides of the heel, one side of the forward portion 14 comprising the medial sidewall 16 and another side of the forward portion 14 comprising the lateral sidewall 18. Each of the medial and lateral sidewalls 16 and 18 extends at least a predetermined amount of the longitudinal distance from a pivot point on the rigid heel enclosure 12 forward from heel toward toe. The medial and lateral sidewalls 16 and 18 run beside (and thus at least partially surround) a predetermined percentage of a total longitudinal length of the user's shoe that is in the range of 5-40%, more particularly about 10-35%, and more particularly about 20-30% of the longitudinal distance between the user's ankle and the end of the user's toe. It is contemplated that an external ankle brace 50 will, for most use environments, include medial and lateral sidewalls 16 and 18 that extend beyond the talus T (as opposed to a straight “stirrup” on the sides of the ankle with little/no extension), to accommodate placement of a lower connecting strap 26 that fits under the shoe's arch gap.

The rigid heel enclosure 10 also has an upper end 36 (FIG. 2) for receiving the upright extensions 20 and 22, and a lower end 38 for surrounding the bottom of the shoe. The rigid heel enclosure 10 can be configured as desired, such as by orienting the lower end 38 in a substantially “straight” non-contoured configuration (parallel to, and fitting closely about, the base of the shoe), and/or the upper end 36 including a curved contour as shown in FIG. 2 for mechanical, manufacturing efficiency, aesthetic, and/or any other considerations.

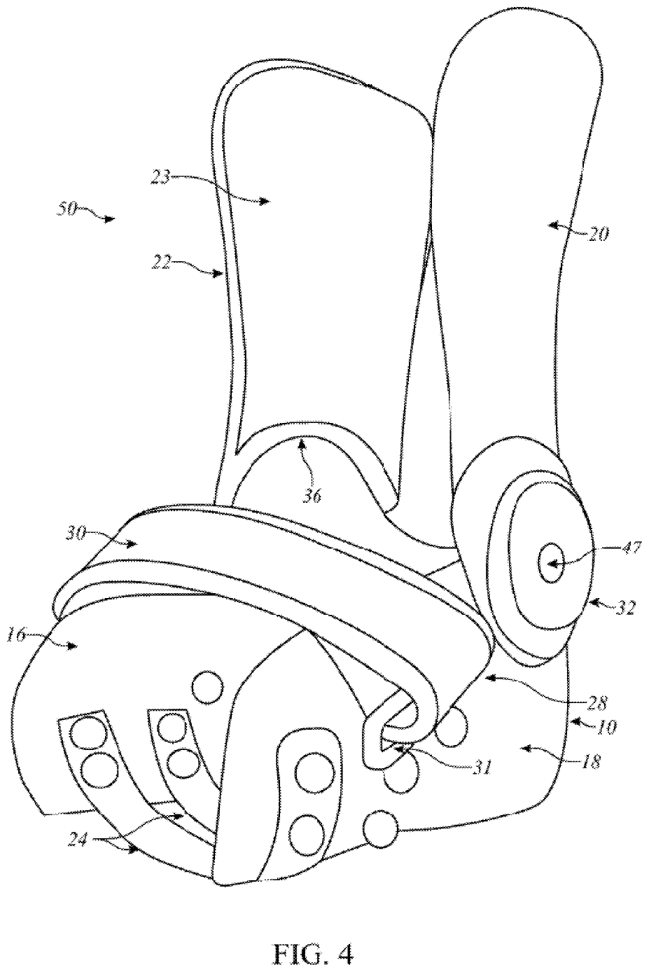

The lateral upright extension 20 is oriented generally selectively perpendicular to at least the lateral sidewall 18 of the rigid heel enclosure 10 and is pivotally attached to the lateral sidewall 18 at the upper end 36 by a lateral ankle joint 32 (FIG. 4). The joint allows the lateral upright extension 20 to rotate during motion giving the external ankle brace a less restrictive feel compared to previous braces—thus, the lateral upright extension 20 is perpendicular to the lateral sidewall 18 during certain portions of the pivoting process, such as when the ankle is in a neutral position, in neither planar flexion nor dorsiflexion.

In other words, the lateral sidewall 18 is both substantially planar in a vertical fashion (up and down) and substantially planar in a longitudinal fashion (back of shoe to front of shoe), including some minor contours to fit the curves of a shoe and/or a user. The lateral upright extension 20 selectively pivots forward and backward to allow for plantar flexion and dorsiflexion while still providing inversion/eversion and rotational support. As desired, the lateral upright extension 20 may be configured for permanent or temporary fixation with respect to the lateral sidewall 18, which maintains perpendicularity therebetween and does not allow for plantar flexion or dorsiflexion. When the fixation screw (or screws) are removed as discussed below to reverse a temporary fixation situation, then the lateral and medial upright extensions 20 and 22 can move forward and backward to allow for plantar flexion and dorsiflexion.

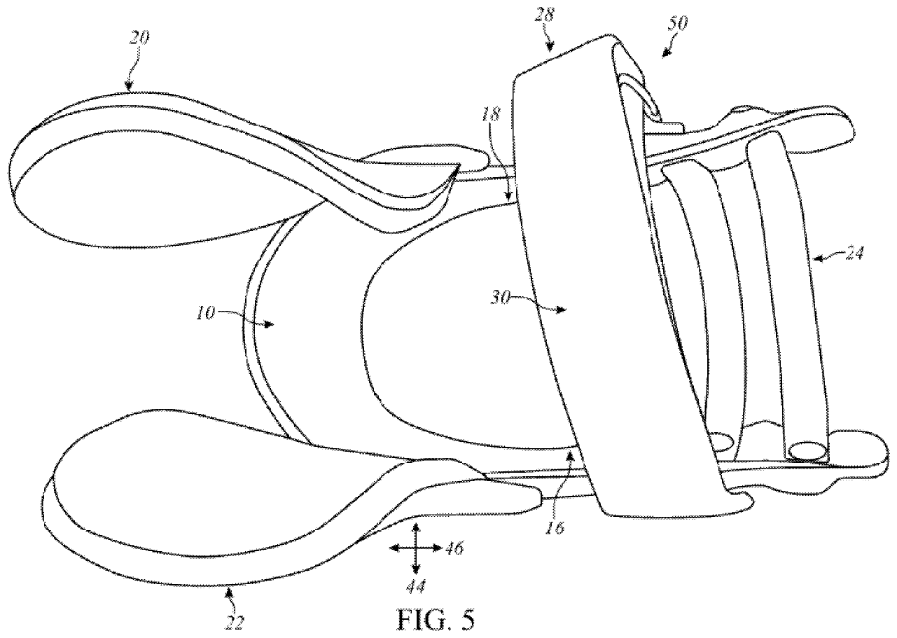

The lateral upright extension 20 may be made from plastic and/or any other suitable material. The lateral ankle joint 32 includes a fastener 47 and allows the lateral upright extension 20 to rotate relative to the lateral sidewall 18. Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the lateral upright extension 20 may have a concave shape for increased comfort for the user. The lateral upright extension 20 can also include foam or other suitable padding on the interior side 21 (FIG. 2) of the lateral upright extension 20 to increase comfort and to allow a better fit for the user.

The medial upright extension 22 is oriented generally selectively perpendicular to at least the medial sidewall 16 of the rigid heel enclosure 10 and is pivotally attached to the medial sidewall 16 at the upper end 36 by a medial ankle joint 34, with the perpendicularity and pivotal properties being similar to those of the lateral upright extension 20. The medial upright extension 22 may be made of rigid plastic and/or any other suitable material. The medial ankle joint 34 has a fastener 47 and allows the medial upright extension 22 to rotate relative to the medial sidewall 16—thus, the medial upright extension 22 is perpendicular to the medial sidewall 16 during certain portions of the pivoting process.

To adjust for anatomical positioning of the ankle, the medial ankle joint 34 may be positioned somewhat closer to the upper end 36 than the position of the lateral ankle joint 32, since the medial malleolus (that bone that protrudes from the inside of the user's ankle) may be slightly higher (more cephalad) than the lateral malleolus (the bone that protrudes from the outside of the ankle), in most anatomically normal wearers. The pivot points between the lateral and medial upright extensions 20 and 22, and their respective lateral and medial sidewalls 18 and 16 on each side of the external ankle brace 50 could be desirably aligned with the medial-higher anatomy of the body, although it is contemplated that versions of the external ankle brace 50 could have equal-medial-lateral or medial-lower pivot points for any reason(s), such as, but not limited to, manufacturing considerations or the anatomy of a particular user.

In addition, the medial ankle joint 34 of the external ankle brace 50 may be offset, as desired from a 180-degree directly opposing position relative to the lateral ankle joint 32. For example, the medial ankle joint 34 may be rotated in the range of about 5-20 degrees, and more particularly about 10-15 degrees, forward (towards the toes) from a 180 degree position relative to the lateral ankle joint 32. Similar to the ankle joint height discussed immediately previously, this is anatomically correct. To clarify, imagine a circle that is parallel to the ground (on the same plane). Instead of having the medial and lateral ankle joints 34 and 32 being 180 degrees apart on that circle, the medial ankle joint 34 could be moved (and rotated to keep facing the center of the circle, if desired), some distance toward the toes along that circle because the medial malleolus is closer to the toes than is the lateral malleolus. It is contemplated that versions of the external ankle brace 50 could instead have 180-degree-spaced or lateral-forward pivot points for any reason(s), such as, but not limited to, manufacturing considerations or the anatomy of a particular user.

Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the medial upright extension 22 may have a concave shape for increased comfort for the user. The medial upright extension 22 can also include foam or other padding on the interior side 23 (FIG. 4) of the medial upright extension to increase comfort and to allow a better fit for the user.

The lower fastening system 24 has at least one lower connecting strap 26 and at least one strap fastener 48 for connecting the lateral sidewall 18 to the medial sidewall 16 (FIG. 2) and extending underneath the sole of the shoe. Although the current embodiment uses a rubber strap, one having ordinary skill in the art would appreciate that plastic, nylon, or any other suitable strap type that is commonly known in the art could be used. Similarly, although the current embodiment uses rivets to fasten the straps to each of the lateral and medial sidewalls 18 and 16 respectively, any other fastener could be used.

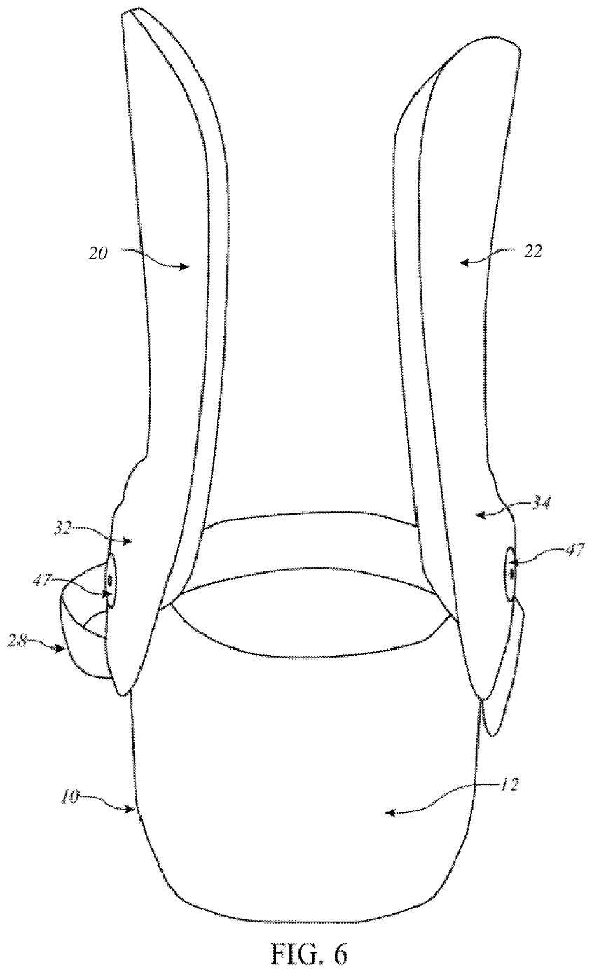

The upper fastening system 28 has at least one upper connecting strap 30 for selectively connecting the lateral sidewall 18 to the medial sidewall 16 while passing over the top of the shoe. The upper fastening system 28 further includes a D-ring 31 which is fixed on the lateral sidewall. A hook and loop fastener type upper connecting strap 30 is fixed to the medial sidewall and is looped through the D-ring 31 to overlaps back itself. This allows for an adjustable fastening system to accommodate various sizes without compromising support. The term “hook and loop fastener” is used herein to reference a type of fastening device such as, but not limited to, VELCRO® brand fasteners, available from Velcro USA Inc. & Velcro Group Corporation of Manchester, New Hampshire. Although the current embodiment uses a hook and loop fastener upper connecting strap 30 to removably connect the sidewalls 16 and 18 and/or adjust the tightness of their mutual connection across the wearer's instep, one having ordinary skill in the art would appreciate that any kind of removable and/or adjustable strap can be used. Similarly, although the embodiment of FIGS. 1-6 only uses one upper connecting strap 30, any number of straps can be used to removably and/or adjustably connect the sidewalls 16 and 18 over the top of the shoe, as will be discussed further below. As shown in FIGS. 1-5, the upper connecting strap 30 is located longitudinally between the lower connecting strap 26 and the lateral and medial upright extensions 20 and 22, though one of ordinary skill in the art could readily configure an external ankle brace 50 having lower connecting strap(s) 26 in a desired location to fit a particular wearer, a selected shoe model, a certain cleat spacing, or for any other reason. The term “longitudinally between” is used herein to relate to the relative positions of two or more structures in the longitudinal direction—the two or more structures need not intersect, in whole or part, the same longitudinal line as one another but could be offset along different, substantially parallel longitudinal lines.

As shown in FIG. 5, the external ankle brace 50 restricts movement of the ankle in the first directions indicated by arrows 44 (the eversion and inversion directions) and permits ankle movement in the second directions indicated by arrows 46 (the plantar flexion and dorsiflexion directions). The external ankle brace 50 also restricts rotation of the ankle (“windshield wiper” motion of the toes with a stationary heel, or vice versa.

The external ankle brace 50 may include an upright fastening system 40 (FIG. 1), which would have at least one upright connecting strap 42 for selectively connecting the lateral upright extension 20 to the medial upright extension 22 above the ankle. This upright connecting strap 42 could include hook and loop fastener or any other type of strap that would allow for an adjustable and/or removable connection.

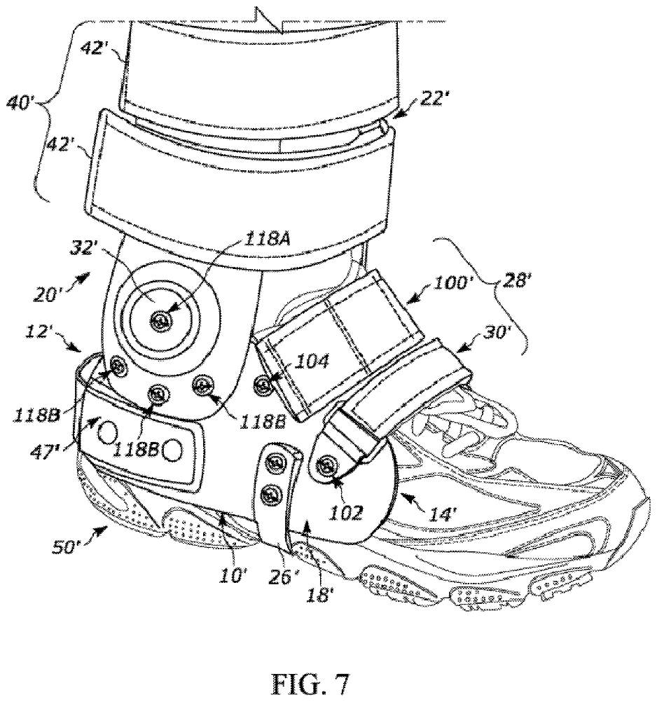

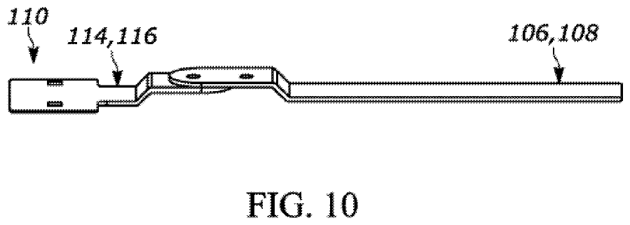

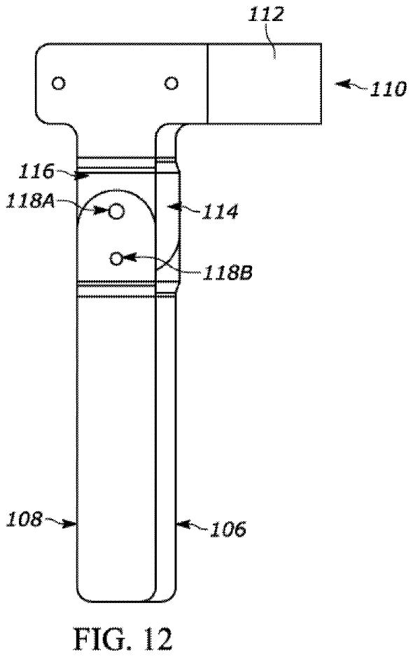

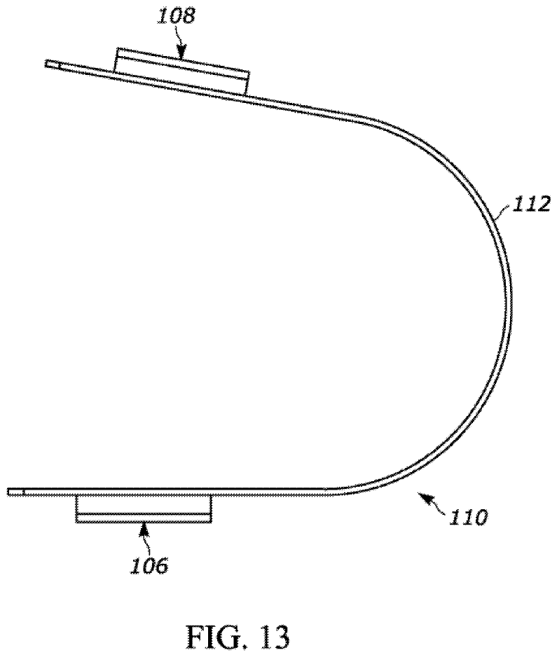

FIGS. 7-13 illustrate a second embodiment of an external ankle brace 50′. The external ankle brace 50′ of FIGS. 7-13 is similar to the external ankle brace 50 of FIGS. 1-6 and therefore, structures of FIGS. 7-13 that are the same as or similar to those described with reference to FIGS. 1-6 will be shown and/or described as having the same reference numbers with the addition of a “prime” mark. Description of common elements and operation similar to those in the previously described first embodiment will not be repeated with respect to the second embodiment or vice versa, but should instead be considered to be incorporated below/above by reference as appropriate.

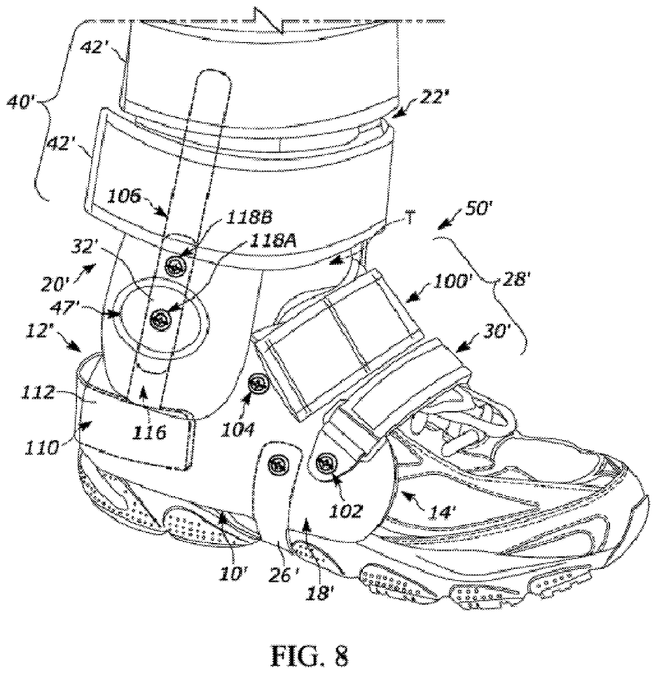

As shown in FIGS. 7-8, the external ankle brace 50′ may include an upright fastening system 40′ comprising at least one upright connecting strap 42′ (two shown) for selectively connecting the lateral upright extension 20′ to the medial upright extension 22′ above the ankle. It is contemplated that at least one of the upright connecting straps 42′ (or any other straps of the external ankle brace 50′) could have elastic properties to allow slight motion of the lateral and medial upright extensions 20′ and 22′ relative to one another as desired, as the external ankle brace 50′ is doffed and donned by the wearer.

Also as shown in FIGS. 7-8, the upper connecting strap 30′ is a first upper connecting strap 30′, and the upper fastening system 28′ includes a second upper connecting strap 100 for selectively connecting the lateral sidewall 18′ to the medial sidewall 16′ across the top of the shoe. The second upper connecting strap 100, when present, may be located longitudinally between the first upper connecting strap 30′ and the lateral and medial upright extensions 20′ and 22′. When rotation of the medial and lateral upright extensions 20 and 22 is “fixed” in a perpendicular position (an option discussed elsewhere herein), the second upper connecting strap 100 may be helpful to prevent plantar flexion and dorsiflexion, thus making the external ankle brace 50′ in this “non-pivoting mode” similar in overall stability to a fixed walking boot.

It is also contemplated that the first upper connecting strap 30′ (as shown in the Figures) could be omitted from the external ankle brace 50′, allowing the component shown and described herein as the second upper connecting strap 100 to serve as the (only) upper connecting strap, substantially in the position shown and described herein.

The second upper connecting strap 100 may be longitudinally wider than the first upper connecting strap 30′, as shown, for any reason. For example, in some use environments, the second upper connecting strap 100 will doing the majority of the support work (of the two upper connecting straps 100 and 30′) and be subject to the majority of the forces generated in-use. A wider strap in such circumstances distributes those forces and facilitates increased comfort for the wearer as opposed to a narrower strap (which might be more inclined to “cut in” at the edges to the wearer's foot/leg). Because the force is spread out by the wider strap, the wearer can use the external ankle brace 50′ for a long period of time without pain. The narrower first upper connecting strap 30′ is narrower, as shown, since there will not be enough room for another strap having a similar width to the second upper connecting strap 100 in many use configurations of the external ankle brace 50′.

As can be seen in at least FIG. 7, the first upper connecting strap 30′ may be permanently or adjustably attached to the respective lateral and/or medial sidewall 18′ or 16′ via a first upper fastener 102. As shown in the Figures, the first upper fastener 102 may attach the first upper connecting strap 30′ to an outer surface of the respective lateral and/or medial sidewall 18′ or 16′, such that the lateral and/or medial sidewall 18′ or 16′ is at least partially interposed between the body of the first upper connecting strap 30′ and the shoe.

Additionally, the second upper connecting strap 100, as shown in the Figures, may be permanently or adjustably attached to the respective lateral and/or medial sidewall 18′ or 16′ via a second upper fastener 104. As shown in the Figures, and in contrast to the first upper connecting strap 30′, the second upper fastener 104 attaches the second upper connecting strap 100 to an inner surface of the respective lateral and/or medial sidewall 18′ or 16′, such that the body of the second upper connecting strap 100 is at least partially interposed between the lateral and/or medial sidewall 18′ or 16′ and the shoe. In the case of the external ankle brace 50′ shown in FIGS. 7-13, this respective inner/outer fastening of the straps, at their respective positions, is considered to give a tighter and more secure fit of the external ankle brace 50′ around the wearer's shoe.

It is contemplated that one of ordinary skill in the art could similarly configure the strap placements and fastening types (number and kind of fasteners, inside/outside placement, and the like) for a particular use environment and to facilitate economical manufacture balanced with desired bracing results. For example, and as shown in FIGS. 7-8, the lower connecting strap 26′ of the external ankle brace 50′ may be located longitudinally between the first upper connecting strap 30′ and the lateral and medial upright extensions 20′ and 22′, and/or the second upper connecting strap 100 may be located longitudinally between the lower connecting strap 26′ and the lateral and medial upright extensions 20′ and 22′. As another example, in the case of the inside fastening of the second upper connecting strap 100, testing has shown that the malleability of the plastic of the external ankle brace 50′, and “lifting” of an externally-fastened upper connecting strap 100 away from the shoe by the respective lateral and/or medial sidewall 18′ or 16′, permit an undesirable degree of laxness in the fastening scheme for some use environments. That is, attaching the second upper connecting strap 100 to the inside of the lateral and medial sidewalls 18′ and 16′ may, in some use environments, assist with providing desired properties of fit, comfort, and stability. If the second upper connecting strap 100 were to be fastened outside the lateral and medial sidewalls 18′ and 16′, at the talus-adjacent position on the shoe shown in FIGS. 7-8, it may be more difficult to achieve a desired amount of comfort and stability for the wearer. As another example, in some use environments, the upper connecting strap 30′, 100 may include a variable length fastening operable for manual adjustment by a wearer to a predetermined length. As another example, the lower connecting strap 26′ may either include a variable length fastening operable for manual adjustment by a wearer to a predetermined length, or may have a constant length and not be manually adjustable by a wearer (e.g., to provide durability against a wearing surface of the shoe sole).

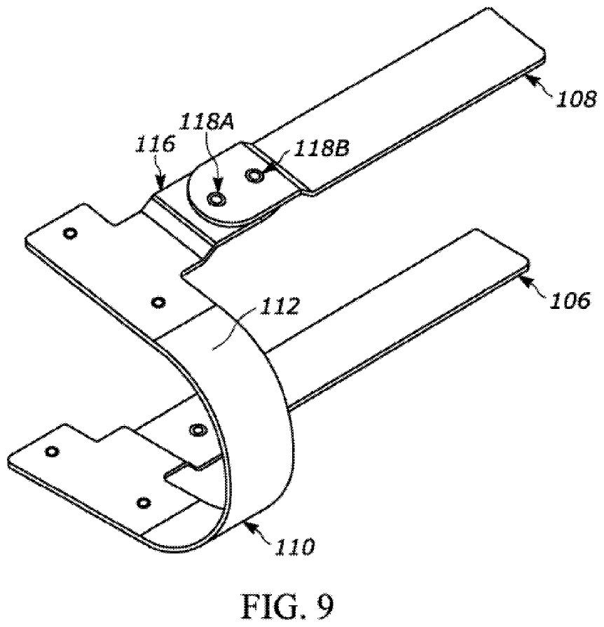

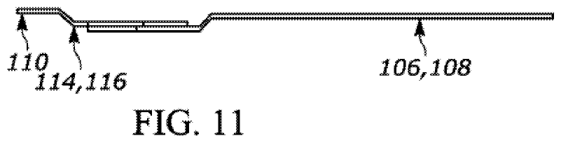

With reference now to FIGS. 8-13, the lateral upright extension 20′ may include a lateral reinforcing strut (shown in phantom view as 106 in FIG. 8), and the medial upright extension 22′ may include a medial reinforcing strut (shown as 108 in FIG. 9). The lateral and medial reinforcing struts 106 and 108, when present, may be malleable and configured to accept and maintain a nonplanar shape profile. A “nonplanar shape profile” is used herein to indicate that the component may be bent—manually and/or automatically—out of the substantially planar orientation shown for the lateral and medial reinforcing struts 106 and 108 in at least FIGS. 9 and 11. When the lateral and/or medial reinforcing struts 106 and 108 are in the nonplanar shape profile, they are configured to at least partially impart the nonplanar shape profile to the corresponding lateral or medial upright extension 20′ and 22′. In practical application, the lateral and/or medial reinforcing struts 106 and 108, and other, similar structures, can be used to help plastically and/or elastically deform the external ankle brace 50′, by a user/prescriber and/or machine, to fit the contours of a wearer's body in a desired manner. For example, if the wearer has very large or very small calf muscles, an orthotic or prosthetic assistance device, or any other feature that is different than that contemplated by a stock external ankle brace 50′, the external ankle brace 50′ can be configured (before, during, or after purchase/use) for that wearer or a like class of wearers.

The lateral and medial reinforcing struts 106 and 108 help with stability of the external ankle brace 50′ during use and may also facilitate custom fitting, or shaping the brace to the contours of an individual ankle and lower calf. Without the lateral and medial reinforcing struts 106 and 108, a prescriber would likely have to heat the plastic to mold it properly, and then it may not retain its shape under wear forces during use. The lateral and medial reinforcing struts 106 and 108 assist the respective lateral and medial upright extensions 20 and 22 in being shaped easily but then also holding a shape and providing stability.

It is contemplated that the lateral and medial upright extensions 20′ and 22′ may be made of a first material, and the lateral and medial reinforcing struts 106 and 108 may be made of a second material which is more ductile than the first material. For example, the first material may be a polymer and the second material may be a metal, such as, but not limited to, aluminum. It is also contemplated that the lateral and medial reinforcing struts 106 and 108 could be made from carbon-fiber and custom-manufactured to fit a particular user or class of users.

The lateral and medial upright extensions 20′ and 22′ may wholly encapsulate the lateral and medial reinforcing struts 106 and 108. This could be accomplished, for example, by the lateral and medial upright extensions 20′ and 22′ being molded around, or otherwise fabricated to encompass, the lateral and medial reinforcing struts 106 and 108. The material of the lateral and medial upright extensions 20′ and 22′ could be significantly larger in cross-sectional size than the respective lateral and medial reinforcing struts 106 and 108, or could instead be a relatively thin “skin” (e.g., a vinyl coating) interposed between the lateral and medial reinforcing struts 106 and 108 and the ambient space. Additionally, it is contemplated that the lateral and/or medial reinforcing struts 106 and 108 could be left bare to themselves serve directly as lateral and/or medial upright extensions 20′ and 22′.

With reference now to FIGS. 8-13, the rear portion 12′ may include a rear reinforcing strut 110. The rear reinforcing strut 110 may be malleable and configured to accept and maintain a nonplanar shape profile, to at least partially impart the nonplanar shape profile to the rear portion 12′, although it is contemplated that the rear reinforcing strut 110 could also or instead be relatively rigid and configured to maintain the lateral and medial reinforcing struts 106 and 108 in a predetermined spacing and mutual orientation relationship (e.g., to avoid “torquing” or “twisting” of the lateral and medial upright extensions 20′ and 22′. despite any reconfiguring of the lateral and/or medial reinforcing struts 106 and 108).

As with the external ankle brace 50 of FIGS. 1-6, the rear portion 12′ of the external ankle brace 50′ of FIGS. 7-13 encircles the heel, but leaves the heel of the shoe exposed so that the user's shoe can directly contact the ground. The rear portion 12′ of the rigid heel enclosure10′ is cut out to leave the bottom of the heel, and a portion of the side of the heel, of the shoe exposed. As depicted in at least FIG. 7, the back of the external ankle brace 50′ may fit or rest on the base of the heel counter of the shoe, which is not the base of the shoe. The external ankle brace 50′ does not touch the ground at the heel of the shoe and there is a gap (e.g., about a half inch, for some users) between the sole of the shoe and the rigid heel enclosure 10′.

Whether or not deformation of the rear reinforcing strut 110 is contemplated, the rear portion 12′ may be made of a first material and the rear reinforcing strut 110 may be made of a second material which is more ductile than the first material. For example, the first material may be a polymer and the second material may be a metal. For example, the first material may be a polymer and the second material may be a metal, such as, but not limited to, aluminum. It is also contemplated that the rear reinforcing strut 110 could be made from carbon-fiber and custom-manufactured to fit a particular user or class of users.

The rear portion 12′ may wholly encapsulate the rear reinforcing strut 110. The rear portion 12′ may be molded around the rear reinforcing strut 110. The material of the rear portion 12′ could be significantly larger in cross-sectional size than the rear reinforcing strut 110, or could instead be a relatively thin “skin” (e.g., a vinyl coating) interposed between the rear reinforcing strut 110 and the ambient space. Additionally, it is contemplated that the rear reinforcing strut 110 could be left bare to itself directly serve as a rear portion 12′. A material selection (e.g., a high-friction material) and/or surface treatment (e.g., knurling) may be used on the rear portion 12′ (or directly on the rear reinforcing strut 110, when serving as the rear portion 12′) to increase frictional surface area contacting the heel counter of the shoe to provide desired fixation to help restrict plantar flexion and dorsiflexion. (As with several components of the external ankle brace 50′, including, but not limited to, the lateral and medial upright extensions 20′ and 22′ and the rear and forward portions 12′ and 14′, it may be desirable to balance manufacturing considerations, friction of surfaces, area of surfaces, and weight of components in seeking comfort, stability, security/tightness, motion restriction, and usability for the wearer.)

The rear reinforcing strut 110 may include a curved rear strut body 112 extending around the heel portion of the shoe and lateral and medial strut stubs 114 and 116, respectively, extending substantially perpendicularly from the rear strut body 112, at opposed locations on the rear strut body 112. The lateral and medial reinforcing struts 106 and 108 are directly pivotally connected to the lateral and medial strut stubs 114 and 116, respectively. The lateral, medial, and/or rear reinforcing struts 106, 108, and 110, when present, may provide desired rigidity and/or strength, such as to permit a lower-profile size, to the respective lateral and medial upright extensions 20′ and 22′ and/or rear portion 12′.

As shown in the Figures, the rear reinforcing strut 110 may be directly pivotally connected to the lateral and medial reinforcing struts 106 and 108 in any desired manner. For example, at least one restraining bolt 118 may be connected to a chosen one of the lateral and medial upright extensions 20′ and 22′ and be operative to selectively restrict pivoting of the chosen upright extension 20′ and 22′ respective to a corresponding lateral or medial sidewall 18′ or 16′. In FIG. 7, this configuration is shown as a primary restraining bolt 118A and a plurality (three shown) of secondary restraining bolts 118B. The primary restraining bolt 118A in FIG. 7 is located at a pivot point of the lateral ankle joint 32′. The secondary restraining bolts 118B, when present, resist pivoting of the respective lateral or medial upright extension 20′ and 22′ with respect to a corresponding lateral or medial sidewall 18′ or 16′. Thus, restraining bolts 118A and/or 118B may be connected to a chosen one of the lateral and medial reinforcing struts 106 and 108 and may be operative to either assist/facilitate or selectively restrict pivoting of the chosen upright extension 20′ and 22′ respective to the rear reinforcing strut 112.

As another example, FIGS. 8-13 depict a configuration of the external ankle brace 50′ shown as having one restraining bolt 118A at a center pivot location of the lateral or medial ankle joint 32 and 34, and at least one second restraining bolt 118B at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween. For example, and as shown in at least FIGS. 9-12, the second restraining bolt 118B may selectively mutually connect the lateral or medial strut stub 114 or 116 to a respective lateral or medial reinforcing strut 106 or 108.

It is contemplated, however, that one or more second restraining bolts 118B could instead be used at a center pivot location of the lateral or medial ankle joint 32 and 34, and at least one restraining bolt 118A could be placed at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween as desired. In this alternate situation, the second restraining bolt 118B shown in FIG. 8 could be located for pivotal alignment with the malleolus and the first restraining bolt 118A could be off-center for selectively preventing pivoting. That is, the off-center restraining bolt(s) 118—regardless of specific configuration—could be manipulated by a prescriber or user to prevent pivoting of one or both of the lateral or medial upright extensions 20′ or 22′ with respect to the respective lateral or medial sidewalls 18 and 16 at a desired “restrict second direction pivoting” time, and could be manipulated to allow at least partial pivoting of one or both of the lateral or medial upright extensions 20′ or 22′ with respect to the respective lateral or medial sidewalls 18 and 16 at a desired “allow second direction pivoting” time. One of ordinary skill in the art will be able to provide one or more restraining bolts 118 having a desired type, location, and other physical configuration for a particular use environment of the external ankle brace 50′.

Regardless of how the restraining bolts 118 are configured and located on the various other components of the external ankle brace, it is contemplated that the restraining bolts 118 could be manipulated by any suitable party, at any desired time before, during, and/or after wear of the external ankle brace 50 and 50′, and for any desired reason. For example, the pivoting could be further restricted once an already tender ankle is further stressed, or the pivoting could be further permitted if a previously tender ankle responds well to light, restricted-pivoting duty. It is contemplated that one or more components of the external ankle brace 50 and 50′ could include a slot (e.g., a curved slot) within which a corresponding restraining bolt 118 can relatively travel or slide during use, in order to permit a limited amount of pivoting. It is also contemplated that one or more of the restraining bolt(s) could be a Chicago screw/bolt type, include a post and/or sleeve feature, or otherwise be configured to facilitate smooth rotation (and/or avoid wear) between two or more components of the external ankle brace 50 and 50′, whether or not they are permitted to selectively pivot relative to one another.

Via the aspects of the external ankle braces 50 and 50′ shown and described herein, a user can place the external ankle brace 50 and 50′ around an existing shoe (thus obviating the expense and inconvenience of special and/or mismatched shoes to accommodate an inside-the-shoe brace), tighten as desired, and accordingly selectively restrict movement of an ankle in a first direction and selectively permit movement of the ankle in a second direction (e.g., through selective use of the pivoting restriction schemes described above). The external ankle brace 50 also restricts rotation of the ankle (“windshield wiper” motion of the toes with a stationary heel, or vice versa). Accordingly, an ankle can receive a desired amount of support—capable of changing very quickly, even during a single wear session (e.g., an athletic or daily-activity event) via use of the restraining bolt(s) 118—and thus avoid initially or additionally injuring an ankle, foot, leg, or other portion of the wearer's body.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Padding or other cushioning material could be placed on any desired surface(s) of the components of the external ankle brace 50, 50′ to assist with force absorption, spacing, abrasion resistance, or for any other reason. A restraining bolt 118 could be selectively associated with only a chosen one of the lateral and medial upright extensions 20′ and 22′ to prevent with respect to the respective lateral and medial sidewall 18 and 16 at a desired “restrict second direction pivoting” time, with pivoting of the other of the lateral and medial upright extensions 20′ and 22′ being indirectly limited via the connection to the other of the lateral and medial upright extensions 20′ and 22′ through the upright connecting strap(s) 42. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Embodiments include an external gauntlet ankle/foot orthosis which applies over the shoe providing ankle and foot stability prophylactically or following acute or chronic trauma. Embodiments relate to an orthotic that limits or prevents ankle inversion, eversion, dorsiflexion and plantar flexion and well as providing mid foot support.

Embodiments can minimize ankle inversion and eversion during physical activity and/or minimize ankle medial and later rotation during physical activity and/or minimize ankle plantar flexion and dorsiflexion during physical activity and/or provide stability to the mid foot in limiting pronation and supination of the foot.

Embodiments can include a foot/ankle orthotic that includes a lateral sidewall, a medial sidewall, a heel enclosed backing connecting the sidewalls, a lateral upright extension, a medial upright extension and a bottom strapping system connecting sidewalls. The lateral and medial upright extensions are attached to the sidewalls with an overlapping ankle joint off-set to accommodate for medial and lateral malleolus anatomical positioning. The lateral sidewall coincides with the outer or exterior portion of the foot/ankle and the medial sidewall coincides with the inner portion of the foot/ankle. The lateral upright extension coincides with the outer or exterior portion of the lower leg and the medial upright extension coincides with the inner portion of the lower leg. Lateral and medial extension walls are configured to rise above the ankle of the wearer of the orthotic by approximately 8-9 inches (from the bottom of the hinge to the top of the extension walls). When donned by the wearer, lateral and medial side walls also partially wrap over the top or dorsum of the foot leaving a gap of approximately 3 to 4 inches between the sidewalls. The width of the medial and lateral upright extensions is approximately 3-4 inches wide.

A feature of an embodiment is to have the securing mechanism include a hook and loop strap across the dorsal (top) of the foot. This Velcro securing strap is riveted to the in place on both the medial and later side walls. A D ring is utilized on the lateral fixation in which the Velcro strap can be fed through and secured back upon itself. The lateral and medial upright extensions are secured by two removable Velcro straps and D rings. Male component Velcro is adhesively attached to each upright and the female component Velcro strap can connect to the uprights are desired positions for appropriate fitting. As an option, the brace may also be applied with various types of athletic adhesive tape in conjunction with or instead of the Velcro strapping and D ring system.

Another feature of an embodiment is an overlapping ankle joint hinge to allow the ankle to move freely through plantar flexion and dorsiflexion. The overlapping ankle joint is located on the medial and lateral aspects of the gauntlet where the medial and lateral side bodies attach with the medial and lateral uprights respectively. The ankle joint hinge components are off set to produce a more anatomically correct gauntlet for a more fluid mobility.

Foam padding (approximately ¼ inch) is attached to the inside of both the medial and lateral uprights to provide additional comfort and protection for the wearer. The gauntlet is sized so that one size can fit multiple size shoes. A separate gauntlet is needed to accommodate both right and left ankles.

A sheet of vacuum formable thermoplastic large enough to cover the entire mold is cut and placed in an oven to be heated to a formable temperature. These are several types and thicknesses of plastic that may be used for this fabrication including orthotic grade polypropylene, polyethylene, and copolymer.

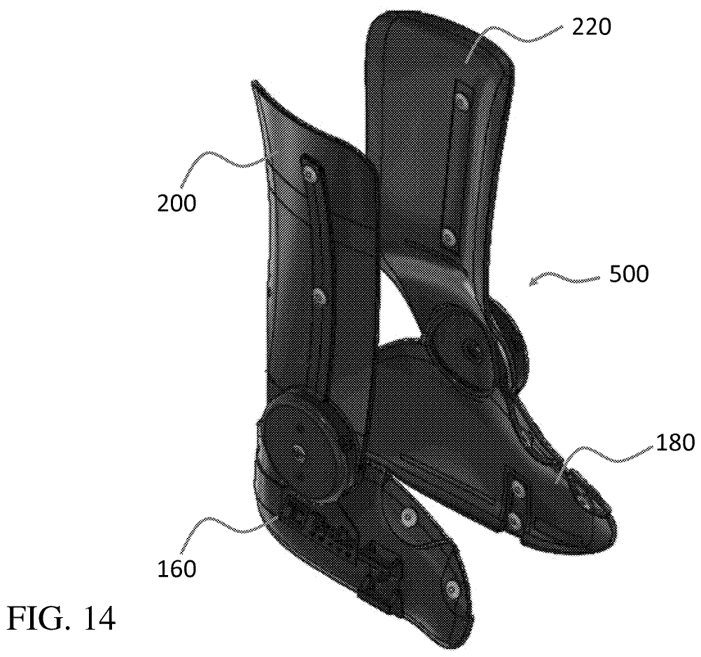

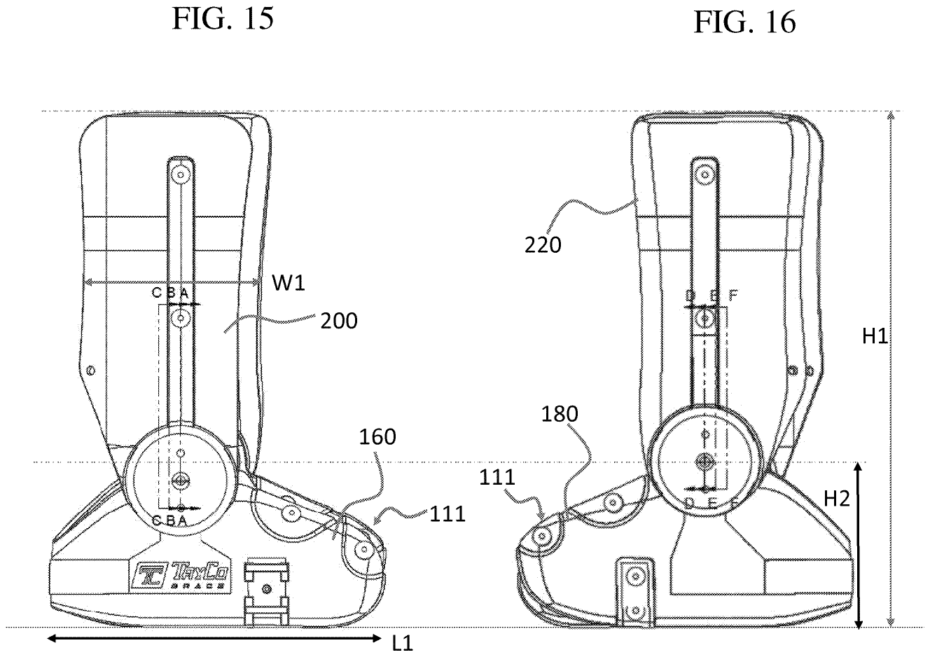

FIGS. 14 and 15 present another, different embodiment, from the embodiments of FIGS. 1-13 described above. By way of example only and not by way of limitation, the embodiments of the external ankle brace 500 of FIGS. 14 to 20 include an adjustable bottom strap. Moreover, embodiments of the external ankle brace 500 of FIGS. 14 to 20 include a medial upright extension that is anatomically correct. For example, the upright is contoured to accommodate the medial malleolus.

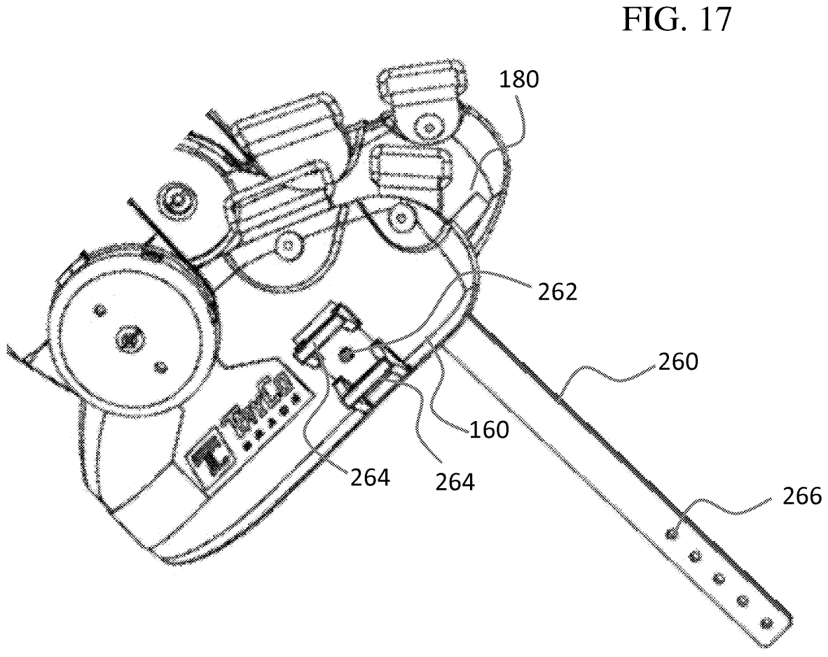

FIGS. 15 and 16 presents side views of the extra level brace 500 according to an exemplary embodiment. Here, it can be seen that there are lateral upright extension 200 and medial upright extension 220. In an exemplary embodiment, these uprights can have one or more of the features detailed above with respect to the uprights of the embodiments of FIGS. 1 to 13. These uprights are jointly connected to the rigid heel enclosure 96. The rigid heel and closure 96 includes a lateral sidewall 160, and a medial sidewall 180. Again, the sidewalls can have one or more of the features detailed above with respect to the sidewalls of the embodiments of FIGS. 1 to 13. As will be described in greater detail below, the joints of the respective lateral and medial upright extensions have a male-female joint assembly, some respect to portions of which are monolithic with the remainder of the bulk of the extensions and sidewalls respectively. Again, additional details of this will be described below.

FIG. 16 shows a dimension H1, which is the height from the bottom to the top of the brace 500. In an exemplary embedment, H1 can be less than and/or equal to and/or greater than (meaning that it can be less than one of the following numbers and greater than another of the following numbers for example) 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75 or 15 inches or any value or range of values therebetween in 0.0001 inch increments (to achieve English units for example). And note that the sides of the brace can be different heights (e.g., the top of the lateral upright extension can be at a different height than the top of the medial upright extension), and thus one side can have one of the aforementioned values, and the other side can have another of those values. Indeed, embodiments are such that that is the case. In an exemplary embodiment, H1 on one side, such as the medial side, can be less than, equal to and/or greater than 0.1, 0.2, 0.3, 0.4, 0.5, 0.6. 0.7, 0.8, 0.9, 1.0, 1.1, 1.2, 1.3, 1.4, 1.5, 1.6. 1.7, 1.8, 1.9, 2.0, 2.1, 2.2, 2.3, 2.4, 2.5, 2.6. 2.7, 2.8, 2.9, 3, 3.1, 3.2, 3.3, 3.4, 3.5, 3.6, 3.7. 3.8. 3.9, 4.0, 4.25, 4.5, 4.75, 5, 5.25, 5.5, 5.75, 6, 6.25, 6.5, 6.75, 7, 7.25, 7.5, 7.75, 8, 8.25. 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18% or more or any value or range of values therebetween in 0.01% increments than the H1 on the opposite side (where the smaller one is the denominator).

FIG. 15 shows a dimension W1, which is the width of the upright 200 (and can be a width of the upright 220) at its narrowest portion in the parabolic portion. In an exemplary embodiment, H1 can be less than and/or equal to and/or greater than 2.5, 2.75, 3, 3.25, 3.5, 3.75, 4, 4.25, 4.5, 4.75, 5, 5.25, or 5.5 inches or any value or range of values therebetween in 0.0001 inch increments (to achieve English units for example). And note that these drawings herein (all drawings unless otherwise noted) are to scale, and thus disclosures constitute values that are scaled relative to other specific values for purposes of textual economy.

FIG. 16 shows a dimension H2, which is the height of the axis of rotation of the medial joint/hinge from the bottom of the medial sidewall. In an exemplary embodiment, H2 can be less than and/or equal to and/or greater than 2, 2.25, 2.5, 2.75, 3, 3.25, 3.5, 3.75, 4, 4.25, 4.5, 4.75 or 5 inches or any value or range of values therebetween in 0.0001 inch increments (to achieve English units for example).

FIG. 15 shows a dimension L1, which is the length of the lateral sidewall (and can be the length of the medial sidewall) from the rearmost end to the foremost end. In an exemplary embodiment, L1 can be less than and/or equal to and/or greater than 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75 or 11 inches or any value or range of values therebetween in 0.0001 inch increments (to achieve English units for example).

It is noted that the dimensions disclosed herein and the values disclosed herein are values and dimensions associated with the brace in a completely relaxed state unless otherwise noted. In this regard, tensioning of the straps and/or the action of wearing the brace can result in deformation of the structures of the brace, even though those structures are rigid (more on this below). Accordingly, the dimensions and values disclosed herein can be dimensions that corresponded components in a completely disassembled state and/or components in a completely relaxed state and/or a state where all straps are completely removed. In an exemplary embodiment, the dimensions and values disclosed herein are those that exist when the brace is positioned in exact alignment of the X, Y and Z axes. In an exemplary embodiment, the dimensions and values disclosed herein are those that correspond to the dimensions and values when the lower connecting strap is completely removed and in no way attached to the sidewalls and the brace is positioned so that the bottoms of the sidewalls are located on a flat level surface without any other forces of the gravity acting on the brace.

These dimensions are given so that the other portions of the brace can be scaled to within 10, 9, 8, 7, 6, 5, 4, 3, 2 or 1%. Herein, the figures of FIG. 14 and thereafter are to scale (more on this below).

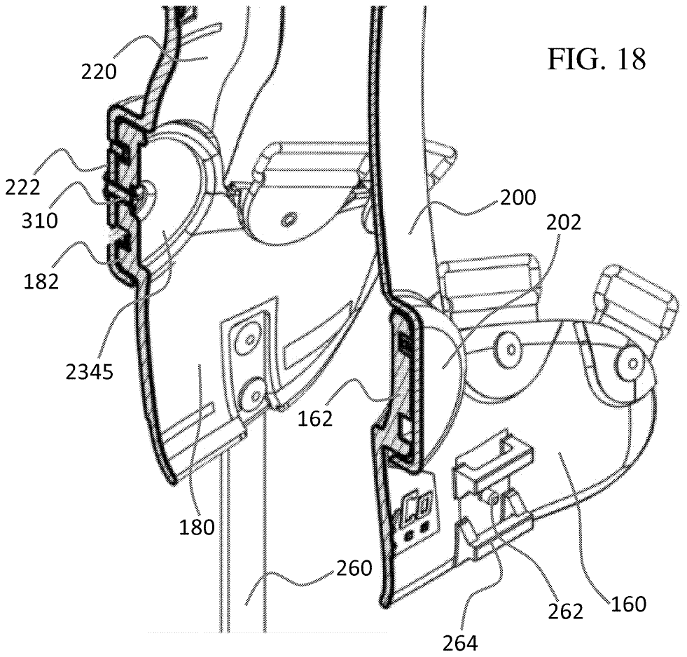

FIG. 18 depicts a cross-sectional view of the external ankle brace 500 on a plane lying on and parallel to the rotation axis of the medial joint and/or the longitudinal axis of the Chicago screw 310 that establishes the pivot of that joint. Owing to the offsets of the medial and lateral joints as will be described in greater detail below, the cross-section does not extend through the axis of rotation of the lateral joint and/or the longitudinal axis of the Chicago screw of that joint (more on this below). The cross-sectional view shows that the lateral and medial joints are made up of a male portion (respectively 162 and 182) that is monolithic with the bulk of the structure that makes up the respective sidewalls, and a female portion (respectively 202 and 222) that is monolithic with the bulk of the structure that makes up the respective uprights.

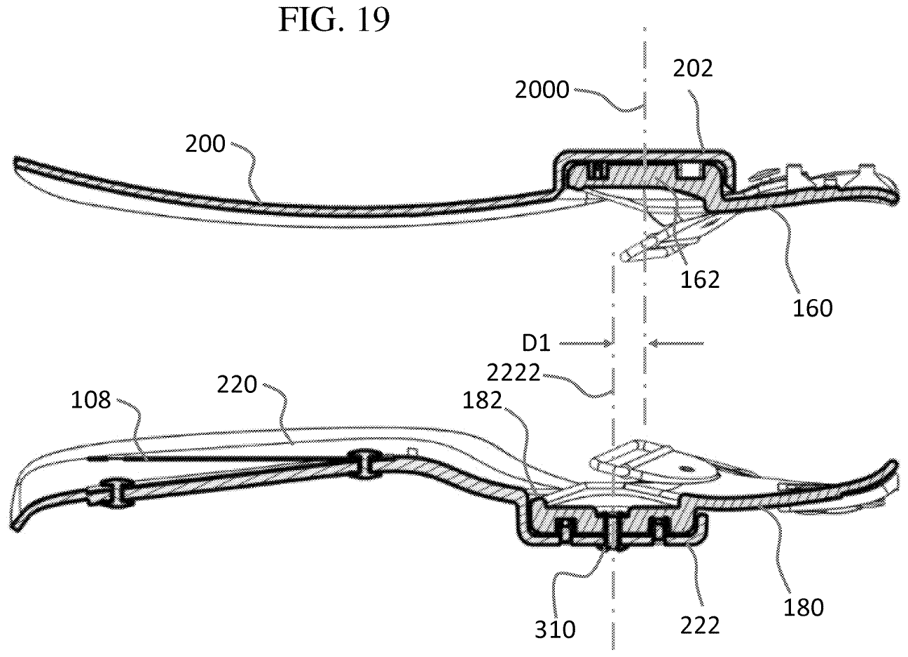

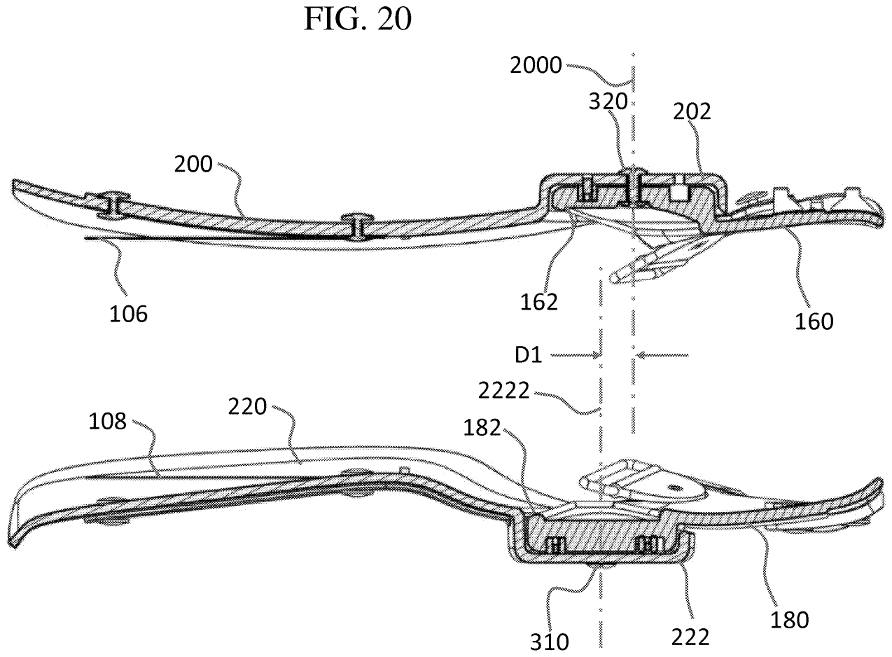

In an exemplary embodiment, on a per mass and/or per volume basis, the respective male and female portions are monolithic with structure that makes up at least 70, 75, 80, 85, 90, 95, 96, 97, 98 or 99 or 100% of the respective upright and/or the respective sidewall and/or the rigid heel enclosure structure (the control would not include the straps or the rivets, etc.—the control would include for example the reinforcing structures such as elements 108, 106 and/or 110). In an exemplary embodiment, the joint portions are monolithic with a structure of the respective sidewalls and/or heel enclosure and/or the uprights, which structure extends in a monolithic manner from the bottom most portion of that component to the top most portion of that component and/or from the forward most portion of that component to the rearward most portion of that component and/or a distance that is 70, 75, 80, 85, 90, 95, 96, 97, 98 or 99 or 100% of the overall topmost distance and/or the forward and/or reward-most distance. By way of example only and not by way of limitation, FIG. 19 depicts the cross-sections of the bulk of the structure of the uprights (the crosshatched portions with major components extending horizontally—this excludes the element 108 and the rivets therefor and the Chicago screw 310 for example) and the bulk of the structure of the sidewalls and the rigid heel enclosure. FIG. 20 depicts the cross-sections of the bulk of the structure of the uprights (excluding the element 106 and the rivets therefore and the Chicago screw 320 for example) and the bulk of the structure of the sidewalls and the rigid heel enclosure. As seen in FIG. 19 with respect to the lateral upright extension 200, the cross-section extends unbroken from the top to the bottom most portion thereof, the structure that establishes such extension is monolithic with the female portion 202 of the lateral joint. As seen in FIG. 19 with respect to the lateral sidewall 160, the cross-section extends unbroken from the top to the bottom most portion thereof, the structure that establishes such extension is monolithic with the male portion 162 of the lateral joint. As seen in FIG. 20 with respect to the medial upright 220, the cross-section extends unbroken from the top to the bottom most portion thereof, the structure that establishes such extension is monolithic with the female portion 222 of the medial joint. As seen in FIG. 20 with respect to the medial sidewall 180, the cross-section extends unbroken from the top to the bottom most portion thereof, the structure that establishes such extension is monolithic with the male portion 182 of the medial joint. But again, in an exemplary embodiment where the structure is not monolithic, the distance could be 70 or 80 or 90% as detailed above. And note that the just described features are not excluded because there are rivets for example in the lateral upright as shown in the cross-section of FIG. 20. Because the structure extends monolithically about those rivets, the aforementioned features are still present. That is, the distance need not lie in an exact plane to meet the aforementioned features. Still, by taking the cross-section in a plane that is offset and otherwise away from the rivets and/or Chicago screw, one can have an unbroken cross-section from the top to the bottom that includes the hinge portions as seen in FIGS. 19 and 20.

It is noted that in an exemplary embodiment, a circular foam pad is placed on the inboard face 2345 of the male joint portions so as to pad between the joint portion and the respective malleolus (or, more accurately, the skin over the respective malleolus).

It is noted that in an exemplary embodiment, there is a washer that is located between the faces of the male and female portions that are normal to the pivot axis. The washers has a hole through which the Chicago screws extend. The washers can be low friction washers that are made from a polymer or any other material that is suitable. In an exemplary embodiment, the washers maintain a space between the aforementioned faces of the male and female joint portions that is at least and/or equal to and/or no more then 0.005, 0.01, 0.015, 0.02, 0.25, 0.03, 0.04, 0.05, 0.06, 0.07, 0.08, 0.09, 0.1, 0.125, or 0.15 inches or any value or range of values therebetween in 0.0001 increments (or less), at least to achieve the standard English measurements. In an exemplary embodiment, the aforementioned values are the thickness of the washer. In an exemplary embodiment, the outer diameter of the washer (it can be circular outside diameter and/or with respect to the inside diameter) and/or maximum outer diameter of the washer can be less than and/or equal to and/or greater than 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75 or 3 inches or any value or range of values therebetween in 0.001 increments (or less) to achieve the English measurement units at least.

In an exemplary embodiment, the interfacing or otherwise facing surfaces of the male and female joint portions are circular, concomitant with the fact that the portions rotate relative to one another. It is also noted that in an exemplary embodiment, the figures are drawn to scale in totality and/or on a percent basis (e.g., if a view is shown in a shrunken manner, that is still to scale on a percent basis).

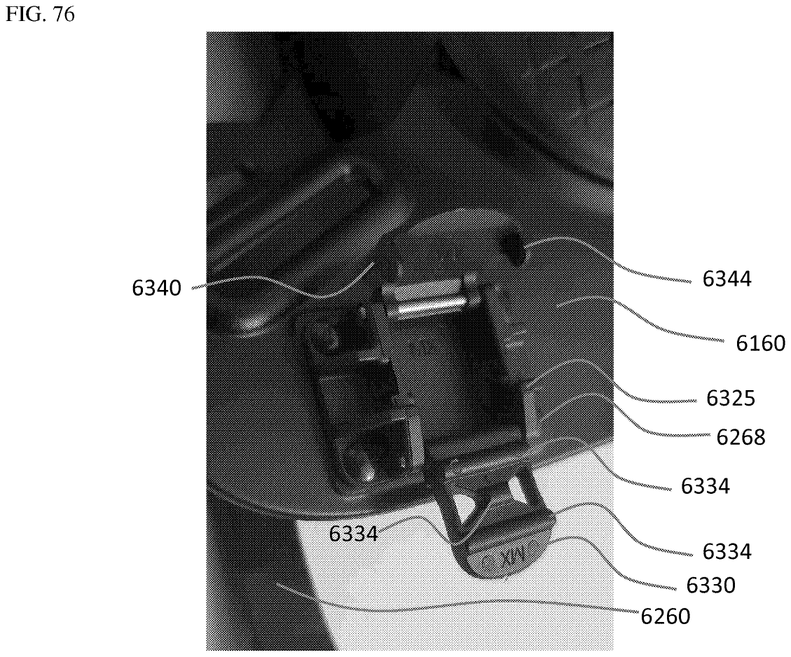

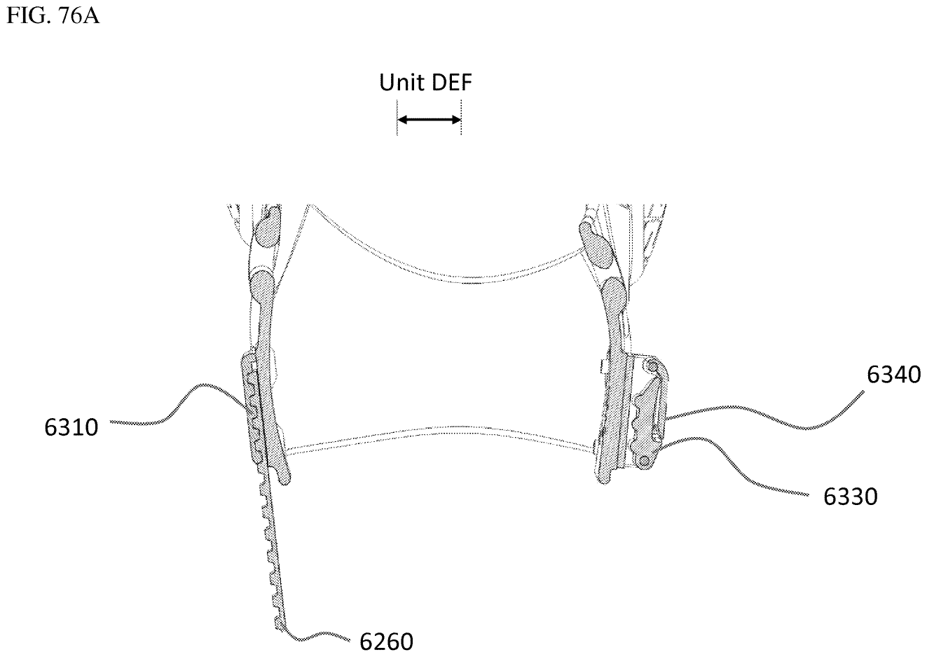

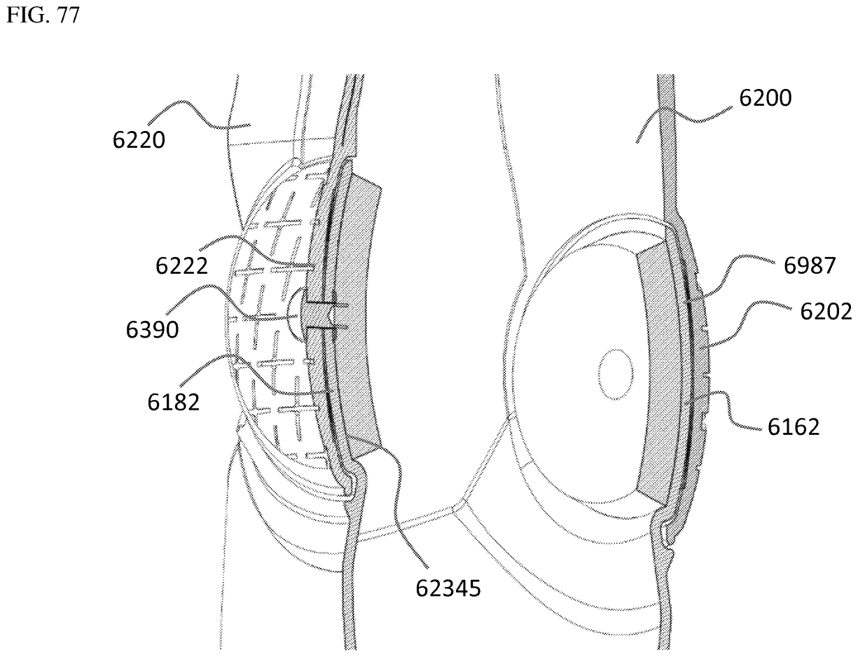

As can be seen in FIGS. 19 and 20 (and FIGS. 76 and 77), the respective axes 2222 and 2000 of joint rotation/joint pivot for the medial joint and the axial joint are offset by a distance D1 in the Z axis (the axis normal to the ground/bottom of the brace). In an exemplary embodiment, D1 can be less than and/or equal to and/or greater than 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9 or 2 inches or more or any value or range of values therebetween in 0.0001 increments (or less), at least to achieve the standard English measurements.

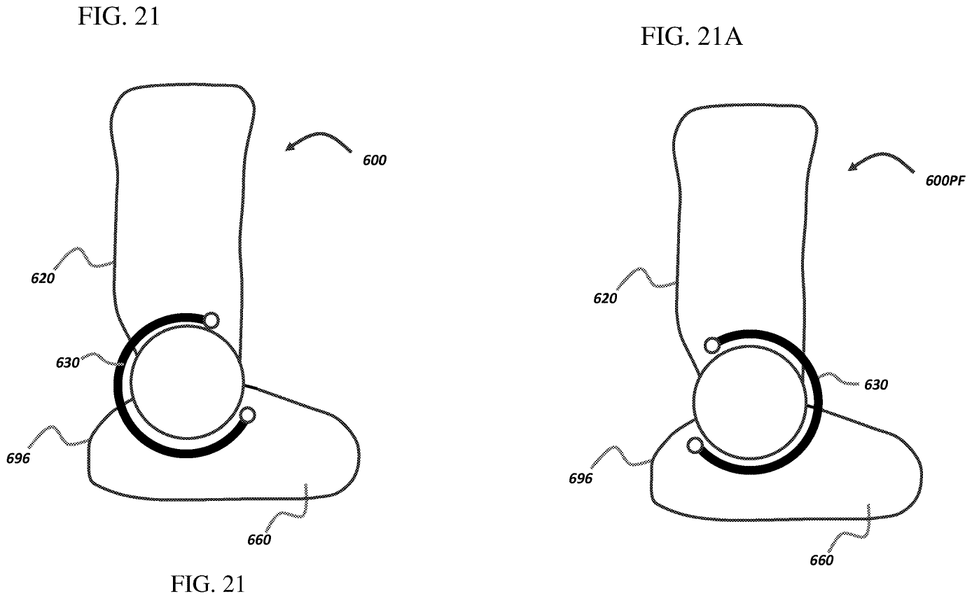

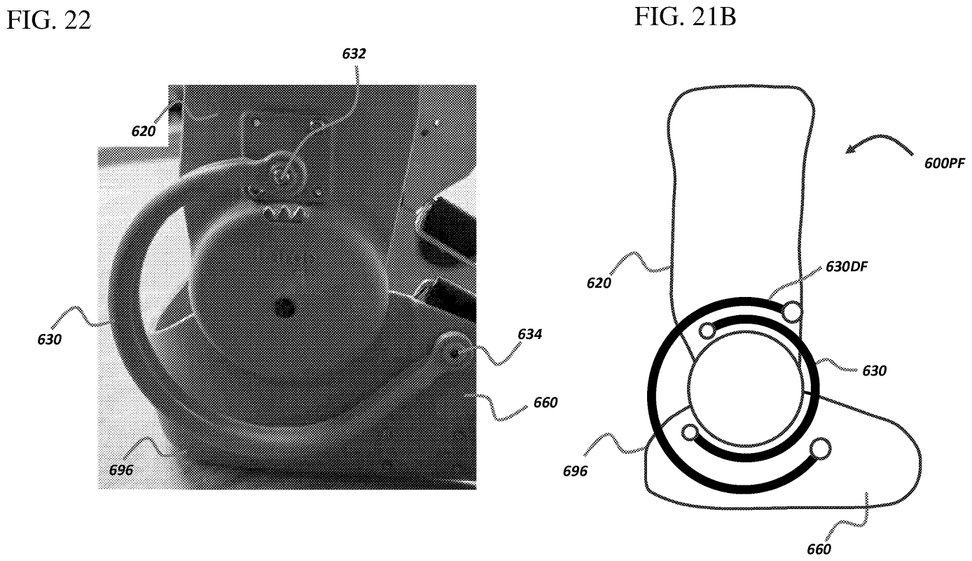

FIG. 21 presents an exemplary external ankle brace 600 according to another exemplary embodiment. This exemplary embodiment has a dorsiflexion assist feature. In this exemplary embodiment, the basic structure of the external ankle brace can correspond to any of those detailed herein or variations thereof, and features will not be repeated in the interest of textual economy. In this regard, upright 620 can correspond to any of the uprights detailed herein and heel enclosure 696 can correspond to any of the heel enclosures detailed herein. Embodiments will be described in a manner agnostic as to which side of the brace is under discussion (lateral or medial). Any disclosure herein of a feature on one side of the brace with respect to the dorsiflexion assist embodiment can correspond to a feature on the other side as well. In some embodiments, the features associated with the dorsiflexion assist are duplicative on each side, while in other embodiments, the features associated with the dorsiflexion assist are only located on one side. It is noted that in some embodiments, one side can have one or more features associated with the dorsiflexion assist while in other embodiments can have some of the other features detailed herein.

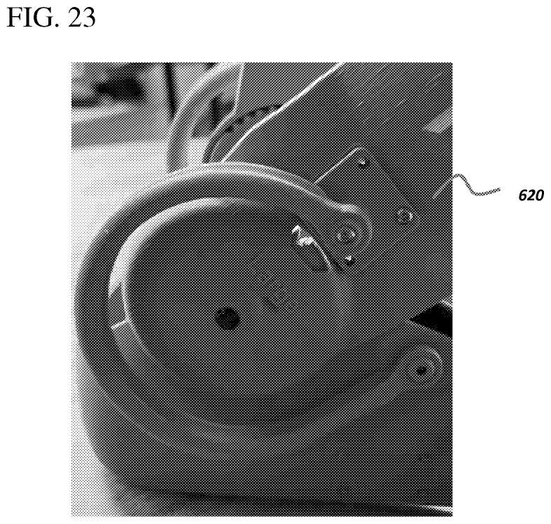

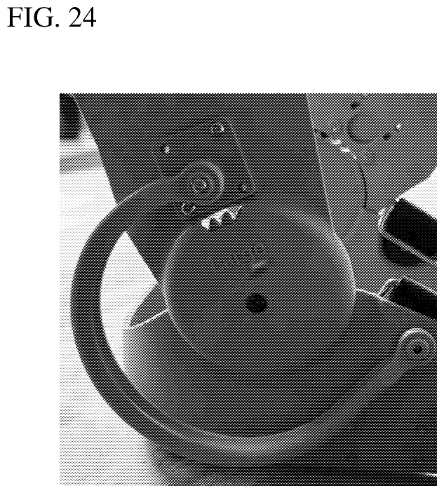

Brace 600 includes a spring 630 that provides a force on the heel enclosure 696 that forces the heel enclosure to rotate, with respect to the upright 620, with respect to the frame of reference of FIG. 21, counterclockwise in the absence of a restraining force. More specifically, FIG. 22 presents a more detailed view of the portions of the brace associated with the dorsiflexion assist embodiment. Here, spring 630 is mounted to upright 620 by bearing 632, which allows end of the spring to rotate relative to the upright. The other end of spring 630 is attached to the side wall 660 by way of bearing 634, which also allows that end of the spring to rotate relative to the sidewall 660. FIGS. 21 and 22 present the heel enclosure 696 in the position relative to the upright 620 that would result from the person wearing the brace standing vertically, where the muscles of the foot force the heel enclosure to the horizontal position. FIG. 23 shows the effect of the spring when the heel enclosure is almost totally unrestrained (in this embodiment, the upright 620 strikes the lug for the top strap of the sidewall before the spring is totally relaxed—in an embodiment, this may not necessarily be the case owing to a different configuration of the spring and/or a different configuration of the sidewall—in this exemplary embodiment, by way of example only and not by way of limitation, the upright 620 could move clockwise an additional amount that is less than, greater than and/or equal to 2, 3, 4, 5, 6, 7, 8, 9 or 10 degrees or any value or range of values therebetween in 0.1° increments) where the spring 630 imparts the force on to the heel enclosure to rotate the heel enclosure counterclockwise with respect to the frame of reference of FIG. 23. FIG. 24 depicts the upper right angled relative to the heel enclosure in a scenario that could occur when the person wearing the brace is walking, where the foot wearing the brace in FIG. 24 has been put forward relative to the other foot, and the foot has forced the heel enclosure to be parallel (effectively parallel) with the ground. As seen in FIG. 24, the general diameter of the spring 630 has increased with respect to that of FIGS. 22 and 23. The relaxed state of the spring is that of a smaller diameter spring, and thus what is shown in FIG. 24 represents the result of impartation of energy into the spring from walking for example. This energy is released when the spring drives the heel enclosure counterclockwise, or otherwise the heel enclosure is permitted to be moved counterclockwise.

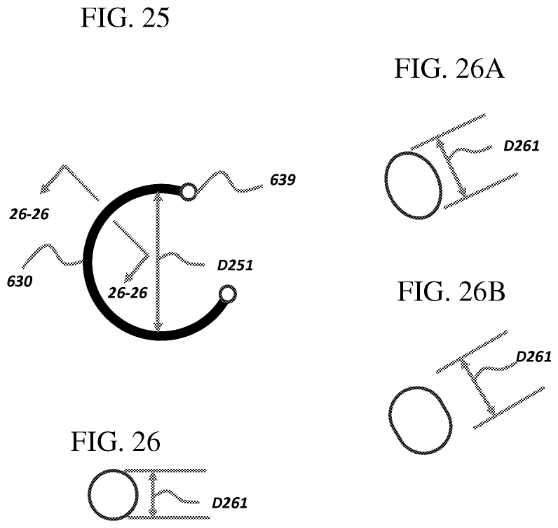

FIG. 25 presents an exemplary macro diameter of the spring 630 with respect to the inside thereof when the spring is fully relaxed (e.g., prior to attachment to the brace for example—the spring stores zero energy). In an exemplary embodiment, D251 can be less than greater than and/or equal to 2, 2.5, 3, 3.5, 4, 4.5, 5, 5.5, 6, 6.5, or 7 or more inches, or any value or range of values therebetween in 0.05 inch increments (e.g., 5.25, 4.30, 4.05 to 6.55, etc.). In an exemplary embodiment, when the brace is articulated to the maximum allowable ranges and/or the ranges detailed herein as will be further detailed below, the diameter D251, which can be considered the maximum interior diameter measured wherever that is located or wherever such occurs (and note that the spring may not be perfectly circular on the inside when in the totally relaxed state—these are but examples), or D251 can be the average (mean, median, and/or mode) of the total curvature (not including the bearing receptacle lugs 639) over 50, 55, 60, 65, 70, 75, 80, 85, or 90% of the uncurved length of the spring or could be the minimum diameter of the spring, can increase by less than, greater than, and/or equal to 5, 10, 15, 20, 25, 30, 35, 40, 45, 50, 55, or 60% or more, or any value or range of values therebetween in 1% increments.

FIG. 26 shows a cross-section through section 26-26, showing the maximum and/or minimum and/or average (mean, median, and/or mode) of the diameter, whether maximum or minimum or overall, or that over the aforementioned percentages of length of the spring 630, where D261 can be less than, greater than, and/or equal to .15, 0.2, 0.25, 0.3, 0.35, 0.4, 0.45, 0.5, 0.55, 0.6, 0.7, 0.8, 0.9, 1.0, 1.1, or 1.2 or more inches, or any value or range of values therebetween in 0.01 inch increments. These measurements can be on a plane about which the spring extends (e.g., a vertical plane and/or a plane or a series of planes normal thereto (e.g., 5, 10, 15, 20, 25, or 30 planes each offset from the other by say 5, 10, 15 or 20 degrees (the planes could intersect each other at the axis of articulation of the upright and the heel enclosure) or whatever value can be utilitarian to measure the values). Note also that the spring need not be totally circular or otherwise have a curved portion over the entire length of the spring. In this regard, FIG. 22 shows that the spring includes a portion that extends linearly from the curve, which portion extends upwards to reach the lug of the upper strap of the sidewall. In an exemplary embodiment, the spring includes a portion that extends linearly, in a relaxed state, for example, by a distance less than and/or greater than and/or equal to 0.5, 0.75, 1, 1.25, 1.5, 1.75, 2, 2.5, or 3 inches, or any value or range of values therebetween in 0.1 inch increments. FIGS. 26B and 26A show different cross-sections of the spring 630, showing the maximum diameter of the spring of the cross-section, although in other embodiments, again, D261 can be the minimum diameter or the average diameter, over a given length, etc., concomitant with the above.

The idea with these embodiments is that the spring 630 can provide a dorsiflexion assist to the user. In this regard, the spring will drive the foot of the wearer, or more accurately the front of the foot of the wearer, upwards with a force greater than that which would exist from the wearer himself or herself lifting his or her foot alone. Corollary to this is that more effort by the wearer is required to move the foot in the opposite direction. But the idea is that the wearer has a problem with respect to lifting his or her foot relative to moving his or her foot downward (or even if there are problems both ways, the ramifications of that problem are greater with respect to the difficulties lifting the foot upwards as opposed to downward).

The spring 630 can be made of various configurations. In an embodiment, a cross-section normal to the direction of the extension of the spring would have a circular outer profile, while in other embodiments, the profile could be oval shaped or rectangular shaped or any other shape that can have utilitarian value. In an exemplary embodiment, the spring 630 is made of a thermoplastic material. In an embodiment, the spring can be made of a metal or can be a combination of metal and thermoplastic. In an embodiment, a thermoplastic resin is used to make the spring. For example, acetal homopolymer, PETG, nylon, polyethylene, polypropylene, TPE, or any other material that can enable the teachings detailed herein.

In an embodiment, the spring is a torsion spring. In an embodiment, the spring is a leaf spring.

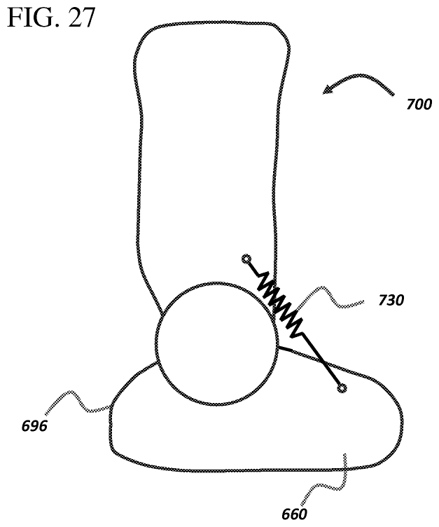

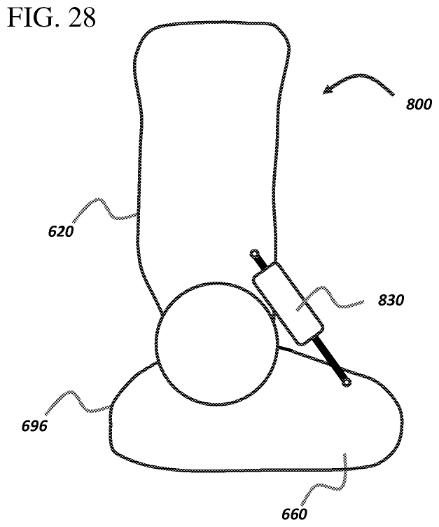

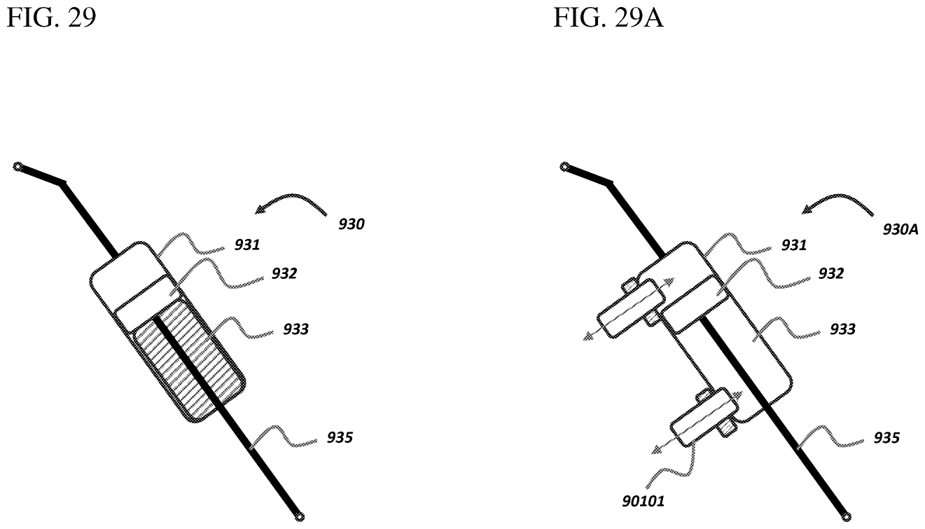

Embodiments are not limited to the spring of FIG. 22 for example. In an alternate embodiment, as seen in FIG. 27, there is a brace 700 that utilizes a pure extension spring 730 as seen. This can be a coil spring as represented in this exemplary embodiment, or could be a spring that deforms in a manner along the lines of a rubber band for example. FIG. 28 shows another exemplary embodiment of a brace 800, that utilizes an internal compression spring (not shown) surrounded by a housing so as to protect the spring from the ambient environment, or ensure that the spring is otherwise isolated from the ambient environment for safety and/or aesthetic appearances. In an embodiment, this could be a piston that compresses an elastomeric material inside the housing. FIG. 29 shows such an arrangement 930, where piston 932 is connected to shaft 935 and an elastomeric material 933 surrounds the shaft (in this exemplary embodiment, while in other embodiments, there could be separate elastomeric bodies located in the housing). Elastomeric material 933 pushes the piston away from the bottom of the housing, thus drawing shaft 935 into the housing 931, and thus pulling the heel enclosure upwards, or at least the front of the heel enclosure upwards, when the energy of the elastomeric material 933 is released. Note also that this exemplary embodiment could be representative of a helical compression spring in an alternate embodiment that is located in a housing, where element 933 can represent the spring. The embodiment of FIG. 29 also shows that the connection points to the brace of the spring need not be exactly in line. Here, a dogleg shaft at the top of the spring is utilized to provide clearance in an embodiment around the female portion of the joint of the brace.

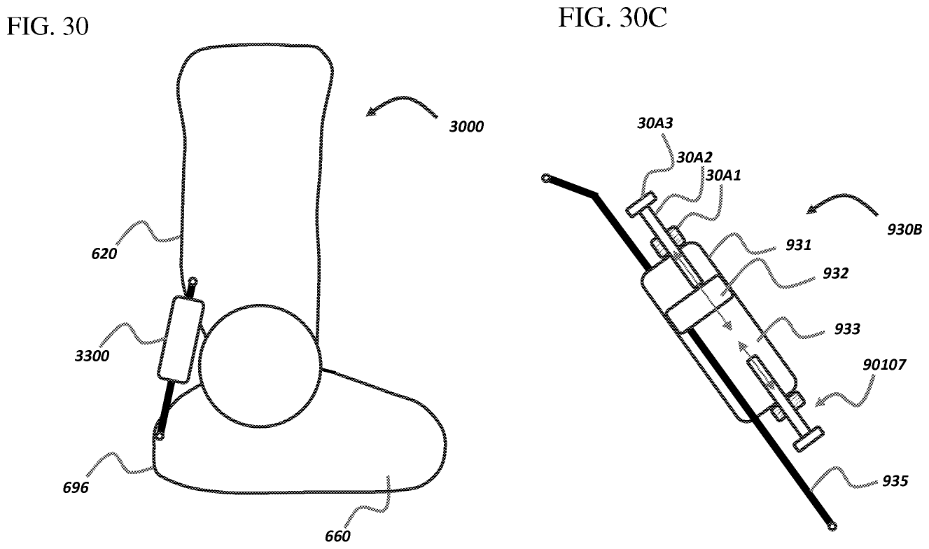

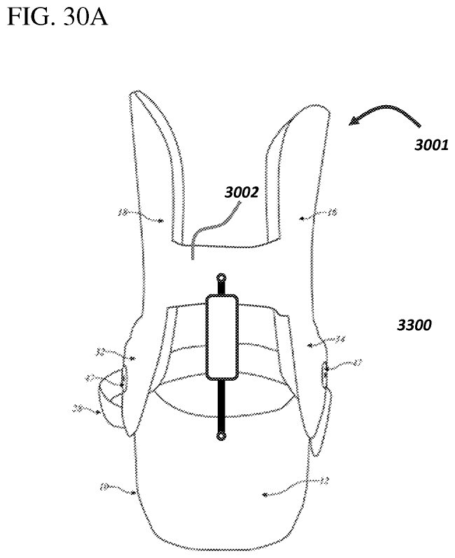

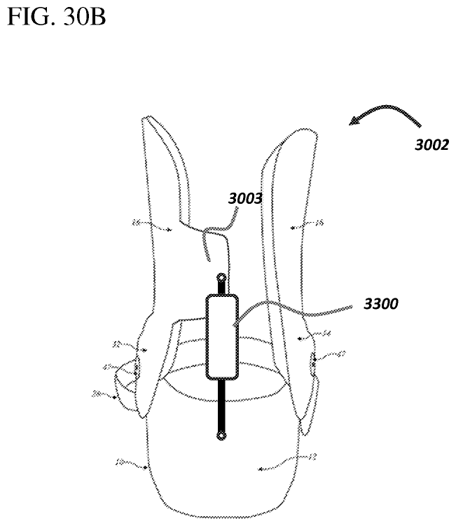

FIG. 30 presents an alternative embodiment of a brace 3000, where the spring assembly 3300, or more generally, the component that provides the force on to the heel enclosure, is mounted at a rear location of the brace. Here, instead of a retracting force or otherwise a force that pulls the bearing lugs towards each other as in the embodiments described above, the force of the spring assembly 3300 expands the distance between the bearing lugs when energy is released by the spring assembly 3300. Note that the springs detailed above could also be utilized in this arrangement, where the spring would desire to expand for example, instead of contract when releasing energy (e.g., spring 630 would increase in diameter when releasing energy/when moving to the unrestrained and/or less restrained state). Any device, system, and/or method that will impart a counterclockwise movement or otherwise will bias the heel enclosure to move in the counterclockwise direction with respect to the frame of reference of FIG. 30 can be utilized in at least some exemplary embodiments providing that the art enables such.

FIG. 30A provides an alternate embodiment where a single spring assembly 3300 is located in the rear of the brace 3001, and is roughly centered in the brace with respect to location between the lateral and medial side. Here, a reaction structure 3002 extends between the uprights, to which the top of the spring assembly 3300 attaches. This can be an extension of the uprights (the uprights here can be monolithic with each other via the reaction structure or the reaction structure can be mechanically attached to the uprights). Note that in an alternate embodiment, the reaction structure 3002 need not extend completely from one of the uprights to the other of the uprights. Instead, the reaction structure can be sufficiently robust that it need not connect to the opposite upright. This can be seen for example in FIG. 30B, where brace 3002 includes reaction structure 3003, which is an overmould of the upright 18 (which can be the case for the embodiment of 30A) or is mechanically fastened to the upright body 18 or is an extension of the basic body establishing such (the latter being depicted in FIG. 30B). Note also that a metal reinforcement can be embedded into and/or bolted onto or otherwise mechanically attached to the reaction structure 3003 and/or into/to the structure establishing the body of the upright 18. Note also that this can be the case with respect to the embodiment of FIG. 30A as well. Note also that reinforcing metal or the like can be embedded or bolted onto the heel enclosure for the reaction point for spring 3300 as well. Indeed, it is noted in the interest of textual economy that reinforcing metal or other reinforcing materials can be utilized to provide or otherwise reinforce any portion of the brace to which the dorsiflexion assist componentry attaches in the interest of textual economy. And note that this need not be a separate material from the material making up the side wall and/or the heel enclosure, etc. In an embodiment, the local material can be “beefed up” to provide sufficient strength to implement the teachings detailed herein. Indeed, with respect to the embodiment depicted in FIG. 22, the forward most bearing 634 is located at the thickened section of the heel enclosure that is utilized for the upper strap attachment point. In fact, the bearing 634 is a rivet that extends through the heel enclosure to secure the portion of the strap that attaches to the heel enclosure in an exemplary embodiment.

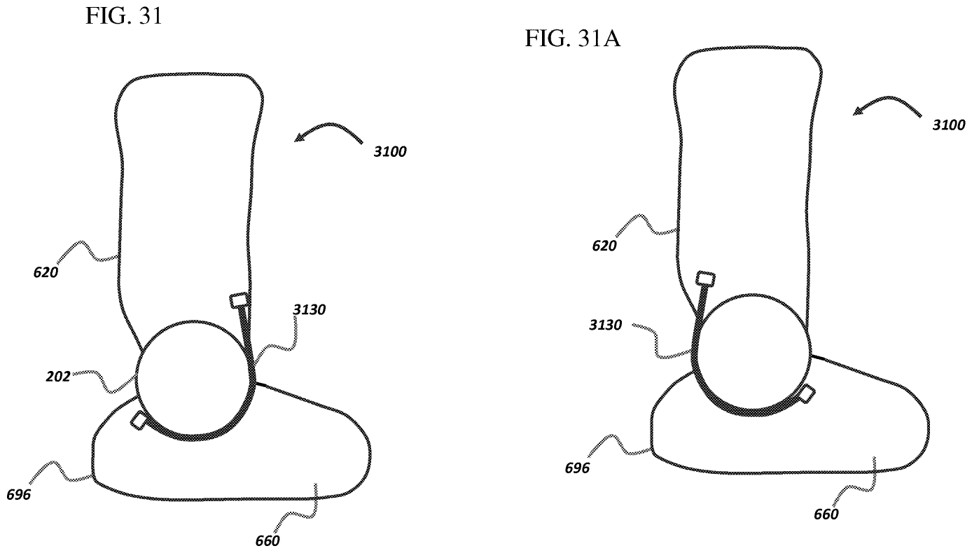

Embodiments have been presented in terms of a device that flexes in a macro sense, embodiments can also utilize devices that operate in pure tension. FIG. 31 shows a brace 3100 that includes an elastomeric component 3130 that is in tension with respect to the alignment of the various components of the brace, where the tension in the elastomeric component 3130 pulls the heel enclosure in the counterclockwise direction. In this exemplary embodiment, elastomeric component 3130 extends about the female portion of the joint 202, so as to impart a moment onto the heel enclosure 696. An elastomeric component is mounted at one end one to the upright 620, and at the other end onto the heel enclosure 696. In an exemplary embodiment, a groove or a track for the elastomeric component 3130 can be molded into the outside of the female portion of the joint 202. In fact, in an exemplary embodiment, almost all of the elastomeric component 3130 that interfaces with the female portion 202 can be enclosed in some embodiments. An opening for where the elastomeric component 3130 extends to the heel enclosure 696 could be provided as utilitarian. In fact, in an exemplary embodiment, the only portion of the elastomeric component 3130 that would be visible or accessible from the outside of the brace would be the portion at the end that interfaces with the heel enclosure and an amount sufficient to allow the lug that attaches the end of the elastomeric component 3130 to the heel enclosure to move relative to the female portion 202.

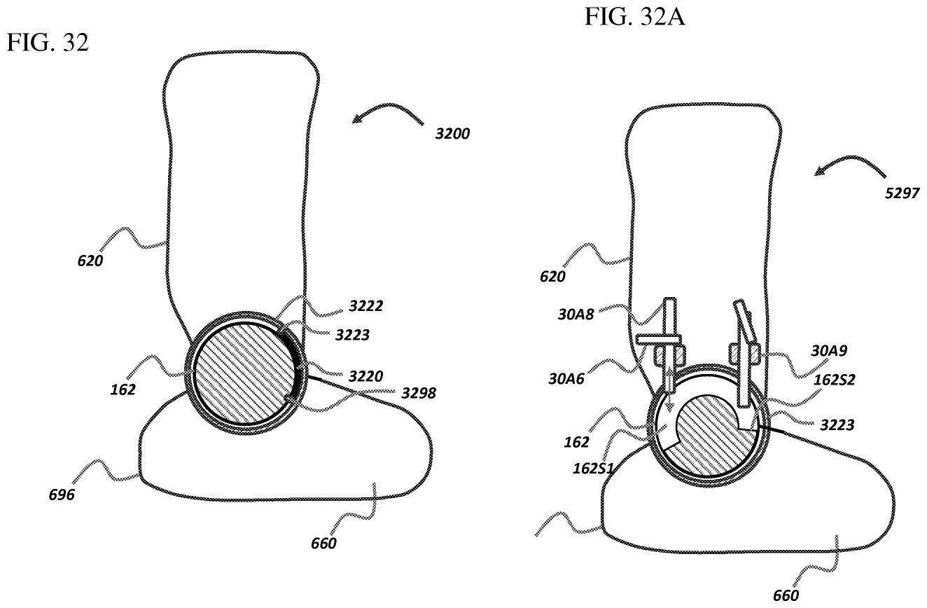

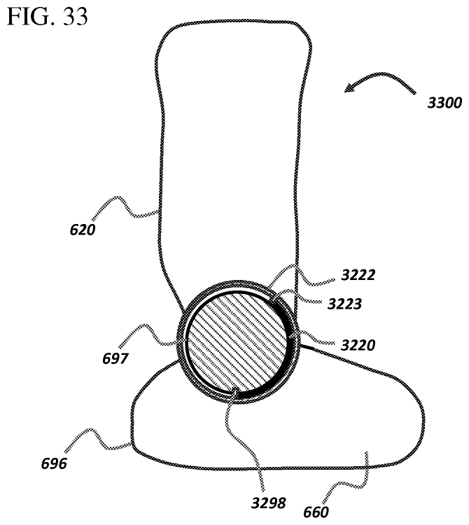

While the embodiment of FIG. 31 depicts the lug that attaches the end of the elastomeric component 3130 on the body portion of the upright 620, in an alternate embodiment, an elastomeric component is attached directly to the female portion 202, and thus does not extend upwards beyond the female portion 202. FIG. 32 presents an exemplary embodiment where the elastomeric component 3220 is completely enclosed with in the brace 3200. Here, a cross-section of the female portion 202 of the joint and the male portion 162 is shown. As seen, there is a lug 3223 that interfaces with the wall 3222 of the female portion of joint 202. The lug 3223 is connected to the elastomeric component 3220, which extends about the male portion 162 of the joint in a curvilinear fashion, to lug 3298, which is embedded in a portion of the male portion 162 of the joint. In the state shown in FIG. 32, elastomeric component 3220 is in tension, and thus applies a moment onto the heel enclosure 696 that would rotate the heel enclosure counterclockwise with respect to the frame of reference of FIG. 32 if the brace was in an unrestrained state. While an elastomeric material is presented in the embodiment of FIG. 32, in another embodiment, an extension spring or a compression spring can be utilized depending on the embodiment and depending on the mounting locations. In an exemplary embodiment, this can be of utilitarian value with respect to completely enclosing the energy absorbing/energy dissipating components of the dorsiflexion assist embodiment. FIG. 33 presents another exemplary brace, brace 3300, with the elastomeric component 3220 has a longer length relative to that of the embodiment of FIG. 32, owing to a different spring constant (or the analogous feature) relative to that of FIG. 32.

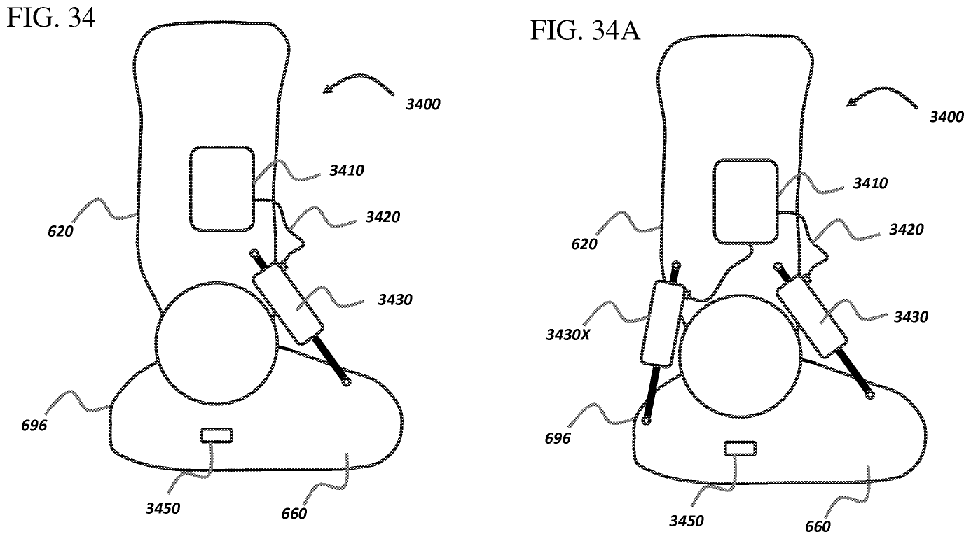

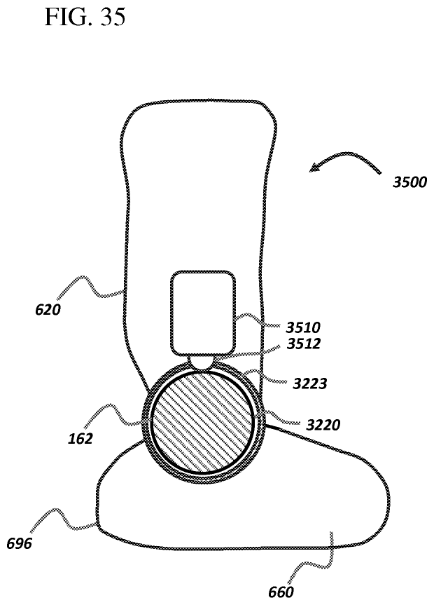

Embodiments associated with passive energy storage and passive energy dissipation devices have been presented. Embodiments also include active energy storage and/or active energy dissipation devices that impart a force onto the heel enclosure 696 relative to or otherwise that is reacted by the upright 620. By way of example, FIG. 34 presents an exemplary brace 3400 that includes a powered actuator 3430. In an exemplary embodiment, the actuator 3430 is configured to retract the bottom shaft and/or the top shaft so as to impart a counterclockwise moment onto the heel enclosure 696 in the frame of reference shown. In this exemplary embodiment, there is a power source and/or control unit 3410 that is in signal communication with the actuator via wires 3420. In an exemplary embodiment, the control unit 3410 includes accelerometers and/or devices that are configured to ascertain directly or via latent variables a spatial orientation of one or more components of the brace. Here, the accelerometers would be ascertaining the spatial relationship of the upright 620 relative to the direction of gravity in an exemplary embodiment. The control unit 3410 can include electronics to receive a signal from the accelerometer, and evaluate that signal to deduce the orientation of the upright 620 relative to the direction of gravity. Based on this deduction, which can be a simple if-then-else circuit or utilize a lookup table of different signal values that correspond to different orientations, the control unit can apply a signal to the actuator 3432 retract the actuator and thus impart the rotation onto the heel enclosure at the desired time and door position. In an exemplary embodiment, the actuator 3430 could be controlled so that little to no force is felt by the wearer of the brace. For example, as the person wearing the brace walks, the actuator 3430 could pull on the side wall with a force that moves the sidewall or otherwise rotates the sidewall, and thus the heel enclosure, relative to the upright 620, in a manner that corresponds to how the foot or more particularly, the shoe, moves relative to the lower portion of the leg as the person walks. That is, the actuator 3430 is simply moving the shoe in a manner that will correspond to how the shoe moves when the person is walking, and thus the person would experience, in some embodiments, little to no sensation that the device is operating to “push” or “pull” the sidewalls, and thus the heel enclosure and thus the shoe, upwards (or downward in other embodiments-embodiments can utilize an actuator that actuates in two directions, to provide a counterclockwise moment and a clockwise moment in an alternating fashion onto the heel enclosure as the person walks).

Embodiments also include the use of sensors, such as accelerometers, on the heel enclosure in addition to and/or as an alternative to utilizing such on the upright. In fact, while the embodiment of FIG. 34 is upright centric with respect to the control unit and sensors, in an embodiment, the control unit and/or sensors can be heel enclosure centric. In any event, FIG. 3400 also depicts an exemplary accelerometer 3450 on the heel enclosure 696. In an alternate embodiment, accelerometer 3450 is located as far forward as possible or otherwise as it is utilitarianly feasible, the idea being that that will provide more sensitive readings of the orientation of the heel enclosure relative to the upright and/or relative to the direction of gravity.

FIG. 35 presents an alternate exemplary embodiment of a brace, 3500, that includes sensor arrangement 3510. Here, sensor arrangement 3510 includes a tooth gear 3512 that interfaces with teeth on the male portion 162 of the joint (teeth not shown). The sensor is fixed to the upright 620, but the gear 3512 rotates with rotation of the male portion 162 as that portion rotates with rotation of the heel enclosure 696 relative to the upright 620. A rotational sensor can be utilized to determine the position or otherwise the number of rotations or the angle of rotation, etc., of gear 3512 as the teeth on the male portion 162 move gear 3512. Alternatively, and/or in addition to this, the sensor arrangement 3510 can evaluate a torque or otherwise a force applied to the gear 3512 and treat that is a latent variable to determine the position of the heel enclosure or otherwise the angular orientation thereof. The sensor can provide input into the control unit of the sensor arrangement 3510, which control unit can correspond to any of those detailed herein or a variation thereof, to determine the relative position and/or the angle of orientation of the heel enclosure 696 relative to the upright 620. Logic circuits can utilize the relative positions to determine when to actuate the actuator and/or how much force to apply on the actuator (the actuator is not shown in FIG. 35). In this regard, sensor 3510 could be in signal communication with the control unit of the embodiment of FIG. 34. Sensor 3510 could utilize a device that determines the position of the shaft that is fixedly mounted to the gear 3512, such as utilizing a varying magnetic field, so as to determine the movement and/or position of the gear 3512, and thus the position of the heel enclosure. Alternatively, and/or in addition to this, an optical sensor can be utilized to “count” the teeth on the gear 3512, or the teeth on the gears could be configured to provide a specific magnetic field so that the orientation of the gear 3512 can be determined, and thus the orientation of the heel enclosure 696 can be determined relative to the upright 620. Any device, system, and/or method of ascertaining the location or the orientation of the rigid heel enclosure 696 relative to the upright 620 and/or vice versa can be utilized in at least some exemplary embodiments providing that the art enables such, unless otherwise noted.

With respect to a control unit, in an exemplary embodiment, there can be circuitry and/or a circuit board or otherwise an arrangement of circuits that results in a logic circuit in total combination that can receive input from sensors or otherwise receive input regarding position or orientation or any other input that can of utilitarian value. The sensors can be accelerometers or position sensors or angular sensors or sensors that can detect the direction of gravity or more accurately the orientation of one or more components relative to the direction of gravity. The sensor's hearing can be magnetic sensors or electric field or magnetic field sensors that evaluate a magnetic field and use information therefrom to make evaluations/determinations or otherwise as input into logic circuits to output a control based on the logic. In this regard, by way of example only and not by way of limitation, the external ankle brace can be configured so that the upper right(s) and/or the heel enclosure include magnets or components that generate magnetic fields and the other can include a sensor that senses the magnetic field, and the magnetic field produced by the various magnets or generators can be specific to an individual magnet, and thus can allow the sensor to detect a field and evaluate that field to determine the position of the heel enclosure relative to the upright based on that magnetic field, where, for example, the magnetic field will be different for different angular orientations of the heel enclosure relative to the upright. By rough analogy, sensors and field generators analogous to that utilized to determine the position with the angular orientation of a shift can be utilized in some embodiments. In any event, an electric and/or optical signal is outputted to the circuits of the control unit, where the circuits can include a microchip or can be broad-based logic circuits, which circuits for example could read a voltage and/or a frequency and/or some other property of the electric signal to deduce the information from the sensor. A lookup table can be utilized where for a given voltage and/or current and/or frequency of an input signal, certain information correlated to such will be present, and the logic circuitry of the control unit will utilize that certain information to control the actuator or otherwise adjust a feature of the active dorsiflexion assist system. Indeed, the control unit can be a closed loop feedback circuit where for a voltage in, the control unit provides a voltage out to the actuator, thus increasing the torque of the actuator or decreasing the torque of the actuator. In an exemplary embodiment, the control unit can be configured so that the control unit applies a voltage to the actuator that is linearly or somehow algorithmically correlated to the voltage input from one or more sensors.

In an exemplary embodiment, strain gauges applied to the surface or otherwise embedded in spring 630 for example can be utilized to provide a signal that can be utilized as a latent variable to indicate the location of the heel enclosure relative to the upright. This can be utilized in other places as well. Any sensor or any sensor relationship, and/or any strain gauge sensor arrangement that can enable the teachings detailed herein or otherwise provide a signal to a logic circuit or some other automated electronic device that can analyze the signal and did use a relative location of the heel enclosure relative to the upgrade, or otherwise control one or more of the actuators detailed herein so as to implement the teachings detailed herein can be utilized in at least some exemplary embodiments.

FIG. 35 can represent an alternate embodiment where gear 3512 is a driven gear. For example, gear 3512 can be mounted to an electric motor or some other motor that rotates the gear 3512 so that the heel enclosure 696 is rotated relative to the upright 620, and thus relative to the leg of the human when the brace 3500 is worn by the human. Thus, apparatus 3510 would be a motor and power arrangement, such as a motor powered by a DC battery, where the output is rotational movement of the gear 3512. That said, in an embodiment, apparatus 3510 can be a combination of a motor and a sensor. In this regard, the position and/or the rotational status of gear 3512 can be monitored by the apparatus 3510 or by another apparatus, and depending on the rotational status of the gear, the motor could impart a rotational force on the gear to rotate the heel enclosure 696 or otherwise impart a moment onto the heel enclosure 696 to implement dorsiflexion assessed in accordance with at least some exemplary embodiments. Here, apparatus 3510 can be configured to energize the electric motor to impart rotational torque or otherwise to rotate here 3512 clockwise with respect to the frame of reference of FIG. 35, so that the gear on the male portion 162 of the joint will rotate in the opposite direction (counterclockwise). In an embodiment, the movements of the gear 3512 are correlated to the orientation of the upright 620 relative to the heel enclosure 696 and/or relative to a walking pattern of the wearer of the brace 3500. In this exemplary embodiment, depending on the orientation, a driving force could be provided to the gear 3512 provided to further move the heel enclosure 696 in the desired direction. For example, if the foot wearing the brace 3500 is located in back of the other foot (which could be determined by direct observation of a system of which the brace is a part or by latent variables, where the upright 620 would be rotated clockwise with respect to the frame of reference of 35 relative to the heel enclosure 696, the motor that turns the gear 3512 can be energized so that a moment in the counterclockwise direction, with respect to the frame of reference of FIG. 35, is applied to the enclosure 696, and thus when the wearer lifts his or her foot to move that foot forward in the walking action, the foot will remain angled upward relative to the lower leg for at least part of the time that the wearer swings his or her foot forward to take the next step, or otherwise the counterclockwise moment will be applied to the heel enclosure so that there is a dorsiflexion assessed for at least a portion of the time that the wearer swings his or her leg forward. It could be that the wearer can overcome this moment so as to move his or her foot downward at the appropriate time as the wearer swings his or her foot forward. It also could be that the arrangement is configured to cease energizing the motor at a certain time so that the wearer of the brace can move his or her foot downward at the appropriate time without having to “fight” the torque applied by the dorsiflexion assist system. This as distinguished from, for example, at least some of the arrangements above where the spring always applies a moment to the heel enclosure, at least when the heel enclosure is moved away from a certain orientation relative to the upright.

In any event, the system just described is a more advanced system relative to some of the other embodiments, and the logic circuitry will be more detailed to implements such, or at least a greater number of sensors or a more sophisticated sensing system will be utilized to determine the position of the foot at issue. Conversely, an arrangement of the brace 3500 where the motor is energized when a counter torque or a sensor determines that the recipient would like to dorsi flex his or her foot, and then is deenergized when the system determines that the wearer would like to move his or her foot in the opposite direction or otherwise does not want or need the assistance (which could be determined by analyzing the current and/or voltage of the power to the electric motor, where the current and/or voltage should fluctuate based on the resistance to movement of the heel enclosure, which would be caused by the wearer trying to move his or her foot in the opposite direction or in the same direction for that matter as that moved by the dorsiflexion assist system) could be implemented with a less sophisticated sensing arrangement and/or logic circuitry.

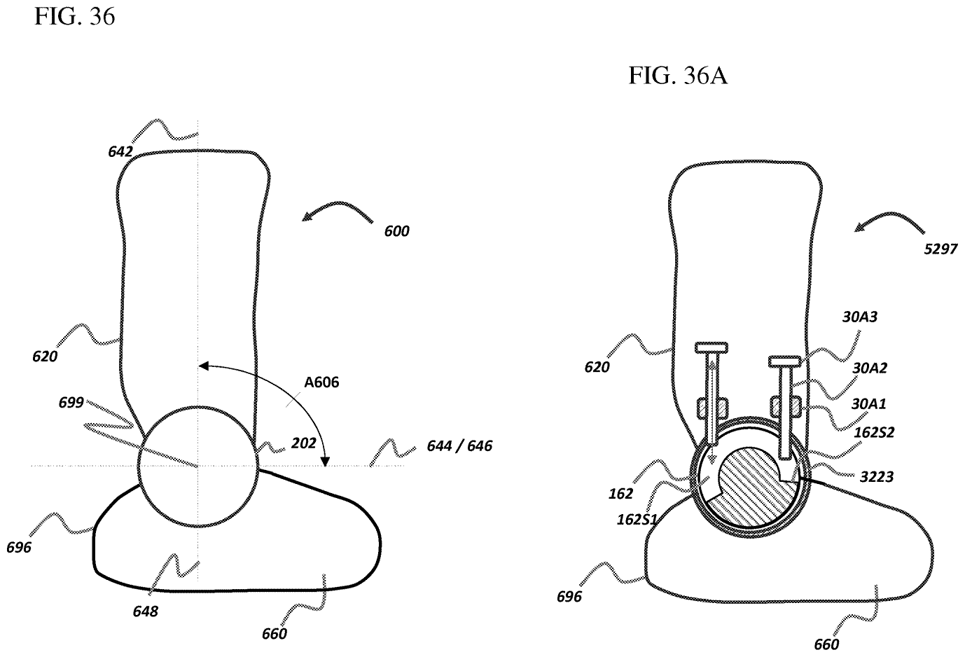

FIG. 36 presents exemplary axes for purposes of discussion. Here, for the upright 620, there is a Y axis 642 and an X axis 644 centered on the axis of rotation of the upright relative to the heel enclosure (axis 699). Also shown for the heel enclosure, is a Y axis 648 and a X axis 646. The Y axis 648 is aligned with axis 642, where the X axis 646 is located at a arbitrary location, here, at the widest point of the side view of the heel enclosure. Providing that the X axis 646 aligned with the longitudinal axis of the heel enclosure, any location of the X axis 646 can be applied. Indeed, these axes are relative to the various components of the brace, and the idea is that for the initial frame of reference, all of the axes will be aligned in a manner that enables the following teachings to be conveyed in a meaningful manner. Indeed, axis 646 can be aligned with axis 644 (they can be coincident with one another).

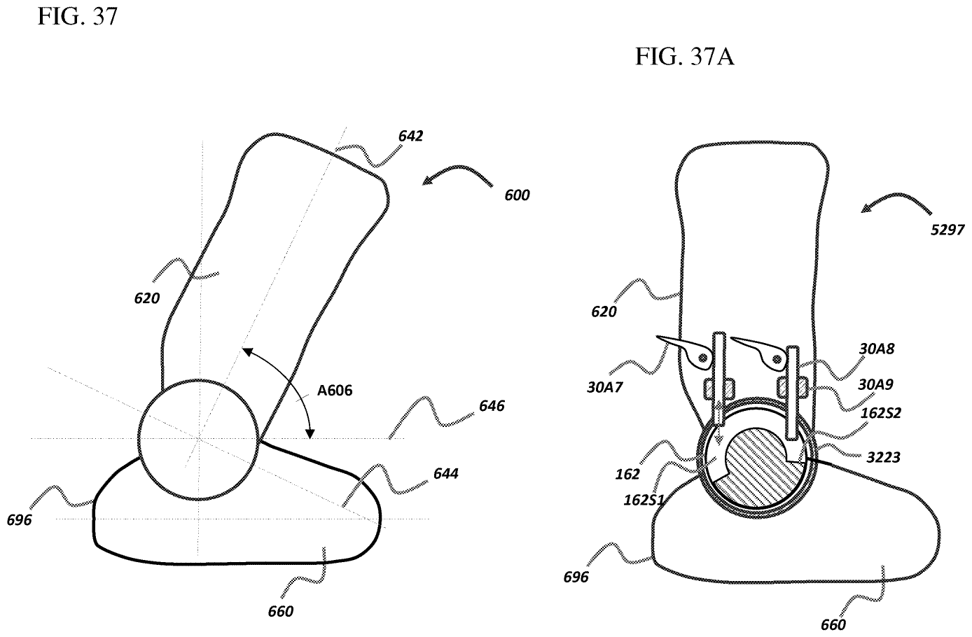

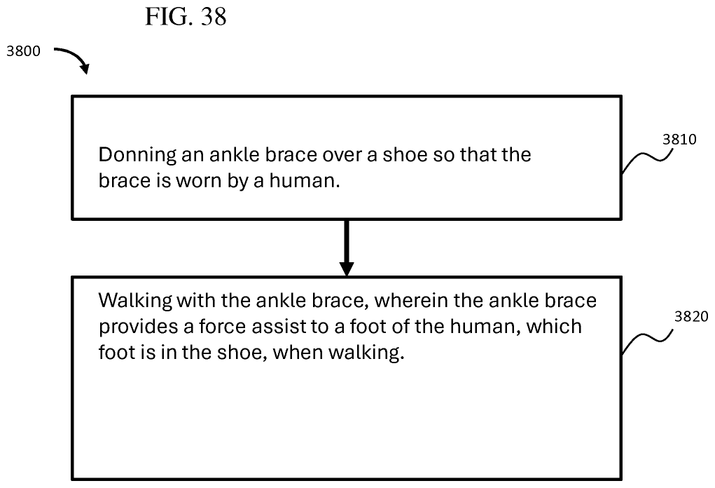

FIG. 37 depicts movement of the upright 620 relative to the enclosure 696, or vice versa for that matter. Here, it can be seen that axis 642 has moved relative to the prior location of that axis. The angle between axis 642 and axis 646 (or the prior location of axis 644 when the upright 620 was in the position of FIG. 36) is angle A606. In an embodiment, a moment applied to the heel enclosure (or to the upright depending on the frame of reference) is less than, greater than and/or equal to 2, 2.5, 3, 3.5, 4, 4.5, 5, 5.5, 6, 6.5, 7, or 7.5 or more or any value or range of values therebetween in 0.05 increments Nm, when A606=90 degrees, and is less than, greater than and/or equal to −1.5, −1, −.5, 0, .5, 1, 1.5, 2, 2.5, 3, 3.5, 4, 4.5 or 5 or more or any value or range of values therebetween in 0.05 increments Nm when A606=45 degrees and is less than, greater than and/or equal to 0.5, 1, 1.5, 2, 2.5, 3, 3.5, 4, 4.5, 5, 5.5 or 6 or more or any value or range of values therebetween in 0.05 increments Nm when A606=65 degrees and is less than, greater than and/or equal to 1, 1.5, 2, 2.5, 3, 3.5, 4, 4.5, 5, 5.5, 6, 6.5, 7, 7.5, 8 or 8.5 or more or any value or range of values therebetween in 0.05 increments Nm when A606=105 degrees.

Below is an exemplary chart for an exemplary arrangement according to an exemplary embodiment, presenting the torque at different angles:

K value (N/mm) 4,868.53

| Angle (from a vertical | | Spring | Moment | |
|---|---|---|---|---|
| axis 642 (where | Chord | Force | Arm | Torque |
| A606 = 90 degrees)) | Length | (N) | (mm) | (N*m) |
| −45.00 | 66.70 | 0.00 | 44.72 | 0.00 |
| −30.00 | 77.87 | 54,381.44 | 40.48 | 2.20 |
| −15.00 | 87.82 | 102,823.27 | 35.38 | 3.64 |
| 0.00 | 96.34 | 144,303.11 | 29.58 | 4.27 |
| 15.00 | 103.26 | 177,993.31 | 23.23 | 4.13 |
| 30.00 | 108.46 | 203,309.65 | 16.45 | 3.34 |
| 45.00 | 111.85 | 219,813.95 | 9.37 | 2.06 |
| 60.00 | 113.35 | 227,116.74 | 2.12 | 0.48 |

K value (pounds/inches) 27.80

| | | Spring | Moment | |
|---|---|---|---|---|
| | Chord | Force | Arm | Torque |
| Angle | Length | (Lbs) | (in) | (in*Lbs) |
| −45.00 | 2.63 | 0.00 | 1.76 | 0.00 |
| −30.00 | 3.07 | 12.23 | 1.59 | 19.48 |
| −15.00 | 3.46 | 23.12 | 1.39 | 32.20 |
| 0.00 | 3.79 | 32.44 | 1.16 | 37.78 |
| 15.00 | 4.07 | 40.01 | 0.91 | 36.60 |
| 30.00 | 4.27 | 45.71 | 0.65 | 29.60 |
| 45.00 | 4.40 | 49.42 | 0.37 | 18.23 |
| 60.00 | 4.46 | 51.06 | 0.08 | 4.26 |

In the interests of textual economy, any of the above numbers can be modified in other embodiments by any value or range of values by 0.2, 0.3, 0.4, 0.5, 0.6, 0.7, 0.8, 0.9, 1, 1.1, 1.2, 1.3, 1.4, 1.5, 1.6. 1.7, 1.8, 1.9, 2, 2.25, 2.5, 2.75, 3, 3.25, 3.5, 3.75, 4, 4.5, 5, 5.5, 6, 6.5, 7, 7.5, 8, or 8.5 times or more or any value or arrange of values therebetween in 0.01 increments.

As seen above, the moment increases with increasing angle up to a point and then begins to decrease. In an embodiment, the transition point of the moment (from increase to decrease) can occur at less than, greater than and/or equal to A606 equals 60, 65, 70, 75, 80, 85, 90, 95, 100, 105, 110, 120, 130, 140, 150, 160 or 170 degrees or more or any value or range of values therebetween in 1 degree increments.

Indeed, owing to the positioning of the bearings 632 and 634, and the kinematics of the upright 620 relative to the heel enclosure (note that herein, sometimes reference to movement is made with reference to one relative to the other-any disclosure of relativeness to one corresponds to a disclosure of relativeness to the other, in the interests of textual economy-note that when walking, it is the heel enclosure that will be considered to articulate relative to the upright(s)) the movement will increase with increasing angle A606 for portion of the sweep and then will decrease in another portion of the sweep with increasing angle A606).

In an exemplary embodiment, the K value of the spring or whatever energy storage/dissipation device that is used is less than, greater than and/or equal to 250, 500, 750, 1000, 1250, 1500, 1750, 2000, 2250, 2500, 2750, 3000, 3250, 3500, 3750, 4000, 4250, 4500, 4750, 5000, 5250, 5500, 5757, 6000, 6250, 6500, 6750, 7000, 7250, 7500, 8000, 8500, 9000, 9500, 10000, 11000, 1200, 13000, 14000 or 15000 or more N/mm or any value or range of values therebetween in 1 N/mm increments.

In an embodiment, using a GDT of a circle with a diameter of 0.025, 0.05, 0.075, 0.1, 0.2, 0.3, 0.4, or 0.5 inches with respect to the upright and the heel enclosure in the configuration shown in FIG. 36, where A606=90 degrees, with respect to cartesian coordinates with X=0 and Y=0 at the intersection of axis 642 and 644, with X=1 inch and Y=1 inch located in the upper right from the intersection, and X=−1 inch and Y=−1 inch being located in the lower left from the intersection, the center of bearing 632 can be within the noted GDC circle with that circle centered at X plus or minus 0, 0.25, 0.5, 0.75, 1, 1.25, 1.5, 1.75, 2, 2.25, 2.5, 2.75, 3, 3.25, 3.5, 3.75 or 4 inches or more (larger values (positive or negative) might utilize an outrigger extending from the upright 620) or any value or range of values therebetween in 0.01 inches, and Y can be −2, 1.75, 1.5, 1.25, 1, 0.75, 0.5, 0.25, 0, 0.25, 0.5, 0.75, 1, 1.25, 1.5, 1.75, 2, 2.25, 2.5, 2.75, 3, 3.25, 3.5, 3.75, 4, 4.5, 5, 5.5, 6, 6.5, 7, 7.5 or 8 inches or more or any value or range of values therebetween in 0.01 inches. Note that for the Y=0, the lug 639 that accepts the bearing would extend away from the center, and would be fixed/sufficiently attached to impart a moment onto the female portion of the joint. The center of bearing 634 can be X plus or minus 0, 0.25, 0.5, 0.75, 1, 1.25, 1.5, 1.75, 2, 2.25, 2.5, 2.75, 3, 3.25, 3.5, 3.75 or 4 inches or more or any value or range of values therebetween in 0.01 inches (again, an outrigger might be used for the “larger” negative values), and with respect to the positive values, 4.25, 4.5, 5, 5.5, 6, 6.5, 7, 7.5 or 8 inches or more or any value or range of values therebetween in 0.01 inch increments.

In an embodiment, the brace is configured to allow angle A606 to be between (inclusive) 30 to 170 degrees or any value or range of values therebetween in 1 degree increments. A606 could be less than 30 degrees or greater than 170 degrees. These values could be with or without the dorsiflexion assist feature. In an embodiment, these values are limited by the interference of the structure of the upright 620 with the structure of the heel enclosure 696, such as, for example, the lug for one of the upper straps of the heel enclosure. And note that these angles may not be the same for the medial upright and the lateral upright, where in at least some exemplary embodiments, the two uprights are configured to articulate independently of one another, but will articulate in an at least somewhat correlated manner owing to the fact that they are both attached to each other by a strap that extends around the leg and otherwise are stationary relative to the leg. In an embodiment, the angles, such as the maximum value or the minimum value, could be limited by the dorsiflexion assist structure or arrangement. Indeed, the embodiment of FIG. 21 could be such that if the heel enclosure is rotated in the counterclockwise direction by a sufficient value where the bearing points of the spring “move past” the rotational axis of the upright and the heel enclosure relative to that shown in FIG. 21, a clockwise moment will then be applied to the heel enclosure 696. Thus, in an exemplary embodiment, there could be a bolt structure that extends from one side of the spring to the other side of the spring, that allows the spring to increase in diameter to a certain amount, but prevents the spring from increasing in diameter past that, to ensure at least reduce the possibility that the heel enclosure could be rotated by that amount.

In any event, in an exemplary embodiment, the moment imparted onto the heel enclosure 696 by the dorsiflexion assist device will increase with increasing value of angle A606. In an embodiment, this increase can be linear with increasing angle. In an exemplary embodiment, this increase can be exponential or geometric (the increase can be an increase in the rate of increase or a decrease in the rate of increase). Indeed, owing to the positioning of the bearings 632 and 634, and the kinematics of the upright 620 relative to the heel enclosure (note that herein, sometimes reference to movement is made with reference to one relative to the other-any disclosure of relativeness to one corresponds to a disclosure of relativeness to the other, in the interests of textual economy-note that when walking, it is the heel enclosure that will be considered to articulate relative to the upright(s)) the movement will increase with increasing angle A606 for portion of the sweep and then will decrease in another portion of the sweep with increasing angle A606). That said, in an embodiment, the moment imparted onto the heel enclosure 696 by the dorsiflexion assist device can be constant and can be something that does not increase with increasing value of angle A606. In an embodiment, the moment is constant relatively constant.

In an embodiment, there is always a moment imparted onto the heel enclosure 696. Such can be the case, for example, in embodiments where, for example, it is the structure of the upright and/or the structure of the heel enclosure that limits the minimum and/or maximum value of the angle A606.

In an exemplary embodiment, the moment imparted onto the heel enclosure increases by 1, 2, 3, 4, 5, 6, 7, 8, 9, 10, 11, 12, 13, 14, 15, 16, 17, 18, 19, or 20%, or any value or range of values therebetween in 1% increments from a baseline value for every 5, 6, 7, 8, 9, 10, 11, 12, 13, 14, 15, 16, 17, 18, 19, or 20 degrees, or any value or range of values therebetween in 1° increments that angle A606 increases, and/or vice versa for decrease in angle A606. The baseline value could be the value of the moment when the angle A606 is the smallest owing to the interference of upright 620 with heel enclosure 696. The baseline value could be the moment when A606 is equal to 90°. In such a case, the moment would instead decrease by any of the aforementioned values with decreasing value A606 from that baseline. The baseline could be at angle A606 equals 30, 35, 40, 45, 50, 55, 60, or 65 degrees, for example.

In an embodiment, the moment imparted onto enclosure 696 at A606 equals 90 degrees is 25 inch-pounds. In an embodiment, the moment imparted onto enclosure 696 at A606 equals 45, 50, 55, 60, 65, 70, 75, 80, 85, or 90 degrees is less than, greater than, and/or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75 or 80 or more inch-pounds, or any value or range of values therebetween in 0.1 inch-pound increments. In an embodiment, the moment increases or decreases from that angle with increasing or decreasing A606, respectively for less, than, greater than and/or equal to 20, 25, 30, 35, 40, 45, 50, 55, 60, 65, 70 or 75 degrees or any value or range of values therebetween in 0.1 degree increments, by any of the just noted amounts depending on the embodiment.

In an exemplary embodiment, the attachment points of the dorsiflexion assist componentry (e.g. the spring 630) to the remainder of the brace can be at any location within 1, 1.5, 2, 2.5, 3, 3.5, 4, 4.5, 5, 5.5, 6, 6.5, 7, 7.5, or 8 inches, or any value or range of values therebetween in 0.05 inch increments from the articulation axis of the pertinent upright and the heel enclosure. In an exemplary embodiment, the dorsiflexion assist componentry does not add any width and/or height and/or length to the brace relative to that which would otherwise be the case in the absence of such componentry. In an exemplary embodiment, a width (e.g., the direction into and out of the page of FIG. 21) of the brace, is increased an amount less than and/or equal to 2, 3, 4, 5, 6, 7, 8, 9, 10, 11, 12, 13, 14, 15, 16, 17, 18, 19, 20, 21, 22, 23, 24, or 25%, or any value or range of values therebetween in 0.1% increments relative to that which would otherwise be the case in the absence of such componentry.

In view of the above, it can be seen that in an embodiment, there is an orthotic, comprising a lower leg interface (e.g., the combination of the uprights and straps detailed herein) and a foot and/or shoe interface (e.g., the heel enclosure and respective straps detailed herein). In an embodiment, the orthotic is configured so that a mechanical force of variable magnitude is applied to the foot interface reacted through the lower leg interface. In an embodiment, this can correspond to the brace 600 where the spring 630 applies a force onto the heel enclosure 696 that is reacted through the upright 620, and in this exemplary embodiment, there can be a spring 630 on the other side of the brace as well. And note again, that in an exemplary embodiment, the componentry that provides the dorsiflexion assist need not be the same on each side of the brace. In an exemplary embodiment, the properties of the spring 630 on the medial side can be different than the properties of the spring on the lateral side. The springs can be completely different, or another type of energy absorbing and/or dissipating device can be utilized on each side. In this regard, any feature of any embodiment detailed herein can be combined with any other feature of any other embodiment detailed herein providing that the art enables such, unless otherwise noted, in the interest of textual economy. In an exemplary embodiment, the orthotic includes a spring, where the spring provides the mechanical force. In an embodiment, the spring is a leaf spring while in other embodiments the spring is a torsion spring. In an embodiment, the spring is a compression spring or an extension spring. In an embodiment, the spring is a linear force spring or otherwise has a linear K value. In an exemplary embodiment, the orthotic applies a torque to the foot and/or shoe interface reacted by the lower leg interface.

In an embodiment, the spring on one side of the brace has a K value of a first value, and the spring on the opposite side of the brace has a K value of the second value that can be greater than or less than the K value on the one side of the brace. This can have utilitarian value with respect to minimizing the sizes of the springs or otherwise controlling the sizes to fit within a certain space regime. For example, the medial side of the brace would potentially be more desirable to be “streamlined” or compact relative to the lateral side of the brace so as to avoid interfering with the other shoe when walking for example. Conversely, it could be that the lateral side of the brace would potentially be more desirable to be streamlined so as to avoid catching the spring for example, on something, where a scenario for catching the spring on something would be less likely to exist with respect to the medial side of the brace. And note further that in an exemplary embodiment, a shroud can be placed over the spring on one or both sides so as to reduce the likelihood of the spring catching on something. In an embodiment, the shroud completely covers the spring. In an embodiment, the shroud increases the width of the brace by no more than any of the amounts detailed above by way of example in the interest of textual economy.

The fact that there is a dorsiflexion assist component on both sides of the brace can be utilized to provide a different degree of torque onto the heel enclosure depending on the angle. In an embodiment, the spring on the medial side can be a spring that will provide increasing force with rotation of the enclosure up to a certain angle (say A606 equals 70 degrees), and then will stop increasing the force resistance. In an embodiment, this can be a result of a mechanical connection that disengages at that certain angle. For example, the bearing 632 could have access to a track that is angled so that when the upright 620 reaches a certain angle, the bearing is forced into that track and thus the bearing can move relative to the uprights 620 so that further rotation of the upright 620 does not move the bearing 632. Conversely, as the upright 620 is rotated back towards the given angle, the bearing 632 will move in reverse along the track until it for example reaches the end of the track, and then becomes fixed relative to the upright 620. In this exemplary embodiment, the track is curved in a manner so that there is always some force that is applied by the bearing 632 to the upright 620. In this exemplary embodiment, the force applied to the upright and thus the enclosure is a constant force after the angle is reached, but in another embodiment, the force can be variable thereafter as well, but varying in a different manner than before, thus limiting or otherwise changing the amount of force that the spring imparts onto the upright, and thus limiting or otherwise changing the moment that is applied to the heel enclosure relative to that which would otherwise be the case.

In an embodiment, the structure of the brace can be utilized to effectively change the K value of the spring. For example, with respect to the embodiment described above, the bearing 632 and/or bearing 634 can be located in a curved track for example, or a track that has a curve section and a linear section, where the bearing can move or otherwise is forced to move with rotation of the heel enclosure relative to the upright. As the angle increases or decreases, the bearing can move within the track, at least for some angles. With reference to the spring 630 for example, this will vary the change in diameter of the spring relative to that which would otherwise be the case over some or all of these angles. In this regard, the K value of the spring is related to the diameter of the spring or for example the distance from bearing 632 to bearing 634. Changing how that distance changes with a change in angle A606 will thus change how the spring deforms, and thus will change how the force varies with increasing or decreasing A606.

Consistent with the teachings above, the mechanical force automatically pushes and/or pulls a portion of the shoe interface upward when the orthotic is worn by a human and the shoe is lifted completely off the ground into the air.

In an embodiment, there is an ankle brace, comprising a human interface structure (the uprights and the heel enclosure—this is a human interface even if clothing (pants/shoe/sock) is interposed between the human tissue and the brace). The brace also includes an energy storage device (e.g., spring, or battery, etc.) In an embodiment, the ankle brace is configured so that the energy storage device moves (directly in the case of the spring, and indirectly in the case of using the battery to power the actuator) a position of the foot of the human when the ankle brace is worn by the human when the energy storage device discharges energy.

In an embodiment, the energy storage device is charged by walking of the human wearing the brace (e.g., the spring is expanded against its K value for plantar flexion movement, or a piezoelectric device charges the battery, or an inertial mass device charges the battery, etc.). The energy storage device can be charged by movement of the foot. In an embodiment, the energy storage device accumulates energy when the foot moves in plantar flexion. In an embodiment, the energy storage device discharges energy at a constant rate and/or at a variable rate (e.g., with angle change A606) to move the position of the foot and/or apply a force and/or moment to the shoe to move the position of the foot or to provide a force to the foot/shoe that would move the foot/shoe unless an equal or greater force (or moment) is provided to resist such. The energy storage device can move or impart a force to move the position of the foot in dorsiflexion when the energy storage device discharges energy.

Embodiment can be configured so that a force applied to one of the bearings of the spring is less than, greater than and/or equal to (or can be considered a maximum in some instances) 3, 4, 5, 6, 7, 8, 9, 10, 11, 12, 13, 14, 15, 16, 17, 18, 19 or 20 or more pounds or any value or range of values therebetween in 0.1 pound increments, when angle A606 is 45, 50, 55, 60, 65, 70, 75, 80, 85, or 90 degrees, or any value or range of values therebetween in 0.1 degree increments (presented in the interests of textual economy). In an embodiment, the drawings are to be considered to scale.

In an embodiment, the distance between (linear direct line) the center of bearing 632 and the center of bearing 634 changes by less than, greater than, and/or equal to 15, 20, 25, 30, 35, 40, 45, 50, 55, or 60% or more, or any value or range of values therebetween in 0.1% increments when the upright is moved from A606 equals 35, 40, 45, 50, 55 or 60 degrees to A606 equals 120 degrees. In an embodiment, the distance between those centers when the brace is at A606 equals 60 degrees is 1.5, 1.6, 1.7, 1.8. 1.9, 2, 2.1, 2.2, 2.3, 2.4, 2.5, 2.6, 2.7, 2.8, 2.9, or 3.0 inches, or any value or range of values therebetween in 0.05 inch increments when A606 equals any one of those initial degree values just noted.

In an embodiment, there is a foot orthosis, comprising a first apparatus configured to grip a shoe worn by a human, and a second apparatus configured to grip a leg of the human (which includes gripping the leg with a pants and/or sock between the tissue of the human and the second apparatus), wherein the first apparatus articulates relative to the second apparatus when the human walks using the foot orthosis. In an embodiment, the foot orthosis is configured to apply a moment to the first apparatus about an axis of articulation. As seen above, the foot orthosis biases a position of the first apparatus relative to another position, which positions can be any of the angles noted above. In some embodiments, the foot orthosis is configured so that the first apparatus articulates through at least 10, 15, 20, 25, 30, 35, 40, 45, 50, 55, 60, 70, 80, 90, 100, 110, 120, 130, 140, or 150 degrees, or any value or range of values therebetween in 0.1 degree increments relative to the second apparatus, and this articulation can be with a force that is constantly applied (as opposed to a constant force—the force, which can be variable, is present) to the first apparatus reacted by the second apparatus over that range of articulation. In an embodiment, the force varies linearly or varies within 5, 10, 15, 20, 25, 30, 35, or 40% (plus or minus), or any value or range of guys therebetween in 1% increments of a linear variation (using the starting force/the force with the spring or whatever device is used to impart the moment in the most relaxed state of the brace and/or the value at the A606 equals 45° or 60° or 90° so as to establish a reference for the calculations) over that range. In an embodiment, the foot orthosis is configured so that the first apparatus articulates through a range that is at least and/or equal to and/or no more than 90, 80, 70, 60, 50, 40, 30, 20, or 10%, or any value or range of values therebetween in 1% increments of the just noted angular range with the force variation just noted. For example, the force could vary linearly or within a range of ±15° from linearly for 67.5° (75% of) 90° and then could vary in a different manner, such as geometrically, etc. In an embodiment, in the interest of textual economy, a nonlinear variation and/or a variation that varies from linearity by more than 15, 20, 25, 30, 35, 40, 45, 50, 55, 60, 65, 70, or 80% (plus or minus), or any value or range of values therebetween in 1% increments utilizing the at rest or most relaxed position or the position where angle A606 equals 45° or the 60° or the 90° as the basis to determine the percentage variation can exist for any of the remainder of the angular ranges or can exist for those ranges instead of the values initially detailed.

As noted above, there can be a structural stop that limits the articulation in one or both directions, and the structural stop can be such that the force remains even when the heel enclosure cannot articulate any further relative to the upright. Alternatively, in an embodiment, the force could be reduced to zero prior to any “bottoming out” of the articulation, and in some embodiments, a force in the opposite direction could start to build before the articulation bottoms out.

In an embodiment, the foot orthosis is configured so that the first apparatus articulates through at least 90 degrees relative to the second apparatus and when the orthosis is worn by the human, the angle of articulation is limited to less than 90 degrees owing to the anatomy of the human. In an embodiment, first apparatus articulates through at least any of the values just detailed in the interest of textual economy, relative to the second apparatus, and when the orthosis is worn by the human, the first apparatus articulates by an angle that is no more than 20, 25, 30, 35, 40, 45, 50, 55, 60, 65, 70, or 80%, or any value or range of values therebetween in 1% increments of the maximum articulation through which the first apparatus can articulate relative to the second apparatus, owing to the anatomy of the human, in the interest of textual economy.

In some embodiments, when the orthosis is at rest and relieved of all external forces, a longitudinal extension of the first apparatus is angled relative to a longitudinal extension of the second apparatus (e.g., angle A606 in the embodiments above) within an angle of 30 to 60 degrees. In an embodiment, the angle is less than, greater than, and/or equal to 10, 15, 20, 25, 30, 35, 40, 45, 50, 55, 60, 65, 70, 75, 80, 85, 90, 95, 100, 105, or 110 degrees, or any value or range of values therebetween in 1 degree increments. In an exemplary embodiment, the foot orthosis is configured to articulate through any of the values or range of values detailed herein, thus subsuming the aforementioned values. In an exemplary embodiment, the first apparatus is configured to articulate relative to the second apparatus by an angle that is greater than and/or equal to 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, or 5 or more, or any value or range of values therebetween in 0.05 increments times the aforementioned angles.

In an exemplary embodiment, the moment is applied by a torsion spring or any of the springs detailed herein, and this torsion spring or any of the springs detailed herein always subtends an angle that is no more than 350, 340, 330, 320, 310, 300, 290, 280, 270, 260, 250, 240, 230, 220, 210, 200, 190, 180, 170, 160, 150, 140, or 130 degrees, or any value or range of values therebetween in 1° increments.

Embodiments include an external ankle brace (as opposed to a brace worn inside a shoe, or at least a portion thereof being inside a shoe), that includes a medial lower leg section configured to interface with at least a medial side of a leg of a human at a lower leg portion thereof, the medial lower leg section including a medial rigid body contoured to a medial side of a lower leg of a human and a lateral lower leg section configured to interface with at least a lateral side of a leg of a human at a lower leg portion thereof, the lateral lower leg section including a medial rigid body contoured to a medial side of a lower leg of a human. The brace also includes a foot section configured to interface with a shoe worn on a foot of the human at locations where the foot of the human is located. Consistent with some of the embodiments above, the foot section is biased to move relative to at least one of the medial lower leg section or the lateral lower leg section. In an embodiment, the foot section is spring loaded relative to at least one of the medial lower leg section or the lateral lower leg section. In some embodiments, concomitant with the teachings above, the brace is configured so that the foot section pivots in a first direction and prevents the foot section from pivoting in a second direction normal to the first direction. The foot section pivots in the one direction and the direction opposite the one direction as seen above in some embodiments.

In some embodiments, the brace is configured so that the foot section pivots in a first direction and prevents the foot section from pivoting in a second direction normal to the first direction and a third direction opposite to the second direction. The brace can be configured so that the foot section pivots about an axis extending through the lateral lower leg section and the foot section is spring loaded relative to the at least one of the medial lower leg section or the lateral lower leg section, which spring loading applies a moment to the foot section to bias the foot section. The brace can be configured so that the foot section pivots about an axis extending through the lateral lower leg section and the spring loading causes the foot section to more easily pivot in one direction than a direction opposite the one direction. In an embodiment, the foot section pivots in the one direction and the direction opposite the one direction.

Embodiments include methods. FIG. 38 presents an exemplary flowchart for an exemplary method, method 3800, that includes method action 3810, which includes the action of donning an ankle brace over a shoe so that the brace is worn by a human. Method 3800 also includes method action 3820, which includes the action of walking with the brace while the shoe is in the brace. In an exemplary embodiment, the brace grips the shoe in a manner concomitant with the teachings above, such as by way of example, because the upper strap is tightened and/or the lower strap is tightened so that the sidewalls gripped the sides of the shoe. In this exemplary embodiment, the brace provides a force assists to a foot of the human, which foot is in the shoe, when walking.

In an exemplary embodiment, method 3800 includes any one or more actions that will result in any one or more of the features detailed above with respect to the dorsiflexion assist being implemented or otherwise associated with such, providing that the art enables such, unless otherwise noted.

In an exemplary embodiment, the force assisted method action 3820 mitigate at least in part group foot of the human. In an embodiment, the force assist biases the foot to dorsiflexion. In an exemplary embodiment, the person executing method action 3820 has suffered a stroke and cannot walk with a normal gait or otherwise has difficulty walking relative to a 50 percentile human factors engineering quantification for a person of that age and/or that weight and/or that sex. In an embodiment, the method is a strength training method. In an embodiment, the method is a habilitation method or a rehabilitation method

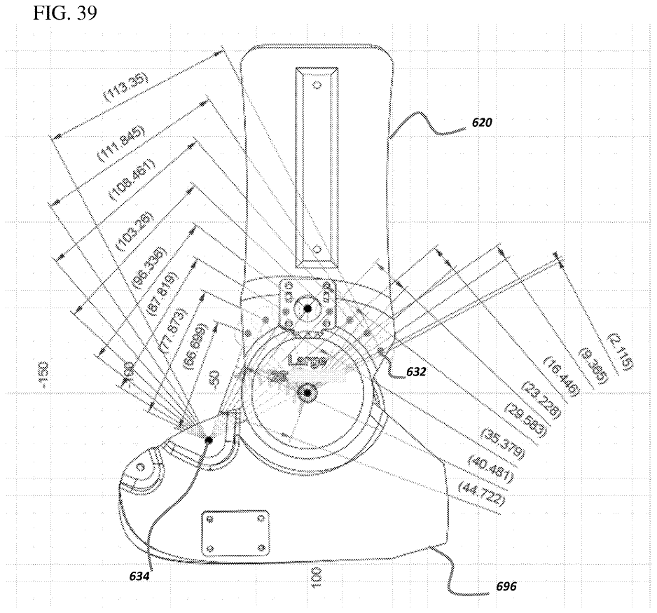

FIG. 39 presents exemplary dimensions by way of example showing how the distance of the line between bearing 634 and bearing 632 from the axis of rotation/pivoting axis of the upright relative to the heel enclosure and the distance between these two bearing points changes over different angles of movement of upright 620 about the pivot axis, where the distance is measured in millimeters. And in this exemplary embodiment, these distances are to scale. In an exemplary embodiment, these can be 10° increment angle changes. As seen, the distance between the two bearing points will increase and then decrease over a sweep of movement of upright 620 in a clockwise direction about the pivot axis. In an embodiment, the distance between the two bearing points (center thereof) falls within the above-noted GDT circle centered at a distance of less than, greater than and/or equal to 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290 or 300 mm or more or any value or range of values therebetween in 1 mm increments, at angle A606 equals 45, 60, 75, 90, 105, 120 or 135 degrees or any value or range of values therebetween in 1 degree increments. In an embodiment, the closest that the line between the two bearing center axes within the noted GDT circles at any one or more of the just noted angles can be less than, greater than and/or equal to 20 5, 10, 15, 20, 25, 30, 35, 40, 45, 50, 55, 60, 65, 70, 75, 80, 85, 90, 95, 100, 105, 110, 115, 120, 130, 140, 150, 160, 170 or 180 mm or more or any value or range of values therebetween in 1 mm increments.

### D

Embodiments have focused on dorsiflexion assist devices and methods. In an alternative embodiment, exclusionary or in addition to the dorsiflexion assist, there can be a plantar flexion assist devices. In an embodiment, such devices would work in the opposite manner as those detailed above with respect to the dorsiflexion assist. Instead of creating or otherwise imparting a torque onto the heel enclosure 696 that tends to drive the distal end of the enclosure upwards, a torque is created or otherwise imparted onto the heel enclosure that tends to drive the distal end thereof downwards. Any disclosure herein with respect to dorsiflexion assist corresponds to an alternate disclosure of plantar flexion assist as would be modified to achieve such providing that the art enables such, unless otherwise noted.

And note that in embodiments, there can be both dorsiflexion assist and plantar flexion assist, and the two need not operate in the exact same manner. For example, the moment applied to the heel enclosure for one for a given angle of change or any other feature detailed herein could be different than that applied in the opposite direction for the other, providing that the art enables. Accordingly, in an embodiment, there can be two separate devices. By way of example, there could be two different springs that have two different spring constants, and the springs can be engaged and/or disengaged based on the direction of movement of the heel enclosure so as to result in the different torques in the different directions.

In an embodiment, the moment imparted onto the heel enclosure 696 by the plantar flexion assist device will increase with decreasing value of angle A606. In an embodiment, this decrease can be linear with decreasing angle. In an exemplary embodiment, this i decrease can be exponential or geometric (the increase can be an increase in the rate of increase or a decrease in the rate of increase). Indeed, owing to the positioning of the bearings 632 and 634, and the kinematics of the upright 620 relative to the heel enclosure (note that herein, sometimes reference to movement is made with reference to one relative to the other-any disclosure of relativeness to one corresponds to a disclosure of relativeness to the other, in the interests of textual economy-note that when walking, it is the heel enclosure that will be considered to articulate relative to the upright(s)) the movement will decrease with decreasing angle A606 for portion of the sweep and then will increase in another portion of the sweep with decrease angle A606). That said, in an embodiment, the moment imparted onto the heel enclosure 696 by the plantar flexion assist device can be constant and can be something that does not decrease with decrease value of angle A606. In an embodiment, the moment is constant or relatively constant.

In an embodiment, there is always a moment imparted onto the heel enclosure 696 upward or downward. Such can be the case, for example, in embodiments where, for example, it is the structure of the upright and/or the structure of the heel enclosure that limits the minimum and/or maximum value of the angle A606.

In an exemplary embodiment, the attachment points of the plantar flexion assist componentry (e.g. the spring 630, but the spring could be such that the spring wants to expand (the two ends move away from each other) to the remainder of the brace can be at any location within 1, 1.5, 2, 2.5, 3, 3.5, 4, 4.5, 5, 5.5, 6, 6.5, 7, 7.5, or 8 inches, or any value or range of values therebetween in 0.05 inch increments from the articulation axis of the pertinent upright and the heel enclosure. In an exemplary embodiment, the plantar flexion assist componentry does not add any width and/or height and/or length to the brace relative to that which would otherwise be the case in the absence of such componentry. In an exemplary embodiment, a width (e.g., the direction into and out of the page of FIG. 21) of the brace, is increased an amount less than and/or equal to 2, 3, 4, 5, 6, 7, 8, 9, 10, 11, 12, 13, 14, 15, 16, 17, 18, 19, 20, 21, 22, 23, 24, or 25%, or any value or range of values therebetween in 0.1% increments relative to that which would otherwise be the case in the absence of such componentry.

Indeed, in an embodiment, there can be two separate springs 630 on opposite sides of the brace, where one is configured to be biased so that the ends are pulled towards each other, thus imparting a moment onto the heel enclosure that moves the distal end upwards, and the other being configured to be biased so that the ends push away from each other, thus imparting a moment that moves the distal and downwards. And FIG. 21A shows an exemplary brace 600PF where spring 630 is reversed from that of FIG. 21, and thus this is a plantar flexion assist (if FIG. 21 is dorsiflex assist—if FIG. 21 is plantar flexion assist (the ends of the spring want to move away from each other), then FIG. 21A is dorsiflexion assist. And in an embodiment, there can be two springs on the same side, such as seen in FIG. 21B, where there is a spring 639DF that extends about spring 630. One can be for dorsiflexion and the other for plantar flexion, and there could be an engagement system that variously locks and unlocks one of the two ends of the different springs as the wear is walking/running to coincide with the desired movement of the foot. In an embodiment, there can be a mechanical/electric actuator that locks and unlocks the ends.

Alternatively and/or in addition to this, there could be an electromechanical actuator that pushes and/or pulls the ends away/towards each other, to counteract the spring force so that plantar flex and/or dorsi flex assist is alternatingly obtained.

Note that instead, spring 730 could be an extension spring to have plantar flex assist. Device 3300 or 830 could be an actuator that can be controlled to provide plantar flex assist and dorsi flex assist. And element 3130 could be moved to the other side of element 202 to obtain the different assist, as seen in FIG. 31A. Any of the embodiments herein that can be modified in an enabled manner to obtain the alternate plantar flex can be modified unless otherwise noted.

In an embodiment, the structure of the brace can be utilized to effectively change the K value of the spring, and this can be used to alternate between dorsiflex and plantar flex as needed, including during a single step. For example, a spring constant could be increased to provide dorsiflexion assist and then decreased when the brace is providing plantar flexion assist whereas another spring can be operated in the opposite manner. That said, one spring can be held constant, and the other spring can be varied so that the spring constant is greater than the other spring constant or less than the other spring constant (where for example, the spring constant can be dropped to near zero or zero thus effectively negating the spring force for one).

Consistent with the teachings above, the mechanical force automatically pushes and/or pulls a portion of the shoe interface upward when the orthotic is worn by a human and the shoe is lifted completely off the ground into the air and/or pushes and/or pulls downward when completely lifted off the ground.

Embodiments that include the active energy storage and/or active energy dissipation devices that impart a force onto the heel enclosure 696 relative to or otherwise that is reacted by the upright 620 can be used for dorsiflexion and/or plantar flexion. By way of example, FIG. 34 presents an exemplary brace 3400 that includes a powered actuator 3430 as noted above. In an exemplary embodiment, the actuator 3430 can be configured to extend the bottom shaft and/or the top shaft so as to impart a clockwise moment onto the heel enclosure 696 in the frame of reference shown. In this exemplary embodiment, there is a power source and/or control unit 3410 that is in signal communication with the actuator via wires 3420. In an embodiment, the actuator can be used for both plantar flexion and dorsiflex assist. The actuator can thus be a two-way actuator that can cause the ends to extend and can also cause the ends to retract, thus imparting clockwise rotation and counterclockwise rotation on the heel enclosure, respectively, in the frame of reference shown. Alternatively, recognizing that devices tend to be less robust when working in tension, the actuators can be actuators that contract only when energized, and thus there could be two actuators for example, as seen in FIG. 34A, where there is actuator 3430, which retracts for dorsiflexion assist, and actuator 3430X, which retracts for plantar flex assist.

The device of FIG. 35 can also be used to implement dorsi flex and/or plantar flex assist, as where the embodiment is such that gear 3512 is a driven gear. For example, gear 3512 can be mounted to an electric motor or some other motor that rotates the gear 3512 so that the heel enclosure 696 is rotated relative to the upright 620, and thus relative to the leg of the human when the brace 3500 is worn by the human. Depending on which way the gear 3512 is rotated, there would be plantar and dorsi flex assist. Embodiments thus include the arrangement of FIG. 35 used to implement dorsi and/or plantar flex assist, and there could be two separate actuators on either side, one for dorsi flex assist and the other for plantar flex assist, so that voltage modification need not be implemented to reverse the direction of the wheels, although in other embodiments, bi-directional powered rotation can reduce the size needed to implement a given moment/torque.

Embodiments can include logic circuitry will be more detailed to implements such, or at least a greater number of sensors or a more sophisticated sensing system will be utilized to determine the position of the foot at issue. Conversely, an arrangement of the brace 3500 where the motor is energized when a counter torque or a sensor determines that the recipient would like to plantar flex his or her foot, and then is deenergized when the system determines that the wearer would like to move his or her foot in the opposite direction or otherwise does not want or need the assistance (which could be determined by analyzing the current and/or voltage of the power to the electric motor, where the current and/or voltage should fluctuate based on the resistance to movement of the heel enclosure, which would be caused by the wearer trying to move his or her foot in the opposite direction or in the same direction for that matter as that moved by the dorsiflexion assist system) could be implemented with a less sophisticated sensing arrangement and/or logic circuitry. And note that the logic circuitry can detect when plantar flex is to be executed and then default to dorsi flex, and/or visa-versa. Two way logic can be implemented if desired that detects plantar flex desire and dorsi flex desire by the recipient.

Note that in an embodiment, a gait monitor can be used, such as a device according to a Fit Bit or the like that measures/monitors steps, and such can use an algorithm to estimate when dorsi flex and/or when plantar flex is desired or will happen. In an embodiment, the data obtained from such or using a system/device/and/or method/algorithm based on such where such can be used to estimate or otherwise serve as a latent variable for when the foot will be in dorsiflexion or when the wearer once such to be the case and or for when the foot will be in plantar flex or when the user once such to be the case (where “wants” has a potentially sub conscious connotation-a person wants to inhale and exhale in a given manner per normal, one does not think about inhaling or exhaling when typically breathing, and such is the case with respect to walking or running—the person wants to run or walk normally, or at least as normal as possible, and thus the system can estimate or otherwise reasonably calculate when the foot will be in plantar flex or dorsiflexion). By way of example only and not by way limitation, if the control system or otherwise the computing system receives input from a smart phone or a smart bit or a fit bit, or otherwise a device that is integrated with the brace that utilizes such an algorithm or equivalent algorithm or equivalent system, or any other system that can enable the number of steps to be calculated or otherwise estimated per time in real-time or near real-time, indicative of when the user is placing his or her foot down onto the ground in the typical manner of walking, the system can be correlated to determine that in close proximity to such indication, the foot will be in dorsiflexion, and then a period of time after such, the foot will be in plantarflexion, or alternatively, the data can be based on the foot on the opposite side where the impact on the ground is indicative of the other foot being removed from the ground within a certain time thereafter or at that time for that matter, and if the scenario is running, it can be estimated how much before that time for that matter, and then the assist features can be implemented in a temporally correlated manner. Still, that said, integrated accelerometers could be utilized and/or sensors that can determine when a torque is imparted onto the heel enclosure due to walking or running, and one such torque is relieved or otherwise reduced relative to a maximum or some other control, the assist can be implemented.

Embodiments detailed above have been directed towards the utilization of the actuators and springs, etc., for dorsiflexion assist and/or plantar flex assist. The idea being that the brace provides a moment in the direction of the given flex. In an alternative embodiment, the teachings detailed herein can be utilized to provide resistance training or the like. This is a counterintuitive approach from dorsiflex and plantar flex assist. Here, this can build strength in the muscles of the user's foot, etc. This can also build stamina or endurance or better control of one's foot, which could be utilitarian after an event such as a stroke or the like. This can be one way/one direction or two ways, concomitant with the embodiments above for the flex assist, but in the opposite direction. Any of the forces, timing, moments, etc., appliable, for flex assist, can be presented in reverse for flex resistance/training. Embodiments thus include devices and systems that apply the forces and moments detailed herein in the opposite direction. Thus, embodiments can be considered to include dorsiflexion resistance and plantar flexion resistance systems. Embodiments thus include methods of walking and/or running wearing the various braces detailed herein or variations thereof where the brace imparts a moment onto the shoe and thus the wearer's foot causing the wearer to have to exert additional force to move his or her foot relative to that which would otherwise be the case. Embodiments include methods of walking and/or running under such circumstances to build strength or build endurance or otherwise to re-network muscle memory, etc. Such can be part of a habilitation and/or rehabilitation regime. By way of example only and not by way of limitation, embodiments include walking and/or running for less than, greater than and/or equal to five, 10, 15, 20, 25, 30, 35, 40, 45, 50, 55, 60, 75, 90, 120, 150, 200, 250, 300, 350, 400, 500 minutes or any value or range of values therebetween in 1 minute increments with such resistance, whether intermittently or continuously (at least for the lower values) as part of a rehabilitation method or otherwise to build strength or improve muscle control.

Embodiments include devices systems and/or methods of providing dorsiflexion assist and/or plantar flex assist. Any disclosure herein with respect to a method of implementing dorsiflexion assist corresponds to an alternate disclosure of a method of applying plantar flex assist as would be modified to implement such providing that the art enable such, and vice versa, unless otherwise noted. Such also is the case with respect to the devices and/or systems, where in a disclosure for dorsiflexion assist corresponds to the alternate disclosure of an embodiment with plantar flex assist, and vice versa.

Also, the embodiments for flex assist can be used for rotation limitation (adjustment of limits as well) of the heel enclosure relative to the upright(s). For example, the powered gear arrangement of FIG. 35 can be configured so that gear 3512 will stop rotation when the heel enclosure reaches a certain angle relative to the upright or relative to any other feature that can have utilitarian value with respect to a reference. In this regard, the motor that drives gear 3512 can be stopped or otherwise even reversed potentially to serve as a break against further rotation of gear 3512, and thus element 3220 and thus heel enclosure 696. In an embodiment, the control unit can be programmed or otherwise input can be received into the control unit, such as by numerical keypad arrangement that provides electronic signals to a processor or to a circuit arrangement, that indicates the range of motion of the heel enclosure relative to the upright, or at least indicates a stop angle at one point (stop angle limited by the actuator—the other stop could be the natural mechanical stop owing to the general geometry of the brace components that would exist irrespective of whether or not the actuator was present). Alternatively and/or in addition to this, the brace can be configured so that the user places the heel enclosure at an angle relative to one or both of the uprights that corresponds to the maximum rotation or minimum rotation for that matter, of the heel enclosure, and then activates input into the control system, which can be the depression of a button that provides an electrical signal to a circuit or to a processor or some other electronic component that can control the actuator, that establishes the limit of rotation. The control system would recognize or otherwise remember the rotational orientation of the gear 3512, or at least the number of rotations and/or partial rotation from a reference point, and identify such as the limit with respect to further rotation of the gear 3512. This technology is somewhat basic, and can be the technology utilized to program a press button radio or television for example, where program is a general term. Dial arrangement, such as a rheostat for example, could be utilized to set the limits. An arrangement analogous to how an automatic garage door opener is set to limit the distance that the garage door travels can be utilized in some embodiments. Components that can be mechanically moved can be adjusted so that a switch is closed or open upon a certain amount of rotation of the heel enclosure. The closure opening of the switch, or circuit, will provide an electrical signal or will halt electrical signal from reaching a controller that will control the actuator. Indeed, this could be a situation where upon a certain amount of rotation, the circuit is opened and the actuator will no longer be powered because the actuator no longer receives electricity.

In an embodiment, the arrangements of device 3300 or 830 detailed above, where such is a powered actuator, can be controlled in an analogous manner. Common techniques and devices that limit the extension and/or retraction of a linear actuator (or a rotational actuator for that matter) can be utilized to control such, and thus control the maximum angle and/or the minimum angle that the heel enclosure can be rotated relative to the upright. In an embodiment thus includes a linear actuator and/or a rotational actuator control device and/or linear actuator and/or rotational actuator limiting device that is adjustable and/or settable as conventional.

Mechanical arrangements can be used as well. FIG. 29A shows such an arrangement 930A, where piston 932 is connected to shaft 935 and plungers 90101 can be moved into and out of the housing that contains the piston 932 to limit the movement of the piston, and thus the rotation of the heel enclosure by limiting the travel of the piston, and thus the shaft 935.

FIG. 30C shows a device 930B that enables a quasi-infinite adjustment to a range of rotation (or a stop in one direction or the other) of the heel enclosure relative to the upright(s). Here, there is threaded rod 30A2 that extends through female thread block 30A1 which is welded or otherwise rigidly attached to the housing 931. Knob 30A3 can be turned by a user's fingers and thus move the rod 30A2 in the direction of the double-headed arrow. This moves the tip of the rod 30A2 towards or away the piston 932. This varies the travel of the piston, and thus varies how much the heel enclosure can rotate in one direction, where the tip of the rod 30A2 hits the piston and thus the piston cannot travel any further in that direction. In an embodiment, a user adjusts the insertion depth of the rods to adjust how much or how little rotation is permitted. This embodiment includes two threaded rods as seen, but an embodiment can only have one to limit rotation in one or the other direction. Because of the nature of the threaded rod being infinitely adjustable (as used in the engineering term), a wide range of angular rotations can be achieved (or a wide range of limitations can be achieved, more accurately). In an embodiment

In an embodiment, with respect to the angle A606 of FIG. 36, embodiments can enable a user to adjust the limits of rotation of the heel enclosure, from a value of A606 equals 90 degrees as the reference (the heel enclosure is effectively normal to the uprights) by plus or minus, less than, greater than and/or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 or more degrees or any value or range of values therebetween in 0.25 degree increments (that this is not the value of A606, this is how A606 would change), where the structure of the device can enable more rotation than such in one or two directions in the absence of the rotation limiting adjustment mechanism. That is, the adjustment mechanism limits the natural rotation that can exist, all other things being equal. And this is not to say that the limits are the same angles in both directions. They can be, but also embodiments can, for example, limit the rotation from A606 equals 90 degrees by plus 30 degrees and minus 15 degrees, for example (so with the frame of reference of FIG. 36, heel enclosure can rotate counterclockwise by 15 degrees, and clockwise by 30 degrees (and thus have a range of rotation of 45 degrees, and can have axis 644/645 be A606 equals 75 to 120 degrees, for example).

Embodiments are such that the limits are set with out components that rely upon tensioning for example in at least some embodiments. For example, there are no cables associated with the brace in some embodiments, or at least there are no cables that are utilized in the arrangement to limit rotation in one or both directions. In an embodiment, the rotation limitations are pure compression arrangements or at least a majority compression arrangements, in that the force or otherwise the physical phenomenon that is utilized to limit rotation is compression as opposed to tension. In an embodiment, there is no tensioning as a result of the utilization of a rotational device for example. While rotational devices are utilized, such as the knobs noted above, those are utilized to set a location of an adjustment as opposed to adjust a retention regime or otherwise pull on the heel enclosure. The arrangements for adjustment can include devices that do not physically contact the moving component until the point at which the rotation should be limited. In an embodiment, the rotation limiter device uses rigid components and does not include flexible components, at least not that which is used to impart resistance forces. That is, any load bearing element is rigid, or at least more rigid than flexible (for characterization purposes). In an embodiment, the rigidity is that of a polymer, such as a hard plastic, or at least raw aluminum, at least when applied in a compressive manner.

Embodiments include methods of adjusting rotation limits after the brace has been placed on a user's shoe/foot, etc. in an exemplary embodiment, there is a method, that includes obtaining a brace according to any of the embodiments herein that have a device that enables rotation limitation adjustments with respect to the heel enclosure angle relative to the uprights. The method includes placing the brace around a shoe worn by a wearer and attaching the brace to the user in a manner that is at least partially attached or fully attached (all the buckles and straps are tightened as would be utilized for full use of the brace). Note that embodiments can include making the adjustments to the rotation limits prior to placing the brace onto the person, and indeed, the adjustments will be set to some level in some embodiments, while in other embodiments, consistent with the features disclosed above, at least some of them, it is possible that the adjustment device could be set so that the natural limits/design limits of the upright and heel enclosure control the amount of rotation (for example, moving the threaded rods outboard so that the tips of the threaded rods are sufficiently far away from the piston that the full travel of the piston is such that the piston never contacts the tip of the threaded rods, and thus the rotation is maxed out owing to the structural design). In an exemplary method, after the braces strapped at least partially or otherwise attached at least partially to the user, and adjustment can be made by the user or another person assisting the user. In an embodiment, the user walks 10 or 100 or 500 steps or thousand or more and/or ½ a mile or mile, or otherwise half hour or an hour or two hours lapse, and then the user adjusts one or both of the rotation limits according to any of the values detailed herein, and then utilizes the brace for any of the time periods and/or distances etc., just noted, or others for that matter. This could happen to 345 times a day or more. The user can make many adjustments so as to refine the rotation limits or eliminate limits, depending on how he or she seeks to utilize the brace.

Other arrangements that can set the limits for the rotation of the heel enclosure/adjust such beyond that which results from the geometry without an adjustment apparatus are described further below.

FIG. 36A shows another exemplary brace 5297. Here, there is a slot 162S1 in the male portion 162, with stops 162S2 at the ends of the slot. The threaded shafts 30A2 can be adjusted upwards and downwards so that the amount of rotation of the male portion 162, and thus the heel enclosure 696, is limited by the location where the tip(s) of the threaded rod contact the stops 162S2. By turning the knob 30A3 to screw the threaded rods upward and/or downward, per the double headed arrow, the “depth” of the tip of the threaded rod can be adjusted, and thus the amount of rotation allowed can be adjusted. In an embodiment, this arrangement can also be used to lock the heel enclosure at a desired angle by causing the tips to contact the respective stops at the same time.

Instead of knobs 30A3 and/or in addition to this, a screwdriver receptacle or Allen wrench receptacle, etc., can be used to adjust the location of the tips.

A gear system can be used where wings extending therefrom are turned by one's fingers to impart rotation onto the gear, which meshes with teeth on the rod 30A2 to move the rod upwards and downwards per the direction of rotation. This can provide a lower profile arrangement. A cam system can be used. FIG. 37A shows cams 30A7 that push against rods 30A8 when rotated to create a moment relative to blocks 30A9 that have a close tolerance through hole through which the rods extend. Here, the rods can be lowered or raised to provide for the desired rotation limits, and then when the cams are rotated, the cam portions pushes against the rod and the resulting moment effectively locks the rod at that location (by pushing to the right). Alternatively, gear teeth could be located on the cams 30A7 that interface with teeth on the rods. The cams can be akin to those used on ski boots. Indeed, FIG. 32A shows a brace that utilizes a handle 30A6 that has a threaded female portion through which a threaded portion of the rod extends, and by rotating the handle 30A6 as is done on a ski boot (the handle on the right side is in position for rotation) the rod is moved up or down, and then the handle is rotated in a second axis (the handle on the left side has been rotated) to lock the rod in place. A rachet system could be used in another embodiment, where the ratchet can be reversed, and each turn of a knob or pump of a handle moves the male portion (the adjustable portion that limits the travel of a component that moves with the heel enclosure rotation, whether that be a component that moves in rotation or in a linear manner (where the rotation of the heel enclosure is converted to linear movement of the component) in one direction a certain amount or in another direction a certain amount, thereby adjusting the limits. In an embodiment, there could be a system that acts as a clutch where after a certain amount of rotation, the clutch locks up and then further rotation is halted (or increased resistance to rotation is applied).

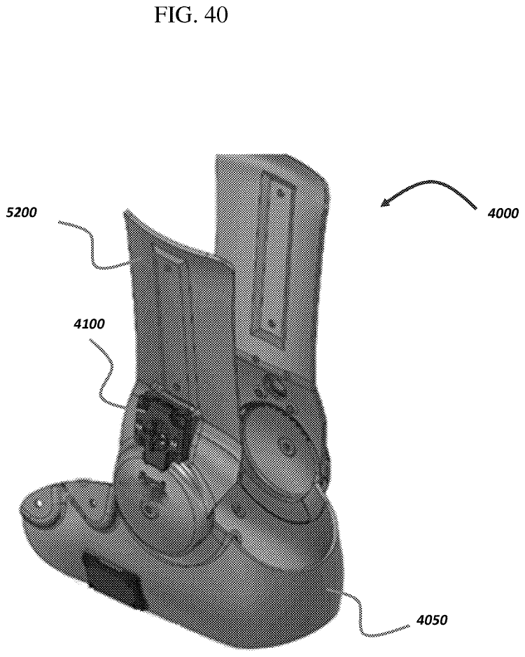

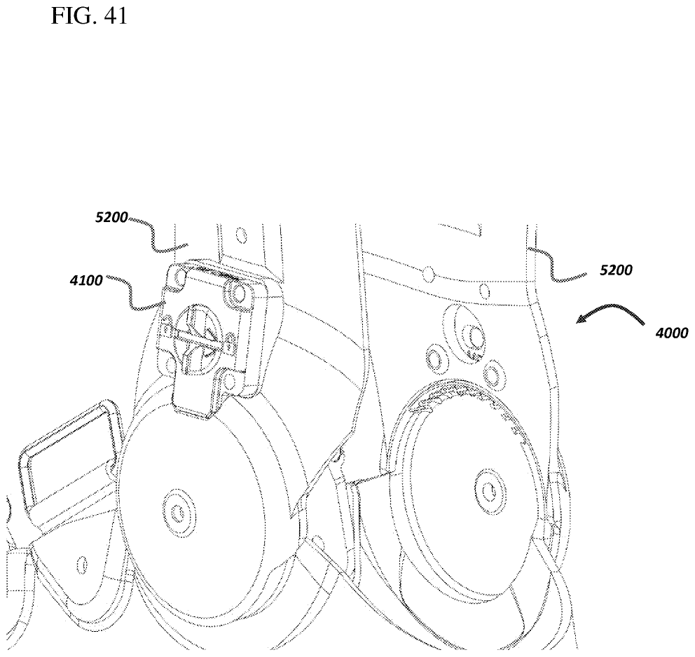

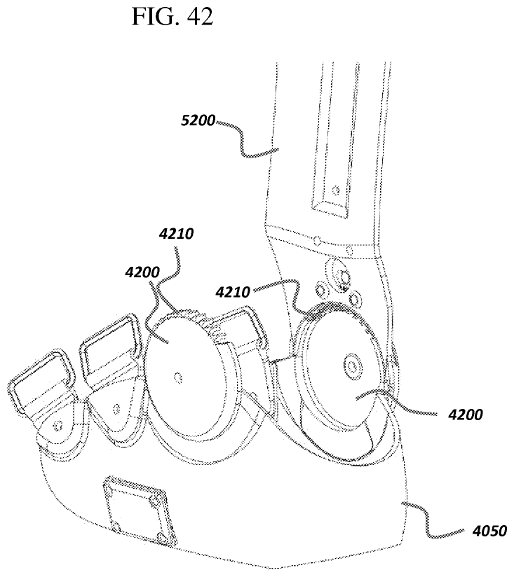

FIGS. 40-50 depict various views associated with the medial joint and lateral joint according to an exemplary embodiment, and a lockable pivot arrangement. FIGS. 40-41 present isometric views of the medial and lateral joints according to an exemplary embodiment, and FIG. 42 presents an exemplary view with one of the uprights removed for purposes of explanation.

More specifically, FIGS. 40 and 41 present an external brace 4000 that includes a heel enclosure 4050, and uprights 5200 (for the locking embodiment, the features will be described in a manner that is agnostic with respect to lateral and medial sides-aside from the mounting lugs on the uprights that take into account the different contours of the uprights, the features of the locking arrangement for the lateral side in at least some embodiments are identical to those of the medial side, and thus in this embodiment, any disclosure relating to one corresponds to a disclosure relating to the other unless otherwise noted in the interest of textual economy). Here, there is an upright locking apparatus 4100 (the qualifier “upright” refers to the portion of the locking arrangement that is “carried” by the upright). The upright locking apparatus can be riveted or screwed or bolted to a lug or thickened portion of the upright. FIG. 42 shows a view of the brace 4000 with one of the uprights removed for the purposes of disclosure. Here, the second part of the locking arrangement can be seen, which is heel enclosure locking apparatus 4200 (the qualifier “heel enclosure” refers to the portion of the locking arrangement that is “carried” by the heel enclosure (here, apparatus 4200 is monolithic with a body making up the heal enclosure, but could be a separate part attached (bolted or glued, etc.) to the remainder of the body)). With respect to heel enclosure locking apparatus 4200, this corresponds to teeth 4210, which can be analogous to gear teeth, arrayed about the outer periphery of the male portion of the joint indeed, element 4200 is the male portion of the joint, where a portion of that has been modified to have the teeth 4210. As seen, there are a number of teeth and corresponding valleys between the teeth on the outer periphery of the top portion of the male portion of the joint. In an embodiment, there are less than, greater than and/or equal to 1 (in which case there is no less than) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 or more or any value or range of values therebetween in 1 increments teeth arrayed about the outer periphery of the male portion of the joint. In an embodiment, the number of teeth are identical on both sides of the brace, while in other embodiments, there could be more or less teeth on one side than the other. The dimensions of the teeth and the number of teeth can be varied depending on the design or otherwise depending on how much “grip” is desired to lock the uprights into a position. And briefly, it is to be understood that the uprights can be locked at different angles, as the locking arrangement is independent on each side in this exemplary embodiment.

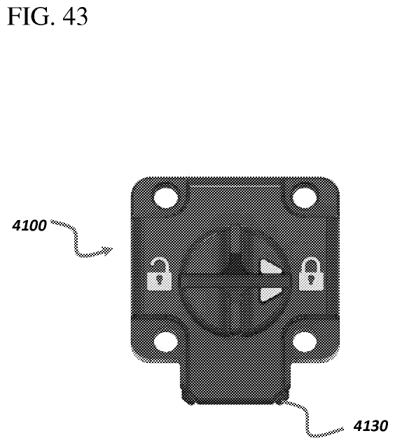

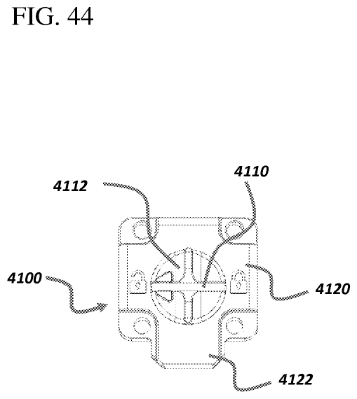

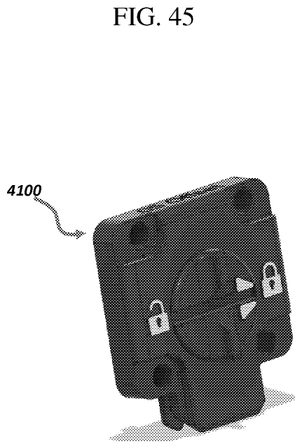

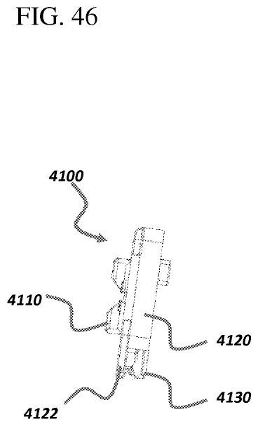

FIGS. 43 and 44 depict a side view of the upright locking apparatus 4100 (a view looking at the apparatus if looking at the brace from the side), and FIG. 45 depicts an isometric view of such. Apparatus 4100 is made of three thermoplastic molded parts: a trolly 4130, a chassis 4120 and a knob 4112 where a handle 4110 is present to enable a user of the brace or a caregiver of the brace to grip the handle with a farm and a finger, such as the forefinger, to enable a torque to be applied to the knob 4112 so as to actuate the locking apparatus and thus engage and disengage the lock arrangement on a given side of the brace. FIG. 46 presents a side view of the locking apparatus 4100, where the trolley 4130 of locking apparatus 4100 can now be seen. In the previous figures, the trolley 4130 was concealed by the female track 4122 for the trolley 4130, which track also provides a barrier between the outside of the brace and the trolly 4130.

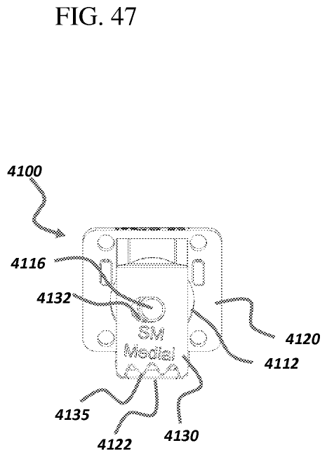

FIG. 47 provides a rear view (opposite that of FIG. 43) of the upright locking apparatus. Here, trolly 4130 can be seen in the “locked position” (extended downward in the frame of reference of FIG. 47). The 4130 has teeth 4135 that engage with the teeth of the heel enclosure locking apparatus so as to lock the locking assembly, and thus lock the angular position of the given upright relative to the heel enclosure. The trolley 4130 can be a monolithic body of a thermoplastic material (all of the components of the locking arrangement can be made of such). The trolley slides linearly within the female track 4122 of the chassis 4120. As seen, there is a race track shaped slot 4132 extending completely through the trolley 4130. This enables the trolley 4130 to be actuated (moved up and down) so as to lock and unlock the locking arrangement. In this regard, as the knob 4112 is turned as a result of applying a torque thereto via the handles 4110, a pin 4116 revolves around the axis of rotation of the knob 4112 when the knob is turned, where pin 4116 is offset from that axis of rotation. This acts as a cam and thus moves the trolley 4130 upwards and downwards with rotation of the knob 4120. The functionality can be that corresponding to a scotch yoke for example. The rotational movement is transferred to a linear movement.

In an embodiment, there are there are less than, greater than and/or equal to 1 (in which case there is no less than) 2, 3, 4, 5, 6, 7, 8 or 9 or more or any value or range of values therebetween in one increments teeth arrayed on the trolly 4130. In an embodiment, the number of teeth are identical on both sides of the brace, while in other embodiments, there could be more or less teeth on one side than the other. The dimensions of the teeth and the number of teeth can be varied depending on the design or otherwise depending on how much “grip” is desired to lock the uprights into a position. A “fine” or a “course” (relatively speaking) arrangement of teeth can be used (and different coarseness can be used on different sides). The plate and/or the angling of the teeth can be different, as well as the curvatures and profiles from that shown. Any arrangement that can enable the teachings detailed herein can be utilized in at least some exemplary embodiments.

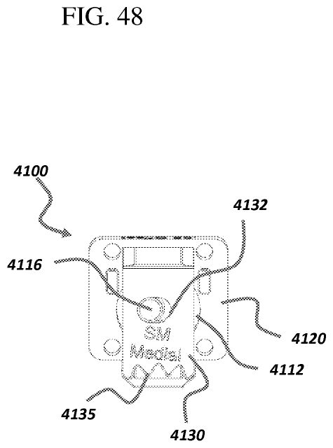

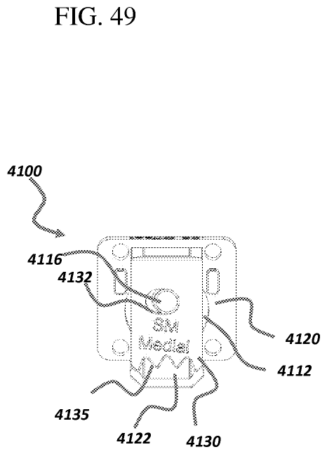

FIG. 48 presents the trolley 4130 midway between the locked position (the teeth being at the bottom with respect to the frame of reference of this figure) and the unlocked position, where FIG. 49 depicts the trolley 4130 at the completely unlocked position. Knob 4112 has made half of a full rotation and thus moved pin 4116 from a lower position to an upper position, with the movement of the pin in the lateral direction allowed for by the slot 4132. (In an embodiment, the knob is rotated by less than, greater than and/or equal to 150, 155, 160, 165, 170, 175 or 180 degrees or any value or range of values therebetween in 0.1 degree increments.) Returning back to FIGS. 43 and 44, the indicator on the knob can be seen to have moved from the locked symbol to the unlocked symbol between those two figures, and at the bottom of FIG. 43, a portion of the trolley can be seen, where that portion cannot be seen in FIG. 44 owing to the fact that the trolley has been moved upwards (behind the chassis). In an exemplary embodiment, detents are utilized to provide a modicum of force retention to the knob 4112 to the locked and the unlocked positions so that the knob will not easily turn from one position to the other, or otherwise a force or a torque more accurately must be applied to the handles or otherwise to the knob, to have the knob move or otherwise rotate, and thus move the trolley. In an exemplary embodiment, the detents can be part of the molded thermoplastic material, where, for example, a female portion is located on the knob and the male portion is located on the chassis, or vice versa, where the plastic material will plastically deform when a sufficient amount of torque is applied so that the knob can be rotated from the locked and unlocked position.

In an exemplary embodiment, friction forces are utilized to “hold” the knob in place, and thus “hold” the trolley in place. In an alternate embodiment, a positive lock arrangement can be utilized that affirmatively prevents rotation of the knob, and thus movements of the trolley, when such is engaged. By way of example only and not by way of limitation, a snap cover with a female receptacle for the “handles” of the knob can be present so that when the snap cover is placed over/across the knob, the knob cannot turn the cause of the handles interfere with the snap cover. Alternatively, a bar can extend across the top of the bottom of the handles, hinged on once side and snap-coupled on the other side (either sides of the knob) so that one end of the handles will hit the bar before any substantial amount of rotation takes place. A pin can extend through a hole in the handles and a hole in a lug component of the chassis, which would prevent rotation until the pin is removed. Still, in an exemplary embodiment, is friction force of the overall system that retains the non- and/or the trolley in location in the absence of a sufficient torque applied to the knob to move the trolley or otherwise to move the knob and thus move the trolley.

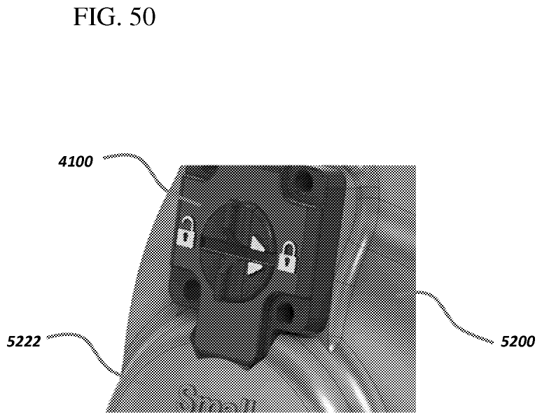

FIG. 50 presents a closeup view of the upright lock apparatus 4100 mounted to the upright 5200, with an opening in the female portion 5222 of the joint for the track to extend into the joint and thus allow the trolley to reach the teeth of the heel enclosure lock apparatus. As seen, apparatus 4100 is mounted to and moves in a one-to-one relationship with upright 5200.

When the trolley 4130 is driven downward by the camming action so that teeth 4135 mesh sufficiently with the teeth 4210 of the heel enclosure locking apparatus 4200, any torque that is applied to the heel enclosure 4050 is transferred to the upright 5200 through the upright locking apparatus 4100. The locking apparatus 4100 is bolted or riveted to the body making up the upright 5200. Thus, the heel enclosure 4050 will move in a one to one relationship with the particular upright when the upright is “locked” in position relative to the heel enclosure. More specifically, when the uprights 5200 are secured to the leg of the person wearing the brace 4000 by way of the straps or other retaining arrangement, the orientation of the shoe to which the heel enclosure is secured will be effectively limited to the ability of the shoe to move within the heel enclosure.

In an exemplary embodiment, the locking arrangement and, in some embodiment, the entire brace, can withstand at least 200, 250, 300, 350, 400, 450, 500, 550, 600, 650, 700, 800, 900, 1000, 1250, or 1500 or any value or range of values therebetween in 10 increments inch-pounds of torque reacted between the upright and the heel enclosure without any plastic deformation and/or without the teeth becoming disengaged (e.g., this could be tested by placing a properly sized model shoe and model leg in the appropriate locations and subjecting the leg to a torque while the shoe is secured to a fixed component (and/or the heel enclosure is secured to the fixed component) when the locking arrangement is in the locked configuration).

In an embodiment, the locking arrangement is configured to provide less than greater than and/or equal to 1 (in which case there is no less than) 2, 3, 4, 5, 6, 7, 8, 9, 10, 11, 12, 13, 14, 15, 16, 17, 18, 19, 20, 21, 22, 23, 24 or 25 or more or any value or range of values therebetween in 1 increments locking positions (locked positions). In an exemplary embodiment, each locking position locks the brace at a different angle of 5° from the previous locking position (Angle A606 for example). In an exemplary embodiment, each locking position locks the brace at a different angle of 10° from the previous locking position. In an exemplary embodiment, each locking position locks the brace at an angle that is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 degrees or more or any value or range of values therebetween in 0.1° increments from the previous locking position. Note that in an exemplary embodiment, the different locking positions need not be equidistant relative to a relationship of two prior locking positions. For example, a first locking position could lock the brace at 5° from the 90° angle, and the second locking position can lock the brace 7.5° from that prior locking position that is 5° off of the 90° position. This can be accomplished by, for example, a complex arrangement of teeth where the teeth have different pitches or have different configurations over the circumference of the trolley and/or of the male portion of the joint. Further, it could be that there are teeth that are missing so that for example, the brace can be locked in a 5° position but cannot be locked at a 10° position but can be locked in a 15° position and in a 20° position for example. That is, the teeth that would be present to lock the brace at the 10° position are not present. It could be that there are no teeth at the 10° position, and, the outer circumference of the male joint is as high as the teeth on either side, so that the trolley 4130 can simply not the transition downward to positively interfere with the male portion of the joint on the heel enclosure. Conversely, it could be that the teeth are not present and there is a cavity into which the trolley can be lowered, but there is no locking per se at a specific angle.

And this concept raises another point. In an exemplary embodiment, the absence of teeth and the utilization of a cavity (on the male joint portion) can enable the brace, more accurately, the upright or the heel enclosure, to be rotated through a series of angles but not beyond limits to those angles. By way of example only and not by way of limitation, it could be that there is a lock at the 90° position for example, where there is one or more teeth on the heel enclosure that allow such locking. However, there are teeth of the heel enclosure missing at other locations, creating a cavity, wherein the trolley, more accurately the teeth 4135 of the trolley, can be located in this cavity, and the upright can rotate so that the teeth can move in this cavity from the location where there are teeth for the 90° position to a location where, for example, there are teeth to lock the brace and a 70° position. Those teeth would stop further rotation beyond the 70° position, or beyond some position that would result from locking the brace or more accurately having structure that would lock the brace at that position. Note also that it could also be that in addition to teeth, there is some form of wall or other structure that more robustly will contact the lateral sides of the trolley and thus provide the more robust regime with respect to halting further rotation in one or both directions.

Embodiments thus include a multi-stage pivot arrangement. In an embodiment, the variations of the teeth can be configured so the brace can be enabled to pivot by an amount that is greater than and/or equal and/or less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 degrees or any value or range of values therebetween in 0.1 degree increments (A606 for example). In an embodiment, the pivoting can be centered or from one or more of any of the recited angles of A606 noted above, in the interests of textual economy.

The device can limit the angles of pivoting as just noted through any of the ranges, and the limited angle for forward pivoting can be different than the limited angle for rearward pivoting by adjusting the position of the teeth/absence of teeth, etc.

Thus, as can be seen, instead of a locking arrangement, there is the genius of a rotation limiting system or a rotation control system, which can have a species of a locking system which locks the brace at a specific angle, and then a species of a rotating limiting system, which allows the upright to rotate but only a limited amount relative to that which would otherwise be the case, or when the system is engaged.

And to achieve the full range of forward and/or rearward pivoting, the trolly can be retracted to clear the teeth. In some embodiments, the utilization of variable teeth size/positioning/gaps in teeth can be implemented so that pivoting of the upright(s) in the forward direction or pivoting in the rearward direction relative to the full range of pivoting in those directions is limited. For example, the arrangement might limit pivoting in the rearward direction to only ⅔rds of the total amount possible, and may not limit forward pivoting by any amount (except that which is limited by the overall structure without the locking/articulation limiting arrangement).

As noted above, the hinge assembly (pivot assembly) can be configured to provide different modes of use. In this regard, in some embodiments, the hinge is a lockable hinge that can be unlocked to enable movement and/or adjustment. In some exemplary embodiments, the hinge is set to be locked at a specific angle (90 degrees or 80 degrees from the horizontal, for example). In some exemplary embodiments, the lock arrangement is adjusted to enable free range of motion over a wide degree of angular movements. By way of example only and not by way of limitation, the amount of angular movement could be, relative to the locked angle, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65 or 70 degrees or any value or range of values therebetween in 0.1 degree increments dorsiflexion and/or plantar flexion (and the forward can be the same or different than the backwards angular movement). In an embodiment, the pivoting enablement and/or limiting can implemented to meet any one or more of the performance features of WO2022204557.

In an exemplary embodiment, the pivot assembly is configured to provide only a limited range of motion, or more accurately, configured to permit only a limited range of motion (when the locking arraignment is engaged, for example). By way of example only and not by way of limitation, limited range of motion can be a fraction of the aforementioned full range of motion. By way of example only and not by way of limitation, the limited range of motion could be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55 or 60 degrees or any value or range of values therebetween in 0.1 degree increments less than and/or equal to those which corresponds of the movements noted above. By way of example only and not by way of limitation, the limited range of motion could be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 30, 31, 32, 33, 34, 35, 36, 37, 38, 39, 40, 45, 50 or 55 degrees dorsiflexion and/or plantar flexion, or any value or range of values therebetween in 0.1° increments, and again, the range of motions can be different (e.g., the hinge can allow for 5° of dorsiflexion and 12° of plantar flexion (from the locked angle)).

To be clear, a forward rotation limit could be greater than the reward rotation limit. For example, the forward rotation could be 1.5, 2, 2.5 or 3 times that of the rearward rotation (or vice versa). It is noted that there might be no rearward rotation in some embodiments. Only forward rotation.

Note that in an embodiment, there could be two or more upright locking arrangements on a given upright, so as to provide various limitation of articulation regimes.

It is noted that some but not all embodiments of the lock arrangement allow a very limited amount of flexure/rotation even when the locking arrangement is locked (engaged). In this regard, for a torque applied to an upright equal to 10, 20, 30, 40 or 50 foot-pounds forward and then in the backward direction (or vice versa), there will be less than and/or equal to 5, 4.5, 4, 3.5, 3, 2.5, 2, 1.5, 1, 0.5, 0.4, 0.3, 0.2 or 0.1 degrees or any value or range of values therebetween in 0.01 degree increments in total rotation (from the most forward location to the most rearward or vice versa). That said, the aforementioned values can be considered.

In an exemplary embodiment, the brace is configured so that a torque applied to an upright equal to 10 foot-pounds forward and/or backward so that the stops are hit in the respective directions will have the upright at a first location (potentially for example 0.1 degrees off of the Z axis), and then an increase from that torque equal to 10, 15, 20, 25, 30, 35, 40, 45 or 50 foot-pounds (e.g. to a total of 60 foot-pounds) will result in no more than and/or equal to 3, 2.5, 2, 1.5, 1, 0.5, 0.4, 0.3, 0.2 or 0.1 degrees or any value or range of values therebetween in 0.01 degree increments in additional rotation (from the most forward location to the most rearward or vice versa).

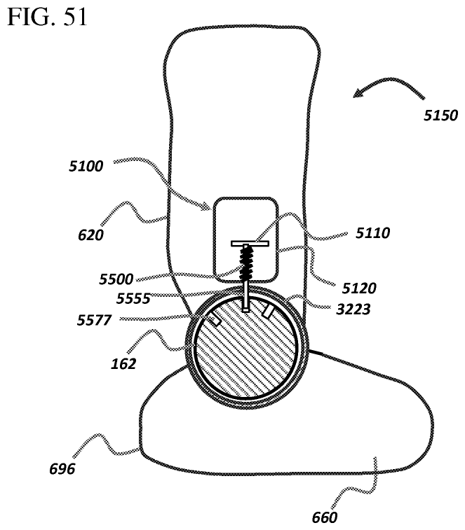

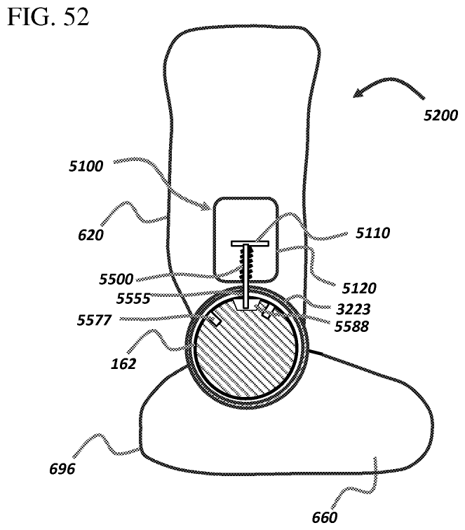

FIG. 51 presents another exemplary embodiment of an exemplary brace 5150 where a blind hole 5577 a plunger 5555 is used instead of teeth to lock the upright into position. More specifically, as seen, there is a upright locking apparatus 5100, with a chassis 5120, that is connected to the upright 620. The locking apparatus 5100 includes a handle 5110 two which is connected the plunger 5555. In this exemplary embodiment, the plunger is spring loaded by spring 5500. In this exemplary embodiment, the handle 5110 can be articulated to lock and unlock the plunger so that the plunger can be moved upwards and downwards. Moving the plunger downward into blind hole 5577 results in the upright 620 being locked at a given angle, and moving the plunger upwards out of the hole 5555 allows the upright to articulate to its “natural” stops.

The blind holes 5577 are located in the male portion 162 of the joint. In this exemplary embodiment, there are three blind holes 5577. Insertion of the plunger into those holes will lock the upright 620 into a given angle relative to the heel enclosure 696. In an embodiment, there can be more or fewer blind holes. In an exemplary embodiment, there can be any number of holes that are noted above with respect to the number of teeth. Also that there could be more than one plunger assembly in fact. Multiple plungers could provide for redundancy or otherwise increased force or otherwise further reinforce a locking regime.

FIG. 52 presents another exemplary embodiment of a brace 5200, where the upright locking apparatus 5100 is utilized, but instead of a constant diameter blind hole, and angled blind hole 5588 is utilized, or more accurately a blind groove 5588 is used for example. This can enable the articulation of the upright 620 to be limited, but not completely prevented relative to the heel enclosure 696. As seen in combination, blind holes 5577 are utilized so as to lock the upright into a given angular position. Note that in an exemplary embodiment, there can be multiple grooves 5588 (e.g., one or more of the blind holes can be replaced), and these grooves need not be centered at the 90° position as shown in FIG. 52. In an exemplary embodiment, there can be a number of grooves that correspond to any of the disclosed number of holes detailed herein for the purposes of textual economy.

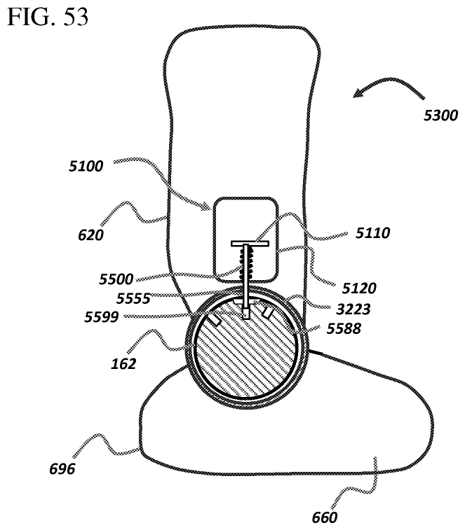

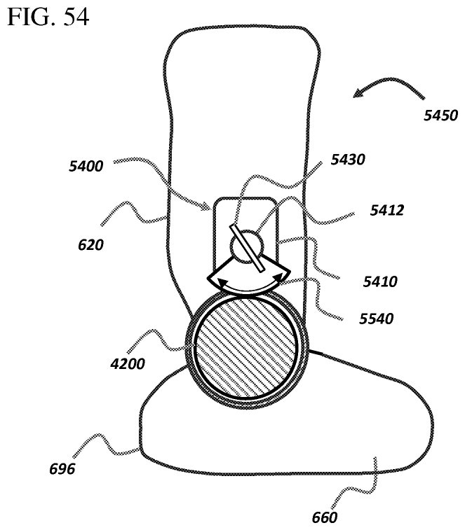

FIG. 53 presents another exemplary embodiment of a brace 5300, where a groove 5588 and a blind hole 5599 are utilized in combination. In this exemplary embodiment, the depth of the plunger 5555 can be limited, where, if the plunger is limited to a depth that keeps the bottom portion of the plunger totally within the groove 5588, a range of angular articulations from one end of the groove 5588 to the other end of the groove, while if the plunger is located or otherwise permitted to travel further downward into hole 5599, the upright 620 is locked in a single position. In an exemplary embodiment, the locking apparatus 5100 is configured to enable a user to control the depth of insertion of the plunger 5555 so as to adjust from one functionality (limited range of articulation) to another functionality (total prevention of articulation). In an exemplary embodiment, a ball détente system could be utilized to control the depth of plunger 5555, where, for example, a downward force on handle 5110 of a first value would result in overcoming the spring force 5500 (if present, in other embodiments, there is no spring, or alternatively, the spring 5500 could bias the plunger downward instead of upwards) and permit plunger 5500 to travel a distance where the ball détente would engage and thus “lock” the plunger into a first position so that the tip stays within the groove 5588. Then, upon application of a force onto handle 5110 of a second value larger than the first value in large enough to overcome the locking of the ball détente mechanism, the plunger 5555 would be able to further travel into hole 5599, where the handle or otherwise the locking mechanism could then be locked into place, and thus lock the upright into a specific angle.

In an embodiment, the inner diameter of the female portion 222 normal to the axis of rotation 2222 of the medial upright extension can be less than, greater than and/or equal to 0.75, 1, 1.25, 1.5, 1.75, 2, 2.25, 2.5, 2.75, 3, 3.25 or 3.5 inches or any value or range of values therebetween in 0.0001 inch increments. The outer diameter of the male portion 182 of the joint normal to the axis 2222 can be less than and/or equal to and/or greater than 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.35. 0.37, 0.38, 0.39, 0.40, 0.41, 0.42, 0.43, 0.44, 0.45, 0.5, or 0.55 or more inches or any value or range of values therebetween in 0.0001 inch increments undersized relative to the female portion. And note that the male portion and/or the female portion need not be completely circular 350 degrees around.

As can be seen in FIGS. 24 and 25, the respective axes 2222 and 2000 of joint rotation/joint pivot for the medial joint and the axial joint are offset by a distance D1 in the Z axis (the axis normal to the ground/bottom of the brace). In an exemplary embodiment, D1 can be less than and/or equal to and/or greater than 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9 or 2 inches or more or any value or range of values therebetween in 0.0001 increments (or less), at least to achieve the standard English measurements. The offset in the forward-backward direction (towards and away from the toe) can be any of these values as well.

In an exemplary embodiment, the pin that moves within the cam slot so as to move the trolley is located on the knob so that the pin is located, or more accurately, the center of the pin or otherwise the longitudinal axis of the pin, where in this exemplary embodiment the pin has a circular cross-section on a plane normal to the longitudinal axis of the pin, is located at the 12 o'clock position when the handle and thus the knob indicates that the upright locking arrangement is at the unlocked position, and is located at the 6 o'clock position when the handle and thus the knob indicates that the upright locking arrangement is at the locked position, these positions being in reference to the orientation of the lock arrangement 4100 shown in FIG. 49 for example, where the top is the 12 o'clock position and the bottom is the 6 o'clock position. In an exemplary embodiment, this is not the case. Instead, when in the locked position/fully engaged position, the center of the pin can be at the 5:30 or 5:45 position for example or at the five o'clock position or at the 5:15 position for example, thus, any upwards force applied to the trolley 4130, such as might be the case when the heel enclosure is subjected to a force that would otherwise cause the heel enclosure to rotate relative to the upright, and thus a moment would be applied to the heel enclosure, which moment would cause a modicum of rotation of the heel enclosure relative to the upright, where because of the angling and the orientation of the teeth, this would drive the trolley 4130 upwards a slight amount, but because the pin is offset from the 6 o'clock position, pin the pin “binds” in the slot or otherwise cannot further rotate counterclockwise with respect to the orientation of FIG. 47 for example, because the pin is completely against the right side portion of the slot, and thus further movement of the pin upwards is prevented. This is opposed to, for example, if the pin was located at the 6 o'clock position, where, the pin could start moving owing to rotation of the knob in the clockwise direction. Put another way, because any upward movement on to the pin or otherwise any upward force applied to the pin when the pin is offset from the 6 o'clock position would drive the pin to move in a direction that has a component to the right with respect to the orientation of FIG. 47, the wall of the slot stops further movement. This is the effect of providing additional redundancy or otherwise a safety feature to further reduce the likelihood that the locking arrangement could become disengaged by accident or as a result of a sufficiently large moment applied to the heel enclosure relative to the upright. It could be that the teeth are not as close as possible to each other when in the locked position. It could be that the cam allows the teeth to bottom out and then retract a bit, so as to get the pin off of the 6 o'clock position to enable the feature just noted.

FIG. 54 presents another exemplary embodiment of an exemplary brace, brace 5450, which includes an articulation adjustment/locking mechanism 5400. Here, there is a non-5412 that includes a handle 5430 that is supported by a chassis 5410. The knob is configured to rotate relative to the chassis as a result of a torque applied to the knob by way of the handle 5430, which is configured to be gripped by a thumb and forefinger for example and thus term by such. Here, the knob 5412 is physically connected in a one to one relationship with a gear segment 5540. This gear segment rotates in a one-to-one relationship with the knob 5412. This will thus move the upright 620 about the pivot axis as the gears 5540 mesh with the gears 4200. This will change the rotational alignment of the upright. In an exemplary embodiment, the friction forces are sufficiently high that once a desired angle is established, very high moments will be required to be applied to the heel enclosure 696 and/or the upright 622 move the orientation away from the set orientation. In an exemplary embodiment, the arrangement can be configured so that a locking device, such as a lock pin that can be inserted in a direction corresponding to a direction into and out of the page of FIG. 54, and thus through gear 5540, so as to prevent further rotation of gear 5540, by way of a strap that goes over the handle 5430. In an exemplary embodiment, there can be a device that is actuated, such as, for example, by folding over the handle 5430 so that the folding of the handle drives out a movable component so as to create an interference fit or otherwise a compression fit between the knob 5412 and the chassis 5410, which compression or interference fit is sufficiently high that friction forces are sufficiently high that it will be very difficult to rotate the upright relative to the heel enclosure. By way of example, this can be analogous to a device that is utilized to reseal a wine bottle or the like, where an artificial cork is placed in the bottle, and componentry expands the outer circumference of that artificial cork so as to create a seal between the bottle and the artificial cork. That said, in an exemplary embodiment, folding the handle down could then drive a pin in a direction that is parallel to the plane of FIG. 54, which pin is carried by the knob 5412, in which pin will travel into an orifice in the chassis 5410.

The embodiment of FIG. 54 can, in some embodiments, provide basically an infinite adjustment regime, in that any given angle however fine can be set.

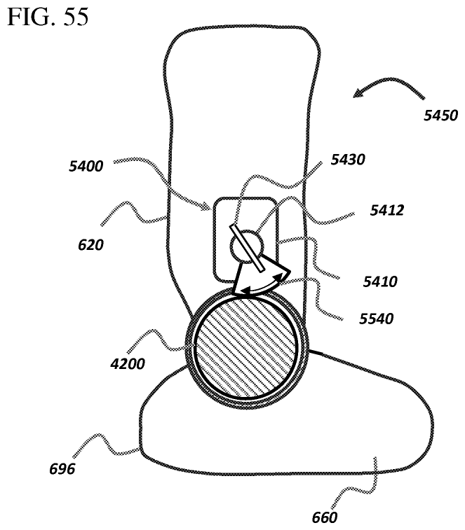

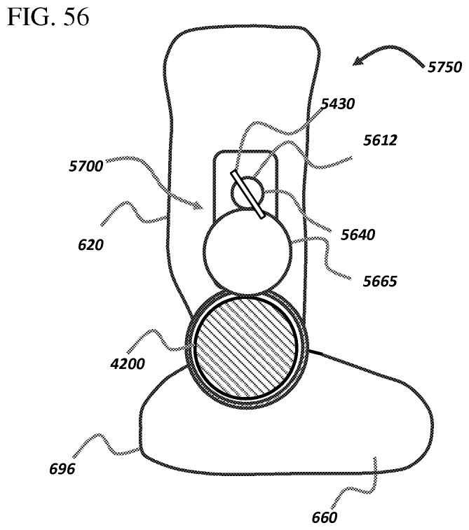

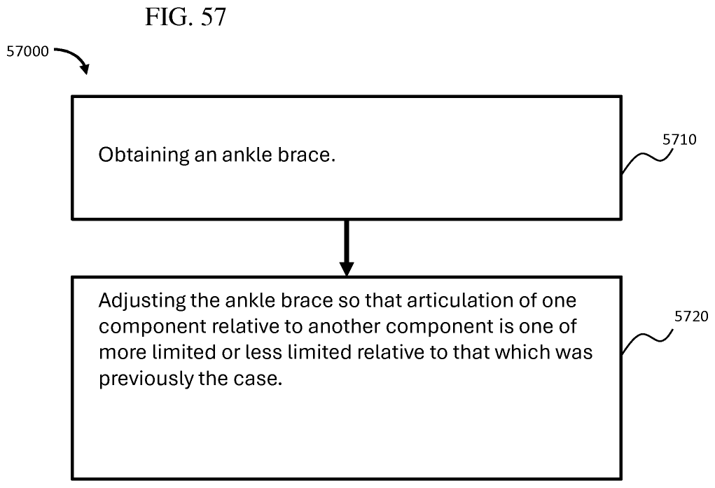

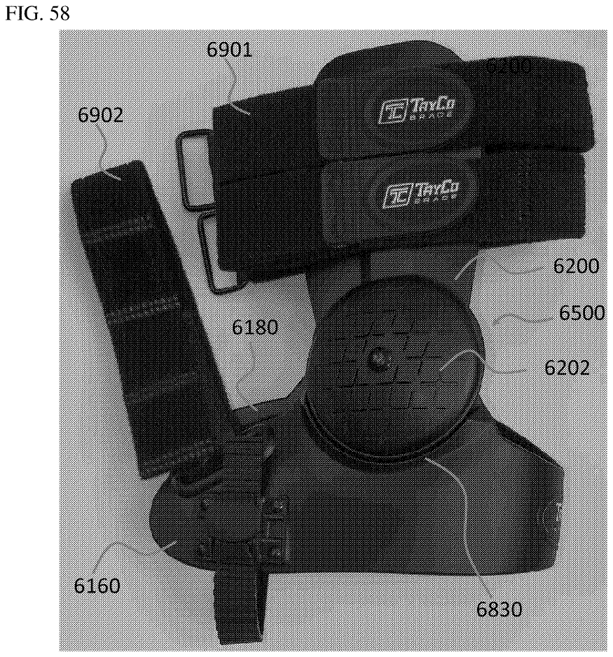

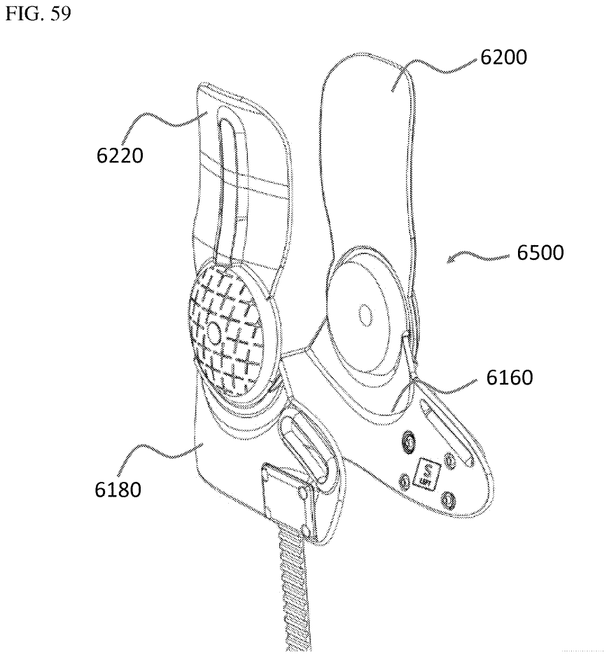

FIG. 55 presents another exemplary embodiment, where gear 5540 has a different configuration than that of the embodiment of FIG. 54. Here, the brace would be configured so that the range of movements of the upright 620 clockwise or greater than that which is the case counterclockwise. The reverse can be true as well. Any arrangement that can enable the teachings detailed herein or otherwise will have utilitarian value can be utilized in at least some exemplary embodiments. FIG. 56 presents another exemplary embodiment that utilizes a system of gears. Here, there is brace 5650, which includes adjustment arrangement 5600, which includes a first gear 5640 mounted on chassis 5610, which gear meshes with a second year 5665, which is attached in a one to one relationship with knob 5612. Handle 5430 is attached to knob 5612. A torque applied to handle 5430 rotates gear 5665, which in turn rotates the gear 5640, which thus causes upright 622 Bennett, or otherwise enables upright 620 dependent. Here, this exemplary embodiment can utilize the differences in size of the gears so as to provide for a finer adjustment angle these are the rotation of the knob 5612. Note that in an alternate embodiment, the larger gear could be on the bottom. That is, the gear 5655 can be swapped with gear 5640. This can have utilitarian value with respect to utilizing the mechanical advantage, or disadvantage, to allow the friction forces to better secure a set angle in place. This is seen in FIG. 57 by way of example where brace 5750 includes adjustment device 5700, where the gear positions have been reversed. Note also that the gear on the male portion 4200 of the joint could also be reduced in size or changed inside relative to that which is shown. Further, while the embodiments disclosed herein have the gear that is carried by the heel enclosure as monolithic with the rest of the body of the heel enclosure or at least the material that makes up the side walls of the hero enclosure, in an alternate embodiment, a separate gear could be mechanically attached to that body. In an embodiment, an opening through the female portion of the joint coaxial with the pivoting axis could be present, through which a gear could extend, which gear is mechanically connected or otherwise carried by the heel enclosure. This would enable a smaller gear or otherwise a gear having a smaller diameter to be utilized or a gear having a smaller radius of curvature to be utilized.

In an exemplary embodiment, any of the gears detailed herein or any of the teeth detailed herein span less than, greater than and/or equal to an arc subtending 5, 10, 15, 20, 25, 30, 35, 40, 45, 50, 55, 60, 65, 70, 75, 80, 85, 90, 95, 100, 110, 120, 130, 140 or 150 degrees or more or any value or range of guys therebetween in 1° increments, all in the interest of textual economy. Again, in an exemplary embodiment, the drawings can be considered drawn to scale. In an exemplary embodiment, the tips of the teeth of the gears fall within a range of a radius of curvature of two arks having respectively two of the following values: 0.2, 0.25, 0.3, 0.35, 0.4, 0.5, 0.6, 0.7, 0.8, 0.9 1, 1.1, 1.2, 1.3, 1.4, 1.5, 1.75, 2, 2.25, 2.5, 2.75 or 3 inches or any value or range of values therebetween in 0.05 inch increments.

In view of the above, it can be seen that in an embodiment, there is a brace, comprising a lower leg interface component (e.g., one of the uprights) configured to interface with at least one side of a leg of a human at a lower leg portion thereof and a foot section (e.g., a section established by the heel enclosure) configured to interface with a shoe worn on a foot of the human at a location where the foot of the human is located. In an embodiment, the foot section is articulatable relative to the lower leg interface component about an axis of rotation, as noted above. In an embodiment, the brace is configured to lock an angle of articulation of the foot section relative to the lower leg interface with a finest increment being less than or equal to 2, 3, 4, 5, 6, 7, 8, 9, 10, 11, 12, 13, 14, 15, 16, 17, 18, 19, 20, 21, 22, 23, 24, 25, 26, 27, 28, 29 or 30 degrees or any value or range of values therebetween in 0.1 degree increments relative to another locking angle (e.g., the brace can be locked at A606 at 45, 50, 60, 70, 90, 110, 115 degrees, and thus the finest increment is 5 degrees, or the brace can be locked at 60, 62.5, 67, 70, 75, 90, 105, 110, 120, and thus the finest increment is 2.5 degrees, etc.). In an embodiment, the brace is configured to provide less than, greater than and/or equal to 1 (and thus there is no less than), 2, 3, 4, 5, 6, 7, 8, 9, 10, 11, 12, 13, 14, 15, 16, 17, 18, 19, 20, 21, 22, 23, 24, 25, 26, 2, 28, 29, 30, 31, 32, 33, 34, 35, 36, 37, 38, 39, 40, 41, 42, 43, 44, 45, 46, 47, 48, 49 or 50 or more or any value or range of values therebetween in 1 increments distinct different locking angles of articulation.

In an embodiment, the lower leg interface and/or the heel enclosure includes less than, greater than and/or equal to 1 (and thus there is no less than), 2, 3, 4, 5, 6, 7, 8, 9, 10, 11, 12, 13, 14, 15, 16, 17, 18, 19, 20, 21, 22, 23, 24, 25, 26, 2, 28, 29, 30, 31, 32, 33, 34, 35, 36, 37, 38, 39, 40, 41, 42, 43, 44, 45, 46, 47, 48, 49 or 50 or more or any value or range of values therebetween in 1 increments female portions and/or male portions.

In an embodiment, there is a foot orthosis, comprising a first apparatus configured to grip a shoe worn by a human and a second apparatus configured to grip a leg of the human, wherein the first apparatus articulates relative to the second apparatus when the human walks using the foot orthosis, and the foot orthosis includes an articulation limiting system where all parts of the system are self-contained and remain attached to the brace when adjusting the integrated articulation limiting system (i.e., no part is removed) to control an angle of articulation of the first apparatus relative to the second apparatus beyond that which can be achieved when the articulation limiting system is not engaged to control articulation (control articulation can be to lock at a specific angle or limit articulation to a range that is less than a full articulatable range without the system being engaged). Thus, in an embodiment, the articulation limiting system is a locking system configured to lock the first apparatus relative to the second apparatus at a distinct and specific articulation angle. Conversely, in an embodiment, the articulation limiting system is a system configured to allow limited actuation, when the system is engaged, of the first apparatus relative to the second apparatus through a range of articulation angles less than a total maximum articulation angle for when the system is not engaged. And as noted above, in an embodiment, the system can be both.

In an embodiment, the first apparatus includes sidewalls that are used to grip the shoe and the system includes a first portion that is carried by the first apparatus and a second portion carried by the second apparatus and the first portion is monolithic with a body making up the sidewalls (e.g. the teeth that make up the male portion of the joint of the heel enclosure are monolithic with the body that establishes such). In an embodiment, when the articulation limiting system is unengaged (e.g., the trolley is completely clear of the teeth of the male portion of the joint) and the foot orthosis is in a state where the first apparatus can fully articulate relative to the second apparatus (as opposed to being limited to an exact angle or limited to a range of angles, as distinguished from full articulation, which is limited only by the structure unrelated to the control system), all parts of the system for when the system is engaged are attached to the orthosis. The articulation limiting system is a locking system in some embodiments and is configured so that when engaged, the system only locks the first apparatus relative to the second apparatus at a distinct and specific articulation angles (as opposed to enabling a range of movements). The articulation limiting system can be a locking system configured so that when not engaged, the system allows the first apparatus to fully articulate relative to the second apparatus beyond that which is permitted by a maximum articulation allowed by the system when engaged (which could be a limited range of articulations).

In an embodiment, the engagement and disengagement of the articulation limiting system is established by moving a component in a direction at least generally parallel to a longitudinal axis of an upright of the second apparatus. In an embodiment, the engagement and disengagement of the articulation limiting system is established by moving a component in a direction that is less than, greater than and/or equal to 5, 10, 15, 20, 25, 30, 35, 40, 45, 50, 55, 60, 65, 70, 75, 80, 85, or 90 degrees or any value or range of values therebetween in 1 degree increments to a longitudinal axis of an upright of the second apparatus. For example, with respect to 90 degrees, the locking arrangement carried by the upright shown in FIG. 49 could be rotated 90 degrees. Of course, as seen above, the articulation limiting includes gear teeth that mesh with each other when the system is engaged.

In an embodiment, there is a foot orthosis, comprising a first apparatus configured to grip a shoe worn by a human and a second apparatus configured to grip a leg of the human, wherein the foot orthosis is configured to control whether and/or how much the first apparatus articulates relative to the second apparatus when the human walks using the foot orthosis, and the control is engaged and disengaged via application of a torque to a component of the orthosis (e.g., applying a torque to the handle and thus the knob). In an embodiment, the torque needed to engage and/or disengage is less than, greater than and/or equal to 0.25, 0.5, 0.75, 1, 1.25, 1.5, 1.75, 2, 2.5, 3, 3.5, 4, 4.5, 5, 5.5, 6, 6.5, 7, 7.5, 8, 9, 10, 11, 12, 13, 14, 15, 20, 25, 30, 35, 40, 45 or 50 inch-pounds or more or any value or range of values therebetween in 0.01 inch-pound increments. In an embodiment, this is an average (mean, median and/or mode) to totally engage from disengage and/or vice versa. In an embodiment, this is the maximum amount needed (e.g., to overcome the detent “locking” between the knob and the handle). In an embodiment, this is after overcoming the detent “locking.” In an embodiment, the orthosis is configured to convert the torque to a linear force. That is, the rotational movement of the knob is converted to a linear movement of the trolly. Thus, the linear force drives a trolley to engage and disengage, depending on the direction of the force, an articulation control system of the foot orthosis. In some embodiments, the torque is applied to a knob carried by the second apparatus. As detailed above, there are no loose parts when the control is completely engaged and when the control is completely disengaged. In an embodiment, there are never any loose parts of the system.

The orthosis can be configured so that the control remains engaged (e.g., locked) when the torque is removed from the knob and the orthosis can be configured so that the control remains disengaged (unlocked for example) when the torque is removed. Also, the orthosis can be configured so that the torque applied in a first direction engages the control and a torque applied in a second direction opposite the first direction disengages the control.

An exemplary foot orthosis can comprise a first apparatus configured to grip a shoe worn by a human and a second apparatus configured to grip a leg of the human, and the foot orthosis can include includes a system configured to control whether and/or how much the first apparatus articulates relative to the second apparatus when the human walks using the foot orthosis. The system can include a first portion that is carried by the first apparatus and a second portion that is carried by the second apparatus. In an embodiment, all moving parts, relative to the first apparatus, of the system are carried by the second apparatus. In an embodiment, all moving parts, relative to the first apparatus, of the system are carried solely by the second apparatus. Also, of the parts carried by the second apparatus of the system, only two move relative to the second apparatus and/or of the parts carried by the second apparatus of the system, there are only three parts of the system. In an embodiment, of the parts carried by the second apparatus of the system, only one moves linearly relative to the second apparatus, and/or of the parts carried by the second apparatus of the system, only one moves rotationally relative to the second apparatus.

In an embodiment, of the parts carried by the first apparatus of the system, the parts so carried are monolithic with a body that establishes sidewalls of the first apparatus. The orthosis can be configured so that the first portion and the second portion form a plunger lock arrangement.

Embodiments include methods that include actions related to the articulation control system. FIG. 57 presents an exemplary flowchart including an exemplary method 57000 that includes method action 5710, which includes obtaining an ankle brace. In an exemplary embodiment, this can entail obtaining any of the exemplary braces detailed herein that have the feature that can selectively limit or otherwise control the articulation range of the heel enclosure relative to the upright. In an embodiment, method action 5710 can also include placing the ankle brace around a show. That said, this sub action can be done later in the method. And in this regard, method 5700 also includes method action 5720, which includes the action of adjusting the ankle brace so that articulation of one component relative to another component is one of more limited or less limited relative to that which was previously the case. In some embodiments, the action of adjusting the ankle brace could occur before the brace is placed around the shoe. Embodiments can include adjusting the ankle brace before placing the ankle brace around the shoe and after placing the ankle brace around the ship. In an embodiment, the action of adjusting the ankle brace can entail any one or more actions associated with any one or more of the features detailed herein, or within the interest of textual economy. In an embodiment, the action of adjusting entails limiting articulation, which can include preventing any articulation or allowing articulation through a range that is more limited than that which was previously the case. In an exemplary embodiment, this can entail engaging the trolley with the teeth of the male portion of the joint, or otherwise lowering the trolley into a gap in the male portion in accordance with the teachings above. In embodiments where method action 5720 entails adjusting ankle brace so that articulation of one component relative to another component is less limited relative to that which was previously the case, this can entail unlocking the locking apparatus, or retracting the trolley away from the male portion of the joint.

In an embodiment, the action of adjusting ankle brace so that the articulation is more limited entails locking the upright relative to the heel enclosure at any of the angles detailed herein. In an exemplary embodiment, the action of adjusting the ankle brace that articulation of one component relative to another component is more limited relative to that which was previously the case entails engaging the articulation limiting system so that the heel enclosure can articulate relative to the upright for a range of motions that is less than that which was previously the case prior to engaging the system. In an exemplary embodiment, action 5720 can be executed one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 times or more or any value or range of values therebetween in one increments in any given day. In an exemplary embodiment, action 5720 can be executed any number of those times after the ankle brace is placed over the shoe with the shoe on a foot of a human and before the ankle brace is removed from the shoe and before the foot is removed from the shoe. In an embodiment, the action of adjusting can include locking to any of the angles or range of angles detailed herein and/or limiting articulation to any of the angles or range of angles detailed herein and then unlimiting.

FIGS. 58 and 59 present another, different embodiment, from the embodiments of FIGS. 1-14 described above. By way of example only and not by way of limitation, the embodiments of the external ankle brace 6500 of FIGS. 58 and 59 and others include an adjustable bottom strap that has ridges or grooves and is made of Kevlar in an embodiment, and has a male-female-male-female interface between the strap and the clasp. Moreover, embodiments of the external ankle brace 6500 of FIGS. 58 and 59 for the most part include an anti-catch bump on the foot portion and/or have a medial side interface that is more pronounced or otherwise different from those noted above. For example, the upright is contoured to accommodate the medial malleolus differently. These aspects and other aspects will be described in greater detail below. Unless otherwise noted, all of the disclosure from here forward corresponds to a disclosure relating to embodiments that are different from the embodiments of the prior embodiments. That said, the embodiments disclosed below can use one or more of the features of the embodiments described above. Indeed, the following description below will not address at least some of the features that are common with the embodiments above (e.g., the upper fastening system, the strap used with the uprights, etc.), but at least some of those features are also present in the embodiments described below. Any interpretation of claim language relating to 35 USC 112, sixth paragraph/(f) corresponds to the embodiments detailed below, although such claims can also include the features detailed above if there is no feature disclosed below that falls within such claim construction. And note that embodiments can include combining one or more of the features below with one or more of the features of the aforementioned patent applications unless otherwise noted providing the art enables such (thus this constitutes additional disclosure made by way of reference to achieve textual economy).

FIG. 58 shows brace 6599 with straps 6901 that are part of a lower leg fastening system, where the straps extend about the outside of the uprights (upright 6200 is shown and the medial upright is eclipsed by the lateral upright 6200), and is attached with a Velcro attachment on the outside of the uprights. The straps are fit through the rectangular rings and then pulled back in the opposite direction and then Velcroed to the outside of the adjacent strap body to secure the straps. Conversely, the strap 6902 of the upper fastening system extends through openings in the sidewalls and thus has a portion that goes inside the brace/inside the sidewalls. In an embodiment, the openings have a height the vertical direction of FIG. 76A, showing a cross-section through the openings) that is less than, greater than and/or equal to 0.05, 0.1, 0.15, 2, 0.25, 0.3, 0.35 of 0.4 inches or more or any value or range of values therebetween in 0.01 inch increments. And the length can be 0.25, 0.3, 0.35, 0.4, 0.45, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4 or 2.5 inches or more or any value or range of values therebetween in 0.01 inch increments. Again, a Velcro fastening system is utilized to secure the strap 6902. In this embodiment, there is no Velcro or adhesive attached to the sidewalls. That is, the Velcro connection of the strap 6902 is all that is used to secure the strap 6902 to the uprights. In other embodiments, adhesive and/or a dedicated Velcro surface can be attached to the sidewalls. In another embodiment, rectangular loop(s) are attached to the sidewalls by a rivet connection or some other connection, and the strap 6902 is fit through the loop(s) instead of through the sidewall(s). A combination of these can be used (one loop on one side (lateral or medial) and one opening on the other side). In an embodiment, there is only one strap over the top of the shoe/for the upper fastening system. In an embodiment, there are two, three or more straps.

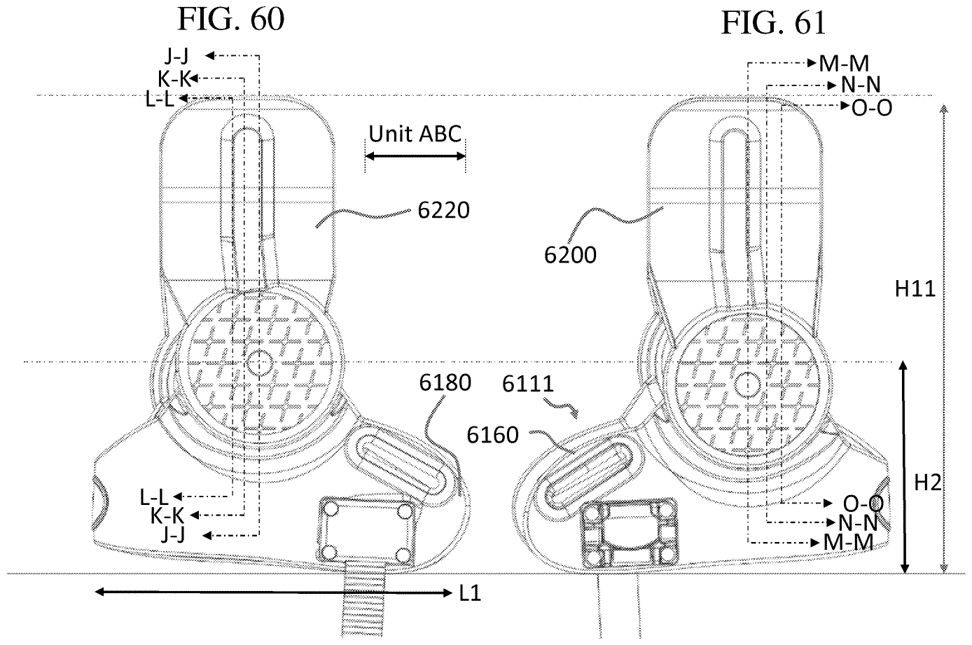

FIGS. 60-61 present side views of the extra level brace 6500 according to an exemplary embodiment. Here, it can be seen that there are lateral upright extension 6200 and medial upright extension 6220. In an exemplary embodiment, these uprights can have one or more of the features detailed above with respect to the uprights of the embodiments above. These uprights are jointly connected to the rigid heel enclosure 6111. The rigid heel and closure 6111 includes a lateral sidewall 6160, and a medial sidewall 6180. Again, the sidewalls can have one or more of the features detailed above with respect to the sidewalls of the embodiments above. As will be described in greater detail below, the joints of the respective lateral and medial upright extensions have a male-female joint assembly, some respect to portions of which are monolithic with the remainder of the bulk of the extensions and sidewalls respectively. Again, additional details of this will be described below.

FIG. 61 shows a dimension H1, which is the height from the bottom to the top of the brace 500. In an exemplary embedment, H1 can be less than and/or equal to and/or greater than (meaning that it can be less than one of the following numbers and greater than another of the following numbers for example) 6, 6.25, 6.5, 6.75, 7.0, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75 or 15 inches or any value or range of values therebetween in 0.0001 inch increments (to achieve English units for example). And note that the sides of the brace can be different heights (e.g., the top of the lateral upright extension can be at a different height than the top of the medial upright extension), and thus one side can have one of the aforementioned values, and the other side can have another of those values. Indeed, embodiments are such that that is the case. H1 or H11 can be any of those detailed herein.

Note that these drawings herein (all drawings unless otherwise noted) are to scale, and thus disclosures constitute values that are scaled relative to other specific values for purposes of textual economy.

FIG. 61 shows a dimension H2, which can be any of those noted above.

FIG. 15 shows a dimension L1, which is the length of the lateral sidewall (and can be the length of the medial sidewall) from the rearmost end to the foremost end, and can be any of those above.

These dimensions are given so that the other portions of the brace can be scaled to within 20, 19, 18, 17, 15, 15, 14, 13, 12, 11, 10, 9, 8, 7, 6, 5, 4, 3, 2, or 1%.

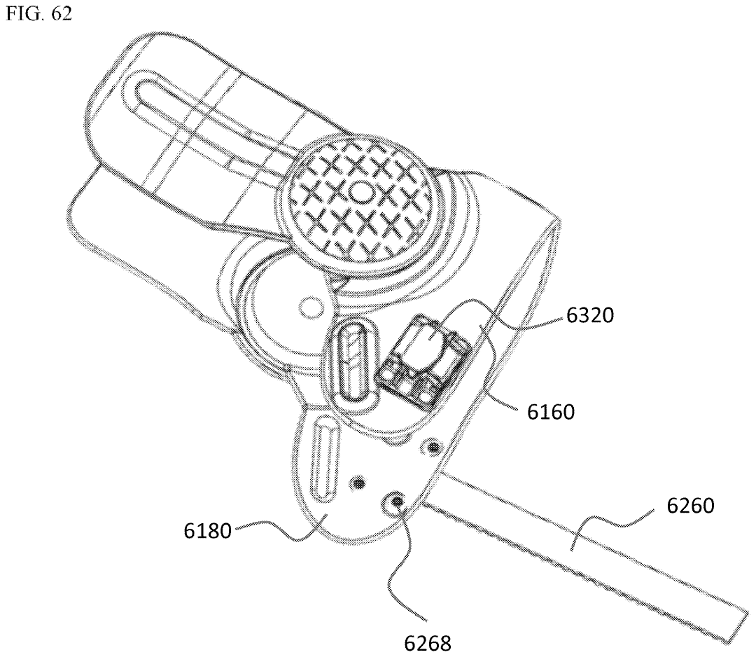

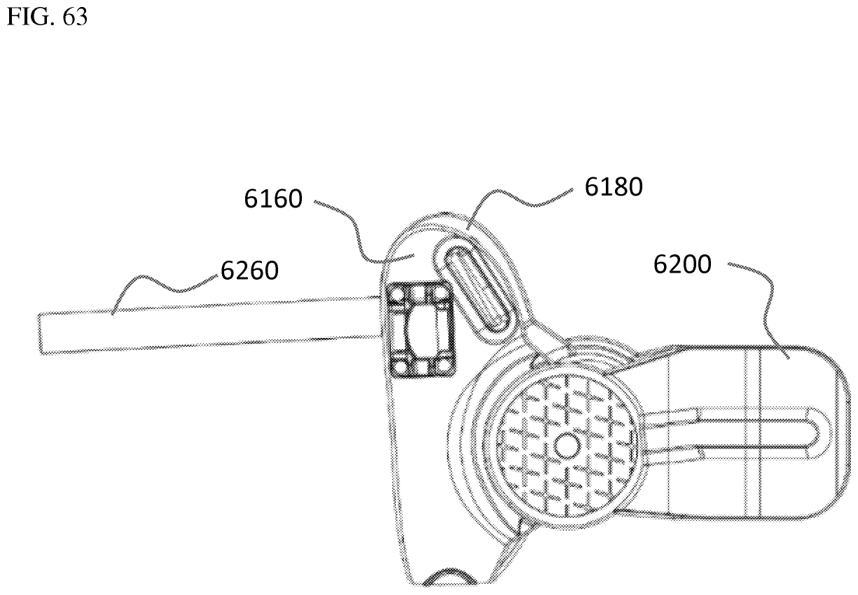

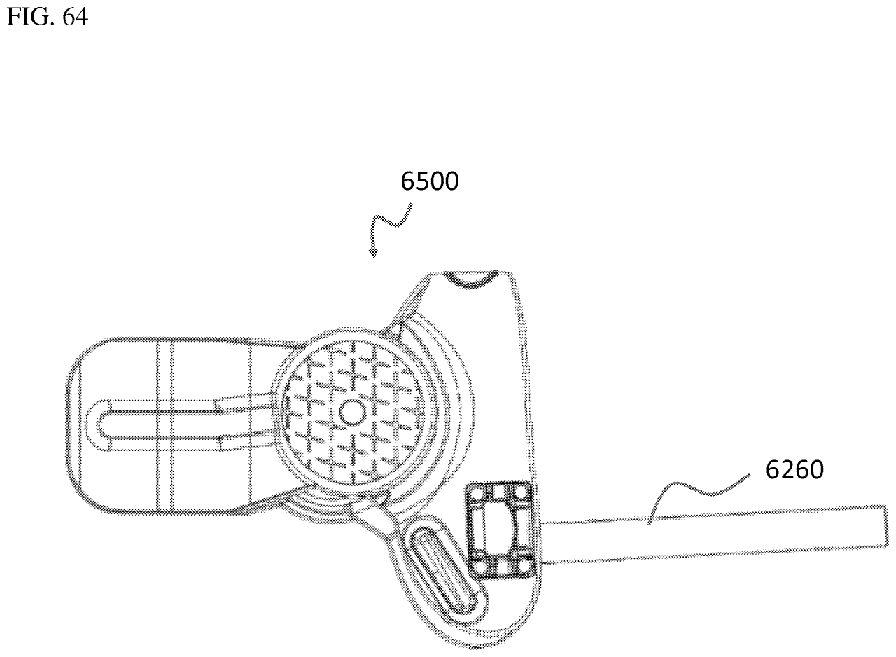

FIGS. 62-67 provide additional views of the external ankle brace 6500 according to exemplary embodiments. FIGS. 62-65, etc., show the lower connecting strap 6260. Like the embodiments of FIGS. 14-18, the lower connecting strap in this embodiment is configured to be adjustable and is configured to have one end that can be freed by a user from connection with the respective sidewall, but unlike those embodiments, in some embodiments, the freeing is difficult, and otherwise is meant for judicious adjustment that is not for daily adjustment. In these embodiments, it is the lateral sidewall where the releasable connection is present, although in other embodiments, it can be the medial sidewall and/or can be both sidewalls in some embodiments. Still, with respect to the embodiment shown in the figures, the lower connecting strap 6260 is fixedly mounted to the medial sidewall 6180 indirectly by rivets 6268. Here, four (4) rivets are used, but one or two or three or five or six or more can be used. Bolts or Chicago screws can be utilized. In an exemplary embodiment, the medial sidewall can be molded around the strap 6260. By way of example only and not by way of limitation, holes through the strap 6260 can be present, and during the molding operation, the material of the sidewalls will flow into the holes and thus create a positive retention between the strap and the sidewall. That said, alternatively and/or in addition to this, the male portions that are present on the strap can be present such that the sidewalls will be molded around the strap thus also creating positive retention, and in some embodiments, the action of molding can be akin to the molding around the top of a T or the bottom of an L—the character cannot be pulled own or up, respectively, because of the material around the horizontal portions, providing that the strap has such an arrangement, where in some embodiments, this is not the case.

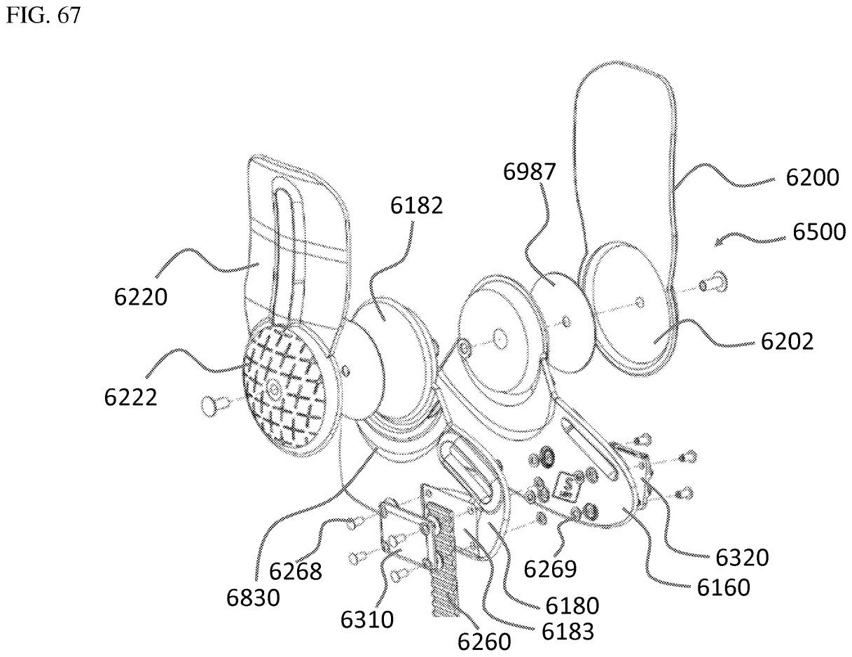

FIG. 67 shows an exploded view of portions of the brace 6500 without the cloth straps/the straps that use Velcro/the upper fastening system and the lower leg fastening system, showing the backing washers 6269 located on the inside of the sidewalls through which the rivets 6268 fit prior to swaging and which provide stress distribution for the fastening of the strap to the sidewall, in this embodiment for example. Also seen are washers located on the outside of the sidewalls that likewise can be used to distribute stress. All of these are optional. Also seen is the boss/thickened area 6183 of the sidewall onto which a securement structure 6310 sits when assembled. The thickened area 6183 is a monolithic part of the sidewall/the foot interface/heel enclosure in some embodiments. In an embodiment, this can also include a depression/cavity into which the structure 6310 can fit. Also seen are depressions in the sidewalls for the rivets/washers. This can have utilitarian value with respect to providing an assembly where the heads of the swaged rivets are not proud/and/or below the inner profile of the inner surface of the sidewalls. In an embodiment, the sidewalls are thickened at locations proximate the washers/rivets. In an embodiment, a berm 6185 of the sidewall material surrounds the washer/depression for the washer so that there is a smoother transition between the remainder of the surface of the sidewall and that area (the rivet would otherwise be proud of the surrounding sidewall material—in embodiments, this still can be the case, it is just less proud). It could be that the tops/crests of the berm(s) establish a relatively level set of highpoints inside the sidewalls in this area in some embodiments. In an embodiment, the pertinent surface are located within two planes separated by less than 0.1, 0.75, 0.5, or 0.025 inches when the structure is in the relaxed state (all disclosure herein corresponds to an embodiment where the structures are in a relaxed state unless otherwise noted, in the interests of textual economy—only gravity acts on the structure in such disclosure). FIG. 67 shows the male portion 6182 of the medial joint.

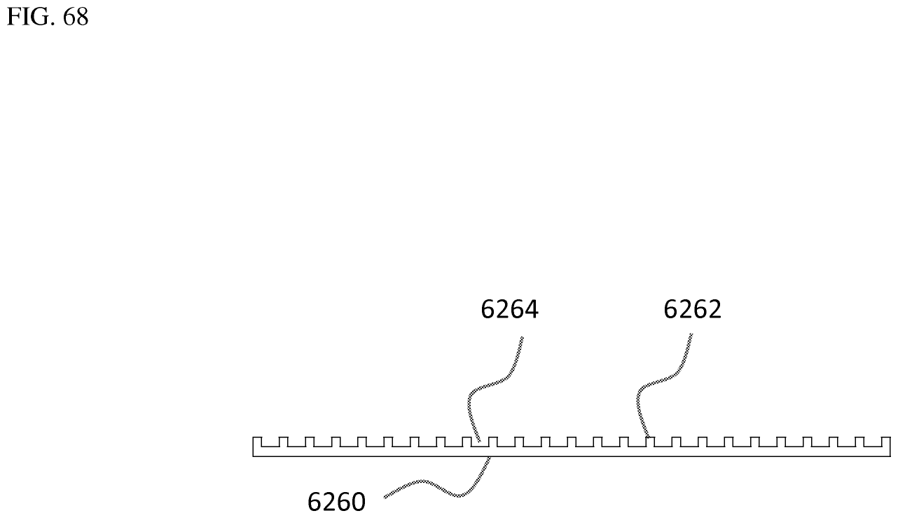

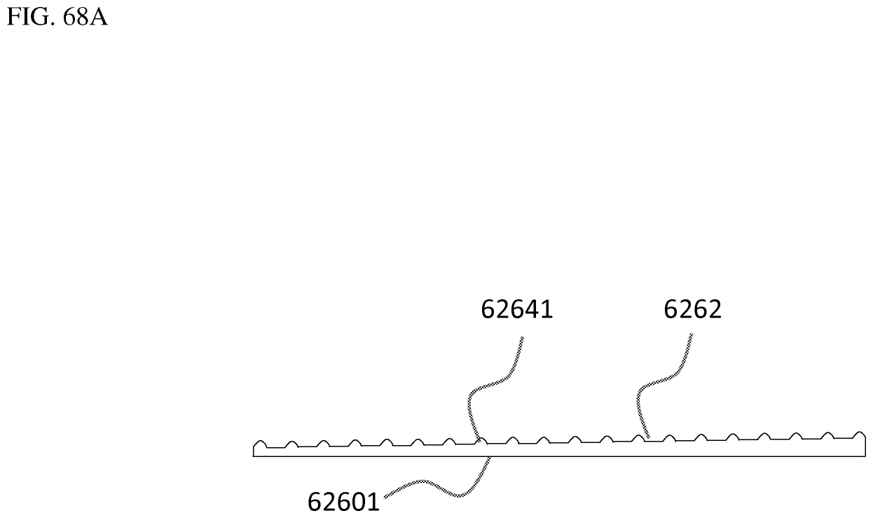

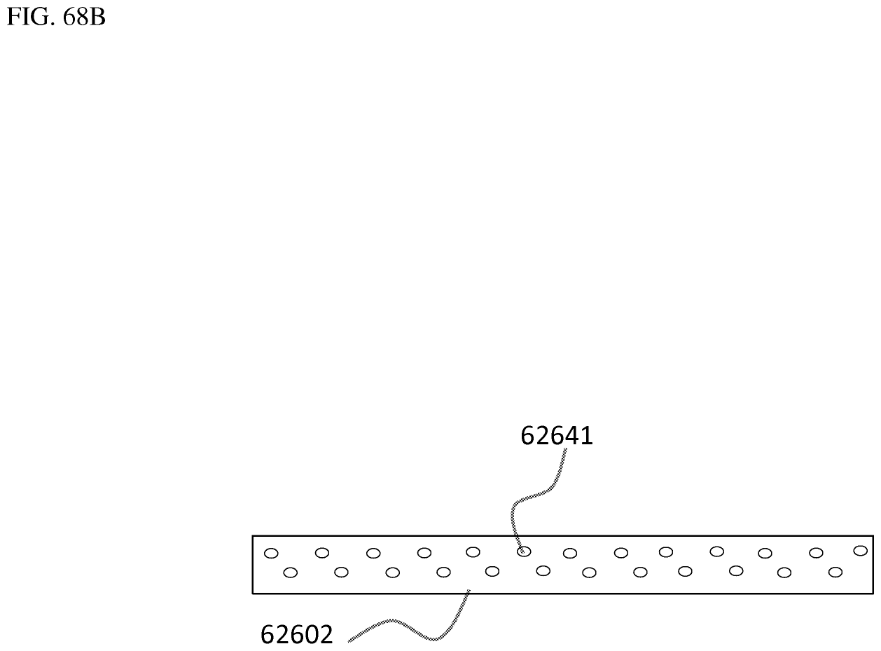

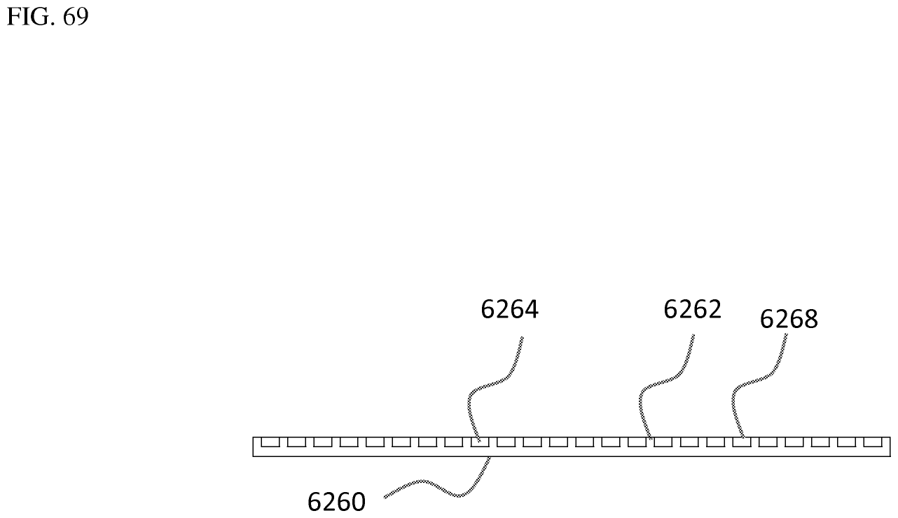

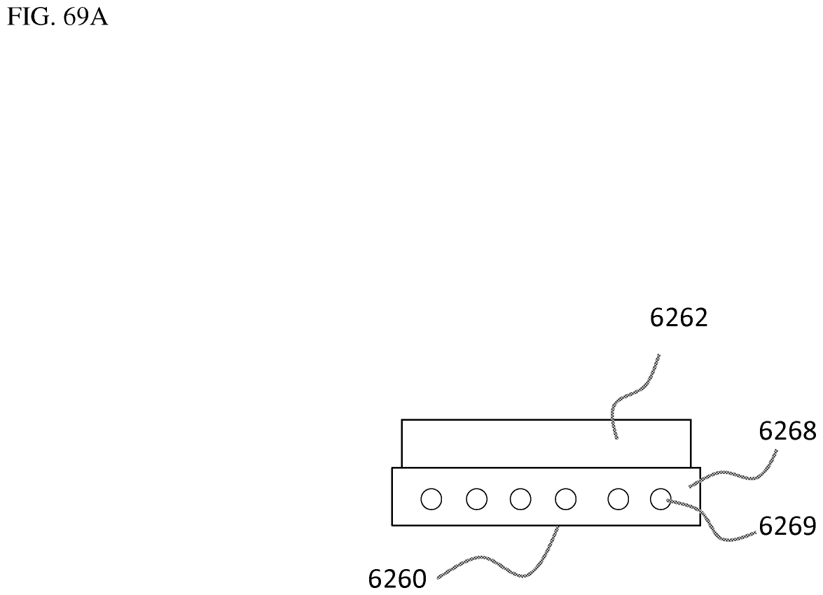

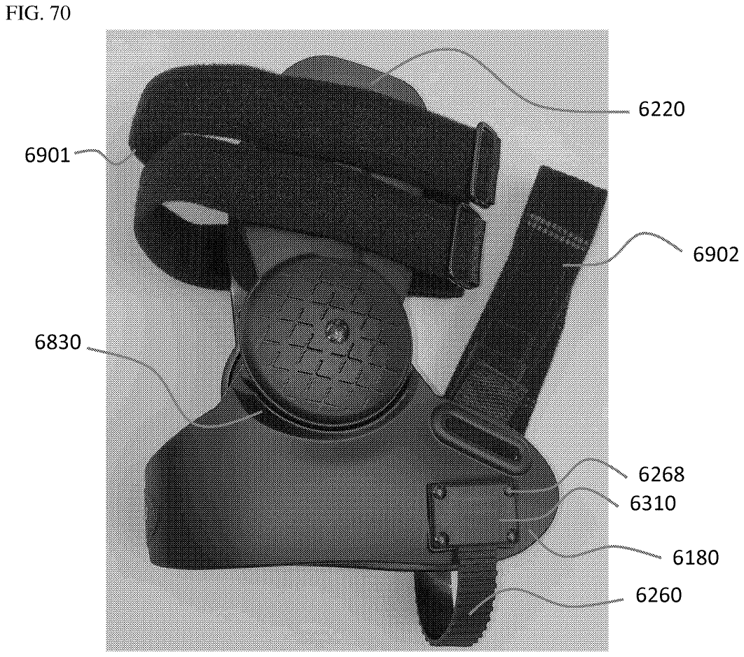

In an exemplary embodiment, the strap 6260 can have a width (in the long direction of the rigid heel enclosure) of less than and/or equal to and/or greater than 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, or 2 inches or more, or any value or range of values therebetween in 0.0001 increments (or less), at least to achieve the standard English measurements (e.g., 0.375 inches, 0.625 inches, 0.0625 to 0.875 inches, etc.). The strap 6260 can have a thickness of less than and/or equal to and/or greater than 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, 0.20, 0.21, 0.22, 0.23, 0.24, 0.25, 0.3, 0.35 inches or more, or any value or range of values therebetween in 0.0001 increments (or less), at least to achieve the standard English measurements (e.g., 0.125 inches, 0.0625 inches, 0.0625 to 0.33 inches, etc.). The thickness is the height of the strap if the strap were laid flat on a table for example, lengthwise. In an embodiment, the thickness oscillates in a uniform pattern, as seen in FIG. 68, which is a side view of the strap laid flat. Here, there is the main expanse (main portion—the portion that extends completely from left to right) from which teeth 6262 or ridges protrude, separated by depressions/valleys 6264. In an embodiment, the height of the teeth from the base/lowest portion of the valley is less than and/or equal to and/or greater than 0.02, 0.025, 0.03, 0.035. 0.04, 0.45, 0.05, 0.06, 0.07, 0.08, 0.09, 0.1, 0.125, 0.15, 0.12, 0.13, 0.14, 0.15, 0.16, 0.17, 0.18, 0.19, 0.2, or 0.25 inches or more, or any value or range of values therebetween in 0.001 increments (or less) and/or the valleys can have a maximum depth from the highest point on the teeth of any of these values, and the two need not be the same, in the interests of textual economy. While the embodiment of FIG. 68 shows a digital rectangular embodiment, in other embodiments, the teeth/ridges could be curved (partial circular shape or partial oval shape), triangular shape, or other shape, and the same for the valleys. Indeed, an oscillatory wave can be used for the cross-section thereof (that lying on the plane of FIG. 68). And note that while FIG. 68 shows the side view of the strap, this can also be the cross section. Thus, in some embodiments, the cross-section can be uniform into and out of the plane of FIG. 68. Conversely, the teeth could be within/bounded by walls of the strap, such as that seen in FIG. 69, which is a cross-section through the longitudinal center of the strap, where there is a sidewall 6268 seen in the background (there could be/would be one in the foreground in some embodiments as well, where the teeth span from each sidewall in some embodiments). And while the embodiment of FIG. 69 shows the side wall having a height that is equal to the height of the teeth, in other embodiments, the height of the sidewall can be lower than the height of the teeth or higher than the height of the teeth. In an embodiment, the height of the sidewall above the bottom portion of the various cavities can be any of those detailed herein for the height of the teeth in the interest of textual economy, and the values need not be the same as the height of the teeth. And note that the height of the teeth and/or the height of the sidewalls can vary along the length of the strap, and thus the heights can be locally any of the points detailed herein in the interest of textual economy. Also, the dimensions of the depressions/valleys 6264 can have any one or more of the dimensions of the teeth disclosed herein, in the interest of textual economy, and the values need not be the same.

In an embodiment, the teeth span a distance in the longitudinal direction of less than, greater than and/or equal to 0.02, 0.025, 0.03, 0.035. 0.04, 0.45, 0.05, 0.06, 0.07, 0.08, 0.09, 0.1, 0.125, 0.15, 0.12, 0.13, 0.14, 0.15, 0.16, 0.17, 0.18, 0.19, 0.2, or 0.25 inches or more, or any value or range of values therebetween in 0.001 increments (or less). This value can be a mean, median, and/or mode value of any portion or all of the teeth as measured from the base of the teeth to the top of the teeth. This can be a maximum thickness (e.g., the base of a triangle) or minimum thickness (e.g. the top of a truncated triangle (where the point is shaved off)). In an embodiment, the span of the teeth can vary into and out of the plane of FIG. 68. The span can be any of these values at any location. Still, in an embodiment, the span is constant with depth into and out of the plane of FIG. 68.

In an embodiment, there are less than, greater than and/or equal to 3, 4, 5, 6, 7, 8, 9, 10, 11, 12, 13, or 14, or any value or range of values therebetween in one increment teeth per inch of length of the strap. The length of the strap can be less than and/or equal to and/or greater than 3, 3.5, 4, 4.5, 5, 5.5, 6, 6.5, 7, 7.5, 8, 8.5, 9, 9.5, 10, 10.5, 11, 11.5, 12, 12.5, or 13 inches or more, or any value or range of values therebetween in 0.0001 increments (or less), at least to achieve the standard English measurements. In an exemplary embodiment less than and/or equal to and/or greater than 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75, 3, 3.25, or 3.5 inches or more, or any value or range of values therebetween in 0.0001 increments (or less), at least to achieve the standard English measurements (e.g., 0.375 inches, 0.625 inches, 0.0625 to 0.875 inches, etc.) of the strap 6260 can be overlapping and/or embedded in the sidewall to which the strap is fixedly attached.

In an embodiment, the teeth span (the direction of extension of an individual tooth) the full width of the strap 6260.

In an embodiment, the teeth span a distance that is less than the full width of the strap. The length of a tooth in the expansion direction can be any of those detailed herein with respect to the width of the strap as measured in a direction parallel thereto and need not be the same in the interest of textual economy.

FIG. 69A shows a cross-section of an embodiment of the strap, showing reinforcement components 6269 embedded in the flexible material 6268 that form the main body of the strap. In an embodiment, the components 6269 can be metal or Kevlar or can be wire strands or sold wires or any reinforcing that can have utilitarian value.

In an embodiment, the strap is a composite strap. The strap can have less than, greater than and/or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more or any value or range of values therebetween in 1 increments distinct reinforcement components, and these can be respectively made up of sub-components (less than, greater than and/or equal to 2, 3, 4, 5, 6, 7, 8,9, 10, 11, 12, 13, 14, 15, 16, 17, 18, 19 or 20 or more or any value or range of values therebetween in 1 increments per each component, and each subcomponent can be a monolithic sub-component). Each component can be monolithic as well. Thus, there is a lower connecting strap includes one or more elongate reinforcement components embedded in a covering material.

In an embodiment, any of the number of reinforcing materials extends for less than, greater than and/or equal to 50, 55, 60, 65, 70, 75, 80, 85, 90, 91, 92, 93, 94, 95, 96, 97, 98, 99 or 100% or any value or range of values therebetween in .5% increments of the length of the strap. In an embodiment, the components are less than, greater than and/or equal to 0.01, 0.015, 0.02, 0.025, 0.03, 0.035, 0.04, 0.045, 0.05, 0.055, 0.06, 0.065, 0.07, 0.075, 0.8, 0.085, 0.09, 0.095, 0.1, 0.11, 0.12, 0.13, 0.14, 0.15, 0.17, 0.18, 0.19 or 0.2 inches or any value or range of values therebetween in 0.005 inch increments, and the spacing need not be the same. In an embodiment, any of the just numbered components can be unbroken (all can be unbroken/uninterrupted from a beginning to end of the strap). In an embodiment, there is no through hole in the strap. The strap is totally unbroken except for the cuts at the ends (if the trap is even cut at that component). The material that is used to form the outer portions of the strap (top and/or bottom and/or sides—the only place in some embodiments where the reinforcement can be seen are the ends (resulting from cutting of the strap, in some embodiments only) are contiguous and unbroken. The teeth are part of the material in an embodiment (although in some embodiments, the teeth can also have reinforcing components). Thus, in an embodiment, there is no room for a male portion to extend from one side of the strap to the other side of the strap. In an embodiment, the largest passage from one side of the strap to the other has a maximum diameter measured in a plane normal to a longitudinal axis of no more than 0.1, 0.075, 0.05, 0.04, 0.03, 0.02, 0.01 or less inches or any value or range of values therebeween in 0.005 inch increments.

In an embodiment, the strap has a length (exposed or unexposed in textual economy) of less than, greater than and/or equal to 3, 3.5, 4, 4.5, 5, 5.5, 6, 6.5, 7, 7.5, 8, 8.5, 9, 9.5 or 10 inches or more or any value or range of values therebetween in 0.05 inch increments.

In an embodiment, a Gates Mectrol timing belt is used to make the strap. An imperial pitch belt can be used. A T pitch belt can be used. An AT pitch belt can be used. An HTD and/or STD pitch belt can be used. The material 6268 can be a polymer material.

In an embodiment, with respect to a cross section of the strap through the longitudinal axis thereof, such as a cross section lying on the plane of the frame of reference of FIG. 68, the shapes of the teeth and/or the corresponding valleys or depressions are constant from one side of the strap to the other side of the strap (for example a series of planes moving above and/or below the frame of reference of FIG. 68) that is, a cross-section at one location is the same as a cross-section at another location. In an alternate embodiment, this is not the case. The shapes and/or the dimensions of the teeth and/or the valleys/depressions can change with location in those directions. And note also this is also the case with respect to the longitudinal direction of the strap. Teeth that one portion of the strap could be different than that of the other portion of the strap. There could be at least 1, 2, 3, 4, 5, 6 or more different configurations of teeth. Bumps, bowties, etc., can be the teeth. FIG. 68A shows a sideview of curved teeth 62641 on an exemplary strap 62601, with a tapering height in an embodiment. FIG. 68B shows a top view of a strap 62602 where the teeth 62641 are oval shaped and staggered on either side of the strap.

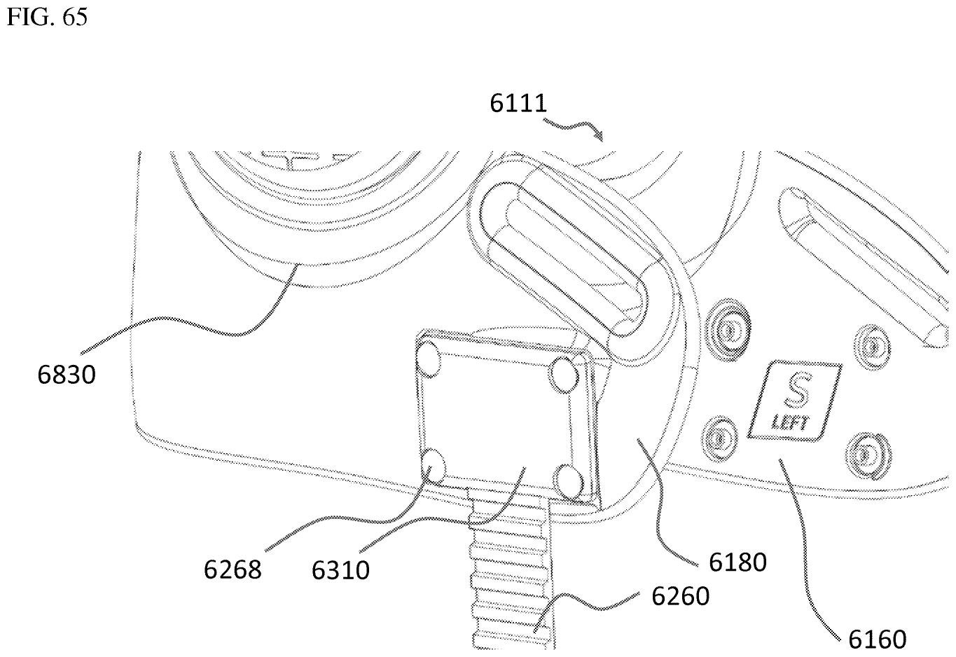

FIG. 65 shows a view of the rigid heel enclosure 6111. This shows the inside of the lateral sidewall 6160, and showing the exterior facing heads of the rivets 6268 that are utilized to secure the strap to the sidewall, albeit in conjunction with the securement apparatus 6310. Also shown are the rivets used to secure the clasp 6320, but not the backing (the rivets have not been swaged, and in practice, there is a washer (backing) that is used to provide additional material to react the force over the area of the body of the sidewall).

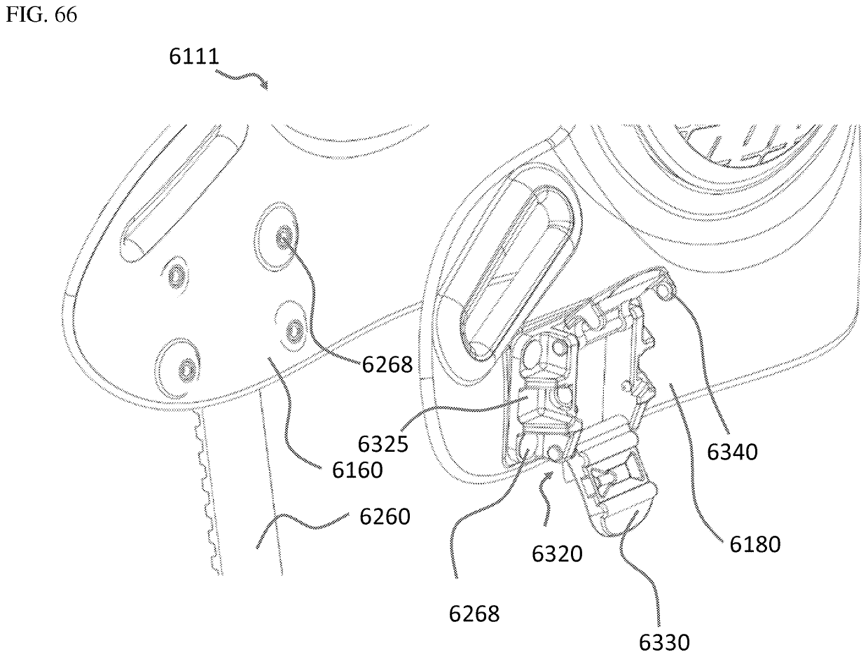

FIG. 66 shows a view of the rigid heel enclosure 6111. This shows the inside of the medial sidewall, and showing the interior facing heads of the rivets 6268 that are utilized to secure the strap to the sidewall, albeit in conjunction with the securement apparatus 6310. As seen, the strap is completely exterior to both sides of the sidewalls, but in an embodiment, it is inside on one of the sidewalls (e.g., the lateral sidewall). In an embodiment, on at least one side (the lateral side for example), there is a cavity in the sidewall on the exterior side thereof (but could be on the interior side in an alternate embodiment where the strap is inside on at least one side) that is sized and dimensioned to accept the end of the strap 6260. In an exemplary embodiment, the cavity is sized and configured so that the strap is at least flush with the surface on either side of the cavity so that the strap does not stand proud of the surface, and thus create a pressure point against the shoe.

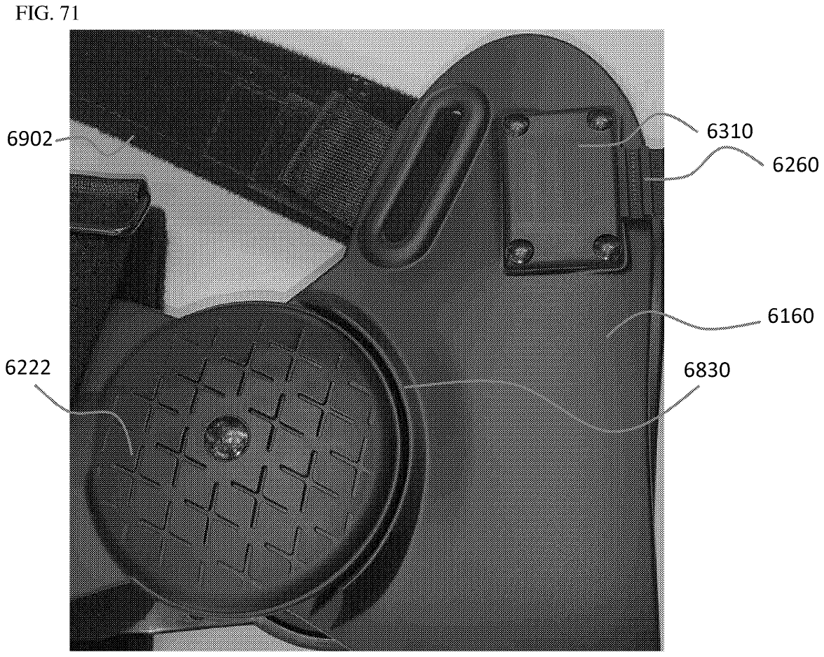

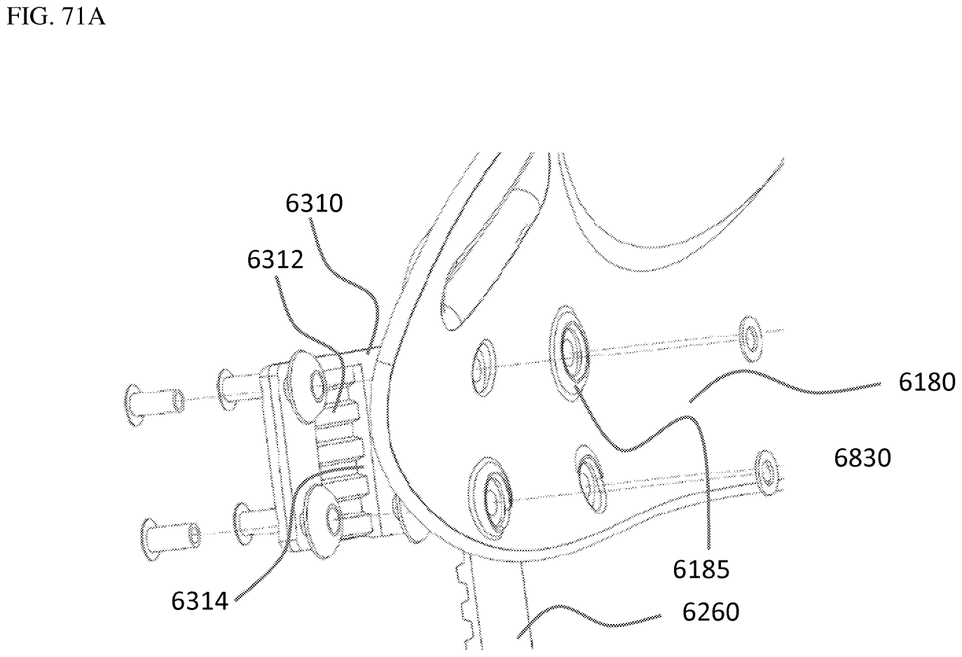

Referring to FIG. 71A, the corresponding mating teeth 6312 of the securement structure 63 10 that interface with the teeth of the strap 6260 can be seen. In an embodiment, the securement structure is a monolithic body that is in the form of a plate that has on one side thereof molded teeth 6312. In an embodiment, the spacing of the teeth and the respective cavities between the teeth corresponds to a spacing that will grip the corresponding teeth and spacing of the strap 6260. In an embodiment, in the interest of textual economy, the teeth 6312 of the securement structure 6310 can have any of the dimensions detailed herein as disclosed with respect to the strap 6260. The dimensions need not be the same in the interest of textual economy. Indeed, in the case where for example the gap or otherwise the space between the teeth of the strap is larger than the width of the teeth in that direction, in at least some exemplary embodiments the corresponding with the teeth of the securement structure 6310 would be or could be more accurately wider than the teeth of the strap so as to “fill” that cavity to provide a desired grip. Indeed, in an embodiment, it is the width of the teeth of the securement structure that are designed to fit into the corresponding gaps or cavities between the teeth of the strap 6260 so that by filling that cavity, the strap cannot move or is otherwise effectively secure or otherwise does not move an appreciable amount when the securement structure 6310 is attached for example by rivets to the sidewall. As seen, there are four depressions or otherwise gaps between the teeth and teeth like structure of the securement structure 6310. There are also four teeth of the securement structure, and one “tooth-like” component at the top. In this regard, in this exemplary embodiment, the top (relative to the orientation of FIG. 71A) of the securement structure 6310 is closed. That is, when looking downward from the top of the brace, the strap 6260 cannot be seen. Accordingly, in this exemplary embodiment, the “last” tooth of the strap fits into the recess or otherwise valley between the tooth like component at the top and the tooth that is closest thereto. In an alternate embodiment, this can be different and the toothlike structure can actually be another tooth and the top of the securement structure can be open. In an embodiment, there are less than, greater than and/or equal to 1 (and thus there is no less than), 2, 3, 4, 5, 6, 7, 8, 9, 10, 11, 12, 13, 14, or 15 or more, or any value or range of values therebetween in one increment teeth and/or valleys of the securement structure 6310, and any one or more of these interface with corresponding teeth and/or valleys of the strap 6260. In an embodiment, the securement structure 6310 includes a recessed portion 6314 in which the teeth 6312 are located. As seen in this embodiment, the tops of the teeth are below the surface of the securement structure that begins the recessed portion. In an embodiment the tops of the teeth can be flush with that surface. In an embodiment, the teeth can be proud of the surrounding surface, and thus the valleys between the teeth would be the recessed portion and there would be no other recessed portion. In an embodiment, the span of the recess from one side to the other (from left to right in the orientation of FIG. 71A) in the direction of extension of a given tooth is the same as the tooth, while in other embodiments, the span could be greater than the span of the tooth or teeth in that recessed person. The span could be just slightly oversized to establish a clearance fit. An interference fit and/or a slip fit in any one or more of the dimensions that interface with the strap could be used. The fact that the strap is made out of a material that has a modicum of compressibility and/or flexibility and/or stretch ability can be relied upon with respect to securing the securement structure 6310 to the strap 6260. In this regard, the teeth of the securement structure could be wider than the valleys or recesses of the strap, and the teeth could interference fit to establish a snug fit. Alternatively, and/or in addition to this, the material of the securement structure in general, and the teeth thereof in particular, could also deform a little bit so as to establish the snug/secure fit. Of course, instead of the securement structure 6310, rivets could also be used to secure the strap to the sidewall. The rivet could extend through a hole in the strap (two rivets could be used and two holes, or more). Still, in an embodiment, the elegance of the securement structure as presented would be utilized. In an embodiment, there is a clamping force applied to at least a portion of the strap by the securement structure 6310 when the securement structure is riveted or otherwise secured to the sidewalls. The clamping force can be due to an interference fit in the direction of the extension of rivets for example. The idea is that the securement structure clamps the strap to the sidewalls (clamped between the securement structure and the sidewall body). It could be that the clamping is only on the tops of the teeth of the strap and the opposite flat surface of the strap, while in other embodiments it is only clamping at the valleys and the opposite surface. The combination of both could exist owing to the flexure or otherwise the plastic (or even plastic for that matter) nature of the materials involved.

In an embodiment, less than, greater than, and/or equal to 1, 2, 3, 4, 5, 6, 7, 8, 9, or 10, or any value or range of values therebetween in one increment teeth and/or valleys of the strap engage with one or more of the teeth and/or valleys of the securement structure, and the values need not be the same, and/or vice versa, again where the values need not be the same, all in the interest of textual economy.

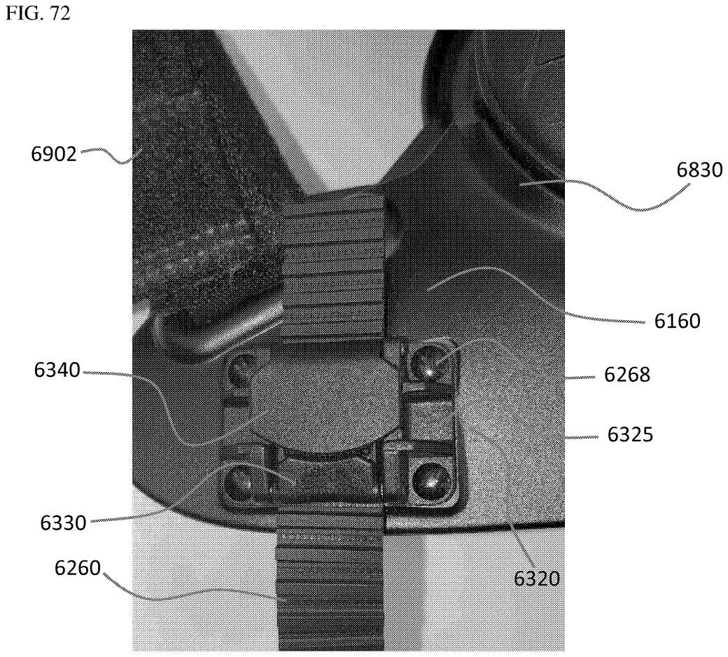

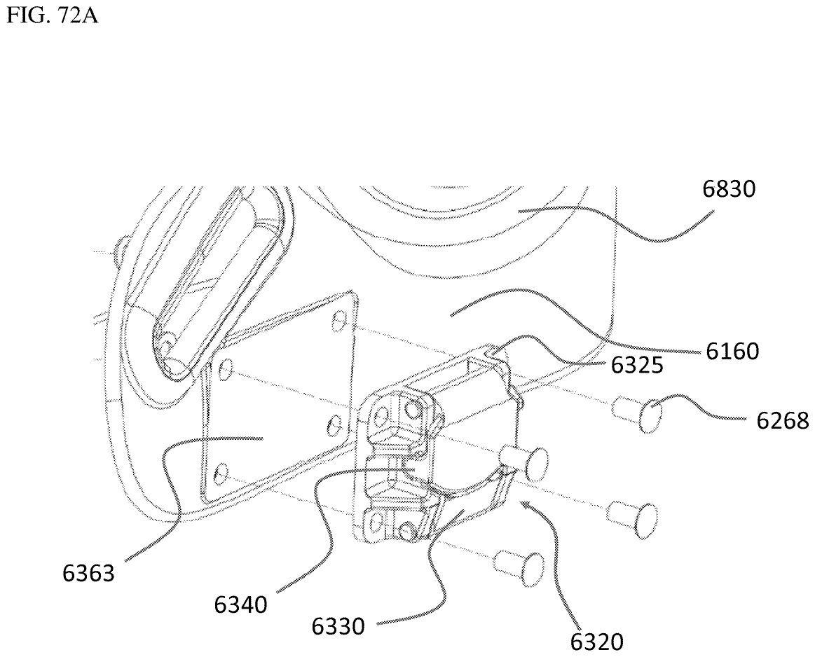

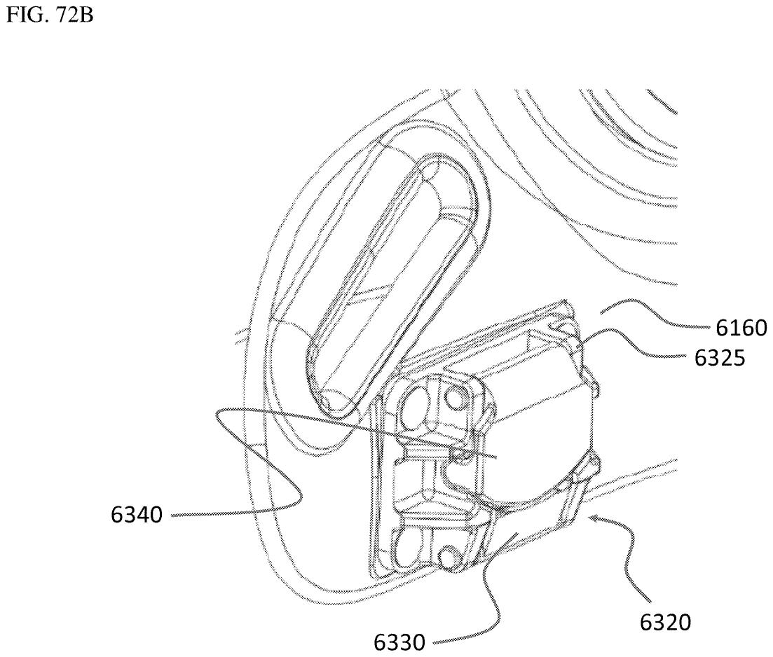

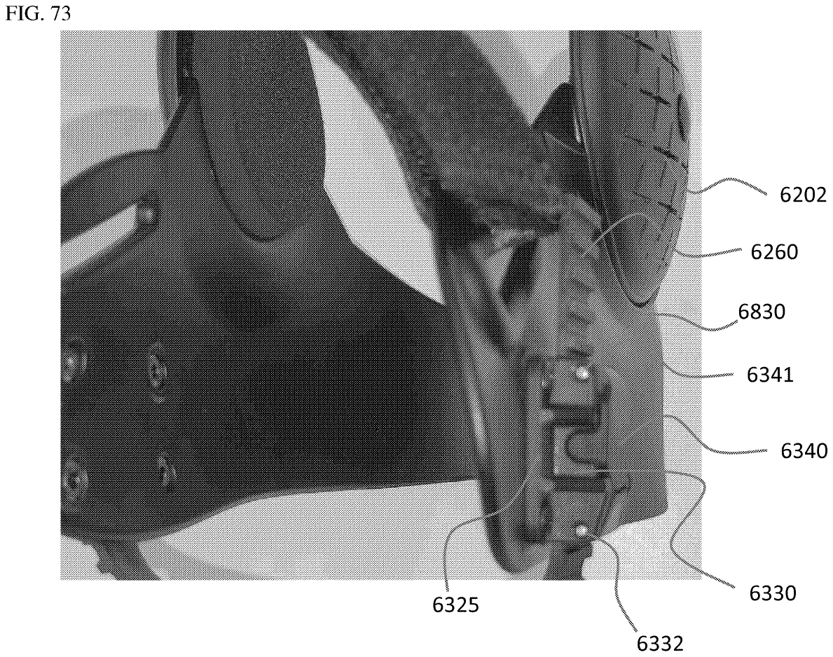

FIG. 66 shows the clasp 6320 of the lower connecting system (that is made up of the strap 6260 and the connecting structure/securement structure 6310, etc.) that enables the releasable connection of the strap 6260 to the lateral sidewall. In this exemplary embodiment, there are two hinged components of the clasp that enable the adjustability and the locking of the strap. As seen, there is a clasp chassis 6325 that is mounted to the lateral sidewall 6165 by rivets 6268. The chassis hingedly supports the mating toothed element 6330 by hinge pin 6332 and hingedly supports the lock component 6340 by hinge pin 6341. The chassis can be considered the component that provides a guide for the strap when the strap is inserted into the clasp. The dynamics of strap insertion and removal in an embodiment will now be described. In an embodiment, the end of the strap is inserted into the bottom of the clasp when the lock component 6340 is in the unlocked position in the mating toothed component 6330 is in the unmated position. This can be seen in FIGS. 76 and 72A (the latter showing an exploded view, and as seen, a thickened boss section 6163 is present to support the clasp 6320—in an embodiment, the maximum thickness of this boss section, which is monolithic with the remainder of the sidewall, is less than, greater than and/or equal to 10, 20, 30, 40, 50, 60, 70, 80, 90, 100, 125, 150, 175, 200, 225, or 250% or more, or any value or range of values therebetween in 1% increments of the maximum thickness and/or minimum thickness of the sidewall immediately adjacent thereto and/or the average thickness (mean, median and/or mode) of the perimeter about the boss)). While the chassis does provide the bulk of the guidance, the way the hinge pin 6332 and the mating toothed component 6330 are located relative to the “bearing surface” of the chassis that supports the flat side of the strap (the side of the strap that faces the medial sidewall) results in a slight interference fit or a slip fit between the top portions of the strap (the teeth or protrusions, or more accurately the top portions thereof) and the portion of the mating toothed component 6330 through which the hinge pin 6332 extends. In an embodiment, the fit is a clearance fit but relatively constrained so that there is a level of precision that must be involved when placing the strap into the class from the bottom of the clasp with respect to the frame of reference of FIG. 73 for example. In an embodiment, the teeth of the strap and the portion of the mating toothed component that interfaces with the strap when the mating toothed component is in the open position and not interfacing with the teeth of the strap as intended to lock the strap in place are configured to provide a quasi-detente functionality. A little bit of force must be applied to the strap to push the strap into the clasp above the force required to simply move the strap or otherwise bend the strap so that will reach the clasp. This provides a level of feedback to the user of the brace or otherwise a person adjusting the brace for a particular user is going to wear the brace. Indeed, in an embodiment, as the strap is being inserted into the chassis or otherwise inserted into the clasp with the two hinged components in the unrestrained position, a ticking sound can be heard at about 300 hertz or so (in an embodiment, the frequency of the clicks centered at any one or more of 100, 150, 200, 250, 300, 350, 400, 450, 500, 550, 600, 650, or 700 Hz, or any value or range of values therebetween in 1 Hz increments and/or a mean, median and/or mode or maximum value of the frequency generated by the clicks is such. The level of force to insert the strap into the “open” clasp is less than, greater than and/or equal to 0.1, 0.2, 0.3, 0.4, 0.5, 0.6. 0.7. 0.8. 0.9, 1, 1.25, 1.5, 1.75, 2, 2.25, 2.5, 2.75, 3, or 3.5 Newtons, or any value or range of values therebetween in 0.01 N increments, but again in at least some embodiments, greater than the force needed to simply move the strap to the chassis and/or otherwise to slide the strap into the chassis if the mating toothed component and/or the lock component 6340 were not present (for example removed for the purposes of evaluation). In an embodiment, at any one or more of the just noted forces of insertion (i.e., upward with the frame of reference of FIG. 73, with an essentially rigid grip one inch away from the bottom of the clasp with the strap passing past both hinge pins and at least one tooth on the other side of the hinge pin 6341, a tooth passes the hinge pin 6332 at a rate of less than, greater than or equal to 0.75, 1, 1.25, 1.5, 1.75, 2, 2.25, 2.5, 2.75, 3, 3.25, 3.5, 3.75 or 4 teeth per second or any value or range of values therebetween in 0.05 increments (e.g., 1.3, 1.6, 1.15 to 2.3 per second, etc.).

FIG. 76A shows a cross-section through the brace through the clasp.

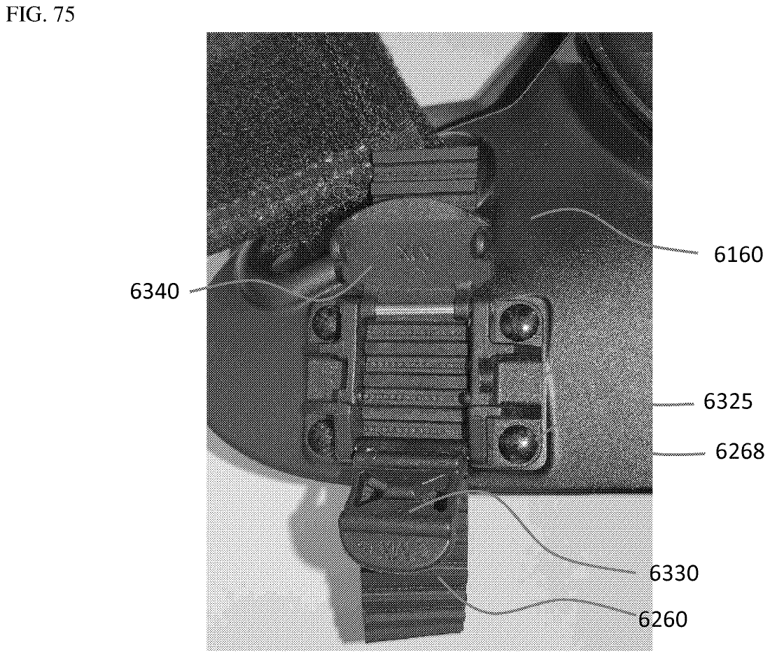

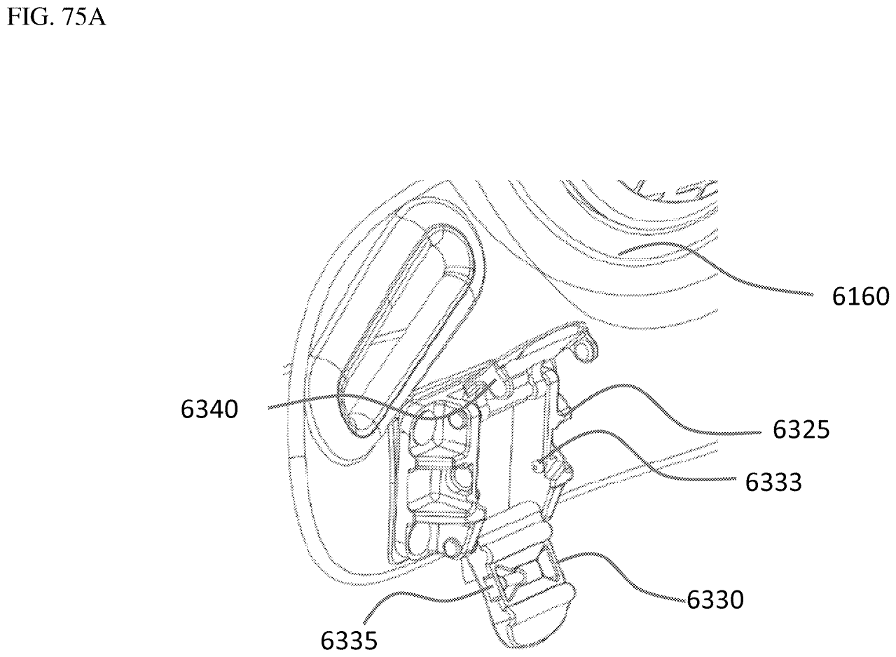

The chassis establishes a channel that has a width that is wider than the width of the strap. That is, the distance from the inside surfaces of the lugs that support the hinge pin and thus hold the mating to component between those lugs and otherwise to the chassis is wider than the corresponding dimension of the strap, and in an embodiment, it is less than, greater than, and/or equal to 0.005, 0.01, 0.015, 0.02, 0.025, 0.03, 0.04, 0.05, 0.075, 0.1, or 0.125 inches or more, or any value or range of values therebetween in 0.005 inch increments. Accordingly, without the hinged components in place, little to no force is required to move the strap relative to the chassis when the strap is below the lugs of the chassis. That said, in an embodiment, a slip fit or an interference fit could be used, where the width of the channel through the chassis is smaller than the strap (and the strap will compress, however slightly. FIG. 75 shows the channel through the chassis, where both the hinged components are “open” (whereas FIG. 72 shows both “closed”).

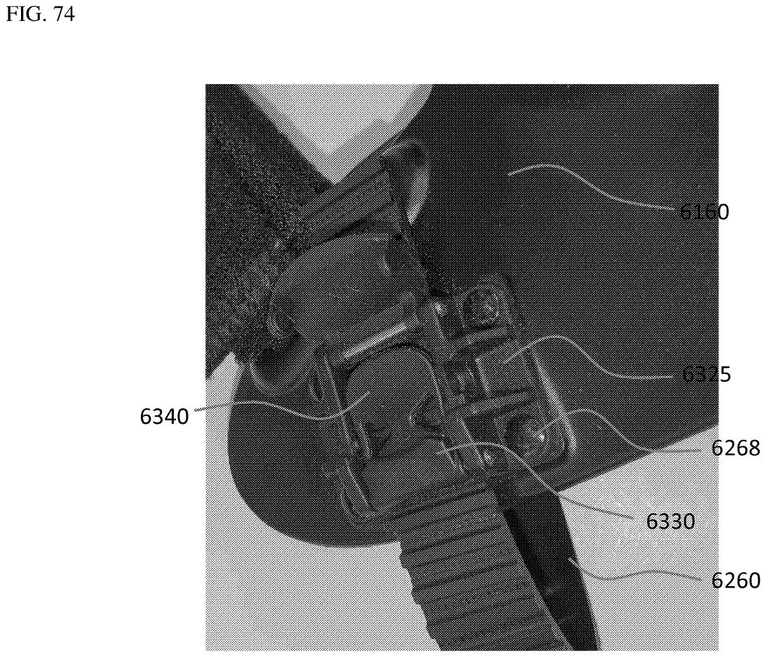

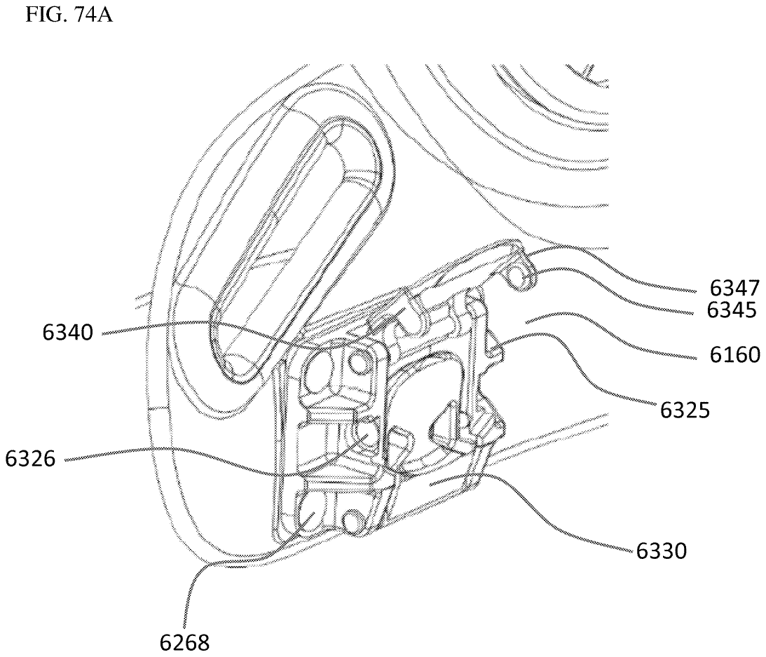

In a method of setting or adjusting the brace for a given user, in use, the mating toothed component 6330 and the lock portion 6340 are moved to the open and unlocked position as seen in FIG. 75A. More specifically, it is starting from the locked and closed position, first, the lock portion 6340 is moved from the locked position as shown in FIG. 73 to the unlocked position as seen in FIG. 74. In this regard, the lock portion 6340 is configured to prevent the mating toothed component 6330 from being opened when the lock 6340 is closed, thus blocking the component 6330 in place in the closed position. In an embodiment, the lock portion 6340 does not contact the strap, and in some embodiments, only contacts the strap at the boss for the hinge pin, at least when in the closed position. In an embodiment, the lock portion can be completely closed, and if the mating toothed component 6330 is in the open position or has been completely removed the purposes of testing or evaluation, strap 6260 can be moved within the class or otherwise through the chassis, and in an embodiment, the force required to do so is the same as that which would be the case if both components were completely open and/or the forces no more than or no less than the amount that is different by less than, equal to and/or greater than 5, 10, 15, 20, 25, or 30%, or any value or range of values therebetween in 1% increments. That is, in an embodiment, the lock portion 6340 does not provide any positive retention in some embodiments, and otherwise does not provide any retention force in some embodiments, while in other embodiments, the retention force that results from the lock component is minimal or otherwise not sufficient to cure the strap in place or otherwise keep the strap from moving in an effective manner.

Lock portion 6340 has the functionality to ensure that the mating tooth component 6330 does not come open or otherwise is harder to be opened than that which would otherwise be the case in the absence of the lock portion 6340. It is to lock the component 6330 closed. In an embodiment, lock component 6340 includes two detents 6345 supported by respective bosses 6347. In an embodiment, when the lock component 6340 is closed, the detents 6345 snap into place and fit into the female component 6326 of the chassis 6325. In this embodiment, the detents essentially “grip” the sides of the chassis or otherwise the components that establish the passageway through the chassis the channel chassis which strap 6260 is snaked. (Note that the male detents can be located instead on the chassis and the female mating portions can be located on the lock 6340, or one can be on each, and more than two detents can be used.) Thus, when the lock 6340 is closed, an unlocking force is required to open the lock portion or otherwise rotate the portion from the closed position relative to that which would otherwise be the case if the friction forces or gravity were utilized to hold the lock 6340 closed or otherwise in place in the closed position. In an exemplary embodiment, a force that must be applied to the lock portion to rotate the lock portion from the closed position is at least 1, 1.5, 2, 2.5, 3, 3.5, 4, 4.5, 5, 5.5, 6, 6.5, 7, 7.5, 8, 8.5, 9, 10, 11, 12, 13, 14, 15, 20, 25, or 30 times or more, or any value or range of values therebetween in 0.1 increments relative to that which would otherwise be the case in the absence of the detent system or otherwise locking features for the forces applied at the same location the same direction, that is, all other things being equal, etc. In an embodiment, a force applied to have the maximum leverage (at the most distal part of the lock) of 2, 3, 4, 5, 10, 15, 20, 25, 30, 35, or 40 or more, or any value or range of values therebetween in 1 N increments must be applied to overcome the detent and rotate the lock/open the lock.

In any event, as seen, the lock portion 6340 is configured to cover at least a portion of the mating tooth component 6330 or otherwise be interposed in the path of rotational movement of that component when the lock component 6340 is closed. This thus prevents the mating component 6330 from rotating from the closed position, and thus secures the strap in the clasp.

In an embodiment, a tool of some sort is needed to open the lock portion 6340, at least without the use of fingernails, for a human factors engineering 50 percentile human of age 20 to 65 years old born in the United States who is a male or female who has lived in the United States or otherwise has been a resident of the United States for the entire period of time. In an embodiment, a flat head screwdriver could be utilized to pry open the lock component 6340. That said, in an embodiment, a fingernail of an index finger might be utilized grab onto an edge of the lock component to unlock the lock component. To be clear, at least some embodiments of the lock component are configured so that the lock component is hard or otherwise difficult to unlock or otherwise open. It is not just that purposeful actions must be taken to unlock the lock component. It is that purposeful actions must be taken in a manner that would be directly intended to make an adjustment to the strap where the adjustment to the strap is not a normal action to be taken with respect to day-to-day use of the brace. That is, embodiments are configured so that once the length of the strap is set or otherwise the adjustment is made, or otherwise the adjustment is settled upon (it could be an iterative process), the strap is not going to be adjusted again, at least for a given to that user will use, or a substantial period of time or otherwise for a number of uses of the brace. In an embodiment, a use of the brace can be executed for 5, 10, 15, 20, 25, 30, 35, 40, 50, 60, 70, 80, 90 or 100 or more or any value or range of values therebetween in 1 increment uses of the brace (or days of uses of the brace (where a donning of the brace during a day, even if used for an hour, is one day of use)) without adjustment. To be clear, it is not that adjustment or otherwise movement of the lock component requires great skill or requires great effort. It is that adjustment or otherwise movement of the lock component requires a modicum of skill or a modicum of effort above that which would be the case for something that was configured for regular adjustment on a daily basis or otherwise when a per use basis, or otherwise an adjustment or an opening that would be required to don the brace every time. By rough analogy, the bindings of a set of skis for snow skiing are configured to be adjusted, and the common user of a set of skis can adjust the bindings with a screwdriver for example or even by adjusting a knob without it, but the adjustment is not something that will be done every time the skis are humans, at least not for the amperage or the large percentage of skiers.

This leads to the mating tooth component 6330. As with the lock component 6340, component 6330 is hingedly fixed to the chassis 6325. The attachment is identical to that of the lock component 6340 in this embodiment, but can be different. Indeed, in an embodiment, the mating tooth component 6330 is attached to the chassis where the lock component 6340 is attached, and vice versa. Note also that instead of the vertical alignment of the components, the components could be attached to a variation of the chassis in a horizontal manner (the swing is in the horizontal plane as opposed to the vertical plane). This would have a different design for the chassis, and the teeth of the mating tooth component would still have to be aligned with the teeth of the strap, or more accurately, the valleys of the strap, but the engineering could be worked to achieve a functional arrangement. In any event, the mating tooth component is the part that actually interfaces with the strap 6260, at least with respect to securing the length of the strap and otherwise holding the strap in place. Again, the teeth 6334 perform the positive interference with the structure of the strap that holds the strap in place and prevents the strap from being pulled out of the clasp 6320 or further inserted into the clasp 6320 when component 6330 is closed, irrespective of whether lock 6340 is closed. In an embodiment, a depth of the teeth 6334 can extend below tops of the teeth (mean, median, and/or mode of the teeth that interface with the mating tooth component 6330, or the tallest or shortest tooth thereof) of the strap by less than, greater than and/or equal to 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2 inches or more, or any value or range of values therebetween in 0.01 increments. This can be for one or more or all of the teeth 6334, and the distance need not be the same for each tooth. In an embodiment, when the plane of the tops of the teeth of component 6330 are parallel with the surface of the channel of the chassis, component 6330 fully secures the strap.

As with lock portion 6340, mating toothed component 6330 is restrained from movement/rotation by detent actions. Here, there are two detents 6333 (male detents) supported by the chassis 6325 (the sidewalls that form the channel in this embodiment). In an embodiment, when the mating toothed component 6330 is closed, the detents 6333 snap into place and fit into the female components 6335 of the mating tooth component 6330. In this embodiment, the detents essentially “grip” the sides of the mating toothed component 6330. Thus, when the lock 6340 is closed, a releasing force is required to open or otherwise rotate the mating tooth portion from the closed position relative to that which would otherwise be the case if the friction forces or gravity were utilized to hold the component 6330 closed or otherwise in place in the closed position. In an exemplary embodiment, a force that must be applied to the lock portion to rotate the lock portion from the closed position is at least 1, 1.5, 2, 2.5, 3, 3.5, 4, 4.5, 5, 5.5, 6, 6.5, 7, 7.5, 8, 8.5, 9, 10, 11, 12, 13, 14, 15, 20, 25, or 30 times or more, or any value or range of values therebetween in 0.1 increments relative to that which would otherwise be the case in the absence of the detent system or otherwise securing features for the forces applied at the same location the same direction, that is, all other things being equal, etc. In an embodiment, a force applied to have the maximum leverage (at the most distal part of the mating tooth component 6330) of 2, 3, 4, 5, 10, 15, 20, 25, 30, 35, or 40 or more, or any value or range of values therebetween in 1 N increments must be applied to overcome the detent and rotate the component 6330 to the open position. (Note that the male detents can be located instead on the mating tooth component 6339 and the female mating portions can be located chassis, or one can be on each, and more than two detents can be used.)

As with the lock 6340, the configuration of the meeting tooth component 6330 and the accompanying chassis is such that it is difficult if not impossible to open the component 6330 without a tool. Of course, some people with sufficiently strong fingernails or the like could do so. In this regard, the features detailed above with respect to the constraints of opening the lock component 6340 are applicable to the mating tooth component 6330 in the interest of textual economy.

In any event, in an exemplary embodiment of adjusting the strap or otherwise setting the strap for use, the free end of the strap 6260 is snaked through the lower female receptacle formed by the chassis and the mating tooth component 6330 and then further into the chassis to the receptacle formed by the lock 6340 and the chassis, with these two components being in the open/unlocked position, such as that shown in FIG. 75A. That said, it could be that the strap 6260 is already through one or both of those receptacles, and the action of setting the brace or adjusting the brace for a given user amounts to only moving the strap in one direction or the other direction without removing the end of the strap through one or both receptacles or otherwise into or out of the chassis. Indeed, it is noted that in some embodiments, the end of the strap is such that the end cannot be pulled through the class with one or both of the hinged components attached to the clasp. A rivet could be located at the end of the strap or near the end of the strap or otherwise a clamping structure could be clamped around the end of the strap which is larger than the female receptacle established by the lock component and the chassis so that the end of the strap cannot be pulled out of the clasp.

A desired tightness (relative, there could be some looseness if desired) is achieved by pulling and/or pushing the strap 6260 further through at least one of the receptacles. Once the desired tightness is achieved, or in practice, sometimes the estimated desired tightness is achieved (adjusting the brace can be an iterative procedure—often, the brace will be adjusted without the user's foot in the shoe, or even without the shoe in the brace—the brace can be slipped on/over the shoe and the user's foot after the adjustment has been set, with the top strap “closing” the brace—more on this in a moment) or otherwise, once the desired position of the strap 6260 within the chassis is achieved, the mating toothed component 6330 is rotated so that the tooth 6334 or teeth 6334 (this embodiment has three (3) teeth 6334, as can be seen in FIG. 76, in another embodiment, more or fewer teeth can be utilized—less than, greater than and/or equal to 1 (and thus there is no less than), 2, 3, 4, 5, 6, 7 or 8 or more teeth can be used) fit into the depressions or cavities 6264 between the teeth 6262, thus gripping the strap and preventing the strap from moving in any meaningful manner in either direction. In an embodiment, the teeth 6334 slip fit into the depressions/cavities 6264, and other embodiments, the teeth interference fit, and in other embodiments, the teeth clearance fit. The teeth are partially circular as seen (the cross-section of the teeth taken through the plane extending in and out of FIG. 61 and extending vertically has a partially circular outer cross-section), but in an alternate embodiment, the teeth can be partially oval shaped or triangular-shaped or rectangular shaped or square shapes, etc.—any shape that can of utilitarian value the can interface with the strap can be utilized in at least some exemplary embodiments). In an embodiment, a radius of curvature of the teeth can be less than, greater than and/or equal to 0.01, 0.02, 0.03, 0.04, 0.05, 0.06, 0.07, 0.08, 0.09, 0.1, 0.11, 0.12, 0.13, 0.14, or 0.15 or more inches, or any value or rage of values therebetween in 0.005 inch increments. And the radius of curvature can change (for a partially oval shape—the oval shape can have any of these in an embodiment at different locations). Owing to the partial circularity of the teeth of the mating toothed component 6330, the teeth thereof can “wedge” their way into the depressions/cavities between the teeth of the strap if an interference fit is utilized for example. This can provide a relatively strong and dimensionally stable positioning of the strap. In an embodiment, when the strap is subjected to a pulling force of less than, greater than and/or equal to 1, 2, 3, 4, 5, 6, 7, 8, 9, 10, 11, 12, 13, 14, 15, 20, 25, or 30 N, or any value or range of values therebetween in 0.1 N increments directly away from the clasp with the clasp fully engaged/closed, with less than/greater than and/or equal to 1, 1.5, 2, 2.5, 3, 3.5, or 4 inches, or any value or range of values therebetween in 0.1 inch increments of strap between the immediate outside of the clasp and immediately outside a comparable clasp used to apply the force for test purpose, the strap moves no more than/the distance between the two clasps expands no more than 0.01, 0.02, 0.03, 0.04, 0.05, 0.06, 0.07, 0.08, 0.09, 0.1, 0.125, 0.15, 0.175, 0.2, 0.225, or 0.25 inches, or any value or range of values therebetween in 0.01 inch increments.

In an embodiment, instead of a partial circle or partial oval, chamfering can be applied to a rectangular shaped tooth, at any angle that is utilitarian, such as less than, greater than, and/or equal to 5, 10, 15, 20, 25, 30, 35, 40, 45, 50, 55, 60, 65, 70, 75, 80, or 85 degrees or more, or any value or range of values therebetween in 0.1 degree increments on one or both sides (where the angle is the surface of the chamfer relative to the height of the tooth (and thus 85 degrees would be sharper than 5 degrees)) and in an embodiment, the top surface area is reduced by less than, greater than, and/or equal to 5, 10, 15, 20, 25, 30, 35, 40, 45, 50, 55, 60, 65, 70, 75, 80, 85, 90, or 95% or more, or any value or range of values therebetween in 1% increments relative to that which would be the case in the absence of the chamfering.

In an embodiment, the teeth and/or the valleys of the mating tooth component 6330 can have a span that corresponds to those of the teeth of the strap, or can be oversized or undersized. In an embodiment, the span/of the teeth and/or the valleys of the mating tooth component 6330 can be less than, greater than and/or equal to 5, 10, 20, 30, 40, 50, 60, 70, 80, 90, 100, 125, 150, 175, or 200% or more, or any value or range of values therebetween in 1% increments of the maximum span and/or minimum span of the teeth/valleys of the strap that can will interface with the clasp. This can also be the case with respect to the thickness and/or depth and/or height of the teeth and/or valleys of the mating tooth component, and as usual, the values need not be the same, in the interests of textual economy.

In an embodiment, when the lateral sidewall is rigidly fastened to an effectively immovable test block, or when the clasp is fastened as would be the case for the brace to a test block to stimulate the sidewall, a force applied to the strap at a location immediately adjacent the clasp when the clasp is locked (more on this in a moment) of at least and/or equal to 150, 200, 250, 300, 350, 400, 450, 500, 550, 600, 650, 700, 750, 800, 850, 900, 950, 1000, 1250, or 1500 N, or any value or range of values in 1 N increments without plastic deformation and/or failure of the strap and/or the clasp arrangement (including the rivets) and/or the sidewall. In an embodiment, the clasp and/or the securement structure and/or the rivets and/or the sidewalls is/are configured to fail prior to the strap, and thus in an embodiment, the strap can be subjected to any of the just noted forces with clamping structure akin to the securement structure disclosed herein and mating clamping surface in the form of a test structure that will fail after the strap with a four (4) inch gap between the closest portion of each (the strap is thus 4 inches plus the part that fits into the securement structure fully) and/or 1.5, 2, 2.5, 3, 3.5, 4, 4.5, 5, 6, 7, 8, 9, 10, 11, 12, 13, 14, 15, 20, 25, 30, 35, 40, 45, or 50 times or more without experiencing plastic deformation and/or without expanding more than 0.5, 1, 2, 3, 4, or 5%, or any value or range of values therebetween in 0.1% increments in length of plastic deformation (or elastic deformation in some other embodiments) (e.g., the four inches would expand no more than 1.05 times four inches).

In an embodiment, the clasp is configured so that the mating tooth component 6330 and/or the lock 6340 can rotate, from a full closed/fully locked position, with and/or without the strap being fully located therein, depending on the embodiment, less than, greater than and/or equal to 5, 10, 15, 20, 25, 30, 35, 40, 45, 50, 55, 60, 65, 70, 75, 80, 85, 90, 95, 100, 105, 110, 115, 120, 125, 130, 135, 140, 145, 1550, 155, 160, 165, 170, 175, 180, 185, 190, 195, 200, 205, 210, 215, 220, 225, 230, 235, 240, 245, 250, 255 or 260 degrees or more or any value or range of values therebetween in 0.1 degree increments, and the two values need not be the same in the interests of textual economy. In an embodiment, upon overcoming the detents, a force of less than, greater than and/or equal to 0.25, 0.5, 0.75, 1, 1.25, 1.5, 1.75, 2, 2.25, 2.5, 2.75, 3, 3.25, 3.5, 3.75, 4, 4.5, 5, 5.5, 6, 6.5, 7, 7.5, 8, 8.5, 9, 9.5, 10, 11, 12, 13, 14, 15 or more, or any value or range of values in 1 N increments applied at the most leverage maximizing location is required to rotate the given component more than 10 degrees, to overcome for example friction, with and/or without the strap being located in the clasp, and the values for the lock component and the toothed component need not be the same.

The disclosure herein of features of the strap 6260 with respect to teeth and valleys/recesses from a dimensional standpoint can correspond to comparable features of the clasp and/or the securement structure, and the features need not be the same, in the interests of textual economy and vice versa for the clasp and vice versa for the securement structure.

In an exemplary embodiment, the strap and/or the structure that supports the strap (e.g., the sidewalls and/or the clasp and/or the securement structure), are configured to withstand a tension of at least and/or equal to and/or no more than 20, 25, 30, 35, 40, 50, 60, 70, 80, 90, 100, 125, 150, 175, 200, 225, 250, 275, or 300 N, or any value or range of values therebetween in 0.1 N increments without experiencing a failure mode. In an exemplary embodiment, the aforementioned tensions can be applied without experiencing an effective deleterious degradation in the strap and/or the support structure. And to be clear, the aforementioned values need not be the same for both the strap and the support structure. They can be different. In an exemplary embodiment, the strap can be configured to fail before the support structure/connecting structure or vice versa, for a given tension.

The desired tautness and/or limitation of pullout can be achieved in part based on the material properties of the strap and/or the size and/or dimensions of the strap. In an exemplary embodiment, the strap is made of a polymer that is both flexible and resistant to wear. In an exemplary embodiment, the strap 6260 extends beneath the sole of the shoe and thus interfaces with the ground upon which the user walks and/or runs. In an exemplary embodiment, the strap when utilized with the bottom of a shoe is such that when a standard sneaker and/or a standard shoe is worn, a portion of the sole of the shoe does not contact the ground even though that portion is on a side otherwise spaced away from the strap. That is, the strap supports a portion of the soul of the shoe, and depending on the flexibility of the soul of the shoe, there will be a portion that is held or otherwise supported away from the ground until the sole of the shoe flexes towards contact with the ground.

In an exemplary embodiment, the strap 6260 is sized and dimensioned so that a bending radius of less than and/or equal to and/or greater than 0.01, 0.02, 0.03, 0.04, 0.05, 0.06, 0.07, 0.08, 0.09, 0.1, 0.125, 0.15, 0.12, 0.13, 0.14, 0.15, 0.16, 0.17, 0.18, 0.19, 0.20, 0.21, 0.22, 0.23, 0.24, or 0.25 inches, or any value or range of values therebetween in 0.0001 increments (or less), at least to achieve the standard English measurements, can be achieved 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 700, 800, 900, or 1000 times or more, or any value or range of values therebetween in 1 increment from a completely straight state without stretching and/or without reducing the strength of the strap by an effectively deleterious amount.

In an embodiment, the teeth of the strap 6260 can have utilitarian value in that such can provide a modicum of grouping between the strap and the ground or floor or whatever surface upon which the user is walking or running. In an embodiment, the lower strap improves a grip feature or a friction coefficient of the brace, relative to that which would be the case in the absence of the teeth (e.g., the strap has a thickness of only the main span/base portion), by at least and/or equal to 3, 4, 5, 6, 7, 8, 9, 10, 15, 20, 25, or 30% or more, or any value or range of values therebetween in 0.1% increments. In an embodiment, this is measured for a whole section of strap that spans 3 inches in a flat direction over which is applied a solid block that is effectively rigid with a downward force of 150 pounds.

In an embodiment, the lower connecting strap is releasably attached to the lateral sidewall by way of partial penetrative positive retention (e.g., by way of the teeth of the clasp interacting with the recesses between the teeth, as opposed to a male portion fully penetrating the strap (or entering a passage that extends completely through the strap)). In an embodiment, the lower connecting strap is permanently attached to the medial sidewall by way of a partially penetrative positive retention. In an embodiment, the lower connecting strap is permanently attached to the medial sidewall by way of only gripping forces (as opposed to a rivet or a screw piercing the strap) or only grasping forces (again, as opposed to something that fits through the strap). This is also the case with respect to the releasable attachment feature as well in some embodiments.

In an embodiment, there is an external ankle brace, comprising a medial lower leg section configured to interface with at least a medial side of a leg of a human at a lower leg portion thereof, the medial lower leg section including a medial rigid body contoured to a medial side of a lower leg of a human, a lateral lower leg section configured to interface with at least a lateral side of a leg of a human at a lower leg portion thereof, the lateral lower leg section including a medial rigid body contoured to a medial side of a lower leg of a human and a foot section configured to interface with a shoe worn on a foot of the human at locations where the foot of the human is located, wherein a first strap extends from one side of the foot section to the other side of the foot section on a top of the foot section, the first strap at least one of extending through a body establishing the foot section or having a potion thereof being located outboard of the body and a second strap extends from the one side of the foot section to the other side of the foot section on a bottom of the foot section, wherein at least one of the second strap being entirely located outboard of the body or the body has no indentations to directly accommodate the second strap. In an embodiment where the first strap extends through the body, the strap extends through the body at two separate locations, such as through the lateral sidewall and through the medial sidewall, as detailed above. In an embodiment, there are only two openings in the body for the top strap, one on either side of the heel enclosure. In an embodiment, there are only four openings, where there are two openings on each side for two separate straps.

In an embodiment, alternatively and/or in addition to this, the first strap has a portion that is located outboard of the body. By way of example only and not by way limitation, this is achieved by the use of rivets or the like to hold a D shaped buckle or some other buckle to the body, and then the strap is snaked through the buckle, and the strap is located on the outboard side of the body (a portion of the strap contacts the body on the outboard side). In an alternate embodiment, this is achieved because the strap is snaked through the opening in the body, where the strap extends over the outboard surface after being extended through the opening after extending over the import surface. Thus, in an embodiment, the first strap has a portion that is located outboard of the body and a portion that is located import of the body, on one or both sides of the body. Of course, in an alternate embodiment, the strap and/or the fixing components thereof are located inboard. In an alternate embodiment, no fixing component for the upper strap is located inboard, while in an alternate embodiment, there are no fixing components for the upper strap. The only attachment regime for the upper strap is/are the openings (at least for one side). In an embodiment, there are no rivets that are utilized to secure the upper strap to the body or otherwise that are part of the upper strapping system or otherwise the upper fastening system. In an embodiment, the upper fastening system consists of the strap and through holes or through passageways through the body and nothing else. In an embodiment, the only portion that penetrates the body of the upper fastening system is the strap.

In an embodiment, the second strap is located in its entirety outboard of the body. That is, the portion of the strap on the lateral side is located outboard of the body that establishes the rigid heel enclosure, and the portion of the strap that is located on the medial side is located in its entirety outboard of the body. In an embodiment, the body has no indentations to directly accommodate the second strap. Instead, in an embodiment, there are the thickened sections that are utilized to support the chassis of the clasp and/or the component that permanently attaches the end of the strap to the medial side. That is, there are no channels or indentations for the strap. In an embodiment, there are no piercings of the second strap (as shown above, in an embodiment, there is the gripping of the teeth—the positive retention is now owing to a piercing, but instead the placement of a component adjacent another portion—in an embodiment, the teeth only extend below a topmost portion of the strap no more than 10, 15, 20, 25, 30, 35, 40, 45, 50, 55, 60, 65, 70, 75, 80, 85 or 90% of the total thickness or any value or range of values therebetween in 1% increments). In an embodiment, the brace is configured so that the strap cannot be pierced by a component thereof. (And note that piercing does no require the device to create the hole, but it could be that an existing hole is widened. A hole that is not all the way through does not result in a piercing (unless the component then pushes all the way through).)

In an embodiment, there are no rivets or other components (screws, bolts) that pierce the body associated with the first strap. No adhesive is used in some embodiments to hold the straps in place (for the foot portion). No Velcro is used for one or both straps, and Velcro is used for one strap but not the bottom strap in another embodiment. The straps do not circumnavigate the body in some embodiments or the shoe in some embodiments, while in others, the strap does. In an embodiment, there is only one strap. In an embodiment, the arrangement of the bottom strap is used for the top strap or in addition to such. (Arrangement meaning any one or more of the components of the lower fastening system or the features thereof.)

In an embodiment, there are rivets associated with the second strap that pierce the body (of the foot enclosure), and the second strap is unpierced by any rivet. These can be the four rivets used to mount the chassis or the plate, etc., for example.

In an embodiment, the first strap does not extend underneath a shoe worn in the brace when the brace is worn over a shoe, and the second strap does not extend over a top of the shoe. But in another embodiment, one or both of these is not the case. In an embodiment, the second strap is entirely located outboard of the body (whether or not there is a channel or an indentation for such). But alternatively the second strap is located inboard of the body on one side of the foot interface and located outboard of the body on an opposite side of the foot interface. In an embodiment, there is only 1, 2 or 3 straps extending from one side to the other side of the foot section on top and/or there is only 1, 2 or 3 straps extending from one side to the other side of the foot section on bottom. And one or more of these may or may no be adjustable.

In an embodiment, there are two or more straps extending from one side to the other side of the foot section on bottom. In an embodiment, any one or more or all of the straps are forward of a talus of a foot when worn by a human. In an embodiment, there are two or more straps extending from one side to the other side of the foot section on bottom. In an embodiment, the first strap extends through a body establishing the foot section and/or the second strap does not so extend. The first strap has a potion thereof being located outboard of the body (and a portion inboard) and/or the second strap is entirely located outboard of the body. The first strap surrounds a portion of the body. As seen above, in some embodiments, the body has no indentations to directly accommodate the second strap (but there can be a thickened section and/or holes for the rivets that are used to connect the chassis, etc.) and/or there is no piercing of the second strap and/or the second strap is permanently connected the foot portion on one side and adjustably connected on an opposite side and/or the second strap is releasably connected to one side of the foot portion and releasably connected to an opposite side of the foot portion.

FIG. 105 presents an exemplary flowchart for an exemplary method, method 10500, that includes method action 10510, that includes the action of obtaining an ankle brace, which brace can be any of those detailed herein or a variation thereof or another brace. Method 10500 further includes method action 10520, which includes the action of adjusting a strap that extends across the bottom of the shoe and securing the adjusted strap using a two-stage securement arrangement. In an embodiment, the two-stage securement arrangement according to any of the embodiments detailed herein with respect to the clasp.

In an embodiment, method action 10520 includes any one or more of the actions detailed herein associated with working with the clasp or otherwise securing and/or adjusting the strap. An embodiment, action 10520 includes “threading” the end of the strap 6260 into the bottom portion of the clasp and through the top portion of the clasp so that the strap extends into the clasp, and through the clasp and out of the clasp. In an exemplary embodiment, prior to this, lock 6340 is unlocked and then mating toothed component 6330 is opened. In other embodiments, these are already in the open position. In an embodiment, the actions of opening one or both of these components are executed utilizing a tool for example, such as a screwdriver or some other leverage imparting device, such as the end of a ballpoint pen while in other embodiments this is done utilizing a person's fingernail to reach under the various components so that the component can be opened.

The method can further entail rotating or otherwise closing or otherwise positioning the component 6330 so that there is a positive interface between the component 6230 and the strap. In an embodiment, this is owing to the teeth and the depressions between the teeth. And note that while in some embodiments, a ribbed or tooth strap is disclosed, in another embodiment, the strap can be more of a traditional strap with holes, and there can be one or more male portions such as cylindrical portions that extend from the component 6330 that fit into the hole(s) of the strap to provide positive retention. In this regard, this can be more analogous to the arrangement of FIGS. 14-17, and that strap can be used, and instead of the boss or the cylindrical portion 262 being part of the heel enclosure, that portion is carried by the component 6330. The method can further include the action of with the sub action of rotating the lock 6340 so that the component 6330 is locked in place in accordance with the teachings detailed herein. In an embodiment, these actions entail overcoming the forces associated with the detent systems or otherwise the securement systems that secure the components in place as detailed above.

In an embodiment, method action 10520 is executed by the person who is wearing more is going to wear the brace, while in other embodiments, this is executed by another person who is assisting or otherwise servicing the person who will wear the brace. Or is wearing the brace for that matter. In an embodiment, the actions of adjusting are executed while the person wearing or who will wear the brace has his or her foot into the shoe, and the shoe is located in the brace. In an alternative embodiment, the shoe is in the brace but the person who will wear the shoe is not wearing the shoe at the current time. In another embodiment, the actions of adjusting are done without the shoe or the person who will wear the brace in the brace. This latter scenario could entail more trial and error or repeating method action 10520 or portions thereof a number of times.

FIG. 76 depicts a cross-sectional view of the external ankle brace 6500 on a plane lying on and parallel to the rotation axis of the medial joint and/or the longitudinal axis of the rivet 6390 that establishes the pivot of that joint. Owing to the offsets of the medial and lateral joints as will be described in greater detail below, the cross-section does not extend through the axis of rotation of the lateral joint and/or the longitudinal axis of the Chicago screw of that joint (more on this below). The cross-sectional view shows that the lateral and medial joints are made up of a male portion (respectively 6162 and 6182) that is monolithic with the bulk of the structure that makes up the respective sidewalls, and a female portion (respectively 6202 and 6222) that is monolithic with the bulk of the structure that makes up the respective uprights.

FIG. 77 depicts the cross-sections looking directly backwards. While not shown in FIG. 77, with respect to the lateral upright extension 6200, the cross-section extends unbroken from the top to the bottom most portion thereof, the structure that establishes such extension is monolithic with the female portion 202 of the lateral joint. With respect to the lateral sidewall 6160, the cross-section extends unbroken from the top to the bottom most portion thereof, the structure that establishes such extension is monolithic with the male portion 6162 of the lateral joint. With respect to the medial upright 6220, the cross-section extends unbroken from the top to the bottom most portion thereof, the structure that establishes such extension is monolithic with the female portion 6222 of the medial joint. With respect to the medial sidewall 6180, the cross-section extends unbroken from the top to the bottom most portion thereof, the structure that establishes such extension is monolithic with the male portion 6182 of the medial joint.

It is noted that in an exemplary embodiment, a circular foam pad 6222 is placed on the inboard face 62345 of the male joint portions so as to pad between the joint portion and the respective malleolus (or, more accurately, the skin over the respective malleolus). In this embodiment, the pad is disk shaped with planer tops and bottoms, and then bends to conform when placed against face 62345, while in other embodiments, the pad is pre contoured. In an embodiment, the pad is a non-Newtonian material pad.

It is noted that in an exemplary embodiment, there is a washer 6987 that is located between the faces of the male and female portions that are normal to the pivot axis. The washers has a hole through which the rivet extends. The washers can be low friction washers that are made from a polymer or any other material that is suitable. In an exemplary embodiment, the washers maintain a space between the aforementioned faces of the male and female joint portions that is at least and/or equal to and/or no more then 0.005, 0.01, 0.015, 0.02, 0.25, 0.03, 0.04, 0.05, 0.06, 0.07, 0.08, 0.09, 0.1, 0.125, or 0.15 inches, or any value or range of values therebetween in 0.0001 increments (or less), at least to achieve the standard English measurements. In an exemplary embodiment, the aforementioned values are the thickness of the washer. In an exemplary embodiment, the outer diameter of the washer (it can be circular outside diameter and/or with respect to the inside diameter) and/or maximum outer diameter of the washer can be less than and/or equal to and/or greater than 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.25, 2.5, 2.75, or 3 inches, or any value or range of values therebetween in 0.001 increments (or less) to achieve the English measurement units at least. As seen from FIG. 76, the washer 6987 is dome shaped. This can be a preformed dome or can be a result of the flexibility of the washer when put in place. This can be due to elastic deformation and/or plastic deformation.

In an exemplary embodiment, the interfacing or otherwise facing surfaces of the male and female joint portions are dome shaped with a circular outer periphery, concomitant with the fact that the portions rotate relative to one another. It is also noted that in an exemplary embodiment, the figures are drawn to scale in totality and/or on a percent basis (e.g., if a view is shown in a shrunken manner, that is still to scale on a percent basis).

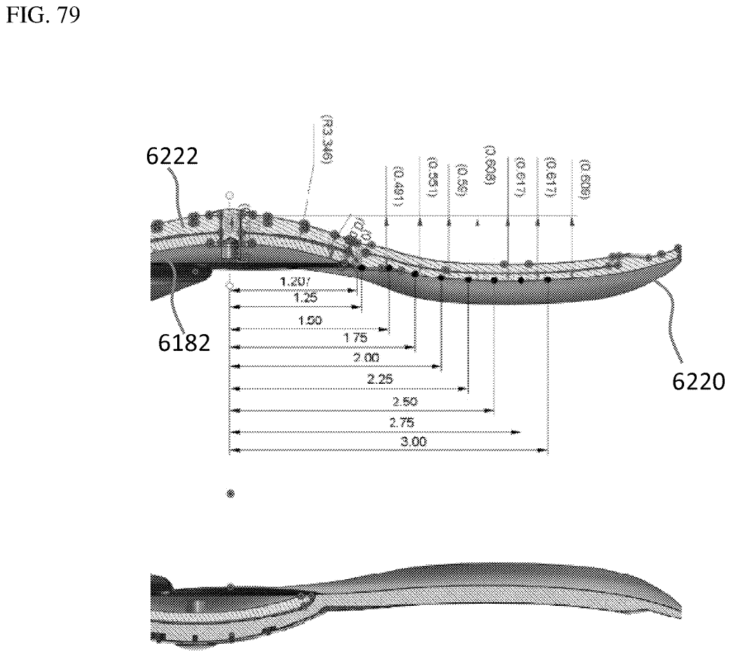

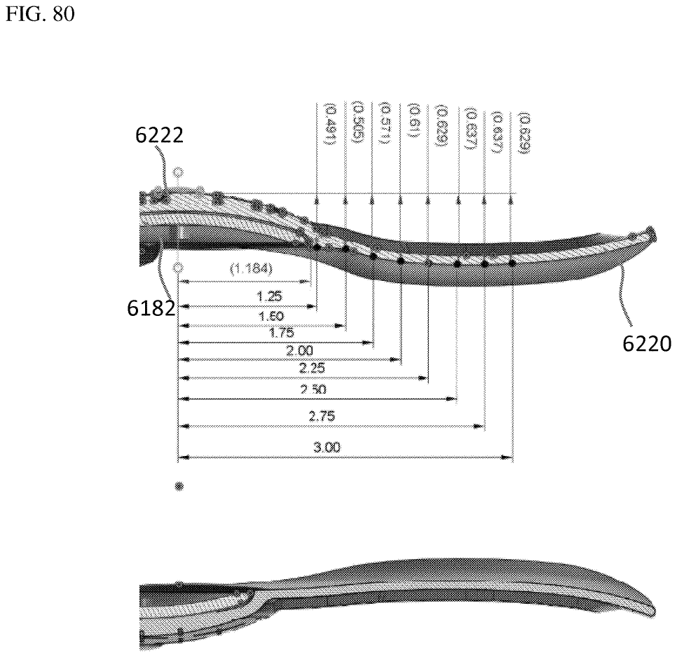

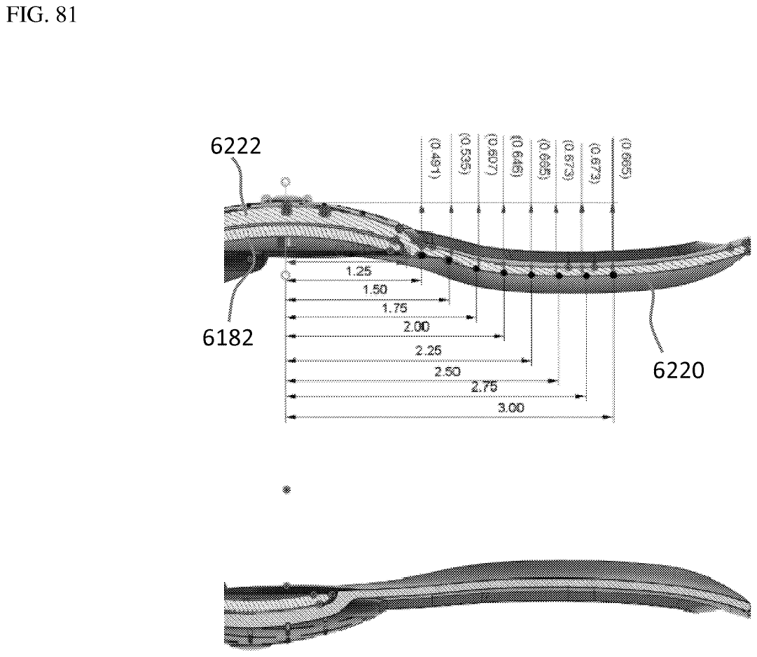

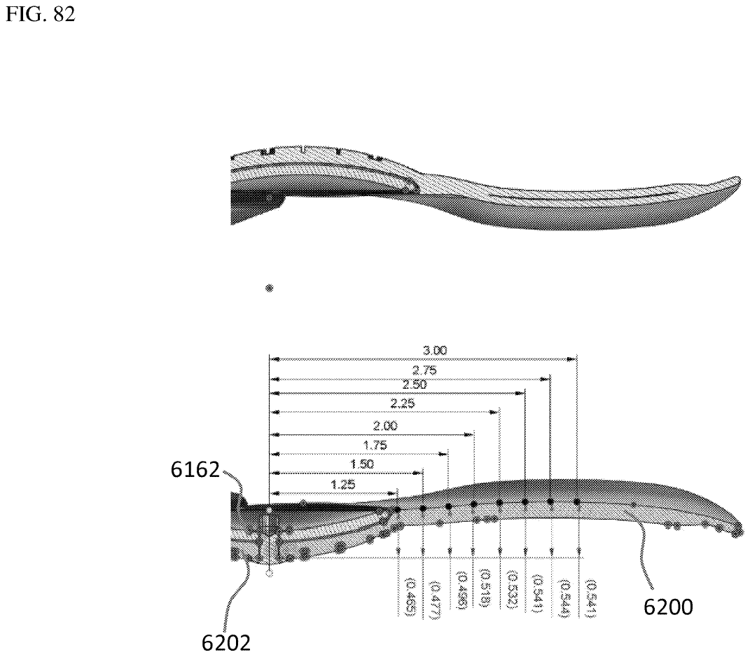

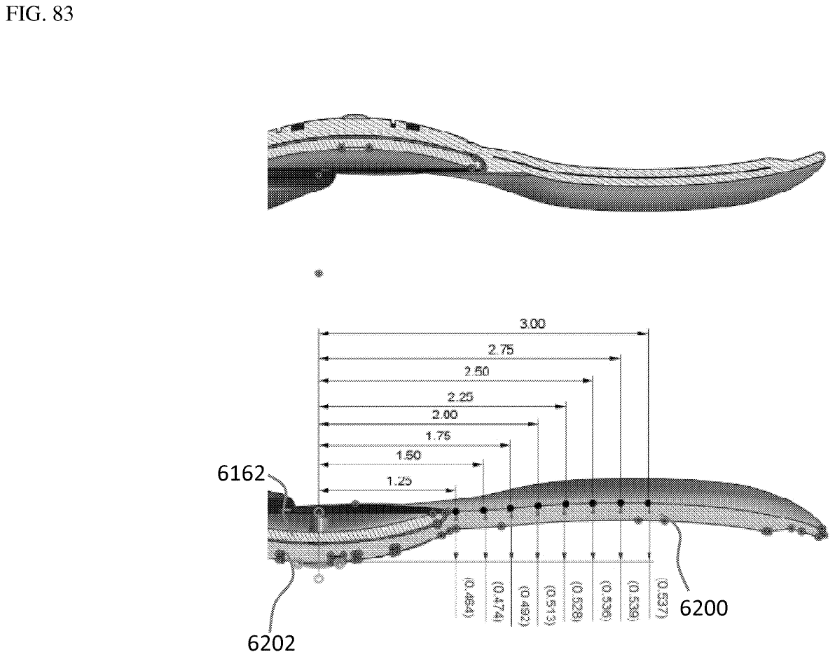

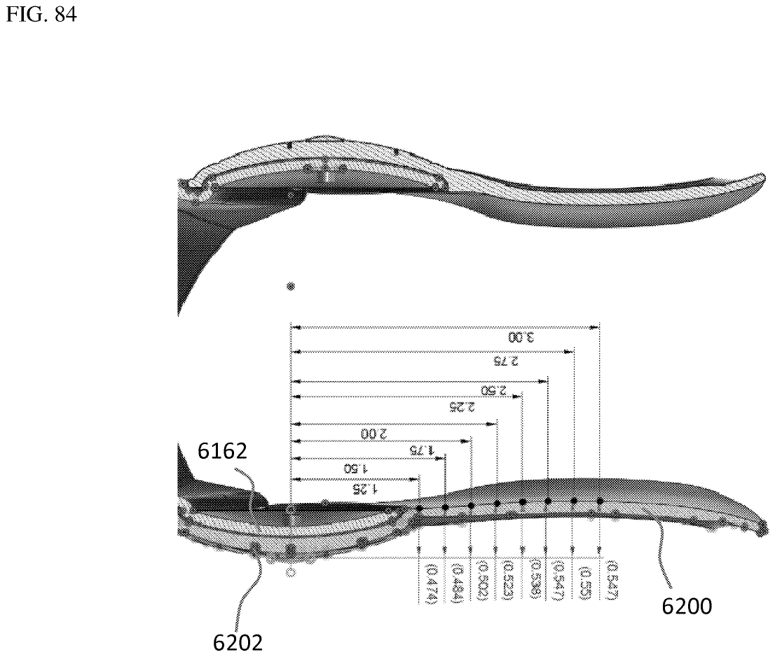

FIG. 79-84 show dimensioned cross-sectional views respectively corresponding to the cross-sections J-J to O-O of FIGS. 60 and 61. Cross-section J-J are taken through the axis of rotation/pivot of the medial upright extension 6220 and/or through the longitudinal axis of the respective joint rivet, which cross-section lies on a plane that is normal to the bottom of the medial sidewall when the medial upright is at the 90° position and/or the medial joint is in the zero pivot state (the multi-mode pivot system is locked so that the lateral upright cannot pivot-more on this below) and/or normal to a flat level surface upon which the user of the external ankle brace would walk. Cross-section J-J (FIG. 79) is taken through the axis of rotation/pivot of the medial upright extension 6220 and/or through the longitudinal axis of the respective joint rivet, which cross-section lies on a plane that is normal to the bottom of the medial sidewall when the lateral upright is at the 90° position and/or the lateral joint is in the zero pivot state (the multi-mode pivot system is locked so that the medial upright cannot pivot-more on this below) and/or normal to a flat level surface upon which the user of the external ankle brace would walk. Cross-section K-K shown in FIG. 80 is on a plane parallel to and 0.25 inches in front of that of cross-section J-J, and cross-section L-L shown in FIG. 81 is on a plane parallel to and 0.5 inches in front of cross-section J-J. FIG. 82 shows the cross-section of section M-M. Cross-section N-N shown in FIG. 83 is on a plane parallel to and 0.25 inches in front of that of cross-section M-M, and cross-section O-O shown in FIG. 84 is on a plane parallel to and 0.5 inches in front of that of cross-section M-M.

It is noted that in an exemplary embodiment, cross-sections K-K, L-L, N-N and O-O are representative of cross-sections that would be in back of the cross-sections J-J and M-M, respectively, by the amounts detailed. That is, the embodiments can be mirrored about the planes through the axes. The values shown can be rounded to the second decimal place/the third decimal place can be deleted in some embodiments.

It is noted that the values of FIGS. 79-84 along with the structural features thereof associated there with can be utilized to scale other structural features disclosed herein in some embodiments. But it is noted that in some embodiments, the structures can have other dimensions and/or configurations.

In an exemplary embodiment, any dimension of any structure disclosed herein corresponds to an alternate disclosure where the dimensions of this structure in the alternate disclosure vary from those presented herein by less than and/or equal to and/or greater than 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 percent, or any value or range of values therebetween in 0.01% increments where the disclosed dimension is the baseline. By way of example only and not by way of limitation, this can be the case with respect to external ankle braces that are utilized for toddlers, where the values disclosed herein are for an external ankle brace that is used for a person wears a standard male size 9 shoe (USA) and/or a size 5, 6, 7, 8, 10, 11, 12, or 13 shoe. Further by way of example only and not by way of limitation, this can be the case with respect to an external ankle brace that is utilized for someone who wears a male size 13 shoe. Also by way of example, this can be the case for someone who wears a male size 9 shoe but where the embodiment has a different shape for whatever reason (accommodate a different anatomical ankle, for example).

It is noted that while the cross-sections presented in the figures stop at 0.5 inches from behind the pivot points, it is to be understood that there will be contours that can be located at cross-sections further behind (and thus further forward) than those cross-sections presented. In an exemplary embodiment, the contours for the cross-sections at 0.75 inches and at 1 inch and at 1.25 inches and at 1.5 inches behind and/or in front of the planes from the pivot points can have the extrapolated values from the values disclosed herein. In an exemplary embodiment, linear extrapolation can be utilized and/or a curve fit can be utilized (cubic (such as obtained via a cubic spline), simple linear, quadratic, polyratio (1,1), (2,2), or any other curve fitting technique that can be utilized, etc.), and those that can be accomplished utilizing three points. Also, values in between these cross-sections can be extrapolated, also utilizing linear and/or curve fitting techniques, such as curve fitting techniques that can be applied utilizing three points. Cubic spline interpolation can be used. And note that in at least some exemplary embodiments, the values in the Z direction are the control values. Thus, in an exemplary embodiment, any disclosure herein corresponds to an alternate disclosure of values for the not specifically stated values just detailed correspond to those that are obtained via the linear interpolation and/or the curve fitting techniques and/or the cubic spline interpolation techniques.

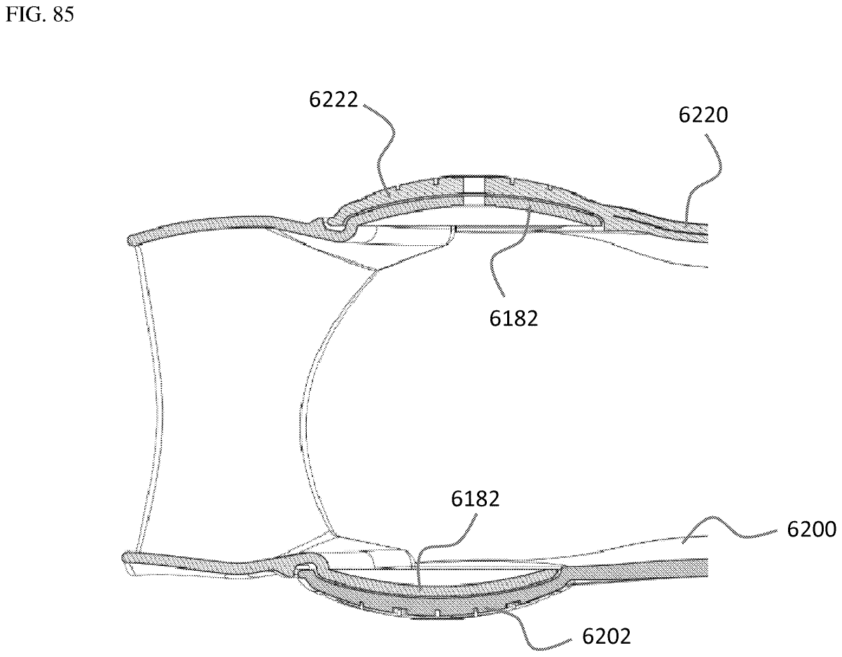

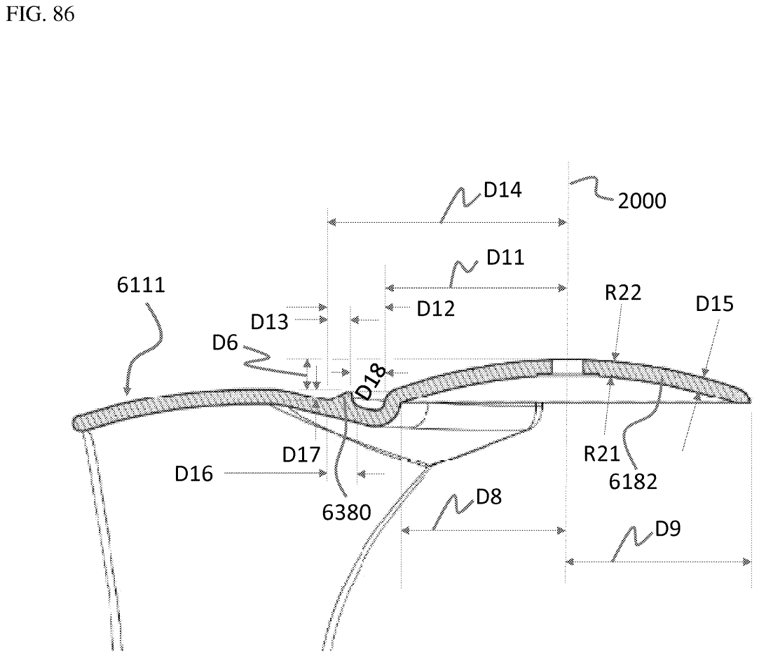

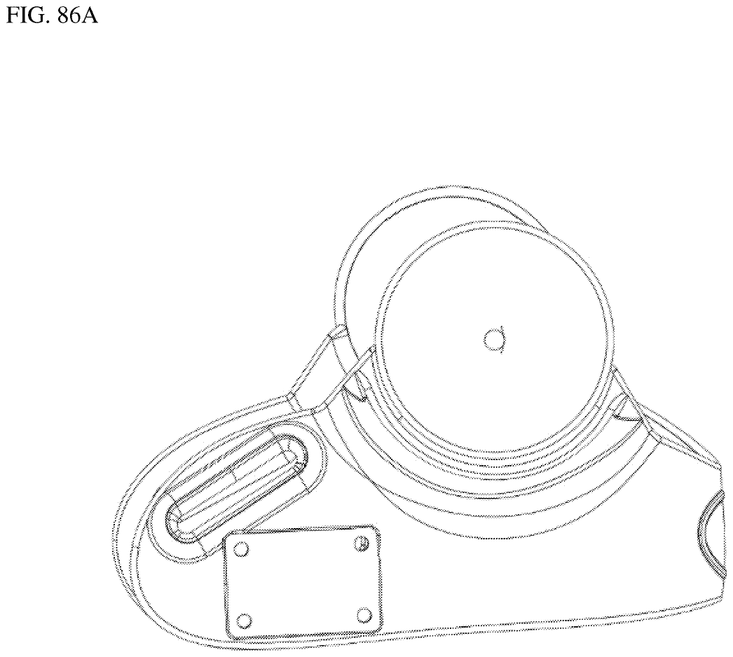

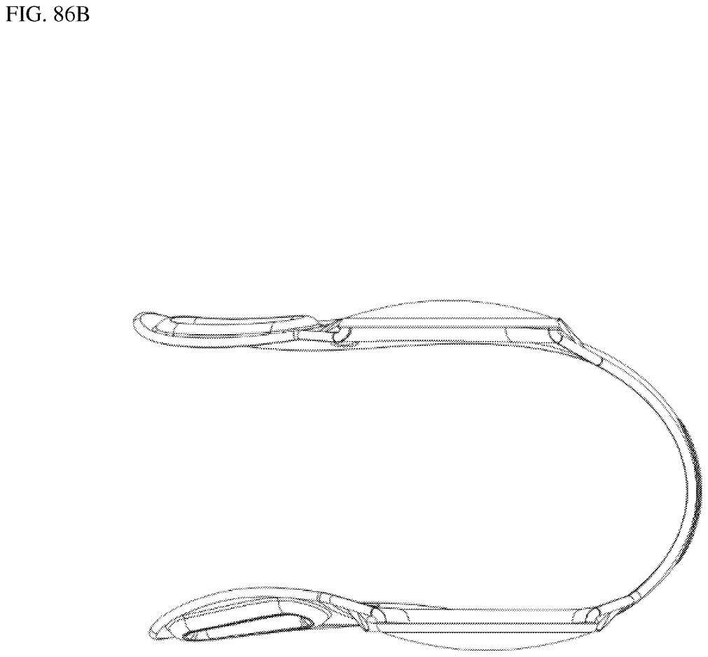

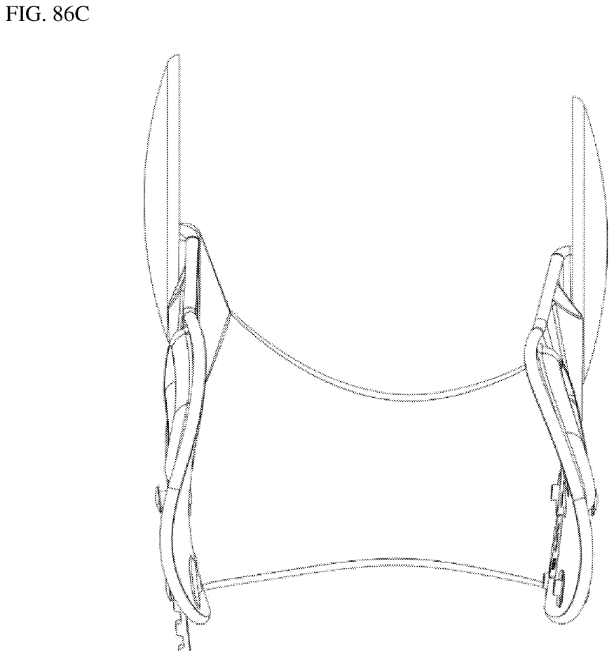

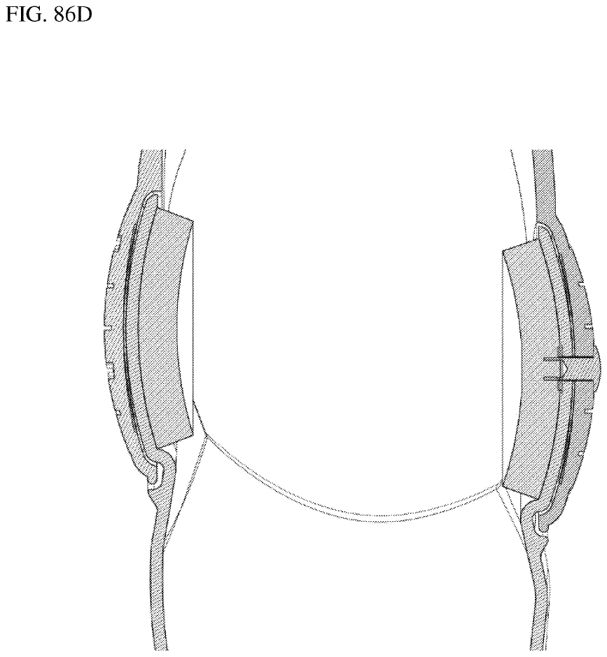

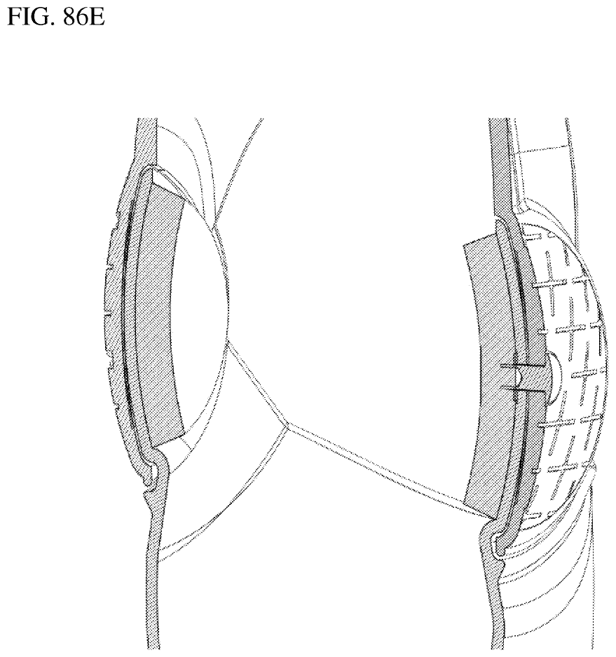

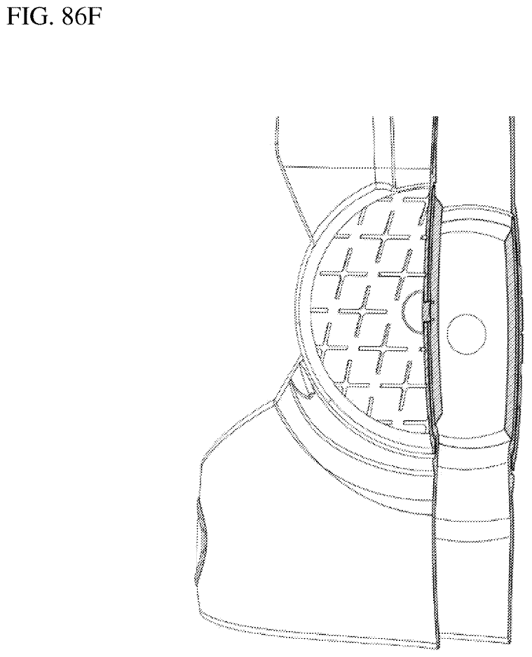

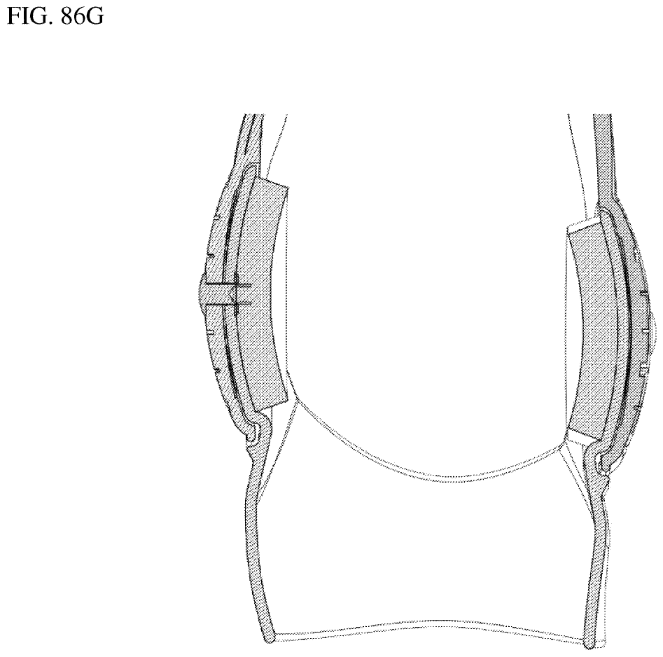

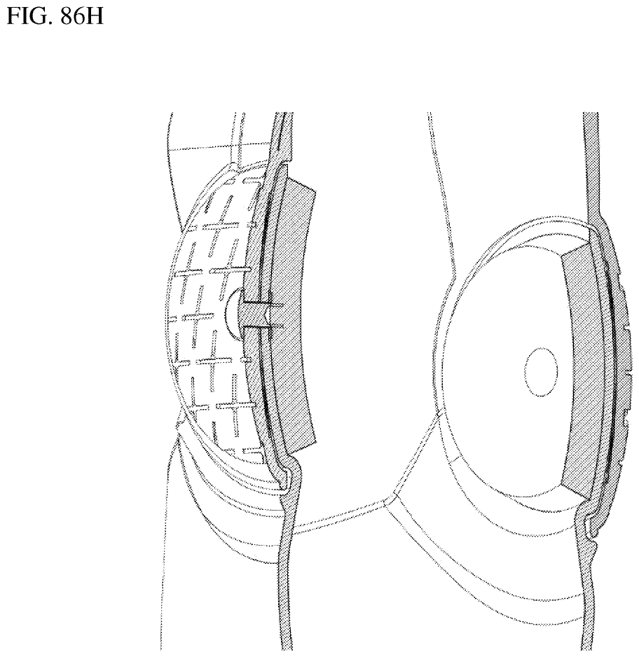

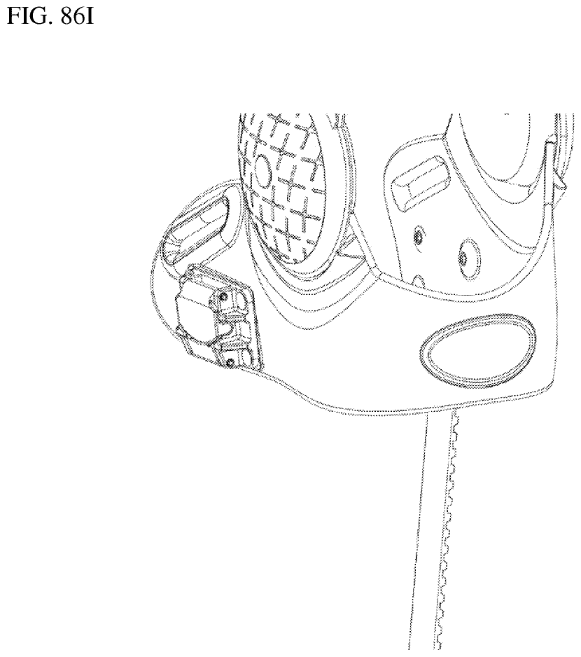

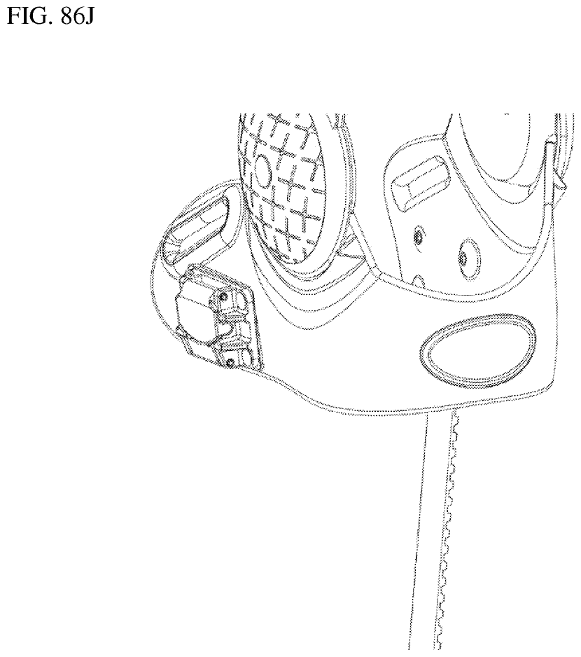

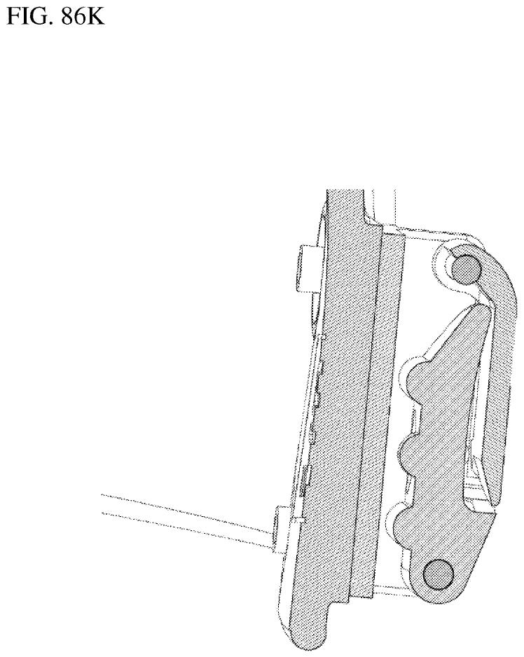

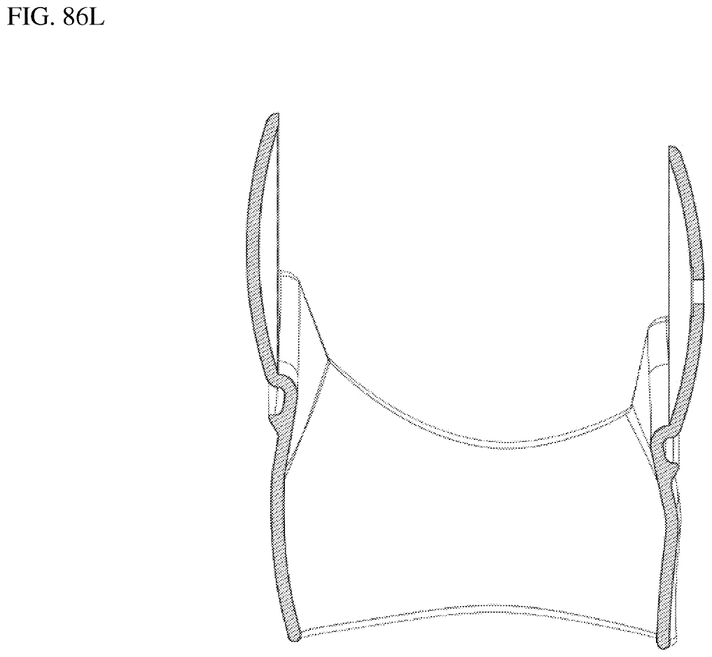

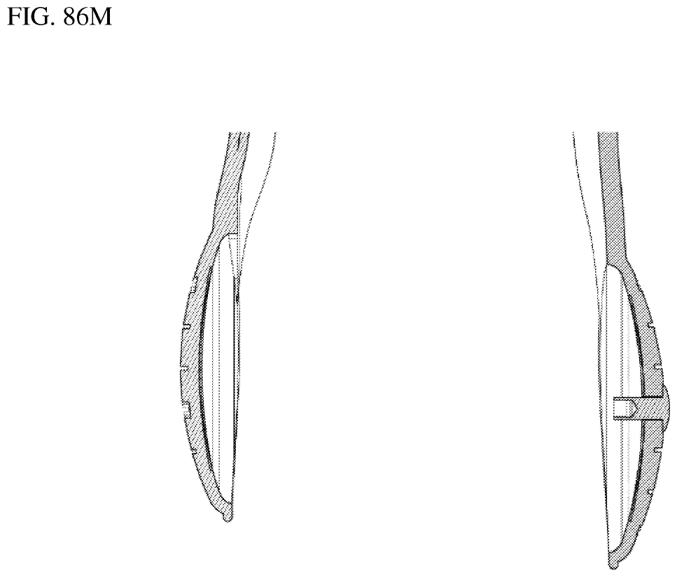

FIG. 85 shows a cross-section through the medial rotational joint axis (cross-section K-K). FIG. 86 shows the cross-section of K-K, with the uprights removed, and focusing on the medial side. That is, FIG. 86 shows a cross-section through the heel enclosure 6111, where features related to the bump 6830 are dimensioned, and other features. D11 is the radius of the male part of the joint, and can be less, than, greater than and/or equal to 0.5, 0.6. 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5 inches or more, or any value or range of values therebetween in 0.01 inch increments. The D14 can be less, than, greater than, and/or equal to 0.6. 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, or 2.6 inches or more, or any value or range of values therebetween in 0.01 inch increments. The left boundary of D14 is where the bump 6830 begins, and here, it is where the surface “rises” (in the frame of reference of FIG. 86 from the contour of the sidewall. It can be measured from the inflection point/the point where the tangent surface has a slope that reverses the trend (the slope changes towards the positive direction from previously increasingly negative). D12 can be D14-D11. D13 is the distance from the inflection point to the highest part of the bump and/or the most distant part of the bump (from left to right) that still has a positive tangent surface (the slope is positive), and can be less, than, greater than and/or equal to 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49, or 0.50 inches or more, or any value or range of values therebetween in 0.01 inch increments. Indeed, as can be seen in the figures, D13 has a variable value with respect to location towards the back of the brace, owing to the inward curvature of the body of the heel enclosure (arcing towards the center). In this regard, while the values just presented are presented in terms of the cross-section through the pivot line on the vertical plane, these values can be for other locations forward and/or backward of that plane. D18 can be D14-D13.

D16 can be any of the values of D13 plus 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2, or any value or range of values therebetween in 0.01 increments.

D15 can be less, than, greater than and/or equal to equal to 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, or 0.38 inches or more or any value or range of values therebetween in 0.01 inch increments, and this need not be uniform at all locations (again, this is shown for this cross-section—in the interests of textual economy, any disclosure of a feature for one location corresponds to a disclosure for another location of the structure under dimensioning unless otherwise noted, providing that the art enables such). But in some embodiments, the dimensions are uniform.

D17 is measured from the inflection point that constitutes the start of the bump as noted above to the highest point on the bump (relative to the arrangement of FIG. 86, and can be can be less, than, greater than and/or equal to equal to 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49, 0.50, 0.55, 0.6, 0.65, or 0.7 inches or more, or any value or range of values therebetween in 0.01 inch increments, and this need not be uniform at all locations.

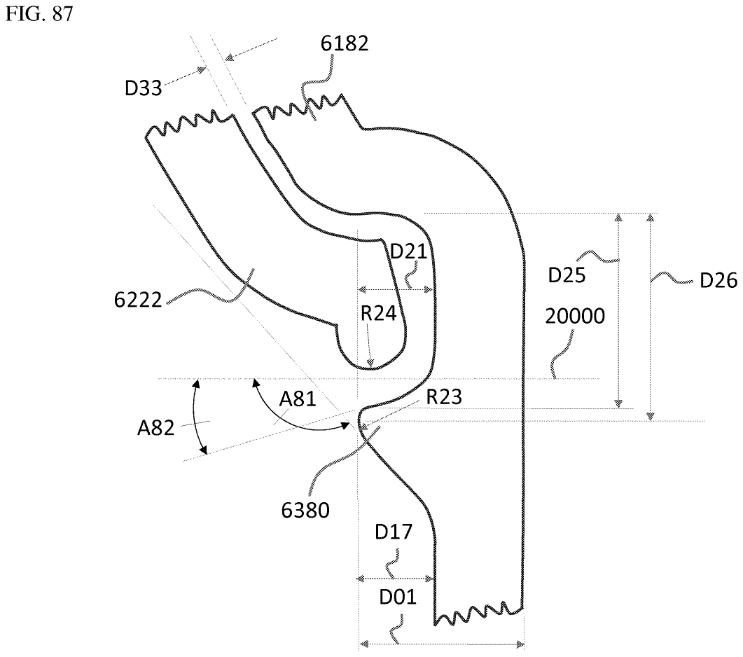

D26 is the distance from the highest part of the bump 6830 and/or the most distant part of the bump (from left to right) that still has a positive tangent surface (the slope is positive), and can be less, than, greater than and/or equal to 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49, or 0.50 inches or more, or any value or range of values therebetween in 0.01 inch increments. D26 is the distance from the inflection point on the upper surface of the bump (upper relative to the frame of reference of FIG. 87) to the inflection point of the male portion of the joint (relative to the rest of the heel enclosure) and/or the most distant portion (from the inflection point on the bump) of the heel enclosure that is the most outboard but still inboard of all portions of the one or more portions of the female portion 6222, and can be any of the just noted values of D26 minus 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, or 0.2, or any value or range of values therebetween in 0.01 increments.

Angle A81 is the angle from the axis 20000 that is parallel to axis 2000 and the plane of the bottom facing surface (relative to FIG. 87) or the surface with a constant tangent surface and/or the mean, median and/or mode of the tangent surface from the beginning of the rise of the bump (with movement from the bottom to the top) to the highest part of the bump (most left portion in the frame of reference of FIG. 87) and/or the inflection points thereof, and this can be different at different cross-sections (all the different cross-sections are taken on planes that extend through and are coincident with axis 2000). A81 can be less, than, greater than and/or equal to 100, 105, 110, 115, 120, 125, 130, 135, 140, 145, 150, 155, 160, 165, 170 or 175 degrees or more or any value or range of values therebetween in 1 degree increments. Angle A82 is the angle from the axis 20000 that is parallel to axis 2000 and the plane of the top facing surface or the surface with a constant tangent surface and/or the mean, median, and/or mode of the tangent surface from the beginning of the rise of the bump (with movement from the top to the bottom) to the highest part of the bump and/or the inflection points thereof, and this can be different at different cross-sections (all the different cross-sections are taken on planes that extend through and are coincident with axis 2000). A82 can be less, than, greater than and/or equal to 5, 10, 15, 20, 25, 30, 35, 40, 45, 50, 55, 60, or 65 degrees or more, or any value or range of values therebetween in 1 degree increments. Note that angle A82 could be negative with the frame of reference of FIG. 87 (and thus the upper surface would extend upwards in the frame of reference of FIG. 86), and can be any of the just noted values in the interests of textual economy. FIG. 87 shows an enlarged view of the measurements of D17 (where the dimension lines are parallel to the extend of the sidewall). Also shown is measurement D21, which is measured from the same point of the bump as D17 on the left, but is measured to the trough surface between the bump 6380 and the male portion 6182, and this can be the most inboard portion between the two (and thus from the most outboard portion of the bump (any of the measures associated with the top or highest point of the bump can be the most outboard portion of the bump, in totality or for a given cross-section or can be the mean/median and/or mode of the outboardness of the trough (between the curved parts) and/or between the inflection points of the trough or the geometric middle, etc.

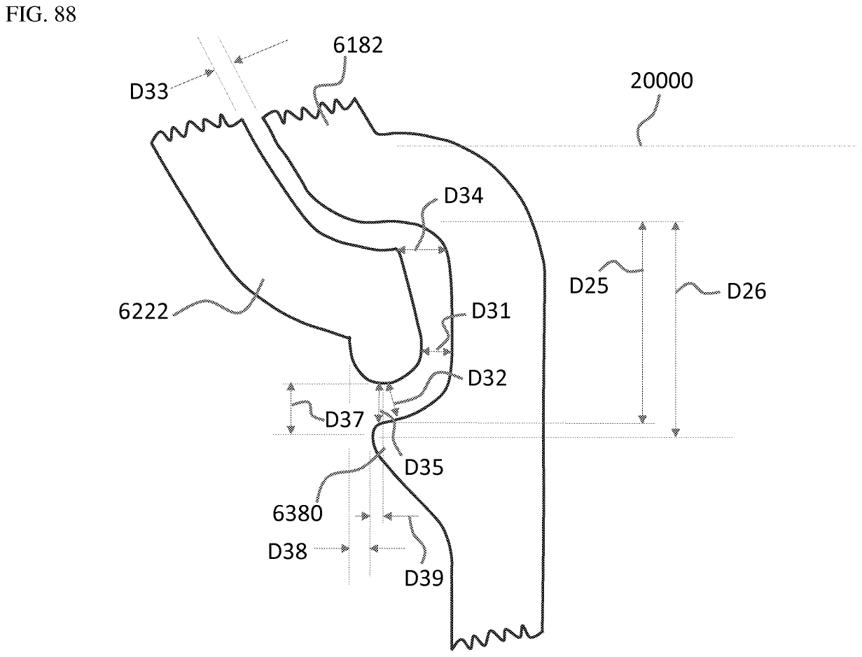

Variously herein it has been noted that the dimensions and features presented herein can be different for different portions of the brace. The measurements herein are presented in terms of FIGS. 86 and 87, which are views of the cross-section on the plane coincident with axis 2000 and is vertical (cross-section JJ). In an embodiment, the cross-sections or at least the dimensions thereof can be for planes that are also coincident with axis 2000, but are angled from the vertical plane, and can be for any one or more of these planes that are angled from the vertical plane by 1, 2, 3, 4, 5, 6, 7, 8, 9, 10, 15, 20, 25, 30, 35, 40, 45, 50, 55, 60, 65, 75, 80, 85, 90, 95, 100, 105, 110, 115, 120, 125, 130, 135 or 140 degrees or more or any value or range of values therebetween in 1 degree increments on either side of the plane and the values need not be the same (and will not be in some embodiments) for a plane that is say at 20 degrees on one side of the vertical plane and 20 degrees on the other side of the vertical plane (owing to, for example, how the heel enclosure arcs inward with location towards the rear of the brace), all in the interests of textual economy. And note that one or more features can be located in mirror image on the opposite side of a plane extending vertically (with respect to FIG. 88) and coincident with axis 2000, with respect to the various planes just noted, again in the interests of textual economy. This is also the case for any of the cross-sections herein unless otherwise noted, providing that the art enables such.

R21 is the interior radius of curvature of the male portion 6182 of the medial joint of the medial joint and R22 is the exterior radius of curvature of such. R22 can be less, than, greater than and/or equal to 2, 2.25, 2.5, 2.75, 3, 3.25, 3.5, 3.75, 4, 4.25, 4.5, 4.75, 5, 5.25, 5.5, 5.75 inches or more or any value or range of values therebetween in 0.01 inch increments. R21 can be R22 minus D15, and the thickness are constant over the span in some embodiments, and are not constant over the span in some embodiments. D33 is the maximum, minimum, mean, median and/or mode of the distance between the inner curved surface of the female portion of the joint and the outer curved surface of the male portion of the joint and/or for all portions below the pivot axis 2000, and can be less than, greater than and/or equal to 0.001, 0.002, 0.003, 0.004, 0.005, 0.006, 0.007, 0.008, 0.009, 0.01, 0.015, 0.02, 0.025, 0.3, 0.035, 0.04, 0.05, 0.06, 0.07, 0.08, or 0.09, or any value or range of values therebetween in 0.001 inch increments, and in an embodiment, this can be controlled or based on the thickness of the “washer” that is interposed between the two, however much that is compressed, if any. D33 can be measured in the horizontal plane of FIG. 87 and can be from the two closest parts of the components below the axis 2000. In an embodiment, D31 can be 0.001, 0.002, 0.003, 0.004, 0.005, 0.006, 0.007, 0.008, 0.009, 0.01, 0.015, 0.02, 0.025, 0.3, 0.035, 0.04, 0.05, 0.06, 0.07, 0.08, 0.09, 0.1, 0.11, 0.12, 0.13, 0.14, 0.15, 0.16, 0.17, 0.18, 0.19, 0.2, 0.225, 0.25, 0.275, or 0.3 or more, or any value or range of values therebetween in 0.001 inch increments, where D31 is the closet approach of a portion of the joint 6222 that is the most inboard. D34 could be the distance from the beginning of the female shape of joint 6222 measured parallel to axis 2000, and D34 could be any of the values of D31 and/or any of those values plus 0.05, 0.01, 0.015, 0.02, 0.025, 0.03, 0.04, 0.05, 0.075 0.1, 0.125, 0.15, 0.175, 0.02 or more inches, or any value or range of values therebetween in 0.01 inch increments and the values need not be the same. In an embodiment, a mean, median, and/or mode of the distances between those two locations could be any value of D34 and the values need not be the same.

D32 represents the closest approach of the joint portion 6222 to the inflection point of the bump facing the joint portion, and D35 represents the distance in the vertical direction from the lowest portion of the joint portion to the part of the bump immediately below such (with respect to the frame of reference of FIG. 88). D37 is the distance from the lowest portion of the joint portion to the crest/inflection point and/or the outermost portion of the bump. The values for these can be, in the interests of textual economy, less, than, greater than and/or equal to 0.02, 0.03, 0.04, 0.05 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49 or 0.50 inches or more or any value or range of values therebetween in 0.01 inch increments and the values need not be the same. D38 is the distance from the crest or inflection point of the bump and the beginning of the curvature portion of the female portion 6222 of the joint, and can be less, than, greater than and/or equal to 0 (more on this in a moment), 0.01, 0.02, 0.03, 0.04, 0.05 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49, or 0.50 inches or more, or any value or range of values therebetween in 0.01 inch increments. Conversely, the value of D38 can be negative when measured from the crest/maximum outboard portion of the bump/the inflection point of the bump. That is, the crest of the bump could be more outboard than the beginning of the curved portion, and can be any of the just noted values of D38 except negative. D39 is the distance from the crest, etc., of the bump to the lowest most portion of the joint portion 6222 or the closest approach in the vertical direction to the heel enclosure and/or an inflection point of the joint portion 6222 and can be less, than, greater than and/or equal to 0 (more on this in a moment), 0.01, 0.02, 0.03, 0.04, 0.05 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46. 0.47, 0.48, 0.49, or 0.50 inches or more, or any value or range of values therebetween in 0.01 inch increments. Conversely, the value of D39 can be negative when measured from the crest/maximum outboard portion of the bump/the inflection point of the bump. D8 is an interior distance of the curvature of the male portion 6182, and can be any of the pertinent values of D11 in the interests of economy, or any of those values minus less than, greater than and/or equal to 0.01, 0.02, 0.03, 0.04, 0.05, 0.6, 0.07, 0.08, 0.09, 0.1, 0.125, 0.15, 0.175, 0.2, 0.225, 0.25, 0.275, 0.3 inches or more or any value or range of values therebetween in 0.005 inch increments. Note that the dimension from axis 2000 to the top (most right portion) of the male portion can be D9, which can be any of the values of D8 plus less than, greater than and/or equal to 0.01, 0.02, 0.03, 0.04, 0.05, 0.6, 0.07, 0.08, 0.09, 0.1, 0.125, 0.15, 0.175, 0.2, 0.225, 0.25, 0.275, 0.3 inches or more, or any value or range of values therebetween in 0.005 inch increments, and the values need not be the same (for textual economy).

FIG. 87 shows a dimension D01, which is the total thickness of the bump plus the heel enclosure body (where here, enclosure body is monolithic with the bump). D01 can be less than, greater than and/or equal to 0.15, 0.2, 0.25, 0.3, 0.35, 0.4, 0.45, 0.5, 0.55, 0.6, 0.65, 0.7, 0.75, or 0.8 inches or more, or any value or range of values therebetween in 0.01 inch increments.

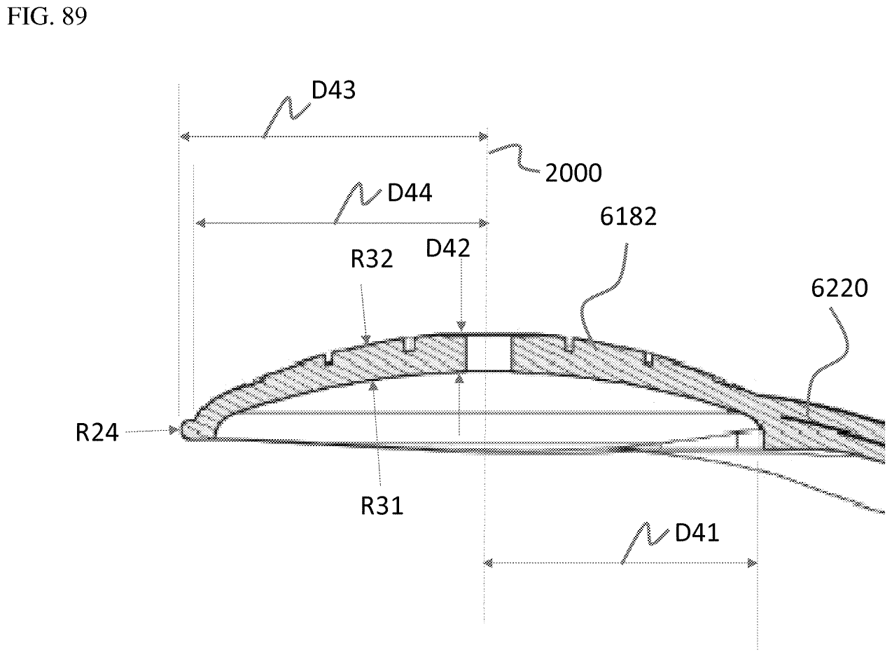

FIG. 89 shows a J-J cross-section of the female portion of the medial joint. D41 is the interior distance (the radius) of the female cavity and can be less, than, greater than and/or equal to 0.55, 0.6. 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, or 2.6 inches or more, or any value or range of values therebetween in 0.01 inch increments. The value of D44, which is from the axis 2000 to the end of the curvature on the outside of the joint portion, or an inflection point and can be less, than, greater than and/or equal to 0.6. 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, or 2.7 inches or more, or any value or range of values therebetween in 0.01 inch increments. D43 is the distance to the lowest portion (leftmost portion) of the joint or the medial upright), and can be any of the values of D44 or any of those values plus less than, greater than and/or equal to 0.01, 0.02, 0.03, 0.04, 0.05, 0.6, 0.07, 0.08, 0.09, 0.1, 0.125, 0.15, 0.175, 0.2, 0.225, 0.25, 0.275, 0.3 inches or more, or any value or range of values therebetween in 0.005 inch increments, and the values need not be the same (for textual economy). D42 is the thickness of the female joint portion at the edge of the through hole for the rivet (and this can be the thickness of extrapolated surfaces using a curve fit for example or a linear extrapolation for example as measured ono axis 2000), and can be less, than, greater than and/or equal to equal to 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40 inches or more, or any value or range of values therebetween in 0.01 inch increments. The thickness changes with location along the span, as seen in the embodiment of FIG. 89, and thus the thickness of the female portion is not uniform at all locations. In an alternate embodiment, the dimensions are uniform. R31 is the interior radius of curvature of the interior of the female joint portion, and can be less than, greater than and/or equal to 2, 2.25, 2.5, 2.75, 3, 3.25, 3.5, 3.75, 4, 4.25, 4.5, 4.75, 5, 5.25, 5.5, 5.75 inches or more, or any value or range of values therebetween in 0.01 inch increments. R32 can be R31 plus D42, where D41 is carriable in some embodiments, and constant in others.

In an embodiment, the bump 6380 has a curved outer profile, and the radius of curvature of the bump is 0.01, 0.015, 0.02, 0.025, 0.3, 0.035, 0.04, 0.05, 0.06, 0.07, 0.08, 0.09, 0.1, 0.125, 0.15, 0.175, 0.2, 0.225, 0.25, 0.275, 0.3, 0.325, or 0.35 or more, or any value or range of values therebetween in 0.001 increments, and in an embodiment n be any of those values and need not be the same in the interests of textual economy.

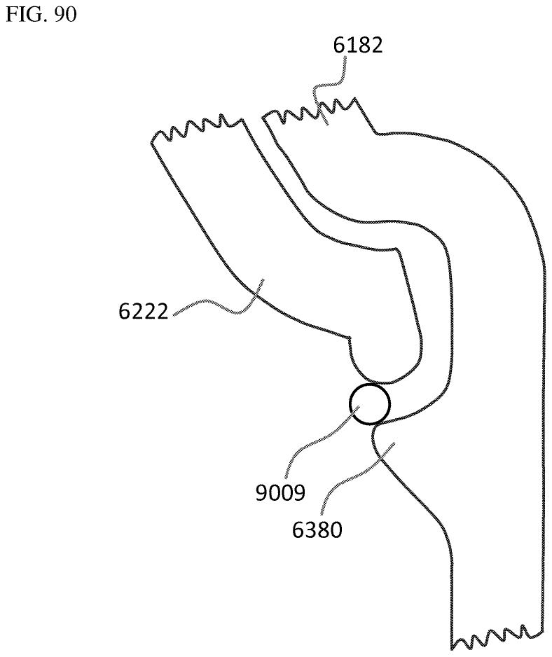

In an embodiment, the passage/channel between the bump 6380 and the outer periphery of the female portion 6222 of the joint (which can be the lowest portion in the frame of reference of FIG. 88—any disclosure herein of the lowest portion corresponds to another disclosure of a feature at another location on the periphery, and any disclosure related to the lowest portion corresponds to an another disclosure related to the lowest portion where such disclosure is at another location on the periphery) is such that a sphere with a diameter of 0.01, 0.015, 0.02, 0.025, 0.03, 0.035, 0.04, 0.045, 0.05, 0.055, 0.06, 0.065, 0.07, 0.075, 0.08, 0.085, 0.09, 0.1, 0.15, 0.2, 0.25, 0.3, 0.35, 0.4, 0.45, or 0.5 inches, or any value or range of values therebetween in 0.005 inch increments cannot fit into the channel or cannot fit past a certain point in the channel. FIG. 90 shows an exemplary sphere 9009, which will be a sphere made of stainless steel 304 having a modulus of elasticity of at least 180 GPa, under testing to see if the brace meets the requirements. The sphere 9009 can have any of the just noted diameters and values. In an embodiment, the half sphere or the geometric center of the sphere does not extend past the top inflection point of the bump (facing the female portion of the joint) and/or does not extend past the bottom inflection point of the female portion of the joint and/or the most narrow portion of the channel in a direction measured vertically from one portion of the bump and/or the closest approach of the bottom portion of the joint portion in the vertical direction to the bump and/or as measured in the vertical direction, the maximum outboard portion of the bump/crest of the bump, etc.

The bump 6380 can have utilitarian value with respect to providing a guard or otherwise preventing the female portion of the lateral and medial joint respectively from being caught on something when a person is walking or running or jumping with the brace worn about the exterior of the shoe. By way of example only and not by way of limitation, it could be that a paracord or the like could become ensnared in between the female portion of the joint and the heel enclosure. The ideas that an object “riding along” the outer surface of the heel enclosure or more accurately movement of the outer surface of the heel enclosure against and relative to an object such as a cord or the like and thus pressing against the cord will result in the cord being pushed outward away from the female portion of the joint and thus over the outside of the female portion of the joint, as opposed to being caught on the outer periphery of the joint. The bump 6380 acts as a ramp when the object is considered moving relative to the brace (of course, both scenarios the brace moves relative to the object) and the object is pushed outward as the object moves from the bottom of the rigid heel towards the top of the rigid heel. Embodiments described so far provide this ramp concept or otherwise provide for a portion of the rigid heel enclosure that is more outboard than a portion of the upright in general, and the female portion of the joint in particular. In at least some embodiments, the ideas that portions of the female portion of the joint it could catch on an object are located in board of the outermost portion of the bump 6380. In the embodiment shown above, the outermost portion of the bump is more outboard than the portion of the female portion at the outer periphery thereof that has a negative slope with respect to say the orientation of FIG. 90. The opposite would be the case for the female portion of the joint for the lateral side. The idea is that with respect to the frame of reference of FIG. 90, a surface having a tangent plane with a negative slope on the medial side would deflect an object, and thus the most outboard extends of the bump 6380 would extend to the point where the tangent plane begins to be negative in some embodiments, or further past that for heightened redundancy or otherwise to further lessen the chance that something would be caught on the upright. Still, various positions can be provided and it may not be that the bump extends all the way to that point for other reasons, such as other design constraints. In this regard, perfection would not be the enemy of good, and adequate or better would be adopted over nothing.

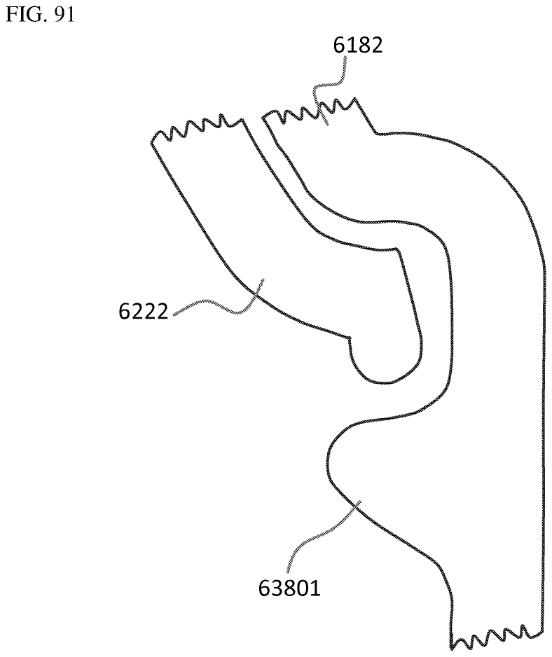

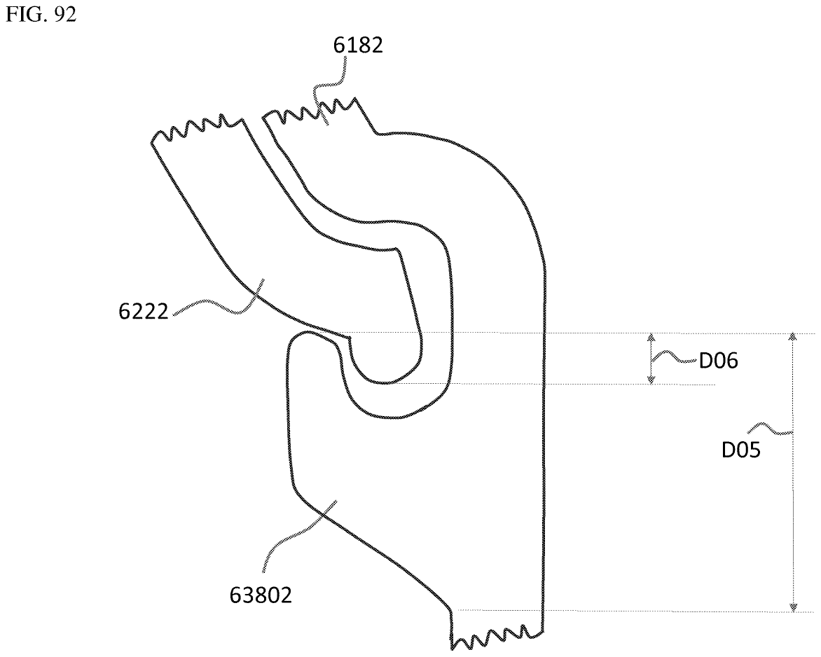

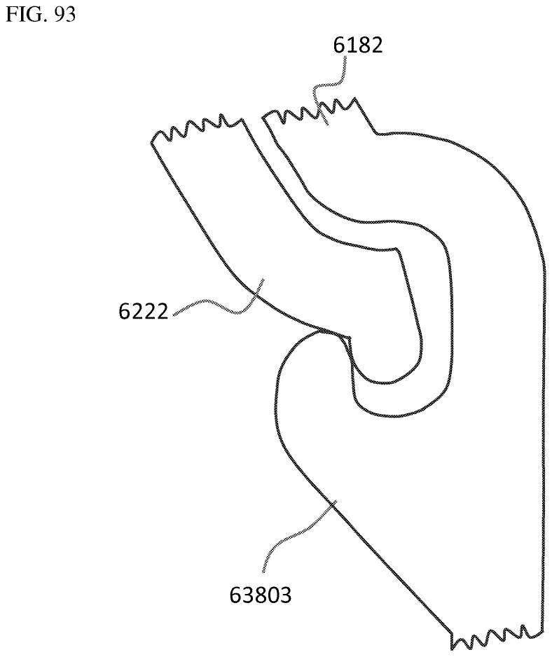

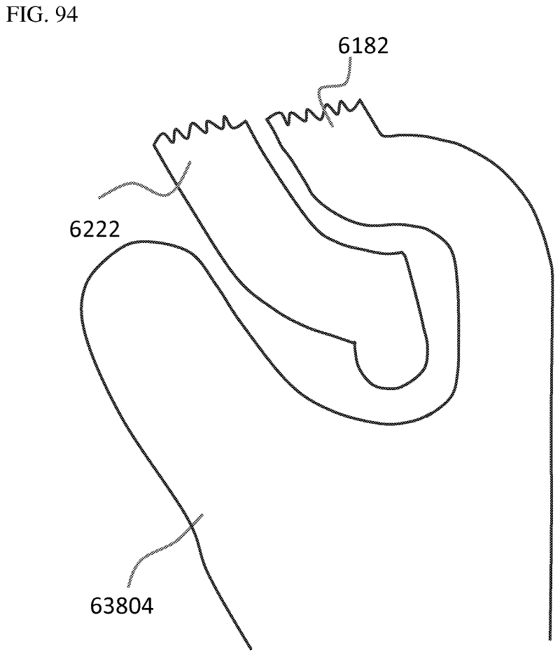

Still, other embodiments are contemplated, such as the bump 63801 of FIG. 91, which extends all the way out past where the tangent surface at the outer periphery begins to be negative. The arrangement of FIG. 92 has a thumb shaped arrangement, where barrier 63802 extends outward and above (from the frame of reference of FIG. 92), the bottom most portion of the female portion of the joint. The embodiments above have provided a clearance between the barrier/pumps and the upright. FIG. 93 shows another embodiment where there is contact between the two components. Here, barrier 63803 is configured to interfere with the female portion of the joint (the barrier elastically and/or plastically deforms so that the contact still permits the heel enclosure to move relative to the upright(s)). The idea is that the material of the brace is sufficiently pliable that the female portion of the joint can be slipped over the male portion and in between the portion of the barrier. Again, deformation of the barrier can be accomplished to permit the attachment of the upright to the heel enclosure. FIG. 94 presents another embodiment with an even more aggressive barrier 63804, but without contact.

D6 shows a height from the crest/outboardmost portion of the bump 6380 to the outermost portion/most outboard portion of the male portion (if from an extrapolated outer profile or from an actual part thereof), and D6 can be less than, greater than and/or equal to 0 (see below), 0.05, 0.1, 0.15, 0.2, 0.25, 0.3, 0.35, 0.4, 0.45, or 0.5, or any value or range of values therebetween in 0.01 inch increments.

Note that the right side dimension of D13 can be the “topmost” (from the orientation of FIG. 90 for example) dimension of the barrier/bump.

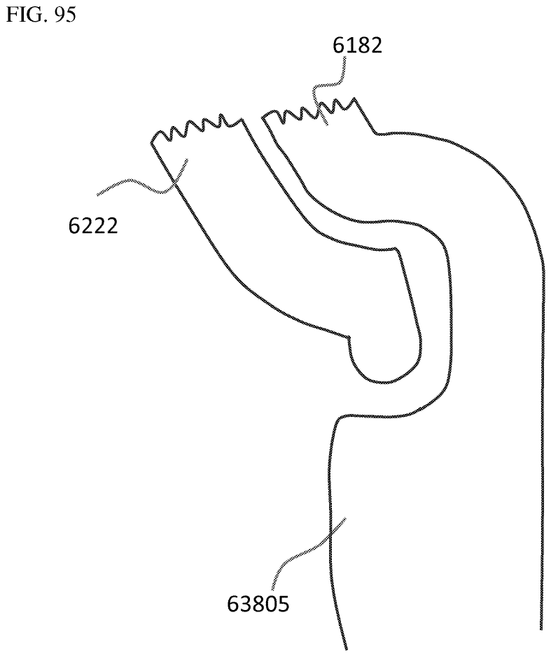

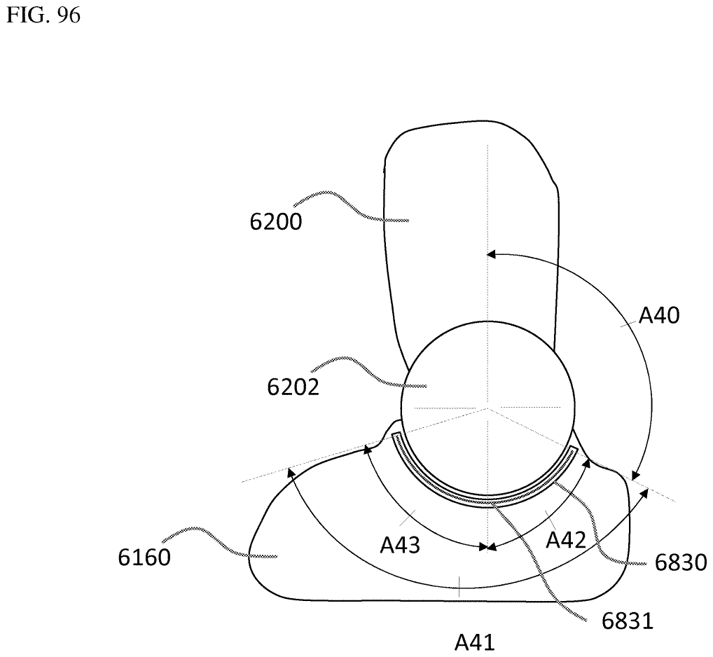

Embodiments above the focused on the concept where the heel enclosure includes the barrier and otherwise provides a protruding portion with respect to local geometry. Alternatively, and/or in addition to this, the heel enclosure could include a recess or otherwise the depression and the surface thereof into which an outer periphery of the female portion of the joint is fitted otherwise is located so as to provide the utilitarian value of reducing the likelihood that the upright will catch on something during use, such as a cord or cable or the side of a step, etc. In an embodiment, there could be a second portion of the heel enclosure in the area proximate the female portion of the joint and otherwise behind the female portion when looking at the side of the brace, or more accurately, the second portion could provide an area for the recess, which recess would be behind the outer periphery and portions thereof of the female portion of the joint. FIG. 95 shows such an embodiment, with thickened portion 63805. In an embodiment, there is a heel enclosure that has a thickness of any of the “heights” of the bumps detailed herein, such as dimension D17, which can be measured relative to an area that does not have the thickened section, or as measured from a location that has a thickness of ½, or ⅓^{rd} or ¼^{th} or 20% or less of the thickness (maximum, minimum, mean/median and/or mode thickness, or thickness over an area of 70 or 80% of the thickened section, etc.). The relative location could be directly below or directly away on a plane (any of the planes detailed above) from the measured location. The thickness of the thickened section can be any of the values of D01, and in an embodiment, the thickness is less than, greater than and/or equal to 7, 6.5, 6, 5.5, 5, 4.5, 4, 3.5, 3, 2.5, 2, or 1.5, or any value or range of values therebetween in 0.1 increments times the thickness of another section that corresponds to any of those meeting the above noted comparison examples. In an embodiment, this is relative to the mean, median and/or mode of the thickness of the entire heel enclosure and/or the thickness of the heel enclosure directly below the section at issue or on any of the planes noted above, as taken 0.25 inches from the bottom periphery of the heel enclosure (simply to provide relative features associated with the thickened section). And note that this can also be the case for the bump/barrier configuration.

And while some embodiments have been presented in terms of the concept of a trough or otherwise a depression within the body of the heel enclosure, in other embodiments, the thickened section ends at a location proximate (effectively below) the outer periphery of the female portion, and then does not return to a thickness and otherwise the remainder of the heel enclosure above that portion or inside the end of the thickened portion or otherwise beneath or behind the female portion is the average thickness or a thickness consistent with other locations of the rigid heel and quote closure, and can have a thickness as measured according to any of the comparison locations just detailed. This is seen in FIG. 95 by way of example.

In an exemplary embodiment, the protrusion or bump 6830 extends in an arcuate manner about most of the outer periphery of the female portion of the joint that overlaps when viewed from the side view with the female portion of the joint. FIG. 96 shows an example of the accurate extension of the protrusion 6830. Here, the lateral side of the braces depicted, and it is noted that any of the features detailed herein with respect to the medial side can correspond to features of the lateral side, and vice versa, providing that the art naval such, unless otherwise noted. While in some embodiments, the medial side rotation axis is higher than the lateral side, in other embodiments this is not the case. All of this is made in the interests of textual economy.

FIG. 96 shows the protrusion 6830, and the crest 6831 of the protrusion, which in this embodiment, corresponds to the portion of the protrusion that extends most outboard from the side of the brace, and otherwise the greatest extension out of the plane of FIG. 96.

In an embodiment, less than, greater than and/or equal to 60, 65, 70, 75, 80, 85, 90, 95 or 100% or any value or range of values therebetween in 1% increments of the crest is located within two parallel planes located less, than, greater than and/or equal to 0.01, 0.015, 0.02, 0.025, 0.03, 0.035, 0.04, 0.045, 0.05, 0.055, 0.06, 0.065, 0.07, 0.075, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25 or more inches any value or range of values therebetween in 0.01 inch increments (again when the brace is in the relaxed state-any measurement herein is disclosed in terms of the relaxed state unless otherwise noted). Note also that this feature could be the case for the outer periphery of the female portion of the joint and/or the noted inflection points or changes in the slopes from positive to negative etc. of the joint portions. Also, this can also be the case with respect to the protrusions or the thickened portions for the barriers with respect to the inflection point thereof.

Returning to FIG. 96, it is shown that there are a series of angles depicted, which angles represent the angular locations of various portions of the barriers and/or thickness portions and/or bumps, etc., disclosed herein. Any disclosure of one corresponds to a disclosure of the other an interest of textual economy, unless otherwise noted. Here, angle a 40 extends from the vertical axis that passes through the rotation axis of the lateral sidewall, or otherwise the plane that extends through and is coincident with the axis of rotation that is vertical. Angle A40 extends therefrom to the beginning of the crest of the bump 6830. In this regard, the bump has a taper at the back (and the front) for that matter from the crest so that when viewed from the rear, a triangular-shaped or a shark fin shape is seen with the top tip extending from the crest. A40 can be less, than, greater than and/or equal to 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245 or 250 degrees or more or any value or range of values therebetween in 1 degree increments. Angle A41 is the total sweep of the crest, and can be less, than, greater than and/or equal to 5, 10, 15, 20, 25, 30, 35, 40, 45, 50, 55, 60 or 65 degrees or more or any value or range of values therebetween in 1 degree increments or any of the values just noted for A40 in the interests of textual economy or the addition of A40 plus A41 (for heel enclosures that extend higher than that shown/where there is more overlap with the heel enclosure and the uprights). A42 is the sweep of the crest from the beginning to the plane through the joint (the plane measured from A40), and A43 is a dimension extending from the plane measured from A40 to the end of the crest. A42 and/or A43 can be (and they need not be the same) less, than, greater than and/or equal to 5, 10, 15, 20, 25, 30, 35, 40, 45, 50, 55, 60, 65, 70, 75, 80, 85, 90, 95, 100, 105, 110, 115, 120, 125, 130, 135 degrees or more, or any value or range of values therebetween in 1 degree increments.

Note that any of the above values detailed herein can also be for the entire protrusion or thickness portion or otherwise the portion that provides the barrier, and not just the crest.

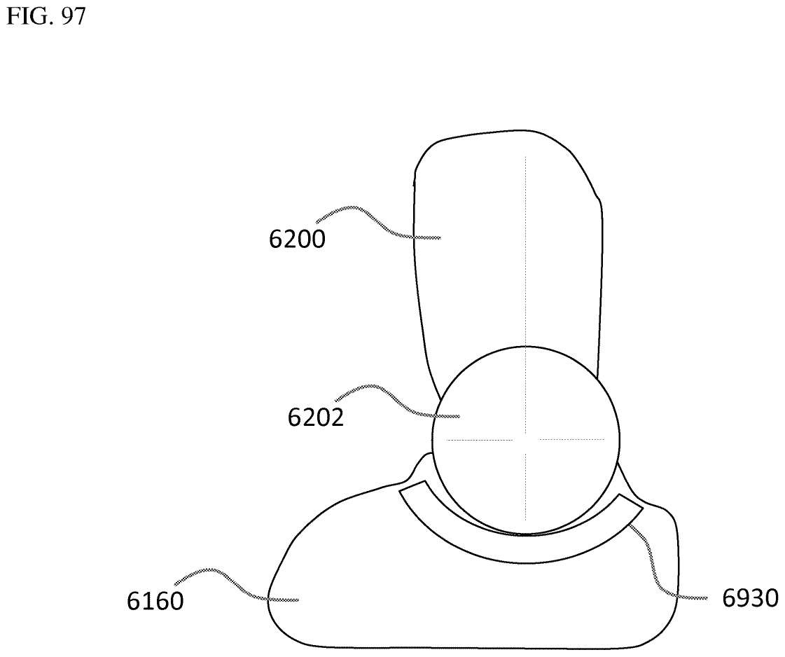

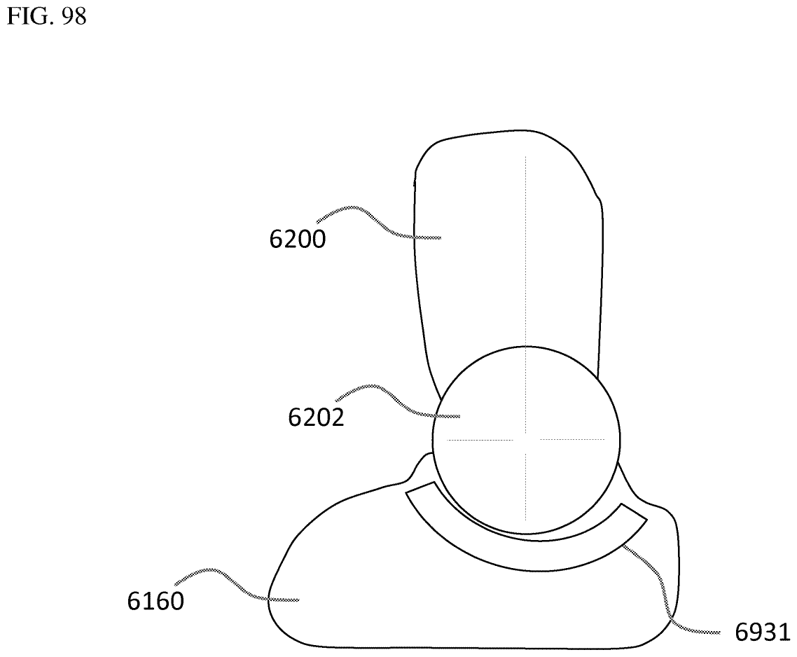

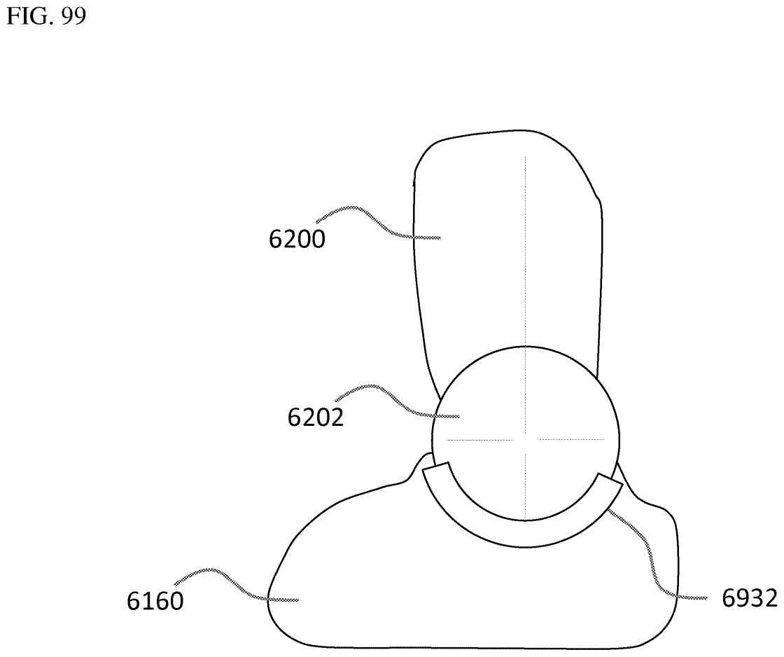

While the embodiments above have been presented in terms of the barrier or thickened portion or otherwise at least the crest being concentric with the outer periphery of the female portion (and the male portion for that matter) of the joint, in an alternate embodiment, this is not the case. FIG. 97 shows a non-concentric barrier 6930, and FIG. 98 shows a barrier 6931 that has a closest approach offset from the vertical plane through the rotation axis. FIG. 99 depicts a barrier 6932 that overlaps the outer periphery of the female portion 6202 of the joint. This can be representative of the embodiment of FIG. 92. And referring back to such, FIG. 92 shows dimension D06 is the overlap of the barrier with the female portion, and dimension D05, which is the total vertical span of the barrier (from the lowest portion that is noticeable to the highest portion—any of the inflection points can be instead used for these values or any other feature disclosed herein in the interests of textual economy. In an embodiment, D06 is less than, greater than and/or equal to 0, 0.05, 0.1, 0.15, 0.2, 0.25, 0.3, 0.35, 0.4, 0.45, 0.5, 0.55, 0.6, 0.65, 0.7, 0.8, 0.9, 1, 1.1, 1.2, 1.3, 1.4, 1.5, 1.6. 1,7, 1.8, 1.9, 2, 2.25, 2.5, 2.75, or 3 inches or more, or any value or range of values therebetween in 0.01 inch increments. And these could be negative values, at least for the lower numbers. D05 can be less than, greater than, and/or equal to 0.05, 0.1, 0.15, 0.2, 0.25, 0.3, 0.35, 0.4, 0.45, 0.5, 0.55, 0.6, 0.65, 0.7, 0.8, 0.9, 1, 1.1, 1.2, 1.3, 1.4, 1.5, 1.6. 1,7, 1.8, 1.9, 2, 2.25, 2.5, 2.75, 3, 3.25, 3.5, 3.75, 4, 4.25, 4.5, 4.75, or 5 inches or more, or any value or range of values therebetween in 0.05 inch increments.

And as will be understood from the above-noted values, some of these are for the non-overlapping barrier (FIG. 92 is simply used to convey some of the values).

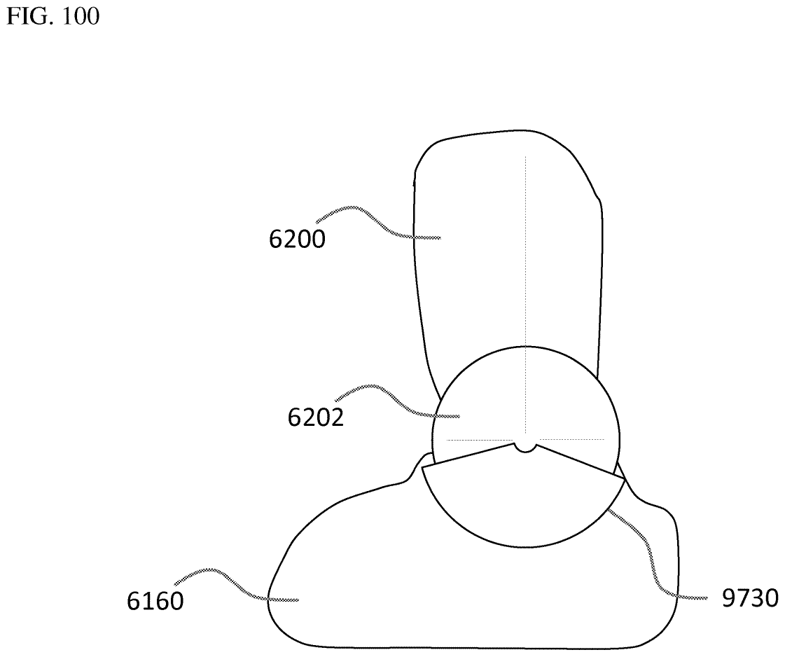

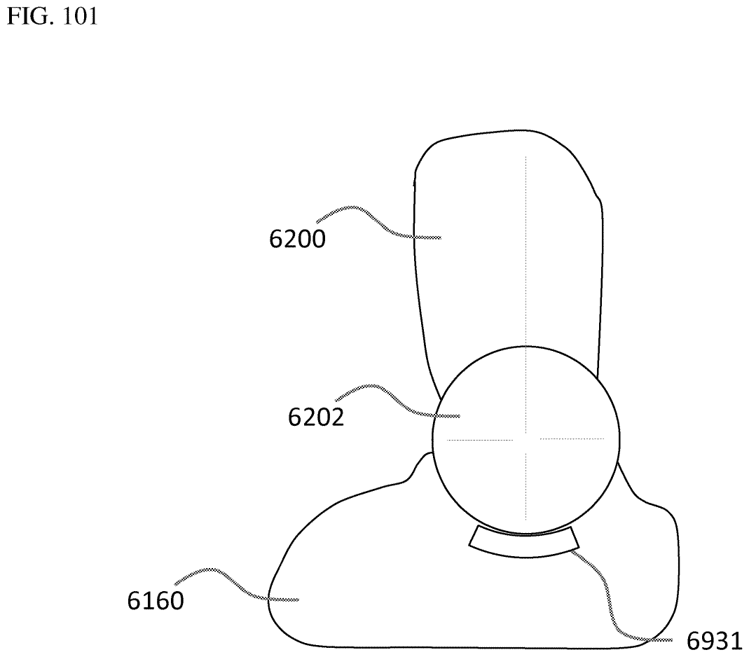

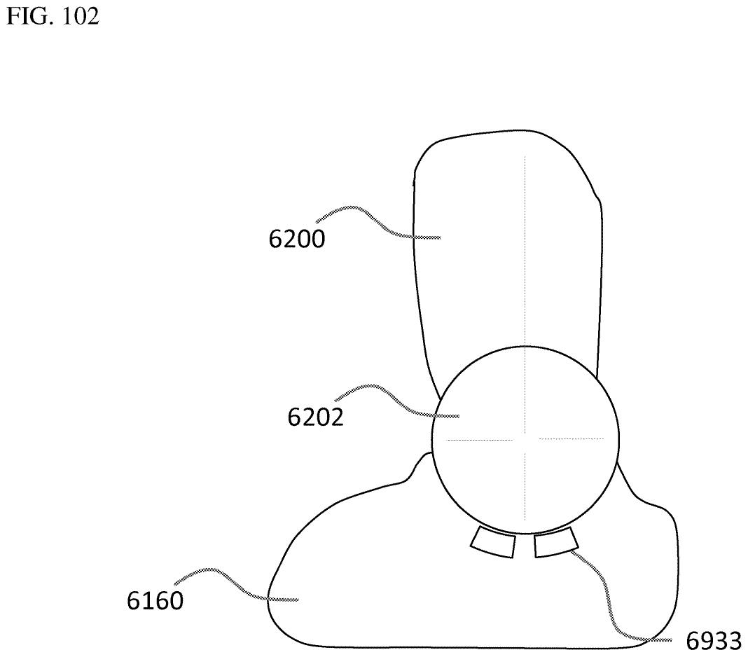

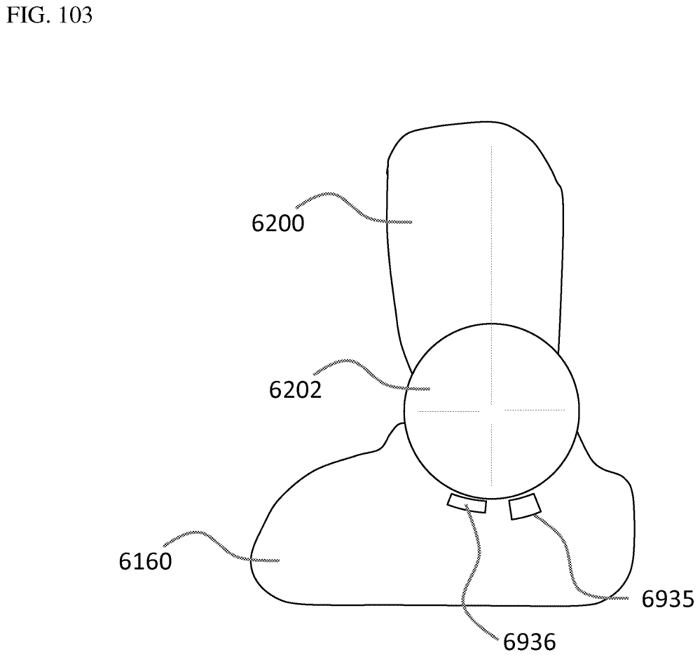

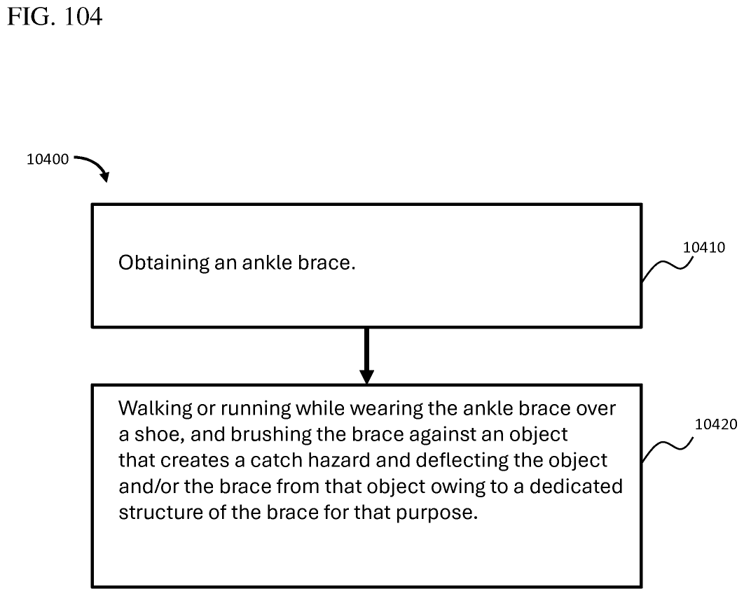

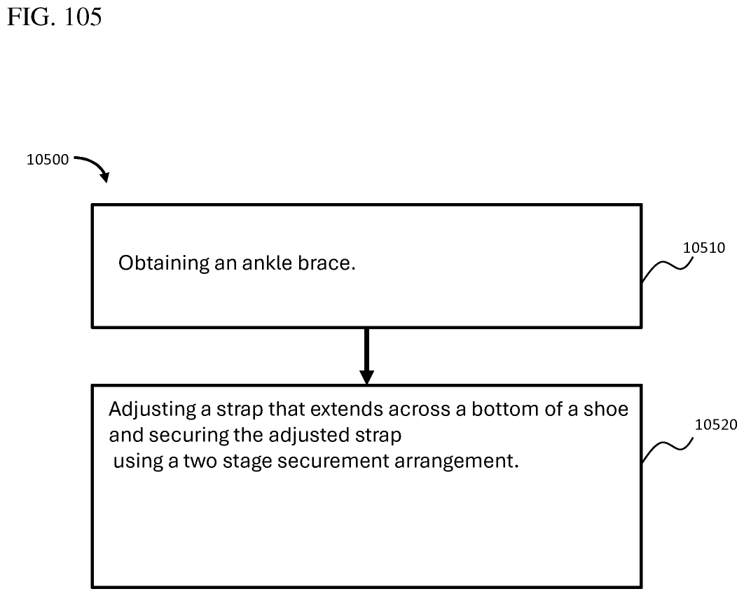

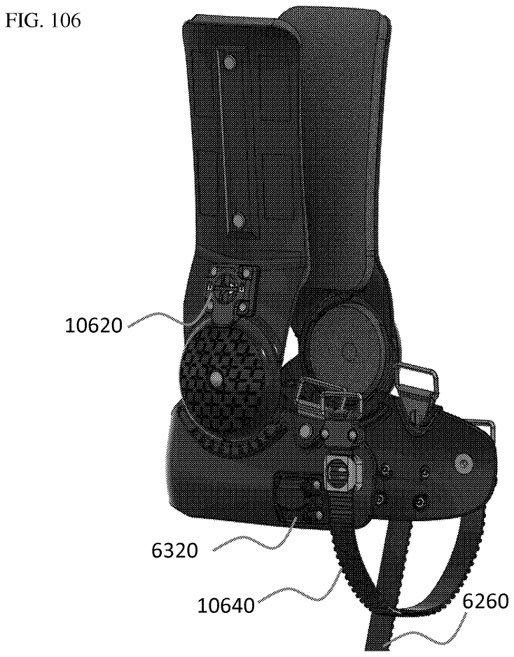

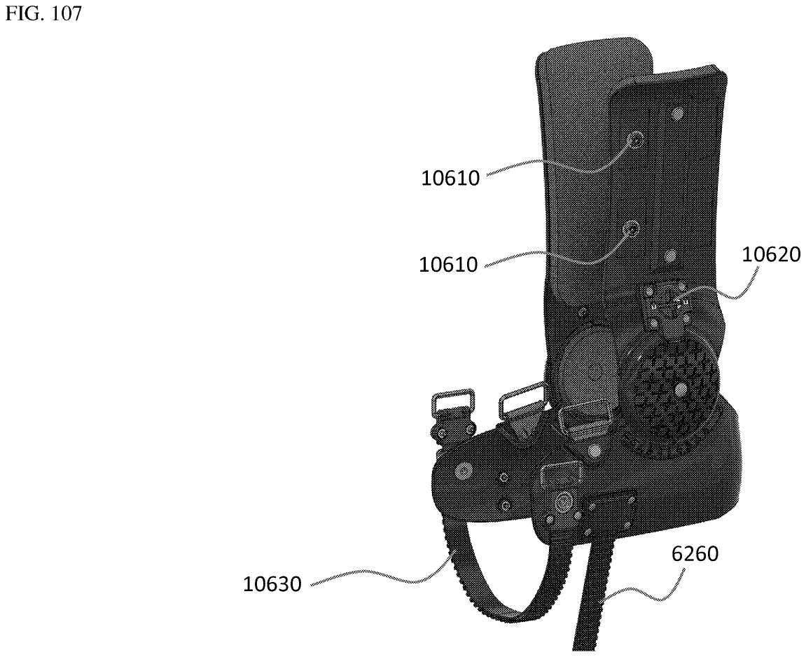

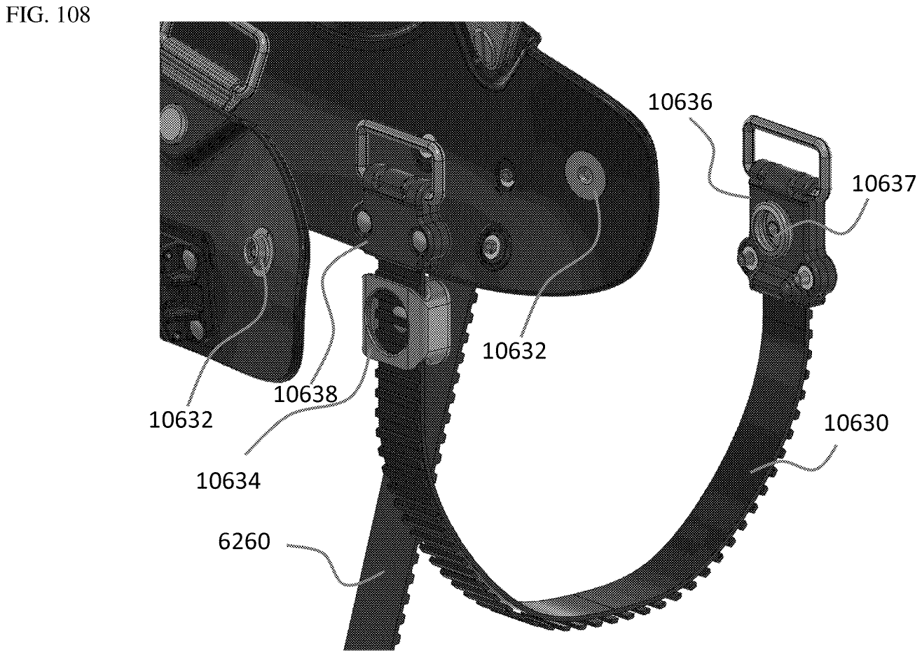

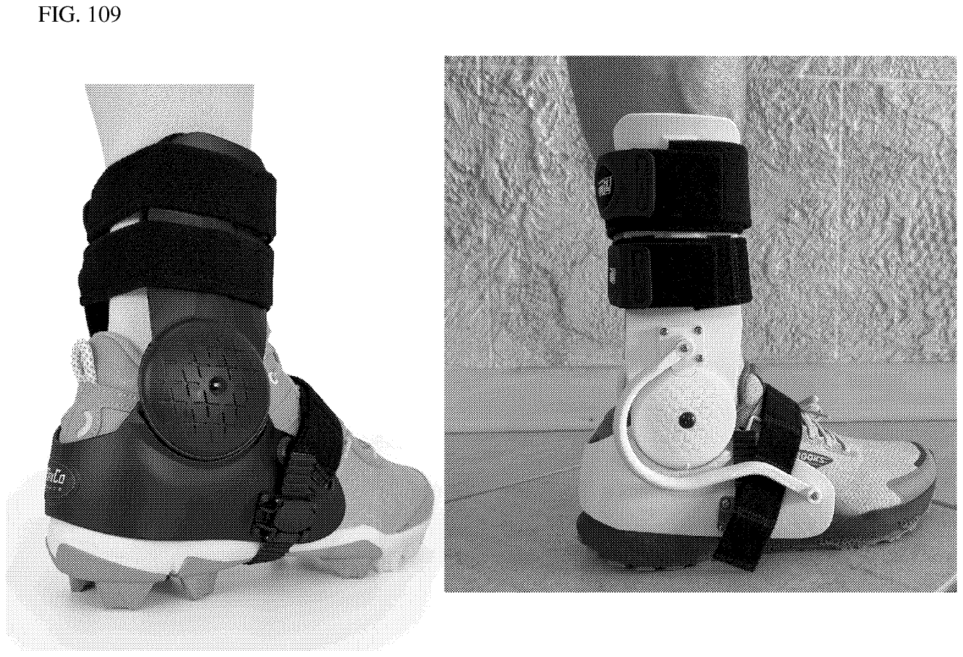

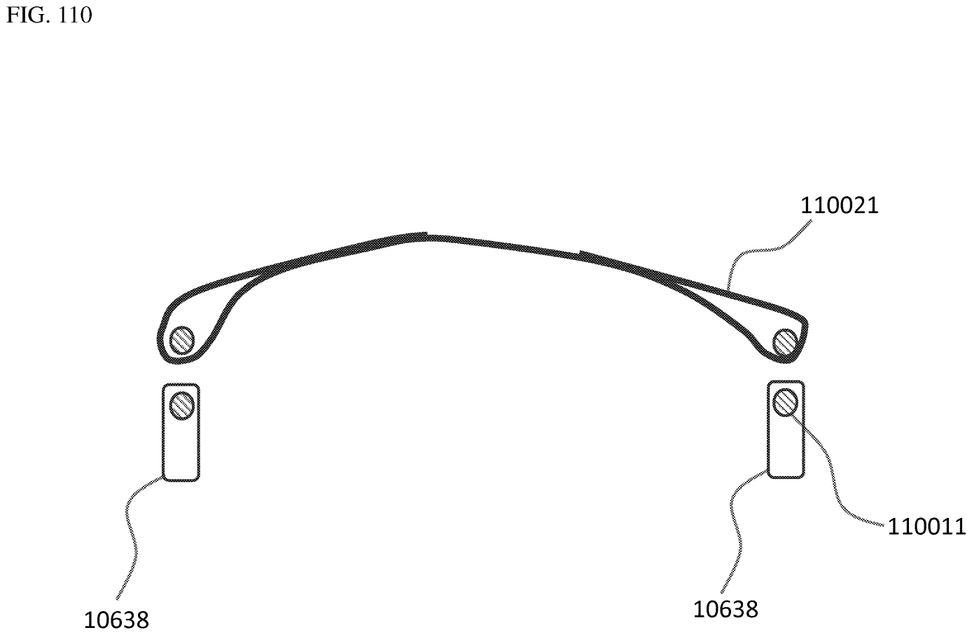

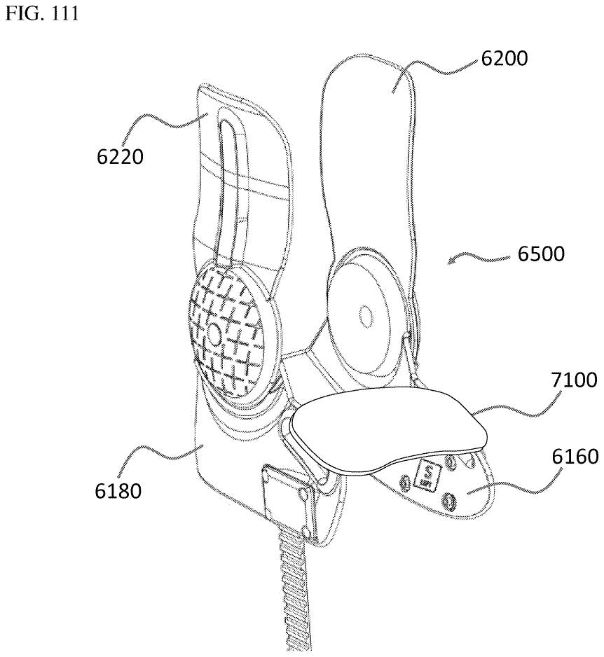

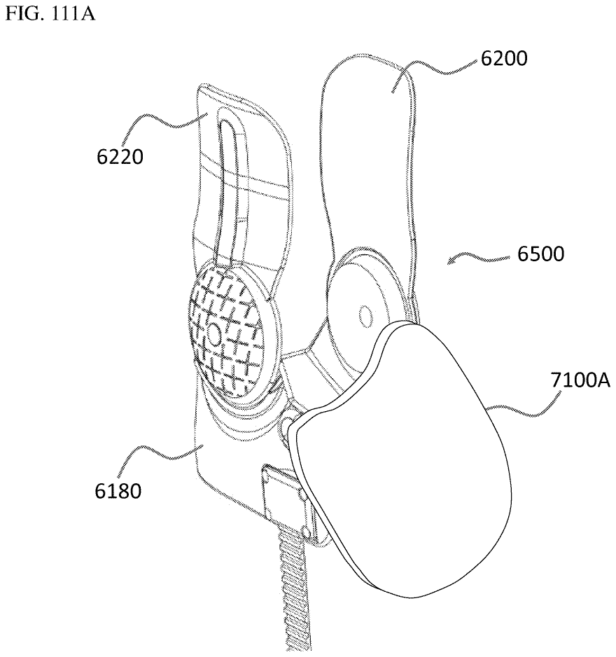

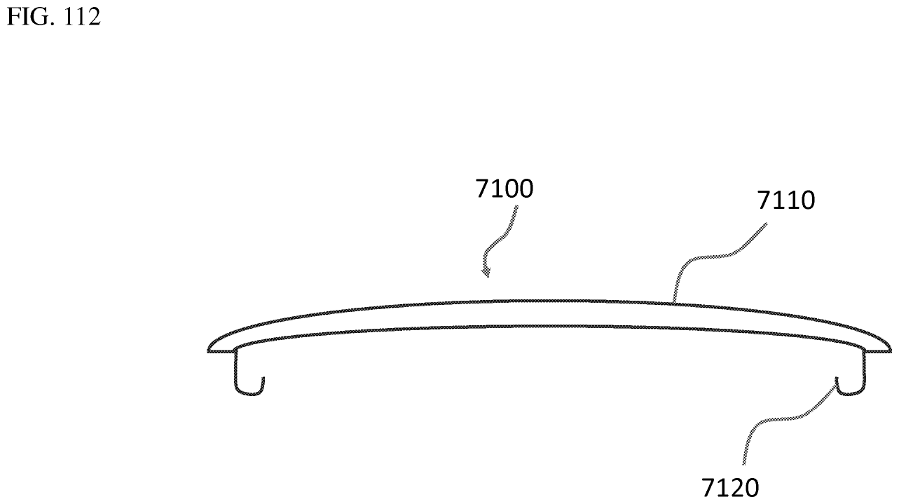

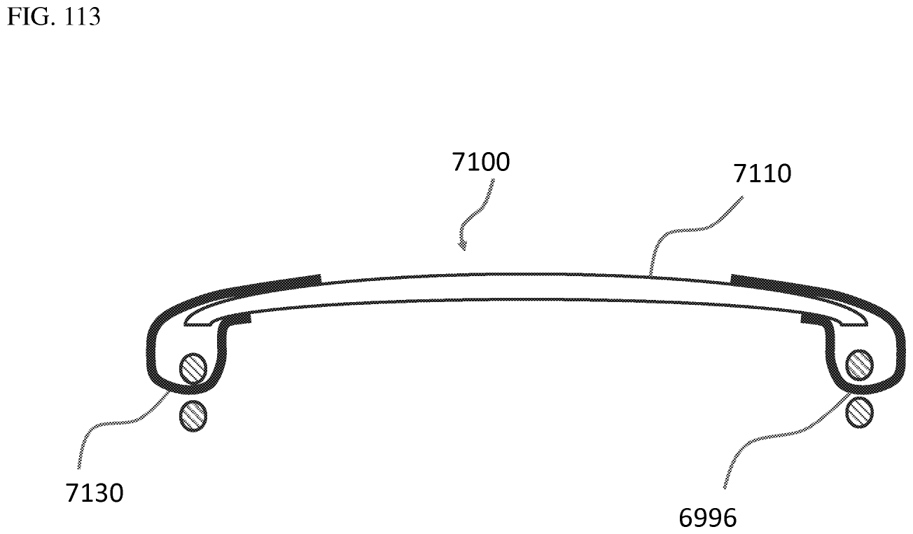

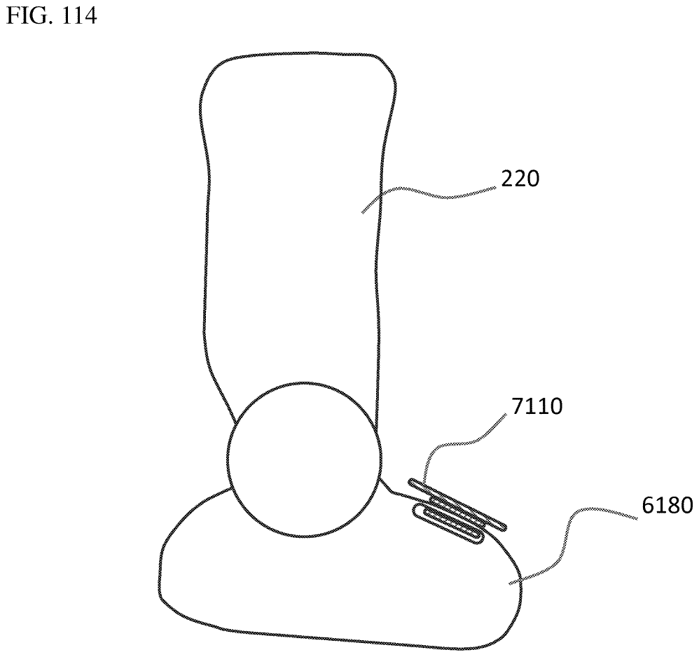

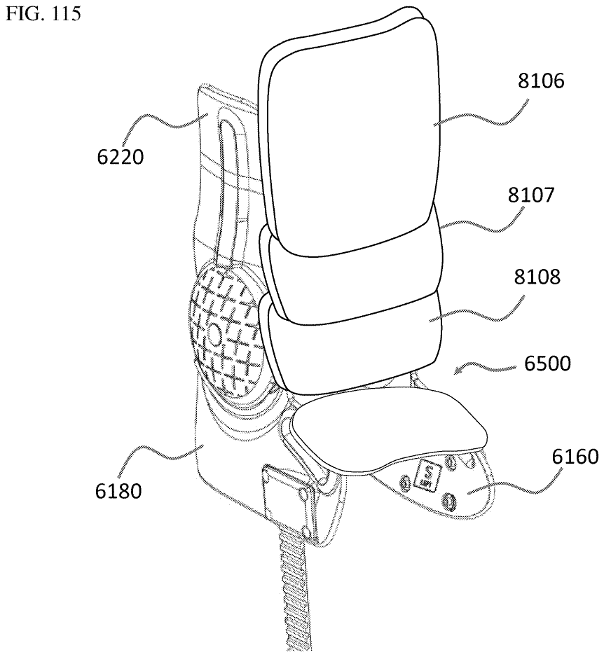

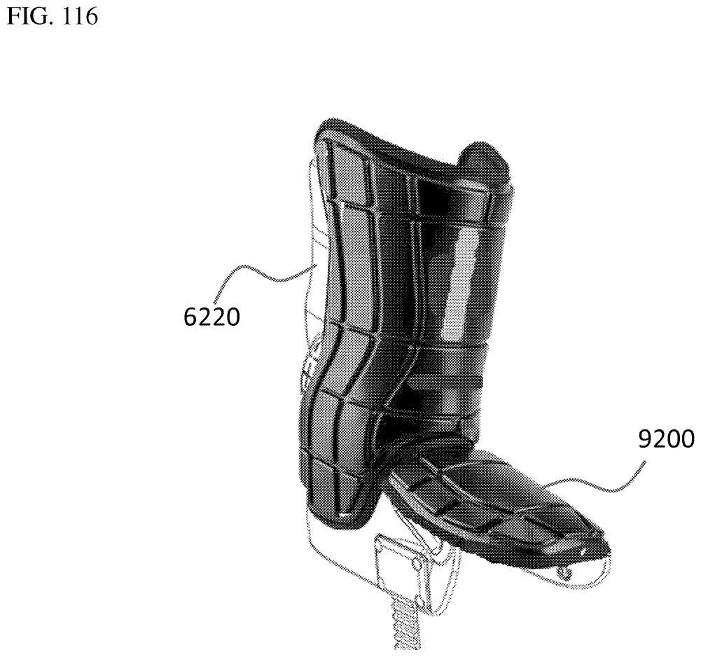

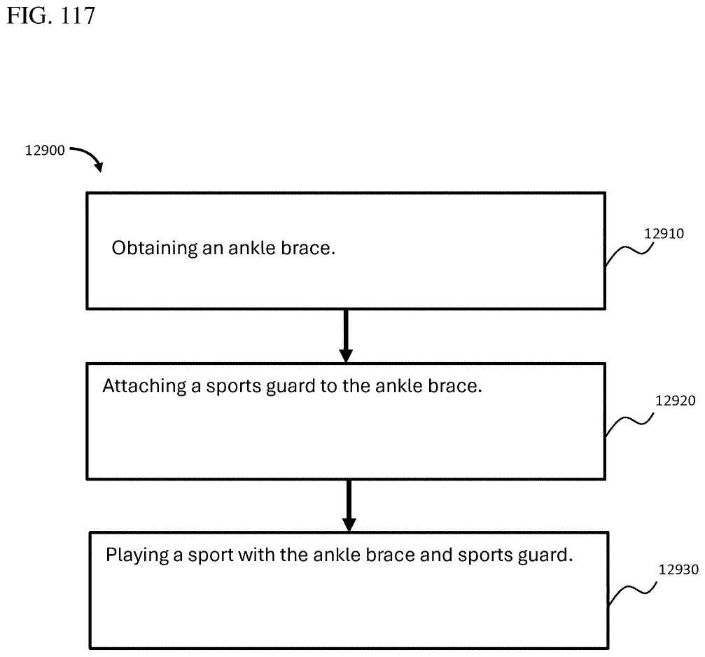

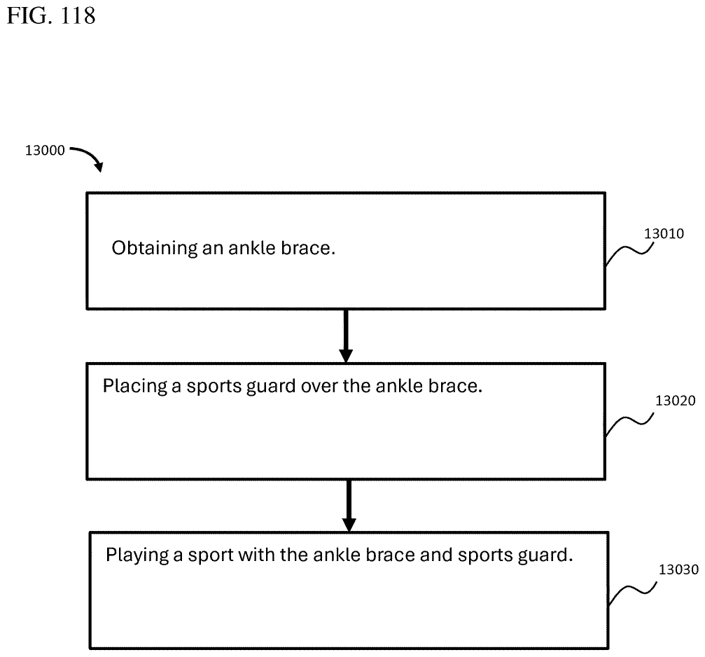

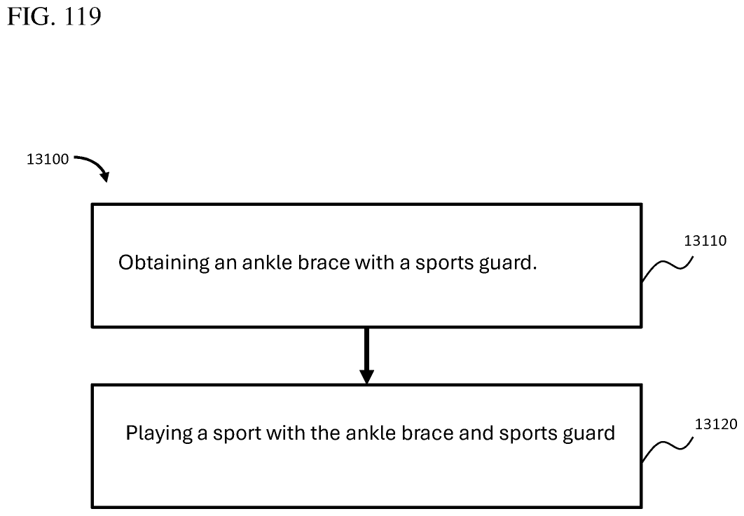

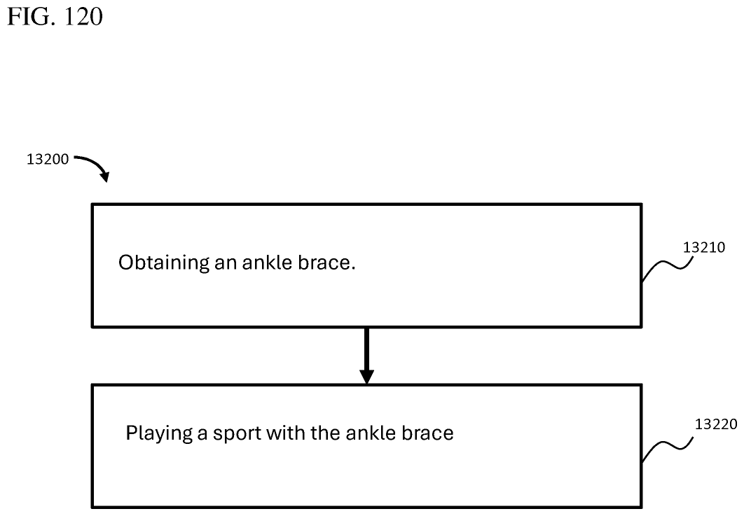

FIG. 99 shows an exemplary barrier 9730 that overlaps a lot of the female portion of the joint (and male portion for that matter). FIG. 100 shows a large barrier 9730 for more extreme scenarios of use. FIG. 101 shows a barrier 6931 that is of shorter span extension (about the joint axis) than other embodiments. FIG. 102 shows an embodiment with multiple barriers 6933. In an embodiment, there are less than, greater than and/or equal to 1 (in which case there is no less than), 2, 3, 4, 5, 6, 7, 8, 9, 10, 11, 12, 13, 14, 15 or more, or any value or range of values therebetween in 1 increments barriers on each side or on one side (there might be no medial barrier in some embodiments, and the values need not be the same. The barriers (or thickened portions) can have any of the dimensions detailed herein, and the dimensions need not be the same. Indeed, FIG. 103 shows another embodiment, where there are two different barriers 6935 and 6936.

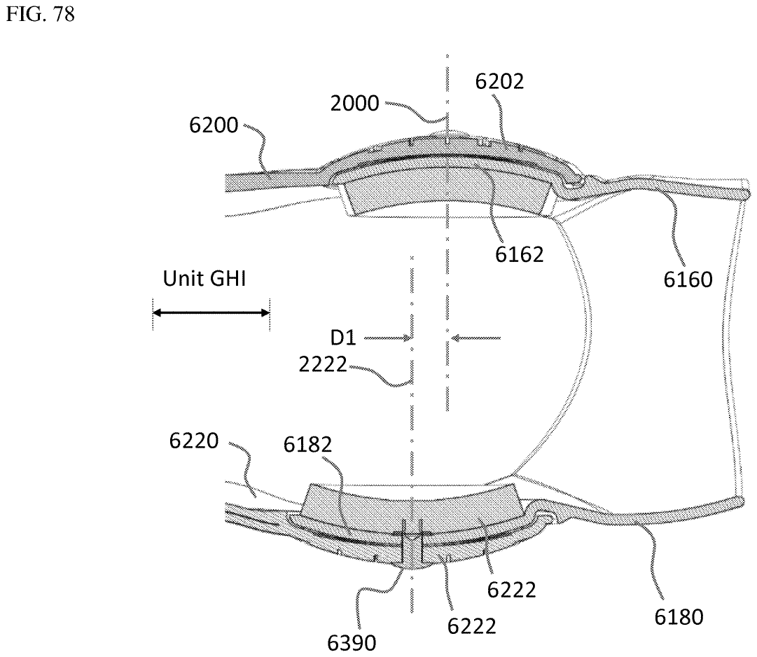

Unit ABC of FIG. 60 is 1 and 7/16^{th} of an inch. Unit DEF of FIG. 76A is 7/16^{th} of an inch. Unit GHI of FIG. 78 is 1 and ¼^{th} inch. For textual economy, any disclosure herein of scaled components herein can be increased and/or decreased by less than, greater than and/or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65% or more, or any value or range of values therebetween in 1% increments, and the accompanying measurements adjusted accordingly, and one component can be adjusted by a different amount than another, if at all.

FIG. 104 shows an exemplary flowchart for an exemplary method, method 10400, that entails action 10410, that entails obtaining an ankle brace, which can be any of those detailed herein or a variation thereof in some embodiments, or different braces. Method 10400 includes action 10420, that entails walking and/or running while wearing the ankle brace over a shoe, and brushing the brace against an object that creates a catch hazard and deflecting the object and/or the brace from that object owing to a dedicated structure of the brace for that purpose. In an embodiment, the object is an object that has an edge that could catch on the female portion of the joint or otherwise the upright, but is prevented by doing so owing to the bumps or the barriers detailed herein. In an embodiment, this is the edge of a step or the edge of an aircraft or the edge of a frame of a building or house or the edge of an opening of a car when the door is open or even brush or tall grass when working outside or walking outside or running outside for the edge of a piece of metal when a construction site, such as an I-beam or some other frame component of a building. In an embodiment, this could be a field hockey stick or some other sport equipment, or this could be a portion of a boat or a ladder or an exercise equipment, such as a Stairmaster or a treadmill or a bicycle or an elliptical device. This could be a stone for example or a rock or some other debris that becomes airborne. This can be a root of a tree or a vine. This can be a court of a parachute arrangement or a bubble or some other fastening component of an aircraft or vehicle or a tense lanyard or a stake in the ground.

In view of the above, it can be seen that in an embodiment, there is an orthotic, comprising a lower leg interface and a shoe interface (e.g., heel enclosure) articulatedly (e.g., rotatably) connected to the lower leg interface wherein the orthotic is configured with a barrier between the shoe interface and the lower leg interface. The barrier can be a protrusion on the shoe interface. The barrier can be monolithic with a main portion of the shoe interface/body that makes up the sidewall(s). The barrier has a cross section of a ramp and the barrier the barrier extends in an arcuate manner (constant radius or a radius that changes as seen herein—in an embodiment, the radius of curvature of the barrier (base on top or bottom or crest or all three) is less than, greater than and/or equal to 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.25, 3.5, 3.75, or 4 inches or more, or any value or range of values therebetween in 0.01 inch increments, and this value can change over the length of the barrier. Still, as seen in some embodiments, the barrier extends in parallel manner with a portion of the lower leg interface immediately adjacent the barrier. The barrier can be established by a first portion of the orthotic that is more outboard than a second portion of the orthotic. In an embodiment, the barrier is a component of the orthotic that has no other functional purpose than to prevent an object from being caught between the foot interface and the lower leg interface. The barrier can be a berm that partially surrounds a portion of the lower leg interface. The barrier blocks movement of an object in a vertical direction along a surface of the foot interface. This can be the case throughout any of the angles where the barrier is present. Any movement must then move outboard before then going inboard in some embodiments.

In an embodiment, there is brace that has a medial lower leg section configured to interface with at least a medial side of a leg of a human at a lower leg portion thereof, the medial lower leg section including a medial rigid body contoured to a medial side of a lower leg of a human and a lateral lower leg section configured to interface with at least a lateral side of a leg of a human at a lower leg portion thereof, the lateral lower leg section including a lateral rigid body contoured to a lateral side of a lower leg of a human and a foot section configured to interface with a shoe worn on a foot of the human at locations where the foot of the human is located and a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section, wherein the lateral ankle joint has a female portion that is part of the lateral lower leg section that interfaces with a male portion of the lateral ankle joint that is part of the foot section, and an outermost edge of the female portion is inboard of a portion of the foot section. The inboardness of the outermost edge establishes a barrier. The inboardness of the outermost edge is located at the lowest portion of the female portion (and in other locations in some embodiments as seen above). The inboardness of the outermost edge extends for more than 30 degrees about the female portion or any of the angles noted herein. In an embodiment, the inboardness of the outermost edge is present through all range of motions of the lateral lower leg section, or at least 80 or 70 or 60 or 50% of those ranges of motions. The inboardness of the outermost edge can be established by a protrusion of the foot section, the protrusion corresponding to at least part of the portion of the foot section. In an embodiment, the inboardness of the outermost edge extends for at least 30, 35, 40, 45, 50, 55, 60, 65, 70, 75, 80, 85 or 90 percent of an overlap area, when viewed from a side of the brace, of the outermost edge with the foot section. The outermost edge extends along a circular trajectory through at least 50, 60, 70, 80, 90, 100, 110, 120, 130, 140, 150, 160, 170, 180, 190, 200, 210, 220, 230, 240, 250, 260, 270, or 280 degrees.

In an embodiment, the portion of the foot section is an arcuately extending berm structure of the foot portion and/or the inboardness is a result of a configuration of the brace that forces a horizontal component of movement of an object with respect to movement of the object to a location behind the female portion of the joint relative to that which would otherwise be present, all other things being equal. That is, for example, combination of the bump and the female portion of the joint establish the above-noted channel or otherwise passageway that requires a horizontal component to the location behind the female portion of the joint. This as opposed to for example of the berm or otherwise the barrier were not present, where, for example, pure vertical movement of the object would result in the positioning. Of course, this is respect to the local geometry of the brace. Accordingly, in an embodiment, the various movements are established within the movement distance being less than a half an inch or a quarter of an inch for the object (the total path taken of the object, including the horizontal component and the vertical component).

An embodiment includes a foot orthosis, comprising a first apparatus configured to grip a shoe worn by a human, a second apparatus configured to grip a leg of the human, wherein the first apparatus articulates relative to the second apparatus, the second apparatus includes a first subportion that articulates relative to the first apparatus and that extends from a location below a top portion of the first apparatus to a location above the top portion of the apparatus, the first subportion has a portion that overlaps with a portion of the first apparatus when viewed from a side of the orthosis, and when viewed looking upwards at the brace along a trajectory that is coincident with the vertical direction (i.e., directly under the brace, but not directly along a longitudinal axis of the extension of the uprights for example), a portion of the first subportion is eclipsed by the first apparatus. In an embodiment, when viewed looking upwards at the brace along a trajectory that is less than, greater than and/or equal to 5, 10, 15, 20, 25, 30, 35 or 40 degrees or any value or range of values therebetween in 1 degree increments from the vertical direction, which view also allows for more of an outer side of the first subportion to be seen (e.g., taking the brace as positioned in FIG. 60 and then turning the brace about a horizontal line so that the bottom of the brace moves into the page and the top of the brace moves out of the page), the portion of the first subportion remains eclipsed by the first apparatus. In an embodiment, the portion of the first subportion is a lowermost portion of the second apparatus. In an embodiment, the first subportion includes a joint portion that includes a surface having an arcuate perimeter, a portion of the surface facing the first apparatus and thus establishing the overlap and at least 10, 15, 20, 25, 30, 35, 40, 45, 50, 55, 60, 65, or 70%, or any value or range of values therebetween in 1% increments of a length of the arcuate perimeter is eclipsed by the first apparatus when viewed looking upwards at the brace along a trajectory that is coincident with the vertical direction.

In an embodiment, the first subportion includes a joint portion that has an arcuate outermost perimeter when viewed from the side, a portion of the outermost perimeter establishing at least a portion of the overlap and at least any of the just noted percentages of the length of the arcuate outermost perimeter is eclipsed by the first apparatus when viewed looking upwards at the brace along a trajectory that is coincident with the vertical direction.

In an embodiment, the brace is such that when viewed looking upwards at the brace along a trajectory that is according to any of those detailed above degrees from the vertical direction, which view also allows for more of an outer side of the first subportion to be seen, the portion of the first subportion remains eclipsed by the first apparatus over a range of views extending from 5, 10, 15, 20, 25, 30, 35, 40 or 45 degrees on one side of a vertical plane to 5, 10, 15, 20, 25, 30, 35 or 40 degrees on an opposite side of the vertical plane, which vertical plane extends through a geometric center of the first subportion and wherein the trajectory is any of those detailed above.

In an embodiment, the portion that is eclipsed is at least 30, 35, 40, 45, 50, 55, 60, 65, 70, 75, 80, 85 or 90% of an outer periphery of the first subportion extending from front to back in the view that would otherwise be visible in the absence of the first apparatus. The portion of the first subportion is a female portion of a joint of the orthosis. When viewed from the side of the brace, the first apparatus has a protruding component that extends about a portion of the first subportion.

In an embodiment, there is an external ankle brace, comprising a medial upright configured to interface with at least a medial side of a leg of a human at a lower leg portion thereof, the medial upright including a medial rigid body contoured to a medial side of a lower leg of a human a lateral upright configured to interface with at least a lateral side of a leg of a human at a lower leg portion thereof, the lateral upright including a lateral rigid body contoured to a medial side of a lower leg of a human an enclosure configured to interface with a shoe worn on a foot of the human at locations where the foot of the human is located, a lateral ankle joint that pivotally connects said lateral upright to said enclosure and allows the lateral ankle joint to move relative to the enclosure, a medial ankle joint that pivotally connects said medial upright to the enclosure and allows said medial upright to move relative to said enclosure, wherein the lateral ankle joint has a first joint portion that is part of the lateral upright that interfaces with a second joint portion that is part of the enclosure, and the brace includes an anti-catch feature adjacent the lateral ankle joint. The anti-catch feature has a wedge or ramp shaped outer profile. The second joint portion is male portion and the first joint portion is a female portion and the enclosure includes a second male portion that extends about a portion of the male portion; and the second male portion establishes at least part of the anti-catch feature. In an embodiment, the first joint portion has an outer circumference, and at least 5, 10, 15, 20, 25, 30, 35, 40, 45, 50, 55, 60, 65 or 70% of an outer periphery of the first joint portion is bounded by the second male portion, the bounding establishing the anti-catch feature. In an embodiment, at least a portion of the first joint portion is inboard of a portion of enclosure immediately adjacent to the first joint portion, the portion of the enclosure not including the second joint portion. A portion of the first joint portion is located in a trough established by the enclosure, the first joint portion being located in the trough establishing the anti-catch feature. In an embodiment, the most inboard portions of the first joint portion are inboard of a portion of enclosure immediately adjacent to the first joint portion that is located outside a profile of the first joint portion when the brace is viewed from a side. In an embodiment, the anti-catch feature is established by a bump portion of the enclosure. In an embodiment, the enclosure includes at least a lateral sidewall and/or the sidewall extends away from a crest of the bump with increasing distance with location in a rearward and/or forward direction. In an embodiment, the anti-catch feature is established by a guard portion of the enclosure, the guard portion includes a crest; and the crest extends for at least 0.25, 0.5, 0.75, 1, 1.25, 1.5, 1.75, 2, 2.25, 2.5, 2.75 or 3 inches within two parallel planes no more than 0.05, 0.1, 0.15, 0.2, 0.25 or 0.3 inches from each other. In an embodiment, the second joint portion is male portion, the first joint portion is a female portion, the enclosure includes a second male portion having an extension that is concentric with an outer profile of the male portion and the female portion and the second male portion establishes at least part of the anti-catch feature.

In an embodiment, the brace includes a lower leg interface and a shoe interface articulatedly connected to the lower leg interface, wherein the orthotic is configured so that adjacent portions of the lower leg interface and the shoe interface overlap in the horizontal direction. The lower leg interface has respective lowermost portions at respective angles of articulation relative to the shoe interface, the respective lowermost portions always being more inboard than a portion of the shoe interface immediately below the respective lowermost portions. The lower leg interface has an outer profile a portion of which is contiguously arcuate over at least any of the values disclosed above, and the foot section includes a portion that is proud of the outermost portion of the contiguous arcuate outer profile at a location adjacent and in the convex side of the accuracy. The portion that is proud extends arcuately over at least 10, 15, 20, 25, 30, 35, 40, 45, 50, 55, 60, 65, 70, 75, 80, 85, 90, 95, 100, 105, 110, 115, 120, 125, or 130 degrees, or any of the values disclosed herein. In an embodiment, a first end of the proud portion is located forward of an axis of rotation of a component of the lower leg interface and a second end of the proud portion is located behind the axis of rotation, and the proud portion extends downward from the first end and then upwards towards the second end. In an embodiment, the foot section includes a thickened wall section of a sidewall of the food section that blends in with a remainder of the foot section to establish the overlap. In an embodiment, the foot section includes a ramp that slopes with respect to location in a vertical direction outward to establish the overlap.

In an embodiment, the shoe interface has a dome shaped male portion (as opposed to a cap shaped portion, such as that which is shown in FIGS. 14-16), the lower leg interface as a dome shaped female portion which is concentric with the dome shaped male portion and the shoe interface includes a berm portion, wherein an outer periphery of the dome shaped female portion is located between the berm potion and the dome shaped male portion, thus establishing the overlap.

In an embodiment, the overlap is established by a portion of the shoe interface that smoothly ramps outboard and then more abruptly extends inboard. In an embodiment, the lower leg interface has a dome shaped surface having a convex side and a concave side; and when viewed from the convex side, the outer periphery of the dome shaped surface is below at least a portion of the shoe interface immediately adjacent the lower leg interface.

Some exemplary embodiments are described herein by way of example only and not by way of limitation with reference to a device referred to in some instances as TayCo, TayCo Brace, TayCo External AFO, etc., and variations thereof. These constitute exemplary braces according to at least some exemplary embodiments. In some instances, the embodiments of the braces disclosed herein and/or variations thereof will be compared to other types of braces and/or other types of devices that provide some utilitarian value with respect to treatments of elements associated with the lower leg/ankle/foot.

Embodiments can include an all external ankle/hindfoot AFO for use in acutely injured football players, for example. The device can be a custom fitted device which can be dispensed for a wide variety of patients with various ankle/hind foot injuries and pathologies at the time of their initial presentation to the clinic. This custom fit, functional AFO can avoid, for example, the significant complications associated with using other ankle/hindfoot immobilization devices such as cam walkers, internal AFOs, and casts. All of these devices also have significant compliance and complications associated with them, and some embodiments can overcome at least one or more of these complications.

Embodiments include a Custom Fit TayCo External Ankle Brace (TayCo XAB) which can be a truly custom fit ankle foot orthosis which allows the patient to be immobilized as if they were in a traditional AFO, but without one or more of the associated complications of prior braces. The custom fit device is constructed of material which can be bent, molded, and trimmed right in the supplier's facility and/or at another location. Spring steel can be used to aid bending. The device has an option to limit range of motion and can also be converted to a free range of motion as per the utilitarian value of a given acutely injured patient.

In an embodiment, the braces/orthoses to which the articulation control/limiting teachings detailed herein and/or the dorsiflexion and/or plantar flexion assist teachings detailed herein are applied to a brace/orthosis as disclosed in WIPO patent application publication No. WO2022204557, entitled External Ankle Brace, to inventors Ferlic, Bean, and Lazenga, published on Sep. 29, 2022. Thus, embodiments include a brace or orthosis having any one or more of the features disclosed in WO2022204557 when combined with any one or more of the teachings detailed herein, and for this purpose, the teachings of the '557 publication are incorporated herein by reference in their entirety. In an exemplary embodiment, there is a brace/orthosis that includes any one or more of the articulation control/limiting features detailed herein and/or any one or more of the dorsiflexion assist features detailed herein that has any one or more of the performance features or design features or material features, or any of the features/elements, etc., of the brace/orthosis disclosed in the 557 publication. To be clear, the teachings herein related to articulation control and to dorsiflexion assist were detailed specifically with application of those teachings to the brace/orthosis disclosed in the '557 publication. Indeed, in an embodiment, there is a method of obtaining a brace/orthosis according to the '557 publication, and retrofitting such to have any one or more of the teachings herein. Thus, in an embodiment, there is a brace that is a rigid brace, that fits over a shoe, which is placed onto a person while the person is wearing the shoe on a foot thereof, and which has any one or more of the teachings herein. In an embodiment, there is a method of using such to walk, where the brace automatically causes dorsiflexion assist, or otherwise moves the toes of the foot wearing the brace upward in a scenario where the muscles of the human are relaxed or otherwise not lifting up the toes. In an embodiment, the foot is limp, or otherwise the nerves are not stimulating the muscles (e.g., due to stroke) or the muscles are operating at less than and/or equal to 5, 10, 15, 20, 25, 30, 35, 40, 45, 50, 55, 60, 65, 70, 75 or 80% of their ability relative to that which would be the case for a human factors engineering comparable human not having muscle or neurological disorder, and/or at such a percentage for a 25, 50 or 75 percentile human factors engineering male or female as the case may be for the age of that person, born in and/or a resident of at that time, the United States of America, Canada, the European Union as constituted on May 13, 2015, or the People's Republic of China, or the United Kingdom, the Republic of France, the Federal Republic of Germany, the Kingdom of Spain, the Italian Republic or the Federative Republic of Brazil or the Argentine Republic. In an embodiment, the brace lifts up the foot at a speed that is at least 50, 55, 60, 65, 70, 75, 80, 85 or 90% as fast as that which would be the case for the pertinent human factors engineering person and/or at an angle upward from the lowest angle of the foot (most plantarflexion location) that is at least 50, 55, 60, 65, 70, 75, 80, 85 or 90% that which would be the case for the pertinent human factors engineering person. In an embodiment, this can be tested by having the foot raised in space without contacting the ground (or more accurately the shoe and/or the brace not contacting the ground) and bending the toes downward (in plantarflexion) to the maximum angle or an angle that is within 25, 20, 15, 10 or 5% of the maximum angle, and then releasing the foot/shoe. In an embodiment, any one or more of the elements, method actions and/or system features, or any features, disclosed in any one or more of U.S. Provisional Patent Application No. 63/804,088, filed on May 12, 2026, in the United States Patent and Trademark Office, and U.S. Provisional Patent Application No. 63/824,623, filed on Jun. 16, 2026, in the United States Patent and Trademark Office U.S. Provisional Patent Application No. 63/827,528, filed on Jun. 20, 2026, in the United States Patent and Trademark Office, U.S. Provisional Patent Application No. 63/829,989 filed on Jun. 25, 2026, in the United States Patent and Trademark Office, and/or the '577 publication can be combined with any one or more of the other elements, method actions and/or system features, or any features, disclosed in any of those documents.

FIGS. 106 and 107 and 108 show views of another embodiment of the brace which uses many of the components noted above and variations thereof. FIGS. 106 and 107 show the lower connecting strap 6260 and the component 6230 to which the strap 6260 is attached. Also shown is a second lower connecting strap 10640. The forward connecting strap 10640 in this embodiment is removable in regular use from the sidewalls. This as opposed to the rear strap 6260, although in other embodiments, the rear strap 6260 can be replaced with a removable strap as well, such as a strap that is the same as or similar to the forward connecting strap 10640. In this exemplary embodiment, forward strap 10630 includes the strap body which can correspond to that of the strap 10640 with respect to its design, albeit with a different length, as well as to lugs 10636 and 10638 which are respectively connected buckles as shown. These buckles can be the same as or similar to the buckles that are attached to the sidewalls, one of which is shown in FIG. 108 and both of which are shown in FIG. 107. The sizes can be different of course, or can be the same. The lugs 10638 and 10636 can be configured to “traps” the strap body there between in a manner analogous to how the strap 6260 is “permanently” attached to the sidewall as described above. Here, the lugs can comprise to have sales that can be bolted or riveted together as shown, where at least one of the clamshells has a male-female arrangement that interfaces with the protrusions on the strap body, again in a manner that is the same as or analogous to that described above for strap 6260. Note also that a claim shall arrangement can be utilized to trap the belt buckles between the two halves.

Also included in the forward lower strap 10630 are the female portion 10637 of step buttons 10632. In this regard, in this embodiment, there are male snap buttons 10632 located on the sidewalls as shown. During use in this embodiment, the lower strap 10630 can be snapped to those male portions so as to attach the forward strap to the sidewalls. In this embodiment, the strap can be readily removed as desired by the user. In an alternate embodiment, the forward strap is “permanently” attached to the sidewalls. In an embodiment, one side is attached to the other side is releasable in a manner analogous to that detailed above with respect to strap 6260. In this embodiment, owing to the geometry of the brace, there is a separate component 10634 that is movably attached to the strap 10630. In other embodiments, this component 10634 is fixedly attached to the strap. Here, component 10634 includes the female receptacle 10637 (not shown as it is on the other side of component 10634 with respect to the view shown in FIG. 108) and is attached to the male snap shown in FIG. 108 in use. Component 10634 permits the strap to be adjusted in use. The strap body 10630 can “slide” through the openings in component 10634. Here, when the upper strap portion (not shown in FIG. 108) is attached to the belt buckles and tightened, by way of for example cinching down on the upper strap, in a manner analogous to the upper strap 30′ for example (a velcro strap can be threaded through the buckles, and the ends pulled towards each other and then velcroed together, thus pulling the belt buckles up and towards each other) the lower strap will be pulled upwards through the component 10634 and thus tightened around the shoe (with the tightening of the comparable upper strap that attaches to the forward strap). Thus, by tightening the upper strap, the lower strap is tightened. The snap buttons or more for general retention to the sidewalls. Because the forward strap, or more accurately, the forward strap assembly in this embodiment is a wraparound strap that completely encircles the shoe and also the sidewalls, the tensioning and otherwise friction associated there with that results from the contact of the forward strap with the shoe provides the majority of the holding force in at least some exemplary embodiments.

And note that while this embodiment is shown as a two-part forward strap assembly, in another embodiment, this can be a single strap that includes two components 10634, or maybe only one component 10634 for that matter. In an embodiment, the other side could free float, or alternatively, there could be a female portion through which the strap is snaked, or at least lugs that trap the other side of the single strap. And note that such an arrangement can also be utilized with the two portion assembly for that matter.

Accordingly, in an embodiment, the strap can include a removable circumferential strap, that can be added to the brace when needed, and then removed when not needed, or otherwise perceived to be not needed, as well as the stirrup strap in the rear, both of which can be made out of Kevlar reinforced components. Two or three or four wraparound straps can be utilized, or in an alternate embodiment, the forward strap is also not a wraparound strap, and there could be three or four such straps as well, and a combination of the different straps can be utilized. There could be one non-wraparound strap into wraparound straps, or 29 wraparound straps in one or two wraparound straps, etc.

FIG. 110 shows a cross-section of the D rings 110011 attached to components 10638, and strap 110021 which is threaded through the D rings, the ends of which are velcroed to the top of the strap to maintain toughness and otherwise complete a circumferential strap arrangement. The Strap 110021 would extend over the top of the shoe.

FIG. 107 shows male snap portions 10610 located on the uprights. In an exemplary embodiment, the leg straps include components analogous to those of the forward strap 10630 detailed above. This can enable one or two upper leg straps to be used as desired. This can also enable the use of a wraparound straps/the circumferential strap again in a manner analogous to that detailed above with respect to forward strap 10630.

In an embodiment, adjustment and/or tightening of the forward strap is the same as or otherwise analogous to that detailed above with respect to any one or more of the other straps, depending on the embodiment, providing that the art enables such. In an embodiment, the tightening and/or adjustment would be varied to take into account the different design of the forward strap relative to the rear strap.

FIG. 109 shows two different designs of different braces according to embodiments in use.

FIG. 111 provides an exemplary brace 6500 that includes attached thereto (or that is a part thereof or is used with—see more on this below) a protection barrier 7100. In this exemplary embodiment, the protection barrier is a thermoplastic material that is configured to at least disperse and/or deflect and/or absorb a force and/or energy applied at or proximate the barrier 7100 over a larger area than that which would otherwise be the case, or otherwise provide more distance for deceleration of the object that is striking the foot or brace or barrier 7100, thus reducing the actual force for a given mass that results from contact with the object, whether such as moving or not moving. Herein, the scenarios associated with the foreign objects that are imparting energy into the foot of the wearer will often be described in terms of the object moving when it strikes the foot. However, as noted above, this could be the wearer swinging his or her foot or otherwise moving his or her foot in a manner where the foot is the subject of the movement, and it is the movement of the foot that is causing the energy that is being absorbed into the anatomical structure (because the foot hits a relatively immovable object for example), or the combination of the two. Accordingly, any disclosure of one corresponds to the disclosure of the other or the disclosure of both objects moving unless otherwise noted, all in the interest of textual economy. Also, reference will also be often made to the object striking the foot or the foot striking the object, etc. In reality, the object, for example baseball, does not directly contact the foot in the absolute sense because there is a shoe and there is a sock (and a portion of the brace) and then there is a barrier between such. It is to be understood that in the interest of textual economy, reference to contact or striking, etc., with an anatomical structure corresponds to the general concept of the anatomical structure being covered by any one or more of the aforementioned structures, such as a sock and/or the shoe and/or the laces of the shoe and/or the portions of the brace and/or the barrier, etc., as will be understood, and thus reference to an object striking or hitting or contacting one corresponds to a disclosure of the object striking all of these components and otherwise importing energy or otherwise the general scenario that would exist as will be understood by one of ordinary skill in the art under the scenario described).

In an embodiment, barrier 7100 is a fiberglass body and/or a body made out of Kevlar. In an embodiment, this can be a hard and/or rigid thermoplastic body, which can be molded to shape, or otherwise cut or machined from a body of thermoplastic material. In an embodiment, the body is a polymer that is hard and/or rigid, and again could be molded or could be, or machined to size. In an embodiment, the body can be made of a metal, such as aluminum, or titanium, or some form of steel. In an embodiment, the edges can be chamfered or smooth or rounded so as to avoid injury or otherwise protect against catching or otherwise damaging other tissue or other components in the event that the body contacts such. In an embodiment, the body of the barrier 7100 has a modicum of flexibility, such as a flexible thermoplastic material, and the utilitarian value of this will be described in greater detail below. All of this said, in an embodiment, the barrier 7100 can be established by a softer material or otherwise flexible padding. The barrier 7100 can be a flexible plastic body in which is located foam padding or plastic components or blocks or hard foam or Styrofoam. Still, the padding could be flexible or soft in other embodiments (soft foam blocks for example). The barrier 7100 could be a container that contains a non-Newtonian fluid. The barrier 7100 could be a container that contains a highly viscous fluid for example, or a semiliquid material. In an embodiment, the barrier 7100 can be made of any material or otherwise can contain any material that will have the effect of dissipating energy or otherwise providing more room for deceleration of an object striking the foot and/or the leg (more on this below) or otherwise enable energy management/force management according to embodiment herein and/or otherwise something that dissipates force over a larger area than that which would otherwise be the case, and/or a combination of these (in an embodiment, a pressure gradient over 0.1, 0.2, 0.3, 0.4, 0.5, 0.6, 0.7, or 0.8 inches, or any value or range of values therebetween in 0.05 inches over a distance 2 or 3 times that amount is reduced by less than, greater than, and/or equal to 90, 80, 70, 60, 50, 40, 30, 20, or 10% or less, or any value or range of values therebetween in 1% increments relative to that which would otherwise be the case). More on this below. In any event, the barrier 7100 or the specific components thereof will often be described in terms of a “black-box” where the makeup of the barrier can be any one or more of the arrangements detailed herein providing that the art enables such. By way of example, referring to FIG. 35, three is a barrier 7101 that has an element 7110 that is arcuate when viewed from the side. The element 7110 could be a thermoplastic shield, or a container that contains a fluid, or could be closed cell foam or a container in which is located open cell foam (or closed cell foam). But below, we will often refer to element 7110 as a body or shield or component or sometimes the barrier (even though it is the assembly 7100 that is the barrier (the shield 7110 plus the hooks 7120). Any disclosure of any feature associated with the barrier can be used in any of these providing that the art enables such, unless otherwise noted, in the interests of textual economy. Any disclosure of a body or element or component herein corresponds to a disclosure of any of the barrier materials/concepts regimes (e.g., pad vs. hard plastic vs foam vs quilting vs. layers of cloth, etc.) herein for use with such in the interests of textual economy.

FIG. 111A shows another exemplary embodiment of a barrier 7100A, which is a longer length than that of barrier 7100, and thus protects a larger area of the foot of the wearer. The arrangement shown in FIG. 111A is such that the barrier is not “supported” by the shoe of the wearer, and thus droops downward, or otherwise because of the mechanics of the connection of the barrier to the brace or the foot (when the foot is in the brace), the barrier is lower. When the shoe is displaced of the brace, the distal portions of the shoe would cause the barrier 7100A to rotate so that the distal end is higher than that presented in the figure. Put another way, FIG. 111A shows an exemplary of the system or an assembly that would exist prior to donning the brace over a foot.

FIG. 112 shows a side view of the barrier. This can also be considered a cross-section of the barrier. In this exemplary embodiment, the barrier is a shell that extends from one side of the brace to the other side of the brace, and otherwise from one side of the shoe to the other side of the shoe, and thus from outboard of the top of one of the sidewalls to outboard of the top of the other of the sidewalls of the brace. As seen, element 7110 is arcuate in this embodiment. Here, this can be a plastic shell to which is attached plastic or metal hooks 7120. In an exemplary embodiment, hooks 7110 fit into the openings 6996 for the top strap and “over” the portion of the strap in the opening (the U shaped or J shaped portion of the strap—in FIG. 112, there would be a J inside the J in fact, the inside J being the strap).

FIG. 113 shows another exemplary embodiment of a barrier 7100 that utilizes strap 7130 to attach element 7110 to the brace. In this exemplary embodiment, straps 7130 extend through the openings 6996 before the top strap 6902 is placed onto the brace. Then, the top strap 6902 is threaded through the openings to secure the brace to the foot so that the bottom “fold” extends completely from one side to the other, and then the strap overlaps the entirety of the barrier.

FIG. 114 shows an embodiment of the barrier used with a brace according to FIG. 14. It is noted that the embodiment of the barrier of FIG. 45 utilizes a different element 7210 than those detailed above. In this exemplary embodiment, which shows a cross-section of the body 7120 of the barrier, along with cross-sections of the top strap 6902 folded over itself in accordance with the teachings above, it can be seen that the forward portion of element 7210 is sloped downward relative to a remainder of the element. Embodiments can thus include barriers that utilize bodies or otherwise the main element thereof that is contoured and otherwise anatomically correct or more correct relative to that which would otherwise be the case with respect to a flat cross sectional body under constant dimensional features etc.

Unless otherwise noted, the attachment regime of the barriers to the brace or otherwise to the foot or to the body of the user of the brace are agnostic with respect to the noted teachings above providing that the art enables such unless otherwise noted. In this regard, any of the attachment regimes detailed herein can be utilized with any of the body structures or otherwise any of the other portions of the barrier unless otherwise noted, again providing that the art enables such (this is also the case with respect to the bodies for the leg barrier as will be described below—any of the teachings of the foot barrier can be used with the leg barrier, and vis-a-versa, and disclosure of one corresponds to an embodiment with the other). All of this is done with respect to textual economy. Thus, the attachment regime of FIGS. 44 and 45 are not shown, but or discloses any of those detailed herein under the just noted constraints.

Also, note that embodiments include a simple barrier to which is attached velcro material (hook or the fuzzy portion or both) that is placed onto the opposite material on the strap and/or the brace. That is, the intertwining with the strap or the openings is not used in some embodiments (or is used in combination with this embodiment).

FIG. 115 shows an embodiment that utilizes the concept of scaling, where barrier body 8108 is partially covered by barrier body 8107 which is partially covered by barrier 8106. The barriers can move relative to one another by sliding for example, with or without friction reducing material or the like, and thus can provide a great amount of protection while also permitting or otherwise at least limiting the restraint of movements that might be the case with respect to that which would exist with a single component extending the distance of the combinations of barriers.

### D

Note that in an embodiment, one or more or all of the various bodies could be attached to each other by way of a strap or some other flexible component that extends from one component to the other, either on top and/or on the bottom. In an exemplary embodiment, the array of barrier bodies could be placed over the leg/in front of the leg and over the top of the shoe, and then the assembly could be attached to the brace or to the user according to any of the attachment regimes herein or modifications thereof that can have utilitarian value or otherwise enable the teachings detailed herein.

Embodiments of the barriers generally focus on a more rigid body or otherwise the concept of an armor like arrangement (cup vs quilted catchers vest, but for that matter, note that in an embodiment, a cage arrangement such as a catchers mask or a goalies mask could be utilized as the “body”). Any disclosure of any of the above bodies disclosed herein corresponds to an alternate disclosure of a softer or more flexible or otherwise an energy absorbing as opposed to energy spreading arrangement (and note that the two are not mutually exclusive). Embodiments can use any barrier material or barrier base product (e.g., cushion, cup body (not the cup per se, but the base concept of a hard plastic molded to cup the part in question), bubble wrap (a ruggedized arrangement of course), fluids, etc. The foam of a bike helmet and/or the plastic cover thereof could be used. The material and/or arrangement of a batter's foot guard could be used. By way of example, “block” foam or “block” plastic components supported by a fabric or plastic sheet can be arrayed over the brace or can make up the body/elements of the barriers. Quilt padding for example can be used (where stitching is used to segment the various padding materials (a cloth material (whether natural or synthetic) overlays padding or quilt material/sandwiches the barrier material). The material inside the two layers could be soft or could be more rigid for that matter. The utilization of stitching over a perimeter to keep the stuffing material or otherwise material between the two layers in place or otherwise to hand a general thickness of that material maintained in an even distribution over the surface (except for the stitching is located for example) can be utilized in some embodiments.

In an embodiment, the various barriers herein are more like clothing than they are armor.

Also, commercially available padding material can be used (and strapped to the brace for example, or attached to the brace or the user. In an embodiment, the pads utilized for horses legs for jumping competitions (e.g., cross-country) can be used or otherwise the pad material thereof can be used. Note also that in embodiments, protective barrier for horses legs also can utilize Kevlar.

In an embodiment, a commercially available batter's foot guard and/or lower leg guard is utilized over the entire brace. In an embodiment, the brace is sized to the shoe and otherwise the straps are adjusted to fit snugly around the shoe, and the brace is thus strapped to the user, and then the batter's foot guard is placed over the entire brace. In this regard, an oversized foot guard might be used or an oversized leg guard might be used to accommodate the fact that the brace is positioned underneath. Accordingly, embodiments include systems and/or devices, etc., that combine any of the various embodiments of the braces detailed herein with any foot guard her leg guard can have utilitarian value. And while embodiments are focused on for example the sport of baseball, in other embodiments, foot and/or leg guards used for American football or soccer, or basketball, or other sports can be used in combination with the brace. FIG. 119 shows a commercially available batter guard (leg and foot that is overlaid over a brace, and is attached to the user in the commercially available manner. As seen, the guard has overlap with many portions of the brace. FIG. 120 shows a commercially available guard 9200 that extends completely around the back of the leg over the brace.

All that said, embodiments also recognize the fact that the brace material and/or the design and structure thereof in and of itself provides a level of protection beyond that which would exist in the absence of the brace. In this regard, embodiments could alleviate the need or otherwise have utilitarian value for the more “full coverage”, for legs and/or for feet that exist. Accordingly, in an embodiment, the barriers, or more accurately, the body components thereof (thus not including straps, etc., unless those are also barriers in a meaningful way in other embodiments) overlap the brace components by an amount that is less than, greater than and/or equal to 0 (no overlap) 5, 10, 15, 20, 25, 30, 35, 40, 45, 50, 55, 60, 65, 70, or 75% or more, or any value or range of values therebetween in 1% increments of the total surface area facing the user.

Embodiments include methods of attaching to the brace and/or using the barriers herein with the braces herein. Embodiments include methods of playing baseball and swinging and hitting a baseball with a bat in combination with any of the teachings detailed herein. Embodiments include doing so where the baseball is duplicate off the bat into the lead and/or the foot and/or the angle of the batter and where the barriers herein just meet the energy or otherwise deflect the baseball or otherwise distribute the force of the baseball in a manner that is more utilitarian and otherwise in a manner that protects the boxer more than that which would otherwise be the case. In an embodiment, the force is distributed over an area that is less than, greater than, and/or equal to 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, or 900% or more, or any value or range of values therebetween in 1% increments more than the area that would have otherwise been the case (relative to the underlying tissue and/or shoe or otherwise the foot or lower leg), and/or the amount of force experienced by the tissue over a given area (e.g., 0.5 by 0.5 inches square or a circle having a diameter of 0.5, 0.6, 0.7, 0.8, 0.9 or 1 inch) or as an absolute is reduced by less than, greater than and/or equal to 10, 15, 20, 25, 30, 35, 40, 45, 50, 55, 60, 65, 70, 75, 80, 85, 90, 91, 92, 93, 94, 95, 96, or 97% or more or any value or range of values therebetween in 1% increments relative to that which would have otherwise been the case and/or the energy imparted into the tissue is reduced by that amount and/or the energy or force imparted onto the outer surface of the shoe, or the sock or the pants leg, etc., is reduced by that amount.

### D

FIG. 116 shows a variation of the barriers herein, using a commercially available guard with the brace.

And note that while many of the embodiments shown herein with respect to the utilization of a commercially available ports guard or the like have portions that extend over the top of the foot or otherwise down beyond the sides of the foot from the location where the foot meets the ankle, etc., thus protecting for example the malleus and other portion of the ankle, in other embodiments, this is not the case for otherwise a more custom sports guard is utilized. This can be because, in at least some exemplary embodiments, the rotational joints or otherwise the ankle joints of the brace or the configuration that provide the energy dissipation and/or deflection and/or force dissipation and deflection, at least partially, according to some of the embodiments herein. And note that in an exemplary scenario, there can be padding located on the inside of the brace, such as with respect to the uprights, and/or with respect to the exterior of the heel enclosure, and/or with respect to the surfaces of the male portions of the joint that face inwards towards the ankle, in accordance with the teachings detailed above for example. These components, whether they be open cell or closed cell foam, can have any one or more of the performance features detailed herein with respect to energy and/or force management. Note also that this can be the case with respect to the bodies that makes up the uprights and/or the heel enclosure, etc., with or without the padding or foam. In an embodiment, any component that is added for the purposes of guarding against impact or otherwise energy management can be monolithic with any other component of the brace providing that the art enables such unless otherwise noted. In any event, the point is that in some embodiments, there might be overlap between the barrier or otherwise the barrier components and the bodies of the brace, while in other embodiments, the overlap is nonexistent and/or the overlap is limited. Embodiments include designing and creating and fabricating and thus utilizing sports guard or an impact card as an outer profile that is generally contoured to the profile of the portions of the brace to take into account the fact that the brace provides some protection as well. The idea is to limit the amount of material that manages the energy to a minimum in some embodiments, or otherwise to reduce the amount for purposes of weight and/or heat transfer, or to avoid any extra material that could catch on something or otherwise interfere with a sport or an endeavor. In other embodiments, the energy management components have overlap, including significant overlap, with the bodies that establish various portions of the brace. In any event, this is why the disclosure above with respect to the dimensions of the outlines or boundaries of the energy absorbing components relative to the features of the brace are presented so as to provide disclosure of various shapes/designs of the guars that are used with/part of the brace.

In an embodiment, a commercially available foot barrier/foot pad is strapped to the brace after the brace is put on a user. The foot pad is obtained potentially in an oversize arrangement to accommodate the brace. Thus, in an embodiment, there is a system that utilizes a commercially available guard for a foot or leg, and a method of doing so, where the commercially available brace is sized and dimensioned in an oversize manner, at least with respect to the attachment components thereof, than that which would otherwise be the case without the brace, all other things being equal. In an embodiment, this entails obtaining commercial guard that is overall larger, or has larger straps or otherwise larger attaching componentry relative to that which would otherwise be the case without the brace. In an embodiment, this can entail utilizing the same guard or the like, except adjusting the connection components or the straps, etc., so that they had a larger circumference relative to that which would otherwise be the case, all other things being equal. In an embodiment, a circumference of straps or otherwise the extension of the strap from one side of the barrier to the other side of the barrier, that extends about the foot and/or extends about the leg, or a portion thereof, is at least 5, 10, 15, 20, 25, 30, 35, 40, 45, 50, 55, or 60% or more or any value or range of values therebetween in 1% increments greater than that which would otherwise be the case without the brace present, all other things being equal.

In an embodiment, the method entails previously utilizing the commercially available guard without the brace, and then utilizing that previously utilized commercial guard for the given person with the brace over the brace, and thus the attachment regime or otherwise the straps, etc., are set at a location or position to provide for a greater girth of the surrounding structure because of the brace. And in an embodiment, the resulting difference in the retention components can correspond to any of the just noted quantities.

In an embodiment, the barriers herein in general, and the body/element thereof in particular, are energy management devices. In some embodiments, these are more ballistic deflection devices, while in other embodiments, these are more energy dissipation devices, and the two are not mutually exclusive (not the qualitative factor). For example, in an embodiment, the barriers herein are configured to deflect a golf ball or a golfclub or a baseball or a baseball bat or a field hockey stick. The object would “bounce” or ricochet off of the body or component that are consistent with the physics associated with the tangent surfaces, and/or the local deformation elastic and/or plastic that would occur under such circumstances. And note that in an alternative scenario, it is the barrier or brace or foot or leg that “bounces” off of the object, such as the scenario where someone wearing the brace strikes a relatively immovable object, such as, for example, a concrete curb or a concrete step or the side of a car door, etc. Also, the two scenarios are not mutually exclusive, because an object that is relatively massive could still move a bit, but in move a fair amount, but the leg or foot would still react as well. Also, in an embodiment, the barrier is configured to provide more room for deceleration the object relative to that which would otherwise be the case upon contacting the wearer or otherwise the barrier. In this regard, a foam cushion decelerates a baseball a certain value over a distance of a quarter-inch whereas without the barrier that equivalent deceleration would occur over ⅛ of an inch or 1/16 of an inch or the deceleration would occur owing to the skin and/or bones and/or muscle and/or fat of the wearer of the brace, and thus all of the force resulting from the deceleration would be imparted into the wearer. This concept can be considered energy absorption. Arguably, the concept of the ricochet is force/energy dissipation (the force/energy is dissipated over a larger area relative to hitting the skin or brace body) and the concept of the quilt or foam is force energy absorption by the brace (the deceleration over the larger area dissipates the force/energy relative to that which would be imparted into the wearer/brace).

In an embodiment, the barrier elements detailed herein are configured to crack or to break upon a significant (unexpected) force or energy application. By rough analogy, this can be like a bikers helmet or the like, which is designed to be used only one time in the event of a fall off a bike. Conversely, the barriers detailed herein are configured for repeated applications of unwanted energy, without failure, and thus can be akin to a football helmet or a hockey helmet, where repeated applications of unwanted forces occur and are expected.

In an embodiment, barriers detailed herein are configured to be utilized to experience unwanted force/energy above a certain threshold of less than greater than and/or equal to 1 (in which case there is no less than), 2, 3, 4, 5, 6, 7, 8, 9, 10, 11, 12, 13, 14, 15, 20, 25, or 30 times, or any value or range of values therebetween in one increment against the exact same spot or other spots under the same trajectory, etc. (all things being equal). In an embodiment, the test object or otherwise the control object is a baseball, such as the baseball that is approved for major league baseball play in the Major League Baseball franchise on Jun. 22, 2025. In an embodiment, the application of the force corresponds to a baseball striking the barrier elements detailed herein at a 90° angle relative to the tangent surface traveling at a speed of less than, greater than and/or equal to 50, 55, 60, 65, 70, 75, 80, 85, 90, 95, 100, 105, 110, 115, 120, 125, 130, 135, 140, 145, or 150 mile-per-hour, or any value or range of values therebetween in 1 mile-per-hour increments. In an embodiment, the angle is less than, greater than and/or equal to 1, 2, 3, 4, 5, 10, 15, 20, 25, 30, 35, 40, 45, 50, 55, 60, 65, or 70 degrees or more, or any value or range of values therebetween in 1 degree increments from the normal line relative to the tangent surface at the impact (a cone of that angle about the normal line from the tangent surface). Note that this tangent surface could be an extrapolated surface (for example, if the area of impact between two foam cushions, where there is a space between the cushions, however small, there might be no surface per se at the exact angle of incidence, but the perfect be extrapolated from the nearest cushions for example). In an embodiment, the energy dissipation and/or deflection and/or absorption from such results in a force and/or energy as measured over an area that is within a circle of radius of 0.1, 0.15, 0.2, 0.25, 0.3, 0.35, 0.4, 0.45, or 0.5 inches or any value or range of values therebetween in 0.01 inch increments or a square having a side having those dimensions on the opposite side of the barrier of less than, greater than and/or equal to 80, 75, 70, 65, 60, 55, 50, 45, 40, 35, 30, 25, 24, 23, 22, 21, 20, 19, 18, 17, 16, 15, 14, 13, 12, 11, 10, 9, 8, 8, 6, 5, 4, 3, 2, or 1% or less, or any value or range of values therebetween in 0.5% increments of the force and/or energy that would otherwise have been measured or otherwise would exist without the barrier and/or would exist without anything over the test area, or the test area being immediately behind the portion of the brace that would be struck (body, joint, strap, rivet/Chicago screw, etc., if the barrier was not there and/or a portion of the shoe and/or sock and/or pants leg, that would be struck, for an apples to apples comparison.

In an embodiment, the brace itself provides some force/energy dissipation and/or absorption.

In an embodiment, the energy dissipation and/or deflection and/or absorption from such results in a force and/or energy as measured over an area that is within a circle of radius of 0.1, . 15, 0.2, 0.25, 0.3, 0.35, 0.4, 0.45, or 0.5 inches, or any value or range of values therebetween in 0.01 inch increments or a square having a side having those dimensions on the opposite side of the barrier of less than, greater than and/or equal to 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, or 5% or less, or any value or range of values therebetween in 0.5% increments of the force and/or energy that would otherwise have been measured or otherwise would exist without the barrier and/or would exist without anything over the test area, or the test area being immediately behind the portion of the brace that would be struck (body, joint, strap, rivet/Chicago screw, etc.), if the barrier was not there and/or a portion of the shoe and/or sock and/or pants leg, that would be struck, for an apples to apples comparison. Indeed, embodiments include utilizing the brace without the barriers detailed herein and/or having an impact at a location where the barrier is not present where the brace provides one or more of the performance scenarios detailed herein under the impact scenarios. And as noted above, embodiments include a brace that includes a barrier component that is monolithic or otherwise is permanently attached to the brace.

In an embodiment, the element can be used in combination with a trauma pad/padding behind the element (or in another embodiment, padding can be located above a rigid/hard element). In this regard, the arrangement can utilize a relatively hard or rigid barrier overtop a more flexible or elastic body that will absorb force and/or energy or otherwise dissipate such over a larger area relative to that which would be the case if only the rigid body were located above the foot and/or ankle and/or shim and/or lower leg. In an embodiment, the trauma pad reduces the force and/or energy by any of the values just noted relative to that which would otherwise be the case (only a rigid barrier for example). The reverse is also the case, where the trauma pad/padding is located above the rigid body. Thus, the two can be used in combination.

In an embodiment, there is an orthotic, comprising a lower leg interface and a shoe interface articulatedly connected to the lower leg interface, wherein the orthotic is configured with a distinct barrier to protect tissue of a wearer of the orthotic. This barrier can be any of those detailed herein, and distinct is distinguished from the overall arrangement of the brace other than the barrier (e.g., a thickened section on one side of the brace vs. the other with no other utilitarian value would be a barrier, or the attached barrier of course, or an extended portion that is not necessary for ankle support). Indeed, in an exemplary embodiment, the barrier is a component that is not needed or otherwise would not be recognized as a component that is provided for ankle support otherwise to implement the therapeutic features and/or orthotic features of the brace.

In an embodiment, the shoe interface includes a main body (e.g., the thermoplastic material making up the sidewalls), and the barrier is made of material (e.g., includes material) that is harder than material making up the main body. In an embodiment, the hardness is less than, greater than and/or equal to 1.25, 1.5, 1.75, 2, 2.5, 3, 3.5, 4, 4.5, 5, 5.5, 6, 7, 8, 9, 10, 11, 12, 13, 14, 15, or 20 times or more, or any value or range of values therebetween in 0.05 increments harder than the material making up the main body, and this can be a maximum hardness or minimum hardness or a mean, median and/or mode hardness, etc., of either the barrier and/or the body (the barrier being the energy absorbing component). Conversely, in at least some embodiments, as noted above, padding or the like can be utilized as the barrier, and thus in an embodiment, the hardness of the body making up the comparison portion of the brace as the aforementioned higher hardnesses relative to the barrier for textual economy.

The barrier can have a thickness that is greater than a wall thickness of the main body. In an embodiment, the thickness is less than, greater than and/or equal to 1.25, 1.5, 1.75, 2, 2.5, 3, 3.5, 4, 4.5, 5, 5.5, 6, 7, 8, 9, 10, 11, 12, 13, 14, 15, or 20 times or more, or any value or range of values therebetween in 0.05 increments than that of the main body (at least proximate the border of the barrier at one or more locations) and this can be a maximum thickness or minimum thickness or a mean, median and/or mode thickness, etc., of either the barrier and/or the body (the barrier being the energy absorbing component).

In an embodiment, the various control references can be references that exist over, for example, 50, 60, 70, 80, 90% or more, or any value or range of values therebetween in 1% increments of a given area or a given portion by weights or size or by displacement or volume, etc., of any of the components detailed herein, in the interest of textual economy.

In an exemplary embodiment, the barrier is monolithic with the main body, while in other embodiments, the barrier is not monolithic with the main body. In an embodiment, the barrier is mechanically attached to the main body. In an exemplary embodiment, the barrier is riveted and/or screwed and/or glued and/or bonded and/or welded to the main body. In an embodiment, the barrier is strapped around the sidewalls of the rigid heel enclosure for example, or around the uprights for example in the case of a barrier that is utilized to protect the shin.

As seen above, in an embodiment, the barrier extends from beyond one side of the orthotic to beyond another side of the orthotic opposite the one side, but in other embodiments, the barrier does not extend beyond the sides, and in other embodiments, the barrier is only located on one side. In an embodiment, the barrier extends past an end of a distal end of the shoe interface.

In an embodiment, such as a barrier that includes a first portion that protects the shin and a second portion that protects the foot, the barrier can have a first portion that articulates relative to a second portion with articulation of the shoe interface, and this can be in a one to one manner, or can be in a manner that is different than the articulation of the shoe interface.

In an embodiment, there is a system, comprising an external ankle brace, and a sports guard. The external ankle brace can include a medial lower leg section configured to interface with at least a medial side of a leg of a human at a lower leg portion thereof, the medial lower leg section including a medial rigid body contoured to a medial side of a lower leg of a human, a lateral lower leg section configured to interface with at least a lateral side of a leg of a human at a lower leg portion thereof, the lateral lower leg section including a lateral rigid body contoured to a lateral side of a lower leg of a human, a foot section configured to interface with a shoe worn on a foot of the human at locations where the foot of the human is located, a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section. In an embodiment, the sports guard is overlaid over the external ankle brace (in an embodiment, all parts, including attachment components (straps for example) are outside of the brace components).

In an embodiment, the sports guard is connected to the external ankle brace. In an embodiment, the sports guard includes 1, 2, 3, 4, 5, or 6 straps (or shrinkys or tubes) configured to extend around at least one of the medial lower leg section, the lateral lower leg section or the foot section (and/or the straps thereof and/or the shoe and/or pants and leg of the user, etc.).

Consistent with the teachings detailed above where a commercially available sports guard is utilized with a brace, in an embodiment, the sports guard is configured to be used independently of the external ankle brace and the external ankle brace is configured to be used independently of the sports guard. In an embodiment, the sports guard is oversized by less than, greater than and/or equal to 5, 10, 15, 20, 25, 30, 35, or 40%, or any value or range of values therebetween in 1% increments relative to that which would be the case if the sports guard was utilized with the user of the brace in the absence of the brace, all other things being equal. This could be the entire guard, or the straps (the circumference of the resulting strapping, and this may not be the design size but instead the resulting circumference of the existing strap). In an embodiment, the next size or two or 3 next sizes up are used relative to that which would be the case all other things being equal. The sports guard extends downward from a location directly in front of the medial lower leg section and the lateral lower leg section and forward to a location directly above the foot section. In an embodiment, the sports guard extends at least 30, 35, 40, 45, 50, 55, 60, 65, 70, 80, 90, or 100% of the length of extension of the uprights above the joint and/or the lowest portion of the uprights or from the enclosure. The sports guard could be connected to the foot section. The sports guard could be connected to at least one of the medial lower leg section, the lateral lower leg section or a strap connecting the medial lower leg section to the lateral lower leg section. In an embodiment, the sports guard extends less than, greater than and/or equal to 30, 35, 40, 45, 50, 55, 60, 65, 70, 80, 90, or 100%, or any value or range of values therebetween in 1% increments of the length of extension of the enclosure in front of the uprights.

Of course, consistent with the exemplary scenarios detailed herein, the sports guard is a baseball sports guard.

In an embodiment, when the system is viewed directly from the front, the sports guard eclipses less than, greater than and/or equal to 5, 10, 15, 20, 25, 30, 35, 40, 45, 50, 55, 60, 65, 70, 80, 90, or 95% or more, or any value or range of values therebetween in 1% increments of the external ankle brace (what is visible from the front). When the system is viewed directly from the front, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of a vertical length of the medial lower leg section and/or the lateral lower leg section. When the system is viewed directly from the front, the sports guard eclipses less than, greater than and/or equal to 5, 10, 15, 20, 25, 30, 35, 40, 45, 50, 55, 60, 65, 70, 80, 90, or 95% or more, or any value or range of values therebetween in 1% increments of a vertical length of the medial upright or the lateral upright. When the system is viewed directly from the front, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of the foot section that extends in front of the forwardmost portion if the medial lower leg section and/or the medial upright and/or lateral upright or lateral lower leg section. When the system is viewed directly from the front, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of the foot section. Again, all of this is with reference to what is visible without the barrier. If a portion is eclipsed by another component, then that is not visible and thus not taken into the calculations—although in another embodiment-call it the naked embodiment—this can be with respect to the bodies that make up the enclosure and/or the uprights without any other eclipsing elements (e.g., just the uprights, just the enclosure—this is how determination of whether the design meets the arrangement can be determined, even if such requires a destruct test for example). When the system is viewed directly from above, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of the foot section that extends in front of the forwardmost portion of the medial lower leg section and/or medial upright and/or lateral lower leg section and/or lateral upright.

In some embodiments, the sports guard extends past a distalmost portion of the foot section, while in others, it does not so extend. When the system is viewed directly from the side, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of the foot section that extends in front of the forwardmost portion of the medial lower leg section and/or joint. When the system is viewed directly from a medial side, the sports guard eclipses less than, greater than, and/or equal to 5, 10, 15, 20, 25, 30, 35, 40, 45, 50, 55, 60, 65, 70, 80, 90, or 95% or more, or any value or range of values therebetween in 1% increments of the medial lower leg section and/or the lateral lower leg section and/or the medial upright and/or the lateral upright.

When the system is viewed directly from the side, the sports guard eclipses less than, greater than, and/or equal to 5, 10, 15, 20, 25, 30, 35, 40, 45, 50, 55, 60, 65, 70, 80, 90 or 95% or more or any value or range of values therebetween in 1% increments of the lateral female joint portion or the medial female joint portion and/or the male portions thereof (in the naked embodiment for example). When the system is viewed directly from the front, the sports guard eclipses less than, greater than and/or equal to 5, 10, 15, 20, 25, 30, 35, 40, 45, 50, 55, 60, 65, 70, 80, 90, or 95% or more, or any value or range of values therebetween in 1% increments of the lateral female joint portion or the medial female joint portion and/or the male portions thereof (in the naked embodiment for example). In an embodiment, the sports guard might not eclipse any one or more of the components disclosed herein when viewed from the side or the front (and note that the side is the side of the component identified).

Again, the eclipse is what is visible all other things being equal, but in other embodiments, the eclipse is with respect to any of the individual components without the other components (if “naked” or “alone” or “in isolation” is specified).

In an embodiment, there is a foot orthosis that includes a first apparatus configured to grip a shoe worn by a human, a second apparatus configured to grip a leg of the human and an energy management apparatus, wherein the first apparatus articulates relative to the second apparatus, the second apparatus includes a first subportion that articulates relative to the first apparatus and that extends from a location below a top portion of the first apparatus to a location above the top portion of the first apparatus, the second apparatus includes a second subportion that articulates relative to the first apparatus and that extends from a location below a top portion of the first apparatus to a location above the top portion of the first apparatus. In an embodiment, the energy management apparatus is an energy dissipating apparatus and/or an energy distributing apparatus. With respect to one or the other or both, under any of the tests detailed herein, the dissipation and/or deflection is less than, greater than and/or equal to 5, 10, 15, 20, 25, 30, 35, 40, 45, 50, 55, 60, 65, 70, 80, 90, or 95% or more, or any value or range of values therebetween in 1% increments of the total amount of energy influenced by the barrier.

In an embodiment, the energy management apparatus is a ballistic object deflection apparatus and/or a ballistic object deceleration apparatus. In an embodiment, the barrier decelerates the object by a speed of less than, greater than and/or equal to 5, 10, 15, 20, 25, 30, 35, 40, 45, 50, 55, 60, 65, 70, 80, 90 or 95% or more or any value or range of values therebetween in 1% increments over a distance that is less than, greater than and/or equal to 0.05, 0.1, .15, 2, .25, . 3, .35, .4, .45, .5, .55, 6, .65, 7, .75, .8, 9, 1, 1.1, 1.2, 1.3, 1.4, or 1.5 inches, or any value or range of values therebetween in 0.01 inch increments, where the speeds can be any of those detailed herein.

In an embodiment, the features detailed herein can be tested utilizing strain sensors and/or stress sensors or deflection sensors, and can be utilized with a body that is very rigid, such as a wood or a steel replica of a shoe, that is fitted into the brace. In an embodiment, any of the above performance features can result from using such a test setup. I set up in the replica of a standard shoe or average two with respect to size and/or shape, etc., that would be used with the brace and the endeavor associated with the use of the brace, such as baseball, football, running, walking, golfing, etc. Standard testing devices are utilized in some embodiments. These performance features or features that would exist in some exemplary embodiments during normal use by a normal person or a typical human factors engineering person of 1, 5, 10, 15, 20, 25, 30, 35, 40, 45, 50, 55, 60, 65, 70, 75, 80, 85, 90, 95, or 99%, or any value or range of values therebetween in 1% increments born in the United States of America for the People's Republic of China or the European Union (current member states were understated the time of birth) or the United Kingdom or the Federal Republic of Germany or the Republic of France who is 10, 15, 20, 25, 30, 35, 40, 45, 50, 55, 60, 65, 70, or 75 years old, or any value range of values therebetween when you are increments. In this human factors engineering person can be in mail or could be a female.

In an embodiment, the energy management apparatus includes a plate and/or an elastic body and/or a fabric-based protection portion and/or a plurality of segmented energy absorption portions, such as individual elastic and/or plastic deformably blocks in the form of quilting-hard of soft foam, etc.). In an embodiment, the energy management apparatus includes a plurality of segmented energy dissipation portions. In an embodiment, there is less than, greater than, and/or equal to 1, 2, 3, 4, 5, 6, 7, 8, 9, 10, 11, 12, 13, 14, 15, 16, 17, 18, 19, 20, 25, 30, 35, or 40 or more, or any value or range of guys therebetween in one increments portions that are segmented from other portions, and this can be collectively for the leg barrier and the foot barrier, or individually for one or both and the two need not be the same.

In an embodiment, there is an external ankle brace, that includes any one or more of the components detailed herein with respect to the brace, also includes the foot enclosure, along with a barrier component that is supported by, for example, the enclosure. The barrier is, in some embodiments, configured to be strapped around the enclosure. The barrier is, in some embodiments, configured to rest on top portions of the sidewalls when supported by the enclosure (which could be the top edges depending on the curvature of the shoe. The barrier could be configured to rest on a top portion of the upper strap when supported by the enclosure and/or to the lower strapping system (e.g., the clasp and/or the fasteners/fastening body, etc.). In some embodiments, the barrier component is configured to attach to a strap of the brace (upper fastening system strap(s) lower fastening system strap(s), straps of the uprights, etc. Again, the barrier component could be configured to attach to at least one of the sidewalls when supported by the enclosure.

Still, consistent with some embodiments, the barrier component is not positively retained to the enclosure (e.g., the barrier could be pulled off if pulled in the horizontal direction without relieving or expanding the straps aside from that used to overcome friction (or clear the shoe)). The barrier component is snapped to the enclosure and/or a strap attached to the enclosure in some embodiments.

In an embodiment, the barrier component is configured to be strapped around at least one of the uprights or both of the uprights and/or the barrier component is configured to abut forward portions of the sidewalls and/or the forward edges when supported by the at least one upright.

In an embodiment where a strap extends from the lateral upright to the medial upright, the barrier component can be configured to rest on a front portion of the strap when supported by the at least one upright and/or the barrier component is configured to attach to the strap when supported by the enclosure. The barrier component is supported by both of the uprights, directly or indirectly. The barrier component is configured to attach to at least one of the uprights when supported by the enclosure in some embodiments, and in some embodiments, the barrier component is not positively retained to either of the uprights (but could be to the brace owing to the straps, if for example the attachment mechanism fits in part in between the two straps, or below the bottom strap, etc.,—this is also the case for the foot guard and the upper and/or lower strap. In an embodiment, the barrier component is snapped to the at least one upright and/or a strap attached to the at least one upright.

In an embodiment, there is an apparatus that includes an external gauntlet ankle/foot orthosis configured for application over a shoe configured to provide ankle and foot stability at least one of prophylactically or following trauma and the apparatus also includes means for reducing trauma from impact (including eliminating of such). The means for reducing trauma is configured to reduce trauma to the foot and/or shin and/or lower leg. The means could be a Kevlar body and/or fiberglass body or plastic body or polymer body.

Note that some embodiments of the barriers herein are configured to elastically deform upon impact and others to plastically deform and/or fracture or otherwise break, depending on the embodiment and/or force. The means for reducing trauma includes a polymer body in some embodiments and/or a metal body and/or a foam body and/or includes padding and/or a quilted barrier.

The means for reducing trauma is a permanent part of the apparatus or is a readily detachable/removable part of the apparatus.

In an embodiment, there is an orthotic, comprising a lower leg interface, a shoe interface; and a strap, wherein the strap extends from one side of the shoe interface to an opposite side of the shoe interface beneath a shoe when a shoe is located in the shoe interface, and the orthotic includes an impact guard. In an embodiment, a surface of the impact guard overlaps less than, greater than and/or equal to 1, 2, 3, 4, 5, 10, 15, 20, 25, 30, 35, 40, 45, 50, 55, 60, 65, 70, 80, 90, or 95% or more, or any value or range of values therebetween in 1% increments of a main body making up one of the sidewalls (take the enclosure naked, and calculate the entire surface area (whether facing outboard or inboard), and then compare the surface area of the guard that overlaps such). This can be for the energy managing portion in some embodiments (as opposed to that portion and the straps, etc.).

In an embodiment, a surface of the impact guard overlaps less than, greater than and/or equal to 1, 2, 3, 4, 5, 10, 15, 20, 25, 30, 35, 40, 45, 50, 55, 60, 65, 70, 80, 90, or 95% or more, or any value or range of values therebetween in 1% increments of a main body making up one of the uprights.

(Note the overlap is along a vector that is normal to the facing surface, and thus there is the outboard surface and the inboard surface of the body if it is on that vector, hence why the area could be more than 50% for example.)

In an embodiment, the shoe interface includes two sidewalls, the lower leg interface includes two uprights and with respect to a view looking downward on the orthotic, the area bounded by a first line normal to the longitudinal axis of the shoe interface at the most forward portion of the two uprights and a second line normal to the longitudinal axis at the most forward portion of the two sidewalls and by the two sidewalls is less than, greater than, and/or equal to 10, 15, 20, 25, 30, 35, 40, 45, 50, 55, 60, 65, 70, 80, 90, or 95% or more, or any value or range of values therebetween in 1% increments covered by the impact guard. With respect to a view looking towards the front of the orthotic, the area bounded by a first line normal to the longitudinal axis of the lower leg interface at the highest portion of the two sidewalls and a second line normal to the longitudinal axis at the highest portion of the two sidewalls and by the two uprights is less than, greater than and/or equal to 10, 15, 20, 25, 30, 35, 40, 45, 50, 55, 60, 65, 70, 80, 90, or 95% or more, or any value or range of values therebetween in 1% increments covered by the impact guard. With respect to a view looking downward on the orthotic, with respect to a first line normal to the longitudinal axis of the shoe interface at the most forward portion of the two uprights, the impact guard is forward or backward of that line by less than, greater than, and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments. With respect to a view looking towards the front of the orthotic, with respect to a first line normal to the longitudinal axis of the lower leg interface at the highest portion of the two sidewalls, the impact guard is above and/or below that line by less than, greater than and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments. With respect to a view looking downward on the orthotic, with respect to a first line normal to the longitudinal axis of the shoe interface at the most distal portion of the two sidewalls, the impact guard extends less than, greater than and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments past that line. With respect to a view looking towards the front of the orthotic, the area bounded by a first line normal to the longitudinal axis of the lower leg interface at the highest portion of the two sidewalls, the impact guard extends less than, greater than and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments past that line. The impact guard extends from less than, greater than, and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments from the top of the brace (above such or not above such). downward to less than, greater than, and/or equal to 0.25, 0.5, 0.75, 1, 1.25, 1.5, 1.75, 2, 2.25, 2.5, 2.75, 3, 3.5, 4, 4.5, 5, 5.5, or 6 inches or more, or any value or range of values therebetween in 0.01 inch increments from the most forward portion of the brace (past the end or not past the end).

In an embodiment, there is a foot orthosis that includes or is mated with an impact barrier. In an embodiment, a total weight (US or mass rest of the world) of the impact barrier, not including connection componentry, is less than, greater than, and/or equal to 10, 15, 20, 25, 30, 35, 40, 45, 50, 55, 60, 65, 70, 80, 90, 100, 105, 110, 115, 120, 125, 130, 135, 140, 145, 150, 160, 170, 180, 190, 200, 225, 250, 275, 300, 350, 400, 450, or 500% or more, or any value or range of values therebetween in 1% increments of any one or more or all of the main bodies of the brace (one or both uprights and/or the enclosure—the main bodies are the polymer molded part, not including the straps/rivets, pads, etc.).

In an embodiment, a hardness of the impact barrier (maximum, minimum, mean, median, and/or mode) barrier is less than, greater than, and/or equal to 10, 15, 20, 25, 30, 35, 40, 45, 50, 55, 60, 65, 70, 80, 90, 100, 105, 110, 115, 120, 125, 130, 135, 140, 145, 150, 160, 170, 180, 190, 200, 225, 250, 275, 300, 350, 400, 450, or 500% or more, or any value or range of values therebetween in 1% increments of any one or more or all of the main bodies of the brace.

In an embodiment, a thickness (maximum, minimum, mean/median, and/or mode) thickness of the impact barrier is less than, greater than, and/or equal to 10, 15, 20, 25, 30, 35, 40, 45, 50, 55, 60, 65, 70, 80, 90, 100, 105, 110, 115, 120, 125, 130, 135, 140, 145, 150, 160, 170, 180, 190, 200, 225, 250, 275, 300, 350, 400, 450, or 500% or more, or any value or range of values therebetween in 1% increments of the thickness of the mean thickness (thickness are the wall thickness, not the mailing/boxing dimensions) of any one or more of the main bodies of the brace. In an embodiment, a maximum thickness of the impact barrier is less than, greater than and/or equal to 10, 15, 20, 25, 30, 35, 40, 45, 50, 55, 60, 65, 70, 80, 90, 100, 105, 110, 115, 120, 125, 130, 135, 140, 145, 150, 160, 170, 180, 190, 200, 225, 250, 275, 300, 350, 400, 450, or 500% or more, or any value or range of values therebetween in 1% increments of times the mean thickness of the main body.

As noted herein, embodiments can include the combination of a leg guard and a foot guard. In an embodiment, these are attached to each other, and in some embodiments, the attachment is undoable in a readily intended manner, such as by utilizing Velcro straps or the like, while in other embodiments, the attachment is permanent nature, requiring a destruct test or otherwise undoing componentry that is not intended to be undone, at least by the user. FIG. 107A shows an example of a leg guard 8101 attached to a foot guard 7100 by way of two spring components 8755. In an embodiment, this can be made of spring steel or a flexible plastic material or could not even be a spring or the like, but instead a cord that is flexible and otherwise permits relative movement between the leg guard and the foot guard. In this regard, when the brace is utilized, the heel enclosure will articulate relative to the uprights. If the leg guard 8101 is attached to the uprights (or to the leg), the foot guard 7100 will thus articulate relative to the leg guard. An arrangement that holds the two together spatially, or otherwise as an assembly, whether the assembly is undoable or not undoable, while permitting one to move relative to the other, thus has utilitarian value. To the extent that a spring material is utilized, this could be a relatively low force spring arrangement meant more to simply maintain a general position, such as the longitudinal axis of the leg portion being that may be 100, 110, or 120 degrees to the longitudinal axis of the foot portion, which might be the angles that would exist when a person is standing upright without walking, where the attachment arrangement allows the foot portion to articulate relative to the leg portion when walking and thus overcomes the resistance of the spring in a manner that is such that any resistance provided by the spring is de minimis with respect to the action of walking and/or running.

In an embodiment, the longitudinal axis of the leg barrier is at an angle that is less than, greater than, and/or equal to 80, 90, 100, 110, 120, 130, 140, 150, 160, 170, or 180 degrees, or any value or range of values therebetween in 1° increments relative to the longitudinal axis of the foot barrier when the assembly of the leg and foot barrier is in a relaxed state and otherwise not attached to the user or to the brace. In an embodiment, the attachment regime can hold the angle to within less than, greater than and/or equal to 2, 3, 4, 5, 6, 7, 8, 9, 10, 11, 12, 13, 14, 15, 16, 17, 18, 19, 20, 21, 22, 23, 24, 25 degrees, or any value or range of values therebetween in 1° increments under any orientation relative to gravity (as a fee body).

Still, in an embodiment, attachment 8755 might not be made of the spring or the like, but instead simply could be more of an attachment lanyard or the like. And again, in an embodiment, there is a Velcro strap 8756 that extends from the leg barrier to the foot barrier, such as that shown in FIG. 107B. In this embodiment, the strap 8756 is shown on the outboard sides of the barriers, but in an alternate embodiment, the strap could be on the inboard sides, and there can be two straps, one on the outboard side and one on the inboard side. Note also that while the embodiments herein have focused on Velcro straps, in an alternate embodiment, the strap could be permanently attached to one or both of the barriers. Note also that in an embodiment, element 8756 can be representative of an elastic component that has spring qualities and otherwise will provide a spring force on the system. Note also that while embodiments have focused on a spring or an elastic attachment system in an alternate embodiment, it could be that the attachment components are more of a malleable arrangement, where movement is permitted, but then the components will generally be held at the new angular orientation resulting from the movement due to the malleable components, until another force applied to move the components back. This can have fatigue problems, and thus a material that has a high fatigue tolerance can be utilized in some embodiments.

With respect to the spring concepts and/or the malleable concepts, in an embodiment, there can be utilitarian value with respect to having a footpad that is maintained in position over the foot without the foot pad having a dedicated attachment regime to the heel enclosure and/or the uprights. In an exemplary embodiment, the attachment mechanism that attaches the leg barrier to the foot barrier is biased so that the foot barrier would be angled downward by an amount equal to or greater than that which would be the case when the barrier is worn by a user or otherwise placed over the brace. Accordingly, if for example the person wearing the brace utilizing his or her normal shoes or whatever shoes he or she would utilize with the brace is standing upright without running or walking, and otherwise the uprights are at a vertical position, or otherwise directly aligned with the direction of gravity, and the angle between the longitudinal axis of the leg barrier and the foot barrier is say 115, in the absence of the brace of the shoe, the angle between the two might be more of 120 or 125 or 130 or 135° when the spring is that the relaxed state. Thus, there is always a modicum of downward force or more accurately, a moment is applied to the foot barrier, which holds the foot barrier over the shoe and/or the heel enclosure, even though the foot barrier has no dedicated attachment to anything other than the leg barrier. Angles at the higher end of the just noted ranges could have utilitarian value with respect to always “holding” or otherwise pressing the foot barrier downward when the user of the brace is running, or otherwise is experiencing plantar flexion (but the K value of the spring or whatever arrangement barrier assembly does not limit or otherwise impede dorsiflexion, enabling the user of the brace to run in a relatively unencumbered manner, if not a totally unencumbered manner).

This can have utilitarian value with respect to having a sports guard or the like that is relatively easy to utilize with the brace and otherwise requires limited attachment efforts. In an embodiment, a method of use would entail placing the leg barrier between the forward portions of the vertical uprights for otherwise the forward facing edges, and deforming the connection between the leg barrier and the foot barrier so that the foot barrier conforms to the general overall shape of the top of the foot and/or shoe and/or heel enclosure, and then strapping the leg barrier in place by wrapping the upright straps around the uprights and/or the leg barrier. This could also entail attaching the leg barrier to the straps or some other component of the brace. The point is here, there is nothing that is attached to the foot barrier other than the connection regime to the leg barrier and the leg barrier rest on the top of the shoe with a downward force that is sufficient to typically always cover the top of the shoe and/or the top of the foot in a manner that can provide utilitarian value with respect to providing a sports guard or the like, even when the person is running and otherwise experiencing plantar flexion.

In an embodiment, the sports guard assembly is configured to enable articulation of the leg barrier relative to the foot barrier or vice versa by at least the range of angles that the brace is configured to enable articulation of the heel enclosure relative to the uprights.

In an embodiment, a flexibility of a 1 inch length of the barrier is less than, greater than and/or equal to 10, 15, 20, 25, 30, 35, 40, 45, 50, 55, 60, 65, 70, 80, 90, 100, 105, 110, 115, 120, 125, 130, 135, 140, 145, 150, 160, 170, 180, 190, 200, 225, 250, 275, 300, 350, 400, 450 or 500% or more or more or any value or range of values therebetween in 1% increments as flexible as the most flexible 1 inch length of any one or more of the main bodies.

FIG. 117 shows an exemplary flowchart for an exemplary method, method 12900, that entails action 12910, that entails obtaining an ankle brace, which can be any of those detailed herein or a variation thereof in some embodiments, or different braces. Method 12900 includes action 12920, that includes obtaining an ankle brace, which can be any of the ankle braces detailed herein and/or variations thereof or any other ankle brace that have the functionality detailed herein and/or have the features detailed herein in at least some exemplary embodiments. Method 12900 further includes method action 12920, which includes the action of attaching a sports guard to the ankle brace. This can be accomplished by any of the actions detailed herein or otherwise utilizing any of the functionalities detailed herein to accomplish such. In an exemplary embodiment, the sports guard is to be utilized for baseball, and thus, in an exemplary embodiment, there is method action 12930, which entails playing a sport, such as baseball, with the ankle brace and sports guard that the ankle brace.

In an embodiment, there is method 13000, as represented by the flowchart in FIG. 118, which includes method action 13010, which includes obtaining an ankle brace, in an embodiment, this can correspond to any of the actions that can be method action 12910 noted above. Method 13000 further includes method action 13020, which includes placing a sports guard over the ankle brace (of course, this entails the precedent action of obtaining sports guard). In an embodiment, there is a foot guard that includes an elastic strap in the strap is pulled out and expanded so that it fits around the sidewalls and/or one or more the straps of the brace, and then the elastic extension is relaxed so that the strap squeezes around the sidewalls and also the foot or the shoe of the wearer. Any of the actions detailed above that can accomplish the action of placing a sports guard over the ankle brace can be executed in some embodiments providing that the art enables such of course. Method 13000 further includes method action 13030, which entails playing a sport with the ankle brace and sports guard attached to the ankle brace. FIG. 119 shows method action 13100, which includes method action 13010, which includes obtaining an ankle brace with a sports guard. The sports guard could be permanently attached or could be removably attached what is already attached at the time of executing action 13010. In an embodiment, method 13100 further includes method action 13020, which entails playing a sport with the ankle brace and sports guard. In an exemplary embodiment, a sport that is played can be any of the sports detailed herein, such as by way of example, baseball, field hockey, golf, American football, soccer, volleyball, track and field, etc.

In an embodiment, the action of playing the sport includes playing baseball, where the person wearing the brace is a batter. In an exemplary method action, the batter struck by a pitched baseball where ball is not struck by the bat, but the ball hits the batter's foot and/or ankle and/or shin and/or lower leg. Otherwise, this would be an event that would result in a “walk.” In an exemplary embodiment, the ball hits the ground and skids or otherwise bounces off the ground into the just noted anatomical part of the batter's body. Note that this is in direct breaking, because in an exemplary embodiment, the batter is wearing a shoe and/or socks and/or the associated pants worn by baseball players, etc., and is also wearing the brace. In an embodiment, there is also the sports guard, and the ball makes contact with the sports guard, and there is energy and/or force dissipation and/or deflection according to any of the scenarios detailed above or variations thereof. In an embodiment, this is due to the sports guard or otherwise the barrier that is utilized with the brace. But note that in an alternative embodiment, there is no action of obtaining a sports guard and/or attaching the sports guard to the brace or putting sports guard over the brace, or otherwise obtaining a brace for the sports guard and putting on a brace for the sports guard. Instead, the brace without a sports guard per se or otherwise a dedicated barrier or the like is obtained, and the brace is worn and the action of the ball striking the batter or otherwise a sports player occurs, where the ball strikes a portion of the brace, and the energy and/or force deflection and/or dissipation occurs owing to the brace. FIG. 120 shows an exemplary method, method 13200, that includes method action 13210, that entails obtaining an ankle brace, where the ankle brace does not have the dedicated sports guard or the like. The ankle brace could be that according to the embodiments herein and the associated disclosure, alone. The brace could have a heel enclosure having a mean, median and/or mode thickness (material wall thickness) of the body thereof that is less than, greater than and/or equal to 0.03, 0.04, 0.05, 0.06, 0.07, 0.08, 0.09, 0.1, 0.11, 0.12, 0.13, 0.14 or 0.15 inches or any value or range of values therebetween in 0.005 inch increments. These could be the values of all polymer bodies of the brace, or at least the three main parts. (Conversely, the addition of the sports guard would be something that could have a thickness larger than any of these or could correspond to any of the thicknesses detailed above.)

Method 13200 further includes method action 13220, which includes the action of playing a sport with the ankle brace, where in an exemplary embodiment, that action entails striking an object or having an object strike the brace and the associated energy/force management occurs in accordance with any of the teachings detailed herein.

Indeed, in an embodiment, this can also be the case with respect to a brace that also includes the sports guard, except where the ball strikes a portion of the brace that is not covered or otherwise not located where the sports guard is located.

In a variation of the exemplary methods herein, the ball first strikes the bat as the batter swings at the ball. The ball essentially ricochets off the bat downward or to the side depending on the swing (where for example the batter is swinging at a low bar) and the ball strikes the batter's leg or foot or shim or ankle, or more accurately, a portion of the brace and/or a portion of the sports guard over the batter's anatomical feature just noted. In an exemplary embodiment, any of the noted energy dissipation and/or force dissipation and/or energy deflection and/or force deflection scenarios can occur.

In a variation of the methods above, or otherwise an expanded version of such methods, there is use of the brace with or without any sports guard, that involves playing baseball. In an embodiment, a batter is wearing the brace, and the batter swings at a ball that is pitched at the batter. In an embodiment, the brace improves the batter's stance and/or swing by a quantifiable and/or qualifiable amount on a statistical basis that is meaningful with respect to the sport.

In an exemplary embodiment, the braces detailed herein, with or without the sports guard or barriers, etc., are utilized to play a sport, such as baseball, field hockey, etc., where the brace is utilized to manage supination. In this regard, the brace can limit and/or prevent over supination. In an exemplary embodiment, the brace can be considered an anti-sublimation brace or otherwise a subornation limiting brace. In an exemplary embodiment, the brace is configured to an methods include reducing a measured subornation by less than, greater than and/or equal to 15, 20, 25, 30, 35, 40, 45, 50, 55, 60, 65, 70, 75, 80, 85, 90, 91, 92, 93, 94, 95, 96, 97, 98 or 99% or any value or range of values therebetween in 0.1% increments. This is subornation of the foot or otherwise the ankle. In an exemplary embodiment, an increase in weight that is distributed on an outer edge of the foot is reduced by any of the aforementioned values relative to that which would otherwise be the case without the brace, all other things being equal. In an exemplary embodiment, this can be measured by taking an average, mean, median and/or mode of for example, five or 10 or 15 or 20 or 25 or 30 or 35 or more or any value or range as therebetween in one increments actions with the brace and without the brace to determine whether or not the reduction in the specified value occurs. By way of example only and not by way of limitation, a batter could swing at a ball, such as a ball pitched by a machine, any of the aforementioned number of times, and weight could be measured. Alternatively and/or in addition to this, photographic analysis can be used so as to evaluate an angle of the shoe and/or the foot or in anatomical structure, or otherwise movements, which angle would be indicative of subornation, and the angle could be reduced by any of the aforementioned percentages utilizing the brace relative to that which is the case without the brace.

Any arrangement of measuring subornation that can be utilized to evaluate in a qualitative manner the reduction in subornation, or otherwise the prevention of over subornation, can be utilized in at least some exemplary embodiments, providing that the enables such.

In an embodiment, the brace used herein can be used in a manner to manage movements of the foot and/or ankle and/or the relationships thereof, and otherwise manage subornation, or otherwise reduce and/or eliminate over subornation, that results in a quantifiable performance characteristic. In an exemplary embodiment, this can result in a velocity of a bat swing, a baseball bat swing, that is different, and in some embodiments, increased relative to that which would otherwise be the case. In an exemplary embodiment, there are methods that include placing the brace over a shoe or a cleated shoe, and playing baseball, including receiving a pitch from the pitcher and swinging a bat, where the speed of the bat as measured at the most distal portion of the bat is increased by at least and/or equal to and/or no more than 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.5, 8, 8.5, 9, 9.5, 10, 10.5, 11, 11.5, 12, 12.5 or 13% or any value or range of values therebetween in 0.05% increments relative to that which would be the case in the absence of the utilization of the brace, all other things being equal. In an exemplary embodiment, performance can be determined by taking a mean, median and/or mode of any of the number of swings detailed herein with and without the brace, and comparing those to each other to determine if the increase in speed exists. In an exemplary embodiment, a radar or a laser system can be utilized to track the speed of the bat. Still photography or high-speed photography can be used (as is the case with respect to evaluating subornation). In an exemplary embodiment, it is the speed of a baseball as it leaves the bat that is influenced or otherwise improved as a result of the utilization of the brace by a batter. In an exemplary embodiment, the speed of a baseball leaving the bat can be increased by at least and/or equal to and/or no more than 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.5, 8, 8.5, 9, 9.5, 10, 10.5, 11, 11.5, 12, 12.5, 13, 13.5, 14, 14.5, 15, 16, 17 or 18% or any value or range of values therebetween in 0.05% increments relative to that which would be the case in the absence of the utilization of the brace, all other things being equal. Performance can be evaluated by having a batter swing at a ball that is thrown to the batter by the machine at a speed that is replicated over a number of times, such as any of the number of times detailed herein, and a mean, median and/or mode of the velocities of the ball after the ball is struck by the bat can be evaluated to determine performance. That is, any values detailed herein can be averages that occur or otherwise results that are statistically significant or otherwise reproducible and not aberrations. Put another way, any of the performance features detailed herein are those that would be able to be pronounced in a manner of an improvement “on average.” In an exemplary embodiment, these can be applicable to an individual person, or to a statistically significant group of people, such as by age and/or by sex and/or by sport activity, or by profession, such as minor league baseball player or major league baseball player or Little League baseball player at a given age group, etc.

An embodiment includes playing golf or otherwise swinging a golf club utilizing any of the braces detailed herein, with or without a sports card, and achieving at least some of the performance features. In this regard, any disclosure herein associated with the speed of a bat can correspond to the alternate disclosure of a speed of a golf club and/or the speed of the baseball can correspond to the speed of a golf ball, all other things being equal. This can also be applicable to the sport of field hockey, with respect to the field hockey stick and the ball, accordingly.

Embodiments include improving the ability of a pitcher of a baseball team to pitch a baseball. In an exemplary embodiment, the speed of the ball, or more accurately, the mean, median and/or mode speed of the ball that a pitcher pitches can be increased by a pitcher wearing a brace according to the teachings herein by any of the aforementioned percentages in at least some exemplary embodiments and scenarios, relative to that which would otherwise be the case without using the brace. Note that in an embodiment, the baseball that is utilized to evaluate the performance as a baseball that qualifies or falls within the requirements for major league baseball in the United States of America and/or the baseball bat is a bat that meets such regulations or otherwise requirements for major league baseball as of Jun. 23, 2025. In an exemplary embodiment, the golf ball is a golf ball that meets the requirements for the PGA and the golf club is a golf club that meets the requirements for that organization as of the aforementioned date. All of this said, in an exemplary embodiment, an apples to apples comparison can be utilized by utilizing the same baseball of the same bat or the same golf club with the same golf ball for comparison purposes. Additional details of the barrier/energy deflecting/absorbing features, etc., are provided in U.S. Patent Application No. 63/827,528, filed on Jun. 20, 2026, in the United States Patent and Trademark Office, and U.S. Provisional Patent Application No. 63/829,989 filed on Jun. 25, 2026, in the United States Patent and Trademark Office, the entire contents of these applications being incorporated herein by reference in their entirety.

The following provide an exemplary embodiment related to the barrier noted above to protect the foot/leg, hereinafter referred to as embodiment X:

Embodiment X 1. An orthotic, comprising:
a lower leg interface; and
a shoe interface articulatedly connected to the lower leg interface, wherein
the orthotic is configured with a distinct barrier to protect tissue of a wearer of the orthotic.

Embodiment X 2. The orthotic of embodiment X 1, wherein:
the distinct barrier is located above a foot of the wearer when the wearer wears the orthotic.

Embodiment X 3. The orthotic of embodiment X 1, wherein:
the distinct barrier is located in front of a leg of the wearer when the wearer wears the orthotic.

Embodiment X 4. The orthotic of embodiment X 1, wherein:
the shoe interface includes a main body; and
the barrier is made of material that is harder than material making up the main body.

Embodiment X 5. The orthotic of embodiment X 1, wherein:
the shoe interface includes a main body; and the barrier has a thickness that is greater than a wall thickness of the main body.

Embodiment X 6. The orthotic of embodiment X 1, wherein:
the shoe interface includes a main body; and the barrier is monolithic with the main body.

Embodiment X 7. The orthotic of embodiment X 1, wherein:
the shoe interface includes a main body; and
the barrier extends from one side of the orthotic to another side of the orthotic opposite the one side.

Embodiment X 8. The orthotic of embodiment X 1, wherein:
the barrier extends past an end of a distal end of the shoe interface.

Embodiment X 9. The orthotic of embodiment X 1, wherein:
the barrier extends above a top end of the lower leg interface.

Embodiment X 10. The orthotic of embodiment X 1, wherein:
the barrier has a first portion that articulates relative to a second portion with articulation of the shoe interface.

Embodiment X 11. A system, comprising:
an external ankle brace, including:
   a medial lower leg section configured to interface with at least a medial side of a leg of a human at a lower leg portion thereof, the medial lower leg section including a medial rigid body contoured to a medial side of a lower leg of a human;
   a lateral lower leg section configured to interface with at least a lateral side of a leg of a human at a lower leg portion thereof, the lateral lower leg section including a lateral rigid body contoured to a lateral side of a lower leg of a human;
   a foot section configured to interface with a shoe worn on a foot of the human at locations where the foot of the human is located; and
   a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section; and
a sports guard.

Embodiment X 12. The system of embodiment X 11, wherein:
the sports guard is overlaid over the external ankle brace.

Embodiment X 13. The system of embodiment X 11, wherein:
the sports guard is connected to the external ankle brace.

Embodiment X 14. The system of embodiment X 11, wherein:
the sports guard is configured to be used independently of the external ankle brace and the external ankle brace is configured to be used independently of the sports guard.

Embodiment X 15. The system of embodiment X 11, wherein:
the sports guard extends downward from a location directly in front of the medial lower leg section and the lateral lower leg section and forward to a location directly above the foot section.

Embodiment X 16. The system of embodiment X 11, wherein:
the sports guard is connected to the foot section.

Embodiment X 17. The system of embodiment X 11, wherein:
the sports guard is connected to at least one of the medial lower leg section, the lateral lower leg section or a strap connecting the medial lower leg section to the lateral lower leg section.

Embodiment X 18. The system of embodiment X 11, wherein:
the sports guard is a baseball sports guard.

Embodiment X 19. The system of embodiment X 11, wherein:
when the system is viewed directly from the front, the sports guard eclipses at least 60% of the external ankle brace.

Embodiment X 20. The system of embodiment X 11, wherein:
when the system is viewed directly from the front, the sports guard eclipses at least 80% of the external ankle brace.

Embodiment X 21. The system of embodiment X 11, wherein:
when the system is viewed directly from the front, the sports guard eclipses at least 70% of a vertical length of the medial lower leg section and the lateral lower leg section.

Embodiment X 22. The system of embodiment X 11, wherein:
when the system is viewed directly from the front, the sports guard eclipses at least 85% of a vertical length of the medial lower leg section and the lateral lower leg section.

Embodiment X 23. The system of embodiment X 11, wherein:
when the system is viewed directly from above, the sports guard eclipses at least 60% of the foot section that extends in front of the forwardmost portion of the medial lower leg section.

Embodiment X 24. The system of embodiment X 11, wherein:
when the system is viewed directly from above, the sports guard eclipses at least 80% of the foot section that extends in front of the forwardmost portion of the medial lower leg section.

Embodiment X 25. The system of embodiment X 11, wherein:
the sports guard extends past a distalmost portion of the foot section.

Embodiment X 26. The system of embodiment X 11, wherein:
when the system is viewed directly from a side, the sports guard eclipses at least 10% of the foot section that extends in front of the forwardmost portion of the medial lower leg section.

Embodiment X 27. The system of embodiment X 11, wherein:
when the system is viewed directly from a medial side, the sports guard eclipses at least 20% of the medial lower leg section.

Embodiment X 28. The system of embodiment X 11, wherein:
when the system is viewed directly from a medial side, the sports guard eclipses at least 50% of the medial lower leg section.

Embodiment X 29. The system of embodiment X 11, wherein:
when the system is viewed directly from a medial side, the sports guard eclipses at least 80% of the medial ankle joint.

Embodiment X 30. The system of embodiment X 11, wherein:
when the system is viewed directly from a medial side, the sports guard eclipses none of the medial ankle joint.

Embodiment X 31. A foot orthosis, comprising:
a first apparatus configured to grip a shoe worn by a human;
a second apparatus configured to grip a leg of the human; and
an energy management apparatus, wherein
the first apparatus articulates relative to the second apparatus,
the second apparatus includes a first subportion that articulates relative to the first apparatus and that extends from a location below a top portion of the first apparatus to a location above the top portion of the first apparatus,
the second apparatus includes a second subportion that articulates relative to the first apparatus and that extends from a location below a top portion of the first apparatus to a location above the top portion of the first apparatus.

Embodiment X 32. The foot orthosis of embodiment X 31, wherein:
the energy management apparatus is an energy dissipating apparatus.

Embodiment X 33. The foot orthosis of embodiment X 31, wherein:
the energy management apparatus is an energy distributing apparatus.

Embodiment X 34. The foot orthosis of embodiment X 31, wherein:
the energy management apparatus is a ballistic object deflection apparatus.

Embodiment X 35. The foot orthosis of embodiment X 31, wherein:
the energy management apparatus is a ballistic object deceleration apparatus.

Embodiment X 36. The foot orthosis of embodiment X 31, wherein:
the energy management apparatus includes a plate.

Embodiment X 37. The foot orthosis of embodiment X 31, wherein:
the energy management apparatus includes an elastic body.

Embodiment X 38. The foot orthosis of embodiment X 31, wherein:
the energy management apparatus includes a fabric-based protection portion.

Embodiment X 39. The foot orthosis of embodiment X 31, wherein:
the energy management apparatus includes a plurality of segmented energy absorption portions.

Embodiment X 40. The foot orthosis of embodiment X 31, wherein:
the energy management apparatus includes a plurality of segmented energy dissipation portions.

Embodiment X 41. An external ankle brace, comprising:
a medial upright configured to interface with at least a medial side of a leg of a human at a lower leg portion thereof, the medial upright including a medial rigid body contoured to a medial side of a lower leg of a human;
a lateral upright configured to interface with at least a lateral side of a leg of a human at a lower leg portion thereof, the lateral upright including a lateral rigid body contoured to a medial side of a lower leg of a human;
an enclosure configured to interface with a shoe worn on a foot of the human at locations where the foot of the human is located, the enclosure including a medial sidewall and a lateral sidewall;
a lateral ankle joint that pivotally connects said lateral upright to said enclosure and allows the lateral ankle joint to move relative to the enclosure;
a medial ankle joint that pivotally connects said medial upright to the enclosure and allows said medial upright to move relative to said enclosure; and
a barrier component supported by the enclosure, wherein
the lateral ankle joint has a first joint portion that is part of the lateral upright that interfaces with a second joint portion that is part of the enclosure.

Embodiment X 42. The external ankle brace of embodiment X 41, wherein:
the barrier component is configured to be strapped around the enclosure.

Embodiment X 43. The external ankle brace of embodiment X 41, wherein:
the barrier component is configured to rest on top portions of the sidewalls when supported by the enclosure.

Embodiment X 44. The external ankle brace of embodiment X 41, wherein:
a strap extends from the lateral sidewall to the medial sidewall; and
the barrier component is configured to rest on a top portion of the strap when supported by the enclosure.

Embodiment X 45. The external ankle brace of embodiment X 41, wherein:
a strap extends from the lateral sidewall to the medial sidewall; and
the barrier component is configured to attach to the strap when supported by the enclosure.

Embodiment X 46. The external ankle brace of embodiment X 45, wherein:
the strap extends across a bottom of the enclosure.

Embodiment X 47. The external ankle brace of embodiment X 45, wherein:
the strap extends across a top of the enclosure.

Embodiment X 48. The external ankle brace of embodiment X 41, wherein:
the barrier component is configured to attach to at least one of the sidewalls when supported by the enclosure.

Embodiment X 49. The external ankle brace of embodiment X 41, wherein:
the barrier component is not positively retained to the enclosure.

Embodiment X 50. The external ankle brace of embodiment X 41, wherein:
the barrier component is snapped to the enclosure and/or a strap attached to the enclosure.

Embodiment X 51. An external ankle brace, comprising:
a medial upright configured to interface with at least a medial side of a leg of a human at a lower leg portion thereof, the medial upright including a medial rigid body contoured to a medial side of a lower leg of a human;
a lateral upright configured to interface with at least a lateral side of a leg of a human at a lower leg portion thereof, the lateral upright including a lateral rigid body contoured to a medial side of a lower leg of a human;
an enclosure configured to interface with a shoe worn on a foot of the human at locations where the foot of the human is located, the enclosure including a medial sidewall and a lateral sidewall;
a lateral ankle joint that pivotally connects said lateral upright to said enclosure and allows the lateral ankle joint to move relative to the enclosure;
a medial ankle joint that pivotally connects said medial upright to the enclosure and allows said medial upright to move relative to said enclosure; and
a barrier component supported by at least one of the uprights, wherein
the lateral ankle joint has a first joint portion that is part of the lateral upright that interfaces with a second joint portion that is part of the enclosure.

Embodiment X 52. The external ankle brace of embodiment X 41, wherein:
the barrier component is configured to be strapped around at least one of the uprights.

Embodiment X 53. The external ankle brace of embodiment X 51, wherein:
the barrier component is configured to abut forward portions of the sidewalls when supported by the at least one upright.

Embodiment X 54. The external ankle brace of embodiment X 51, wherein:
a strap extends from the lateral upright to the medial upright; and
the barrier component is configured to rest on a front portion of the strap when supported by the at least one upright.

Embodiment X 55. The external ankle brace of embodiment X 51, wherein:
a strap extends from the lateral sidewall to the medial sidewall; and
the barrier component is configured to attach to the strap when supported by the enclosure.

Embodiment X 56. The external ankle brace of embodiment X 55, wherein:
the strap extends completely around both uprights.

Embodiment X 57. The external ankle brace of embodiment X 55, wherein:
the barrier component is supported by both of the uprights.

Embodiment X 58. The external ankle brace of embodiment X 51, wherein:
the barrier component is configured to attach to at least one of the uprights when supported by the enclosure.

Embodiment X 59. The external ankle brace of embodiment X 51, wherein:
the barrier component is not positively retained to either of the uprights.

Embodiment X 60. The external ankle brace of embodiment X 41, wherein:
the barrier component is snapped to the at least one upright and/or a strap attached to the at least one upright.

Embodiment X 61. An apparatus, comprising:
an external gauntlet ankle/foot orthosis configured for application over a shoe configured to provide ankle and foot stability at least one of prophylactically or following trauma, wherein the external gauntlet ankle/foot orthosis includes:
   a first structural section including sub-portions configured to respectively coincide with an outer portion of a foot and ankle and an inner portion of the foot and ankle; and
   a second structural section including sub-portions configured to respectively coincide with an outer portion of a lower leg and an inner portion of the lower leg, wherein
the second structural section is attached to the first structural section via a plurality of ankle joints,
the sub-portions of the first structural section are connected by a connector sub-portion,
the first structural section and the second structural section are made of thermoplastic,
the apparatus further comprises a strap connecting the sub-portions of the second structural section and encircling the lower leg when the external gauntlet ankle/foot orthosis is worn by the human,
a first sub-portion and a second sub-portion of the sub-portions of the first structural section are each configured to extend from the connector sub-portion in a longitudinal direction beyond a talus of the foot and towards a toe of the foot when the external gauntlet ankle/foot orthosis is worn by a human, each of the first sub-portion and the second sub-portion being at least partially located adjacent to an instep area of a corresponding medial or lateral side of the shoe when the external gauntlet ankle/foot orthosis is worn by the human worn by the human, human, and
the external gauntlet ankle/foot orthosis limits and/or prevents ankle inversion and eversion, and
the apparatus also includes means for reducing trauma from impact.

Embodiment X 62. The apparatus of embodiment X 61, wherein:
the means for reducing trauma is configured to reduce trauma to the foot.

Embodiment X 63. The apparatus of embodiment X 61, wherein:
the means for reducing trauma is configured to reduce trauma to the lower leg.

Embodiment X 64. The apparatus of embodiment X 61, wherein:
the means for reducing trauma includes a Kevlar body.

Embodiment X 65. The apparatus of embodiment X 61, wherein:
the means for reducing trauma includes a polymer body.

Embodiment X 66. The apparatus of embodiment X 61, wherein:
the means for reducing trauma includes a metal body.

Embodiment X 67. The apparatus of embodiment X 61, wherein:
the means for reducing trauma includes a foam body.

Embodiment X 68. The apparatus of embodiment X 61, wherein:
the means for reducing trauma includes padding.

Embodiment X 69. The apparatus of embodiment X 61, wherein:
the means for reducing trauma is a quilted barrier.

Embodiment X 70. The apparatus of embodiment X 61, wherein:
the means for reducing trauma is a permanent part of the apparatus.

Embodiment X 71. An orthotic, comprising:
a lower leg interface;
a shoe interface; and
a strap, wherein
the strap extends from one side of the shoe interface to an opposite side of the shoe interface beneath a shoe when a shoe is located in the shoe interface, and
the orthotic includes an impact guard.

Embodiment X 72. The orthotic of embodiment X 71, wherein:
the shoe interface includes two sidewalls; and
a surface of the impact guard overlaps at least 10% of a total surface area of a main body making up one of the sidewalls.

Embodiment X 73. the orthotic of embodiment X 71, wherein:
the lower leg interface includes two uprights; and
a surface area of the impact guard overlaps at least 10% of a surface area of one of the uprights.

Embodiment X 74. The orthotic of embodiment X 71, wherein:
the shoe interface includes two sidewalls;
the lower leg interface includes two uprights; and
with respect to a view looking downward on the orthotic, the area bounded by a first line normal to the longitudinal axis of the shoe interface at the most forward portion of the two uprights and a second line normal to the longitudinal axis at the most forward portion of the two sidewalls and by the two sidewalls is at least 80% covered by the impact guard.

Embodiment X 75. The orthotic of embodiment X 71, wherein:
the shoe interface includes two sidewalls;
the lower leg interface includes two uprights; and
with respect to a view looking towards the front of the orthotic, the area bounded by a first line normal to the longitudinal axis of the lower leg interface at the highest portion of the two sidewalls and a second line normal to the longitudinal axis at the highest portion of the two sidewalls and by the two uprights is at least 80% covered by the impact guard.

Embodiment X 76. The orthotic of embodiment X 71, wherein:
the shoe interface includes two sidewalls;
the lower leg interface includes two uprights; and
with respect to a view looking downward on the orthotic, with respect to a first line normal to the longitudinal axis of the shoe interface at the most forward portion of the two uprights, the impact guard is within 1 inch of that line.

Embodiment X 77. The orthotic of embodiment X 71, wherein:
the shoe interface includes two sidewalls;
the lower leg interface includes two uprights; and
with respect to a view looking towards the front of the orthotic, with respect to a first line normal to the longitudinal axis of the lower leg interface at the highest portion of the two sidewalls, the impact guard is within 1 inch of that line.

Embodiment X 78. The orthotic of embodiment X 71, wherein:
the shoe interface includes two sidewalls;
the lower leg interface includes two uprights; and
with respect to a view looking downward on the orthotic, with respect to a first line normal to the longitudinal axis of the shoe interface at the most distal portion of the two sidewalls, the impact guard extends at least 1 inch past that line.

Embodiment X 79. The orthotic of embodiment X 71, wherein:
the shoe interface includes two sidewalls;
the lower leg interface includes two uprights; and
with respect to a view looking towards the front of the orthotic, the area bounded by a first line normal to the longitudinal axis of the lower leg interface at the highest portion of the two sidewalls, the impact guard extends at least 1 inch past that line.

Embodiment X 80. The orthotic of embodiment X 71, wherein:
the impact guard extends from 1 inch of the top of the brace downward to 1 inch from the most forward portion of the brace.

Embodiment X 81. A foot orthosis, comprising:
a first apparatus configured to grip a shoe worn by a human;
a second apparatus configured to grip a leg of the human, wherein
the first apparatus articulates relative to the second apparatus when the human walks using the foot orthosis, the first apparatus grips the shoe by way of top strap and a bottom strap, and
the foot orthosis includes or is mated with an impact barrier.

Embodiment X 82. The orthosis of embodiment X 81, wherein:
the first apparatus includes a main body; and
a total weight of the impact barrier, not including connection componentry, is no more than 50% of the weight of the main body.

Embodiment X 83. The orthosis of embodiment X 81, wherein:
the first apparatus includes a main body; and
a total weight of the impact barrier, not including connection componentry, is more than 50% of the weight of the main body.

Embodiment X 84. The orthosis of embodiment X 81, wherein:
the first apparatus includes a main body; and
a maximum hardness of the impact barrier is more than 50% harder than the main body.

Embodiment X 85. The orthosis of embodiment X 81, wherein:
the first apparatus includes a main body; and
a maximum hardness of the impact barrier is less than 50% as hard as the main body.

Embodiment X 86. The orthosis of embodiment X 81, wherein:
the first apparatus includes a main body; and
a maximum thickness of the impact barrier is at least as thick as the mean thickness of the main body.

Embodiment X 87. The orthosis of embodiment X 81, wherein:
the first apparatus includes a main body; and
a maximum thickness of the impact barrier is at least three times the mean thickness of the main body.

Embodiment X 88. The orthosis of embodiment X 81, wherein:
the first apparatus includes a main body; and
a flexibility of a 1 inch length of the impact barrier is at least three times as flexible as the most flexible 1 inch length of the main body.

Embodiment X 89. The orthosis of embodiment X 81, wherein:
the second apparatus includes at least one upright that includes a main body; and
a total weight of the impact barrier, not including connection componentry, is no more than 50% of the weight of the main body

Embodiment X 90. The orthosis of embodiment X 81, wherein:
the second apparatus includes at least one upright that includes a main body; and
a total weight of the impact barrier, not including connection componentry, is more than 50% of the weight of the main body.

Embodiment X 91. The orthosis of embodiment X 81, wherein:
the second apparatus includes at least one upright that includes a main body; and
a maximum hardness of the impact barrier is more than 50% harder than the main body.

Embodiment X 92. The orthosis of embodiment X 81, wherein:
the second apparatus includes at least one upright that includes a main body; and
a maximum hardness of the impact barrier is less than 50% as hard as the main body.

Embodiment X 93. The orthosis of embodiment X 81, wherein:
the second apparatus includes at least one upright that includes a main body; and
a maximum thickness of the impact barrier is at least as thick as the mean thickness of the main body.

Embodiment X 94. The orthosis of embodiment X 81, wherein:
the second apparatus includes at least one upright that includes a main body; and
a maximum thickness of the impact barrier is at least three times the mean thickness of the main body.

Embodiment X 95. The orthosis of embodiment X 81, wherein:
the second apparatus includes at least one upright that includes a main body; and
a flexibility of a 2 inch length of the impact barrier is at least three times as flexible as the most flexible 2 inch length of the main body.

Embodiment X 96. The orthosis of embodiment X 81, wherein:
the second apparatus includes at least one upright that includes a main body; and
a maximum hardness of the impact barrier is more than 100% harder than the main body.

Embodiment X 97. The orthosis of embodiment X 81, wherein:
the second apparatus includes at least one upright that includes a main body; and
a maximum hardness of the impact barrier is less than 25% as hard as the main body.

Embodiment X 98. The orthosis of embodiment X 81, wherein:
the second apparatus includes at least one upright that includes a main body; and
a maximum thickness of the impact barrier is at least 1.5 times as thick as the mean thickness of the main body.

Embodiment X 99. The orthosis of embodiment X 81, wherein:
the second apparatus includes at least one upright that includes a main body; and
a maximum thickness of the impact barrier is at least two times the mean thickness of the main body.

Embodiment X 100. The system of embodiment X 81, wherein:
the sports guard includes straps configured to extend around at least one of the medial lower leg section, the lateral lower leg section or the foot section.

Embodiment X 101. An orthotic, comprising:
a lower leg interface; and
a shoe interface articulatedly connected to the lower leg interface, wherein
the orthotic is configured with a barrier between the shoe interface and the lower leg interface, and
an impact barrier.

Embodiment X 102. The orthotic of embodiment X 101, wherein:
the barrier is a protrusion on the shoe interface.

Embodiment X 103. The orthotic of embodiment X 101, wherein:
the barrier is monolithic with a main portion of the shoe interface.

Embodiment X 104. The orthotic of embodiment X 101, wherein:
the barrier has a cross section of a ramp.

Embodiment X 105. The orthotic of embodiment X 101, wherein:
the barrier extends in an arcuate manner.

Embodiment X 106. The orthotic of embodiment X 101, wherein:
the barrier extends in parallel manner with a portion of the lower leg interface immediately adjacent the barrier.

Embodiment X 107. The orthotic of embodiment X 101, wherein:
the barrier is established by a first portion of the orthotic that is more outboard than a second portion of the orthotic.

Embodiment X 108. The orthotic of embodiment X 101, wherein:
the barrier is a component of the orthotic that has no other functional purpose than to prevent an object from being caught between the foot interface and the lower leg interface.

Embodiment X 109. The orthotic of embodiment X 101, wherein:
the barrier is a berm that partially surrounds a portion of the lower leg interface.

Embodiment X 110. The orthotic of embodiment X 101, wherein:
the barrier blocks movement of an object in a vertical direction along a surface of the foot interface.

Embodiment X 111. An external ankle brace, comprising:
a medial lower leg section configured to interface with at least a medial side of a leg of a human at a lower leg portion thereof, the medial lower leg section including a medial rigid body contoured to a medial side of a lower leg of a human;
a lateral lower leg section configured to interface with at least a lateral side of a leg of a human at a lower leg portion thereof, the lateral lower leg section including a lateral rigid body contoured to a lateral side of a lower leg of a human;
a foot section configured to interface with a shoe worn on a foot of the human at locations where the foot of the human is located;
a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section, wherein
the lateral ankle joint has a female portion that is part of the lateral lower leg section that interfaces with a male portion of the lateral ankle joint that is part of the foot section, and
an outermost edge of the female portion is inboard of a portion of the foot section, and
the brace includes an impact barrier.

Embodiment X 112. The external ankle brace of embodiment X 111, wherein:
the inboardness of the outermost edge establishes a barrier.

Embodiment X 113. The external ankle brace of embodiment X 111, wherein:
the inboardness of the outermost edge is located at the lowest portion of the female portion.

Embodiment X 114. The external ankle brace of embodiment X 111, wherein:
the inboardness of the outermost edge extends for more than 30 degrees about the female portion.

Embodiment X 115. The external ankle brace of embodiment X 111, wherein:
the inboardness of the outermost edge is present through all range of motions of the lateral lower leg section.

Embodiment X 116. The external ankle brace of embodiment X 111, wherein:
the inboardness of the outermost edge is established by a protrusion of the foot section, the protrusion corresponding to at least part of the portion of the foot section.

Embodiment X 117. The external ankle brace of embodiment X 111, wherein:
the inboardness of the outermost edge extends for at least 80 percent of an overlap area, when viewed from a side of the brace, of the outermost edge with the foot section.

Embodiment X 118. The external ankle brace of embodiment X 111, wherein:
the outermost edge extends along a circular trajectory through at least 180 degrees.

Embodiment X 119. The external ankle brace of embodiment X 111, wherein:
the portion of the foot section is an arcuately extending berm structure of the foot portion.

Embodiment X 120. The external ankle brace of embodiment X 111, wherein:
the inboardness is a result of a configuration of the brace that forces a horizontal component of movement of an object with respect to movement of the object to a location behind the female portion of the joint relative to that which would otherwise be present, all other things being equal.

Embodiment X 121. A foot orthosis, comprising:
a first apparatus configured to grip a shoe worn by a human;
a second apparatus configured to grip a leg of the human, wherein
the first apparatus articulates relative to the second apparatus,
the second apparatus includes a first subportion that articulates relative to the first apparatus and that extends from a location below a top portion of the first apparatus to a location above the top portion of the apparatus,
the first subportion has a portion that overlaps with a portion of the first apparatus when viewed from a side of the orthosis, and
when viewed looking upwards at the brace along a trajectory that is coincident with the vertical direction, a portion of the first subportion is eclipsed by the first apparatus, and
the orthosis includes an impact barrier.

Embodiment X 122. The orthosis of embodiment X 121, wherein:
when viewed looking upwards at the brace along a trajectory that is 10 degrees from the vertical direction, which view also allows for more of an outer side of the first subportion to be seen, the portion of the first subportion remains eclipsed by the first apparatus.

Embodiment X 123. The orthosis of embodiment X 121, wherein:
when viewed looking upwards at the brace along a trajectory that is 20 degrees from the vertical direction, which view also allows for more of an outer side of the first subportion to be seen, the portion of the first subportion remains eclipsed by the first apparatus.

Embodiment X 124. The orthosis of embodiment X 121, wherein:
the portion of the first subportion is a lowermost portion of the second apparatus.

Embodiment X 125. The orthosis of embodiment X 121, wherein:
first subportion includes a joint portion that includes a surface having an arcuate perimeter, a portion of the surface facing the first apparatus and thus establishing the overlap;
at least 30% of a length of the arcuate perimeter is eclipsed by the first apparatus when viewed looking upwards at the brace along a trajectory that is coincident with the vertical direction.

Embodiment X 126. The orthosis of embodiment X 121, wherein
first subportion includes a joint portion that has an arcuate outermost perimeter when viewed from the side, a portion of the outermost perimeter establishing at least a portion of the overlap; and
at least 30% of a length of the arcuate outermost perimeter is eclipsed by the first apparatus when viewed looking upwards at the brace along a trajectory that is coincident with the vertical direction.

Embodiment X 127. The orthosis of embodiment X 121, wherein:
when viewed looking upwards at the brace along a trajectory that is 10 degrees from the vertical direction, which view also allows for more of an outer side of the first subportion to be seen, the portion of the first subportion remains eclipsed by the first apparatus over a range of views extending from 20 degrees on one side of a vertical plane to 20 degrees on an opposite side of the vertical plane, which vertical plane extends through a geometric center of the first subportion and wherein the trajectory that is 10 degrees from the vertical direction lies on the vertical plane.

Embodiment X 128. The orthosis of embodiment X 127, wherein:
the portion that is eclipsed is at least 60% of an outer periphery of the first subportion extending from front to back in the view that would otherwise be visible in the absence of the first apparatus.

Embodiment X 129. The orthosis of embodiment X 121, wherein:
the portion of the first subportion is a female portion of a joint of the orthosis.

Embodiment X 130. The orthosis of embodiment X 121, wherein:
when viewed from the side of the brace, the first apparatus has a protruding component that extends about a portion of the first subportion.

Embodiment X 131. An external ankle brace, comprising:
a medial upright configured to interface with at least a medial side of a leg of a human at a lower leg portion thereof, the medial upright including a medial rigid body contoured to a medial side of a lower leg of a human;
a lateral upright configured to interface with at least a lateral side of a leg of a human at a lower leg portion thereof, the lateral upright including a lateral rigid body contoured to a medial side of a lower leg of a human;
an enclosure configured to interface with a shoe worn on a foot of the human at locations where the foot of the human is located;
a lateral ankle joint that pivotally connects said lateral upright to said enclosure and allows the lateral ankle joint to move relative to the enclosure;
a medial ankle joint that pivotally connects said medial upright to the enclosure and allows said medial upright to move relative to said enclosure, wherein
the lateral ankle joint has a first joint portion that is part of the lateral upright that interfaces with a second joint portion that is part of the enclosure, and
the brace includes an anti-catch feature adjacent the lateral ankle joint, and
the brace includes an impact barrier.

Embodiment X 132. The brace of embodiment X 131, wherein:
the anti-catch feature has a wedge or ramp shaped outer profile.

Embodiment X 133. The brace of embodiment X 131, wherein:
the second joint portion is male portion;
the first joint portion is a female portion;
the enclosure includes a second male portion that extends about a portion of the male portion; and
the second male portion establishes at least part of the anti-catch feature.

Embodiment X 134. The brace of embodiment X 131, wherein:
first joint portion has an outer circumference, and at least 30% of an outer periphery of the first joint portion is bounded by the second male portion, the bounding establishing the anti-catch feature.

Embodiment X 135. The brace of embodiment X 131, wherein:
at least a portion of the first joint portion is inboard of a portion of enclosure immediately adjacent to the first joint portion, the portion of the enclosure not including the second joint portion.

Embodiment X 136. The brace of embodiment X 131, wherein:
a portion of the first joint portion is located in a trough established by the enclosure, the first joint portion being located in the trough establishing the anti-catch feature.

Embodiment X 137. The brace of embodiment X 131, wherein:
the most inboard portions of the first joint portion are inboard of a portion of enclosure immediately adjacent to the first joint portion that is located outside a profile of the first joint portion when the brace is viewed from a side.

Embodiment X 138. The brace of embodiment X 131, wherein:
the anti-catch feature is established by a bump portion of the enclosure;
the enclosure includes at least a lateral sidewall; and
the sidewall extends away from a crest of the bump with increasing distance with location in a rearward and/or forward direction.

Embodiment X 139. The brace of embodiment X 131, wherein:
the anti-catch feature is established by a guard portion of the enclosure;
the guard portion includes a crest; and
the crest extends for at least an inch within two parallel planes no more than 0.1 inches from each other.

Embodiment X 140. The brace of embodiment X 131, wherein:
the second joint portion is male portion;
the first joint portion is a female portion;
the enclosure includes a second male portion having an extension that is concentric with an outer profile of the male portion and the female portion;
the second male portion establishes at least part of the anti-catch feature.

Embodiment X 141. An orthotic, comprising:
a lower leg interface; and
a shoe interface articulatedly connected to the lower leg interface, wherein
the orthotic is configured so that adjacent portions of the lower leg interface and the shoe interface overlap in the horizontal direction, and
the orthotic includes an impact barrier.

Embodiment X 142. The orthotic of embodiment X 141, wherein:
the lower leg interface has respective lowermost portions at respective angles of articulation relative to the shoe interface, the respective lowermost portions always being more inboard than a portion of the shoe interface immediately below the respective lowermost portions.

Embodiment X 143. The orthotic of embodiment X 141, wherein:
the lower leg interface has an outer profile a portion of which is contiguously arcuate over at least 180 degrees; and
the foot section includes a portion that is proud of the outermost portion of the contiguous arcuate outer profile at a location adjacent and in the convex side of the accuracy.

Embodiment X 144. The orthotic of embodiment X 143, wherein:
the portion that is proud extends arcuately over at least 70 degrees.

Embodiment X 145. The orthotic of embodiment X 143, wherein:
a first end of the proud portion is located forward of an axis of rotation of a component of the lower leg interface and a second end of the proud portion is located behind the axis of rotation, and the proud portion extends downward from the first end and then upwards towards the second end.

Embodiment X 146. The orthotic of embodiment X 141, wherein:
the foot section includes a thickened wall section of a sidewall of the food section that blends in with a remainder of the foot section to establish the overlap.

Embodiment X 147. The orthotic of embodiment X 141, wherein:
the foot section includes a ramp that slopes with respect to location in a vertical direction outward to establish the overlap.

Embodiment X 148. The orthotic of embodiment X 141, wherein:
the shoe interface has a dome shaped male portion;
the lower leg interface as a dome shaped female portion which is concentric with the dome shaped male portion; and
the shoe interface includes a berm portion, wherein
an outer periphery of the dome shaped female portion is located between the berm potion and the dome shaped male portion, thus establishing the overlap.

Embodiment X 149. The orthotic of embodiment X 141, wherein:
the overlap is established by a portion of the shoe interface that smoothly ramps outboard and then more abruptly extends inboard.

Embodiment X 150. The orthotic of embodiment X 141, wherein:
the lower leg interface has a dome shaped surface having a convex side and a concave side; and
when viewed from the convex side, the outer periphery of the dome shaped surface is below at least a portion of the shoe interface immediately adjacent the lower leg interface.

Embodiment X 151. An orthotic, comprising:
a lower leg interface; and
a shoe interface articulatedly connected to the lower leg interface, wherein
the shoe interface has a dome shaped male portion,
the lower leg interface has a dome shaped female portion, and
the male portion is received in the female portion, and
the orthotic includes an impact barrier.

Embodiment X 152. The orthotic of embodiment X 151, wherein:
the male portion and the female portion establish a joint that enables the shoe interface to articulate relative to the lower leg interface.

Embodiment X 153. The orthotic of embodiment X 151, wherein:
the male portion has an outboard side and an inboard side, wherein the outboard side and the inboard side respectively establish domed surfaces.

Embodiment X 154. The orthotic of embodiment X 151, wherein:
the male portion has an outboard side and an inboard side, wherein the outboard side and the inboard side respectively establish domed surfaces; and
the inboard domed surface supports a foam pad which is configured to interface with a malleolus of a human when the orthotic is worn by a human.

Embodiment X 155. The orthotic of embodiment X 151, wherein:
the male portion rotates relative to and inside of the female portion to enable the shoe interface to articulate relative to the leg interface.

Embodiment X 156. The orthotic of embodiment X 151, wherein:
the male portion is monolithic with a main body of the shoe interface;
the lower leg interface includes an upright; and the female portion is monolithic with a main body of the upright.

Embodiment X 157. The orthotic of embodiment X 151, wherein:
the male portion is inboard of the female portion.

Embodiment X 158. The orthotic of embodiment X 151, wherein:
the male portion is separated from the female portion by a thin washer.

Embodiment X 159. The orthotic of embodiment X 151, wherein:
the female portion is fastened to the male portion by a rivet.

Embodiment X 160. The orthotic of embodiment X 151, wherein:
the orthotic is a sports orthotic.

Embodiment X 161. An external ankle brace for selectively restricting movement of an ankle in at least one of a first direction and a rotation direction, and selectively permitting movement of the ankle in a second direction, wherein the external ankle brace is disposed on the exterior of a shoe, the shoe having a heel portion, a toe portion longitudinally spaced from the heel portion, a sole, and oppositely disposed sides, the external ankle brace comprising: a rigid heel enclosure having a rear portion and a forward portion, the rear portion configured to receive and at least partially encircle the heel portion of the shoe, the forward portion having a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe, the lateral and medial sidewalls each extending from the rear portion toward a toe of a wearer's foot and each extending beyond a talus of the wearer's foot; a lateral upright extension selectively perpendicular to the rigid heel enclosure and pivotally attached to the lateral sidewall, the lateral upright extension including a lateral reinforcing strut; a medial upright extension selectively perpendicular to the rigid heel enclosure and pivotally attached to the medial sidewall, the medial upright extension including a medial reinforcing strut; a lower fastening system comprising at least one lower adjustable connecting strap for connecting the lateral sidewall to the medial sidewall and extending underneath the sole of the shoe; and an upper fastening system comprising at least one upper connecting strap for selectively connecting the lateral sidewall to the medial sidewall across the top of the shoe, the upper connecting strap being located longitudinally between the lower connecting strap and the lateral and medial upright extensions, wherein the lower adjustable connecting strap has an outer surface that has a variable heigh with length along the strap, and the brace includes an impact barrier.

Embodiment X 162. The brace of embodiment X 161, wherein the lower adjustable connecting strap is toothed.

Embodiment X 163. The brace of embodiment X 161, wherein the lower adjustable connecting strap has a grip surface.

Embodiment X 164. The brace of embodiment X 161, wherein the lower adjustable connecting strap has a first side and a second side, the first side is smooth from one side of the brace to the other side of the brace, and a second side is rough from one side of the brace to the other side of the brace.

Embodiment X 165. The brace of embodiment X 161, wherein the lower adjustable connecting strap fastened to the medial sidewall with a toothed bracket that interfaces with teeth of the lower adjustable connecting strap.

Embodiment X 166. The brace of embodiment X 161, wherein a clasp is attached to the lateral sidewall and the lower adjustable connecting strap is releasably secured to the lateral sidewall by the clasp.

Embodiment X 167. The brace of embodiment X 166, wherein the clasp includes a toothed component that interfaces with teeth of the lower adjustable connecting strap to releasably secure the lower strap to the lateral sidewall.

Embodiment X 168. The brace of embodiment X 167, wherein the clasp includes a locking component that locks the toothed component in a position where teeth of the toothed component interface with teeth of the lower adjustable connecting strap.

Embodiment X 169. The brace of embodiment X 166, wherein the clasp is a two-stage clasp.

Embodiment X 170. The brace of embodiment X 166, wherein the clasp is configured to be relatively difficult to undo to release the lower strap.

Embodiment X 171. An orthotic, comprising:
a lower leg interface;
a shoe interface; and
a strap, wherein
the strap extends from one side of the shoe interface to an opposite side of the shoe interface beneath a shoe when a shoe is located in the shoe interface, and
the strap is releasably connected to the one side of the shoe interface by a clasp, and
the brace includes an impact barrier.

Embodiment X 172. The orthotic of embodiment X 171, wherein:
the clasp is a two stage clasp.

Embodiment X 173. The orthotic of embodiment X 171, wherein:
the strap is releasably connected to the opposite side.

Embodiment X 174. The orthotic of embodiment X 171, wherein:
the strap is releasably connected to the opposite side by a clasp.

Embodiment X 175. The orthotic of embodiment X 171, wherein:
the clasp includes at least one tooth that interfaces with a recess in the strap.

Embodiment X 176. The orthotic of embodiment X 175, wherein:
the recess is established by a gap between two teeth of the strap.

Embodiment X 177. The orthotic of embodiment X 175, wherein:
the at least one tooth includes at least two teeth.

Embodiment X 178. The orthotic of embodiment X 175, wherein:
the at least one tooth is carried by a first articulating component of the clasp; and
the clasp includes a second articulating component that locks the first articulating component in the position where the tooth interfaces with the recess.

Embodiment X 179. The orthotic of embodiment X 171, wherein:
the clasp includes at least one articulating component that is held in place with a detent system.

Embodiment X 180. The orthotic of embodiment X 179, wherein:
the clasp includes a second articulating component that is held in place with a second detent system.

Embodiment X 181. A foot orthosis, comprising:
a first apparatus configured to grip a shoe worn by a human;
a second apparatus configured to grip a leg of the human, wherein the first apparatus articulates relative to the second apparatus when the human
walks using the foot orthosis, and
the first apparatus grips the shoe by way of top strap and a bottom strap, the bottom strap being releasably attached to one side of the first apparatus by a first articulating component that articulates relative to the remainer of the first apparatus to positively retain the strap at a given adjusted position, and
the orthosis includes an impact barrier.

Embodiment X 182. The foot orthosis of embodiment X 181, wherein:
the first articulating component is supported by a chassis that is fixedly attached to a body of the first apparatus that extends about the shoe; and
the strap fits between the first articulating component and the chassis when the strap is positively retained at the given adjusted position.

Embodiment X 183. The foot orthosis of embodiment X 182, wherein:
the first articulating component is supported by a chassis that is fixedly attached to a body of the first apparatus that extends about the shoe; and
the strap fits between the first articulating component and the chassis when the strap is not positively retained at the given adjusted position but is located in the chassis.

Embodiment X 184. The foot orthosis of embodiment X 181, wherein:
the first articulating component is supported by a chassis that is fixedly attached to a body of the first apparatus that extends about the shoe; and
the strap extends through a channel in the chassis when the strap is positively retained at the given position.

Embodiment X 185. The foot orthosis of embodiment X 81, wherein:
the first articulating component is supported by a chassis that is fixedly attached to a body of the first apparatus that extends about the shoe; and
the orthosis includes a second articulating component that is also supported by the chassis, the second articulating component covering a portion of the first articulating component when viewed from a side of the brace when the first and second articulating components are closed.

Embodiment X 186. The foot orthosis of embodiment X 181, wherein:
the first articulating component is supported by a chassis that is fixedly attached to a body of the first apparatus that extends about the shoe; and
the orthosis includes a second articulating component that is also supported by the chassis, the second articulating component locking the first articulating component in a position where the first apparatus positively retains the strap.

Embodiment X 187. The orthosis of embodiment X 181, wherein:
the first articulating component is held in a position to positively retain the strap by a detent system.

Embodiment X 188. The orthosis of embodiment X 181, wherein:
the first articulating component is held in a position to positively retain the strap by a detent system;
the orthosis includes a second articulating component that locks the first articulating component into position, the second articulating component also using a detent system to hold the second articulating component closed.

Embodiment X 189. The orthosis of embodiment X 181, wherein:
the bottom strap includes teeth that interface with the first articulating component to achieve the positive retention.

Embodiment X 190. The foot orthosis of embodiment X 181, wherein:
the first articulating component is supported by a chassis that is fixedly attached to a body of the first apparatus that extends about the shoe;
the orthosis includes a second articulating component that is also supported by the chassis; and
the first articulating component and the second articulating component form respective bridges over a channel formed by the chassis through which the strap extends.

Embodiment X 191. An external ankle brace for selectively restricting movement of an ankle in at least one of a first direction and a rotation direction, and selectively permitting movement of the ankle in a second direction, wherein the external ankle brace is disposed on the exterior of a shoe, the shoe having a heel portion, a toe portion longitudinally spaced from the heel portion, a sole, and oppositely disposed sides, the external ankle brace comprising:
a rigid heel enclosure having a rear portion and a forward portion, the rear portion configured to receive and at least partially encircle the heel portion of the shoe, the forward portion having a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe, the lateral and medial sidewalls each extending from the rear portion toward a toe of a wearer's foot and each extending beyond a talus of the wearer's foot;
a lateral upright extension selectively perpendicular to the rigid heel enclosure and pivotally attached to the lateral sidewall, the lateral upright extension including a lateral reinforcing strut;
a medial upright extension selectively perpendicular to the rigid heel enclosure and pivotally attached to the medial sidewall, the medial upright extension including a medial reinforcing strut;
a lower fastening system comprising at least one lower connecting strap for connecting the lateral sidewall to the medial sidewall and extending underneath the sole of the shoe, the lower connecting strap being a strap having at least one of a composite material or a variable height; and
an upper fastening system comprising at least one upper connecting strap for selectively connecting the lateral sidewall to the medial sidewall across the top of the shoe, the upper connecting strap being located longitudinally between the lower connecting strap and the lateral and medial upright extensions, and
the brace includes an impact barrier.

Embodiment X 192. The external ankle brace of embodiment X 191, wherein:
the lower connecting strap is a composite strap.

Embodiment X 193. The external ankle brace of embodiment X 191, wherein:
the lower connecting strap has the variable height.

Embodiment X 194. The external ankle brace of embodiment X 191, wherein:
the lower connecting strap includes one or more elongate reinforcement components embedded in a covering material.

Embodiment X 195. The external ankle brace of embodiment X 191, wherein:
the lower connecting strap includes one or more elongate reinforcement components embedded in a covering material, at least one of the one or more elongate reinforcement components extends for at least 97% or the entire length of the strap.

Embodiment X 196. The external ankle brace of embodiment X 191, wherein:
the lower connecting strap includes at least eight elongate reinforcement components embedded in a covering material.

Embodiment X 197. The external ankle brace of embodiment X 191, wherein:
the lower connecting strap includes teeth.

Embodiment X 198. The external ankle brace of embodiment X 191, wherein:
the lower connecting strap is releasably attached to the lateral sidewall by way of partial penetrative positive retention.

Embodiment X 199. The external ankle brace of embodiment X 191, wherein:
the lower connecting strap is permanently attached to the medial sidewall by way of a partially penetrative positive retention.

Embodiment X 200. The external ankle brace of embodiment X 191, wherein:
the lower connecting strap is permanently attached to the medial sidewall by way of only gripping forces.

Embodiment X 201. An external ankle brace, comprising:
a medial lower leg section configured to interface with at least a medial side of a leg of a human at a lower leg portion thereof, the medial lower leg section including a medial rigid body contoured to a medial side of a lower leg of a human;
a lateral lower leg section configured to interface with at least a lateral side of a leg of a human at a lower leg portion thereof, the lateral lower leg section including a medial rigid body contoured to a medial side of a lower leg of a human; and
a foot section configured to interface with a shoe worn on a foot of the human at locations where the foot of the human is located, wherein
a first strap extends from one side of the foot section to the other side of the foot section on a top of the foot section, the first strap at least one of:
extending through a body establishing the foot section; or
having a potion thereof being located outboard of the body, and
a second strap extends from the one side of the foot section to the other side of the foot section on a bottom of the foot section, wherein at least one of:
the second strap being entirely located outboard of the body;
the body has no indentations to directly accommodate the second strap; or
there is no piercing of the second strap, and
the brace includes an impact barrier.

Embodiment X 202. The brace of embodiment X 201, wherein:
there are no rivets or other components associated with the first strap that pierce the body.

Embodiment X 203. The brace of embodiment X 201, wherein:
there are rivets that pierce the body associated with the second strap, and the second strap is unpierced by any rivet.

Embodiment X 204. The brace of embodiment X 201, wherein:
the first strap does not extend underneath a shoe worn in the brace when the brace is worn over a shoe, and the second strap does not extend over a top of the shoe.

Embodiment X 205. The brace of embodiment X 201, wherein:
the second strap is entirely located outboard of the body.

Embodiment X 206. The brace of embodiment X 201, wherein:
the second strap is located inboard of the body on one side of the foot interface and located outboard of the body on an opposite side of the foot interface.

Embodiment X 207. The brace of embodiment X 201, wherein:
there is only one strap extending from one side to the other side of the foot section on top.

Embodiment X 208. The brace of embodiment X 201, wherein:
there are two or more straps extending from one side to the other side of the foot section on top.

Embodiment X 209. The brace of embodiment X 201, wherein:
there is only one strap extending from one side to the other side of the foot section on bottom.

Embodiment X 210. The brace of embodiment X 201, wherein:
there are two or more straps extending from one side to the other side of the foot section on bottom.

Embodiment X 211. The brace of embodiment X 201, wherein:
all straps are forward of a talus of a foot when worn by a human.

Embodiment X 212. The brace of embodiment X 201, wherein:
there are two or more straps extending from one side to the other side of the foot section on bottom.

Embodiment X 213. The brace of embodiment X 201, wherein:
the first strap extends through a body establishing the foot section.

Embodiment X 214. The brace of embodiment X 201, wherein:
the first strap has a potion thereof being located outboard of the body.

Embodiment X 215. The brace of embodiment X 201, wherein:
the second strap is entirely located outboard of the body.

Embodiment X 216. The brace of embodiment X 201, wherein:
the body has no indentations to directly accommodate the second strap.

Embodiment X 217. The brace of embodiment X 201, wherein:
there is no piercing of the second strap.

Embodiment X 218. The brace of embodiment X 201, wherein:
the second strap is permanently connected the foot portion on one side and adjustably connected on an opposite side.

Embodiment X 219. The brace of embodiment X 201, wherein:
the second strap is releasably connected to one side of the foot portion and releasably connected to an opposite side of the foot portion.

Embodiment X 220. The brace and/or system of any of Embodiments X 1-219, combined with any one or more or all of any other aspect of any other of Embodiments X 1-219

Embodiment X 221. The method of any of Embodiments X 1-219, combined with any one or more or all of any other aspect of any other of embodiments X 1-219

Embodiments include the combination of any one or more elements/features disclosed herein with any one or more other elements/features disclosed herein, unless otherwise specified, providing that the art enables such. Embodiments also include the combination of any one or more elements/features of any of Ser. No. 15/074,339, filed 18 Mar. 2016, U.S. patent application Ser. No. 15/642,430, filed Jul. 5, 2017, U.S. patent application Ser. No. 16/374,865, and/or Provisional Patent Application Ser. No. 62/135,823, filed 20 Mar. 2015, with any one or more other elements/features disclosed herein, including one or more of the elements/features in any one or more of the Appendices and/or with any one or more other elements/features of the above noted patent applications, unless otherwise specified, providing that the art enables such. Embodiments also include the exclusion of any one or more elements/features disclosed herein with any one or more other elements/features disclosed herein, unless otherwise specified, providing that the art enables such. Embodiments also include the exclusion of any one or more elements/features of any of Ser. No. 15/074,339, filed 18 Mar. 2016, U.S. patent application Ser. No. 15/642,430, filed Jul. 6, 2017, U.S. patent application Ser. No. 16/374,865, and/or Provisional Patent Application Ser. No. 62/135,823, filed 20 Mar. 2015, with any one or more other elements/features disclosed herein, including one or more of the elements/features in any one or more of the Appendices and/or with any one or more other elements/features of the above noted patent applications, unless otherwise specified, providing that the art enables such. Unless defined otherwise, all technical and scientific terms used herein have the same meaning as is commonly understood by one of skill in the art to which the present disclosure pertains.

In an embodiment, the forces/moments applied to the heel enclosure/the upright, for the flex assist, and/or the restrictions to rotation by the devices, for the rehabilitation/exercise embodiments, are about one and only one axis. For example, about the axis parallel to the ground and generally normal to the longitudinal extension of the foot (heel to toe direction). This can be the axis through the two joints, the axis about which the uprights rotate relative to the heel enclosure, or visa-versa. In an embodiment, there is only one axis of rotation, and that is through the joints, or at least there is only one general axis of rotation. The axis of rotation of the heel enclosure is fixed relative to the uprights, and vis-a-versa, in some embodiments.

It is noted that any disclosure of a device and/or system detailed herein also corresponds to a disclosure of otherwise providing that device and/or system and/or utilizing that device and/or system.

It is also noted that any disclosure herein of any process of manufacturing or otherwise providing a device corresponds to a disclosure of a device and/or system that results therefrom. It is also noted that any disclosure herein of any device and/or system corresponds to a disclosure of a method of producing or otherwise providing or otherwise making such.

Any disclosure herein of a device and/or system corresponds to a disclosure of utilizing that device and/or system for the purposes detailed herein. Any disclosure herein of an action that is executed utilizing a device and/or system corresponds to a disclosure of that device and/or system.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

There are both various features disclosed herein associated with various embodiments disclosed herein, as well as various patent applications incorporated by reference herein that respectively disclose respective various features associated with respect to various embodiments of those patent applications. Any one or more or all of the various features referenced in the preceding sentence can be combined with any one or more or all of other various features referenced in the preceding sentence unless otherwise specifically stated and/or unless the art does not enable such combination. Also, any one or more or all of the various features referenced in the beginning sentence of this paragraph can be explicitly excluded from combination with any one or more or all of the other various features referenced in the sentence at the beginning of this paragraph unless otherwise specifically stated and/or unless the art does not enable such exclusion of combination.

Any disclosure herein of a method action corresponds to a disclosure of a related product and/or system that is used in that method action. Any disclosure herein of any device and/or system corresponds to a disclosure of a resulting method of utilizing that device and/or system. Any disclosure herein of a device and/or system corresponds to a disclosure of a method of making that device and/or system. Any disclosure herein of a method action of making a component and/or a device and/or system corresponds to a disclosure of a resulting component and/or device and/or system that results from such method action.

1-200. (canceled)

201. An orthotic, comprising:
a lower leg interface; and
a shoe interface articulatedly connected to the lower leg interface, wherein
the orthotic is configured with a barrier between the shoe interface and the lower leg interface.

202. The orthotic of claim 201, wherein:
the barrier is a protrusion on the shoe interface.

203. The orthotic of claim 201, wherein:
the barrier is monolithic with a main portion of the shoe interface.

204. The orthotic of claim 201, wherein:
the barrier has a cross section of a ramp.

205. The orthotic of claim 201, wherein:
the barrier extends in an arcuate manner.

206. The orthotic of claim 201, wherein:
the barrier extends in parallel manner with a portion of the lower leg interface immediately adjacent the barrier.

207. The orthotic of claim 201, wherein:
the barrier is established by a first portion of the orthotic that is more outboard than a second portion of the orthotic.

208. An external ankle brace, comprising:
a medial lower leg section configured to interface with at least a medial side of a leg of a human at a lower leg portion thereof, the medial lower leg section including a medial rigid body contoured to a medial side of a lower leg of a human;
a lateral lower leg section configured to interface with at least a lateral side of a leg of a human at a lower leg portion thereof, the lateral lower leg section including a lateral rigid body contoured to a lateral side of a lower leg of a human;
a foot section configured to interface with a shoe worn on a foot of the human at locations where the foot of the human is located;
a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section, wherein
the lateral ankle joint has a female portion that is part of the lateral lower leg section that interfaces with a male portion of the lateral ankle joint that is part of the foot section, and
an outermost edge of the female portion is inboard of a portion of the foot section.

209. The external ankle brace of claim 208, wherein:
the inboardness of the outermost edge establishes a barrier.

210. The external ankle brace of claim 208, wherein:
the inboardness of the outermost edge is located at the lowest portion of the female portion.

211. The external ankle brace of claim 208, wherein:
the inboardness of the outermost edge extends for more than 30 degrees about the female portion.

212. The external ankle brace of claim 208, wherein:
the inboardness of the outermost edge is present through all range of motions of the lateral lower leg section.

213. The external ankle brace of claim 208, wherein:
the inboardness of the outermost edge is established by a protrusion of the foot section, the protrusion corresponding to at least part of the portion of the foot section.

214. The external ankle brace of claim 208, wherein:
the inboardness of the outermost edge extends for at least 80 percent of an overlap area, when viewed from a side of the brace, of the outermost edge with the foot section.

215. An external ankle brace, comprising:
a medial lower leg section configured to interface with at least a medial side of a leg of a human at a lower leg portion thereof, the medial lower leg section including a medial rigid body contoured to a medial side of a lower leg of a human;
a lateral lower leg section configured to interface with at least a lateral side of a leg of a human at a lower leg portion thereof, the lateral lower leg section including a lateral rigid body contoured to a lateral side of a lower leg of a human;
a foot section configured to interface with a shoe worn on a foot of the human at locations where the foot of the human is located;
a lateral ankle joint that pivotally connects said lateral lower leg section to said foot section and allows said lateral lower leg section to move relative to said foot section and a medial ankle joint that pivotally connects said medial lower leg section to said foot section and allows said medial lower leg section to move relative to said foot section, wherein
the lateral ankle joint has a female portion that is part of the lateral lower leg section that interfaces with a male portion of the lateral ankle joint that is part of the foot section, and
an outermost edge of the female portion is inboard of a portion of the foot section.

216. The external ankle brace of claim 215, wherein:
the inboardness of the outermost edge establishes a barrier.

217. The external ankle brace of claim 215, wherein:
the inboardness of the outermost edge is located at the lowest portion of the female portion.

218. The external ankle brace of claim 215, wherein:
the inboardness of the outermost edge extends for more than 30 degrees about the female portion.

219. The external ankle brace of claim 215, wherein:
the inboardness of the outermost edge is present through all range of motions of the lateral lower leg section.

220. The external ankle brace of claim 215, wherein:
the inboardness of the outermost edge is established by a protrusion of the foot section, the protrusion corresponding to at least part of the portion of the foot section.
